(19) United States
(12) Patent Application Publication
(10) Pub. No.: US 2026/0355340 A1
(43) Pub. Date: Nov. 12, 2026

(54) Conjugates of Biomolecules and Use Thereof

(71) Applicant: Yafei Shanghai Biolog Medicine Science & Technolog, Shanghai (CN)

(72) Inventors: Yuan Liu, Shanghai (CN); Haiyang Wang, Shanghai (CN); Renke Li, Shanghai (CN); Rui Zhang, Shanghai (CN); Cheng Liu, Shanghai (CN)

(21) Appl. No.: 19/425,421

(22) Filed: Dec. 18, 2025

Related U.S. Application Data
(63) Continuation-in-part of application No. 16/761,477, filed on Nov. 7, 2018, now Pat. No. 12,564,632.
(60) Provisional application No. 62/583,410, filed on Nov. 8, 2017.

(51) Int. Cl. A61K 47/68; A61K 47/64; A61K 47/65

(52) U.S. Cl. CPC A61K 47/6849; A61K 47/642; A61K 47/65; A61K 47/6889

(57) ABSTRACT

Disclosed are conjugates of biomolecule and use thereof. The disclosed conjugates of biomolecule contain a biomolecule and a functional moiety covalently linked to the biomolecule. The functional moiety contains a group that prevents the biomolecule from binding to its ligand or receptor, a cleavable linker arm that can be activated by proteolytic enzymes or can be acidically activated in a microenvironment of a disease, a linker arm that will automatically shed after the cleavable linker arm is cleaved, and a group that maintains or promotes the binding capacity of the biomolecule to its ligand or receptor. The conjugates of biomolecule of the present disclosure can effectively reduce immunogenicity of the biomolecule, increase it's half-life, and break through the immune barrier of an individual, reach a pathologic microenvironment and be activated and released in the pathologic microenvironment, selectively promoting proliferation or killing effect of T cells and the like in the tumor, thereby preventing on target off tumor toxicity or in the inflammatory microenvironment of autoimmune disease and achieving a low autoimmunity and high efficacy.

### RELATED APPLICATIONS

This application is a Continuation-In-Part application claiming priority from U.S. patent application Ser. No. 16/761,477 filed May 4, 2020, now U.S. Pat. No. 12,564,632, which is a U.S. National Stage application under 35 U.S.C. § 371 from International Patent Application No. PCT/CN2018/114266 filed Nov. 7, 2018, which claims the benefit of priority from U.S. Patent Application No. 62/583,410 filed Nov. 8, 2017.

### TECHNICAL FIELD

The present invention relates to conjugates of biomolecules and uses thereof.

### REFERENCE TO SEQUENCE LISTING

The material in the sequence listing text file submitted herewith, titled “863.203C1_17Dec2025.xml,” created on Dec. 17, 2025, and having a size of 154,000 bytes, is hereby incorporated by reference pursuant to 37 C.F.R. § 1.835(a)(2).

### TECHNICAL BACKGROUND

Conjugating agents to antibodies by linking to native cystine has been used to further advance the use of antibody. Molecules such as toxins and drugs have been conjugated to antibodies to generate antibody-drug conjugates (ADCs). Fusion of a cleaving, masking peptide sequence to an antibody to generate a probody has been used for a local activation in Tumor. But the peptides are limited in terminal antibody, and the activation sequence is limited with peptide with low activation rate and high immunogenicity from masking peptide.

The common side effect of the currently commercial macromolecular drugs is immunotoxicity. Immunotoxicity includes immunosuppression, generation of immunogenicity, hypersensitivity, autoreaction and adverse immune stimulation. These side effects are mainly caused by extraneous macromolecules. After entering into the body, heterogenous macromolecules will elicit an immune response in the patient due to its immunogenicity. In normal tissues, immunity also will be stimulated and autoimmunity response will be generated after a macromolecule drug, such as antibody or cytokine, binds to the antigens or receptors. At this time it is very dangerous to the patient. For example, during the treatment of non-small cell lung cancer with the current PD-1 antibodies, Keytruda® (pembrolizumab) and Opdivo® (nivolumab), serious pneumonia may occur, which may even lead to death of patients. Similar effects were also reported in the clinical use of CTLA4 antibodies (Yervoy®; ipilimumab), 41BB, IL-2, IL-10 etc. In combination therapy of Opdivo® (nivolumab) and Yervoy® (ipilimumab) to non-small cell lung cancer, 55% of patients showed the high grade3-4 AEs and 36% patients had to stop drug treatment due to drug toxicity. Therefore, intelligent conjugates of biomolecule with new functions are required in this field to decrease the overall toxicity and immunotoxicity by blocking drug activity in blood and normal tissue and to enhance the active drug in pathologic microenvironment, to adjust DMPK and half-life in serum, to decrease immunogenicity of Fab of antibody, to adjust the binding affinity of active biomolecule, and to enhance the efficacy.

### SUMMARY OF INVENTION

The present disclosure provides conjugates of a biomolecule having the following structure:

R1-R2-R3-R4-S-cys-R5

wherein,
R5 represents a biomolecule with one or more cysteine residues introduced by mutation;
cys represents the cysteine residue(s) contained in R5;
S represents sulfur atom(s) of the cysteine residue(s);
R1 is a group that prevents R5 from binding to its antigen, ligand or receptor;
R2 is absent, or R2 is a cleavable linker arm capable of being activated by one or more proteolytic enzymes or a chemical bond capable of being acidically activated in a pathologic microenvironment;
R3 is absent, or R3 is a linker arm capable of automatically shedding after R2 is cleaved or a chemical bond capable of being acidically activated in a pathologic microenvironment; with the proviso that when R2 is absent, R3 is the chemical bond capable of being acidically activated in a pathologic microenvironment; and
R4 is a group covalently linked to R5 via the sulfur atom(s) of the cysteine residue(s) contained in R5 that recovers, maintains or promotes the binding capacity of R5 to its antigen, ligand or receptor after the moiety R1-R2-R3 is cleaved.

In the above formula, when R1-R2 is cleaved from R3-R4-S-cys-R5 by a proteolytic enzyme or under an acid condition in the pathologic microenvironment, R3-R4-S-cys-R5 is released. And the binding capacity of R4-S-cys-R5 to the antigen, ligand or receptor of R5 will be recovered, maintained or improved after R3 automatically sheds and R4-S-cys-R5 is released.

In one or more embodiments of the present disclosure, R1, R2, R3, R4 and R5 of the conjugates of biomolecule are described as in other parts of the present disclosure.

The present disclosure also provides a compound having the following structure:

R4-S-cys-R5

wherein,
R4 is represented by R₄₋ₐ-R_{4-b}-R_{4-c}—, wherein R4 is represented by R₄₋ₐ-R_{4-b}-R_{4-c}—, wherein R₄₋ₐ is selected from the group consisting of:

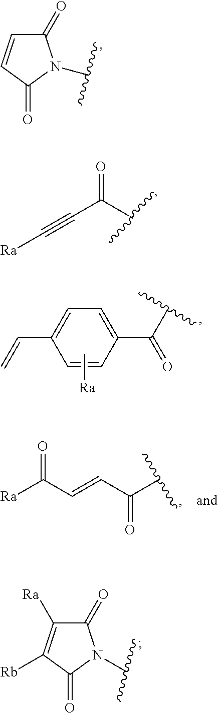

wherein Ra and Rb are each independently selected from the group consisting of H and C₁₋₆ alkyl or C₁₋₆ alkoxyl;
R_{4-b} is selected from the group consisting of:

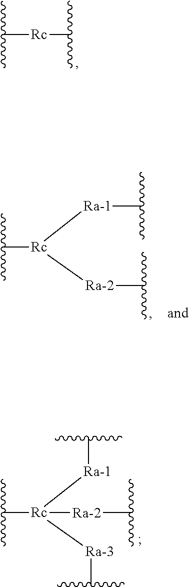

wherein in formula R4-b1, Rc is absent, or is selected from the group consisting of C₁₋₁₂ alkyl, C₁₋₁₂alkoxy-C₁₋₁₂alkyl, C₁₋₁₂alkyl-C₃₋₈cycloalkyl, (C₁₋₄alkyl-O)ₚ—C₁₋₁₂alkyl, C₁₋₁₂alkylcarbonylamino-(C₁₋₄alkyl-O)ₚ—C₁₋₁₂alkyl, phenyl-C₁₋₁₂alkyl, C₃₋₈cycloalkyl, C₁₋₁₂alkyl-C₃₋₈cycloalkyl-C₁₋₁₂alkyl, and C₁₋₁₂alkyl-phenyl-C₁₋₁₂alkyl; in formula R4-b2, Rc is a C₁₋₁₂alkylamino with Ra-1 and Ra-2 substituted at N atom of the amino group, and in formula 4-b3, Rc is a C₁₋₁₂alkyl with the last C atom at the end of the alkyl being substituted by Ra-1, Ra-2 and R2-3, wherein Ra-1, Ra-2 and Ra-3 are each independently selected from the group consisting of C₁₋₁₂alkyl, C₁₋₁₂alkyl-OH, and C₁₋₁₂alkyl-NR″R′″, wherein R″ and R′″ are each independently selected from the group consisting of H and C₁₋₁₂alkyl; wherein in formulae R4-b2 and R4-b3, R4-b links to R4-c via at least one of the Ra-1, Ra-2 and Ra-3;
R_{4-c} is selected from the group consisting of:

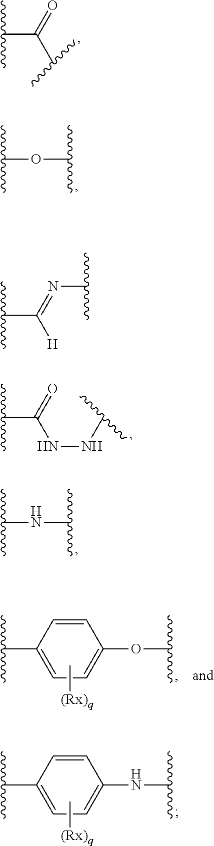

wherein Rx is selected from the group consisting of H, halo and C₁₋₄alkyl; p is an integer in a range of 1 to 10, such as an integer in a range of 1 to 5; and q is an integer in a range of 1 to 4, such as an integer in a range of 1 to 2; with the proviso that R4-c is absent when R4-a is selected from the group consisting of formulae R4-a2, R4-a3 and R4-a4;
wherein R3 links to R4 via the R_{4-c} of R4, and the wave line shown in each formula of R4-a indicates the position at which R4-a links to R4-b.

Also provided is a compound represented by R1-R2-R3-R4, wherein R1, R2, R3 and R4 are defined as in any embodiments of the present disclosure.

The present disclosure provides use of the conjugates of biomolecule or the R4-S-cys-R5 compound as described herein in the manufacture of an anti-tumor drug.

The present disclosure provides a pharmaceutical composition comprising the conjugates of biomolecule as described herein.

The present disclosure provides a method for treating or preventing tumors, comprising providing to a subject in need thereof a therapeutically effective amount of the conjugates of biomolecule or the R4-S-cys-R5 compound as described herein.

The present disclosure also provides compounds, including compounds S1-S64 and compounds described in other parts of the present disclosure, and antibodies and cytokines containing mutation as described herein.

### BRIEF DESCRIPTION OF DRAWINGS

FIG. 1: Alignment of the amino acid sequences of the light chains of antibodies (From top to bottom: SEQ ID NO:95, SEQ ID NO: 93 and SEQ ID NO: 96).

FIG. 2: Co-IP screening for point mutation in the light chain of anti-PD1 antibody.

FIG. 3: Structure of antibody.

FIG. 4: The sequences of loop 4 in the variable region are conservative (From top to bottom: SEQ ID NO: 32, SEQ ID NO: 14, SEQ ID NO: 30, SEQ ID NO: 16, SEQ ID NO: 18, SEQ ID NO: 89, SEQ ID NO: 63 and SEQ ID NO: 90).

FIG. 5: Variable regions of two antibodies may come from the variable region framework of antibodies of same germline (From top to bottom: SEQ ID NO: 93, SEQ ID NO: 95, SEQ ID NO: 64 and SEQ ID NO: 80).

FIG. 6: Alignment of amino acid sequences of heavy chains of several antibodies (From top to bottom: SEQ ID NO: 60, SEQ ID NO: 27, SEQ ID NO: 31, SEQ ID NO: 29, SEQ ID NO: 13, SEQ ID NO: 17 and SEQ ID NO: 91).

FIG. 7: Alignment of amino acid sequences of light chains of several antibodies (From top to bottom: SEQ ID NO: 93, SEQ ID NO: 95 and SEQ ID NO: 96).

FIG. 8: Conjugation of PD1 Ab-C28.

FIG. 9: Binding of the PD1 Ab-C28 conjugate to PD1 before and after activation.

FIG. 10: Schematic drawing of conjugate of biomolecule which contains an activatable and binding arm and is activated in a pathologic microenvironment.

FIG. 11: Secretion of IFN-gamma.

FIG. 12: Survival curve.

FIG. 13: Death curve.

FIG. 14: Proliferation of T cells.

FIG. 15: Conjugate of anti-PD1 antibody inhibits growth of tumor on mice suffering from MC38 colon cancer.

FIG. 16: Conjugation of IL2-C41 mutant.

FIG. 17: Binding of the IL2-C41 conjugate to IL2 receptor alpha before and after activation.

FIG. 18: Effect of IL2 mutant conjugation (IL2-Thr41Cys, i.e., IL2-C41) on proliferation of T cells.

FIG. 19: Effect of IL2 conjugate on CD4/CD8 proliferation.

FIG. 20: Conjugate of IL2 inhibits growth of B16F10 tumor.

FIG. 21: Conjugate of IL2 in combination with anti-PD-1 antibody inhibits growth of MC38 tumor.

FIG. 22: The SDS-PAGE results of mutant IL2, IL2 TMEAkine and the recovery active IL2 after enzyme cleavage in vitro.

FIG. 23: ELISA results indicating the binding activity of IL2 TMEAkine to IL2Rα or Rβ before and after enzyme cleavage in vitro.

FIG. 24: The amount of IL2-T41C-S47 detected by western blot and the corresponding results.

FIG. 25: The pharmacokinetic study in vivo indicating that IL2 TMEAkine has a long half-life and a high exposure in plasma when compared with IL2.

FIG. 26: The measurement of pulmonary (wet weight) and the sections stained with hematoxylin and eosin indicating that IL2 TMEAkine induces less toxicity to lung than wild-type IL2.

FIG. 27: The measurement of pulmonary indicating that IL2-T41C/S87C-S47 and IL2-R38D/E61R/S87C-S47 induce almost no toxicity to lung.

FIG. 28: Tumor volumes after treatment.

FIG. 29: High exposure of active IL2 in tumor, which is consistent with high efficacy of anti-tumor effect, and low exposure of active IL2 in lungs and heart, which is consistent with low toxicity of pulmonary edema.

FIG. 30: Reduced SDS-PAGE gel results showing that all five positions are conjugated with S13 linker in a high efficiency.

FIG. 31: The 8 selected antibodies sequences (Upper from top to bottom: SEQ ID NO: 99, SEQ ID NO: 101, SEQ ID NO: 64, SEQ ID NO: 19, SEQ ID NO: 97, SEQ ID NO: 92, SEQ ID NO: 102 and SEQ ID NO: 105; lower from top to bottom: SEQ ID NO: 98, SEQ ID NO: 100, SEQ ID NO: 65, SEQ ID NO: 20, SEQ ID NO: 96, SEQ ID NO: 93, SEQ ID NO: 103 and SEQ ID NO: 104).

FIG. 32: By site-specially conjugating different R4 groups to the selected mutant site of antibody, the R4 group could be selectively changed for screening.

FIG. 33: Conjugating to different sites shows different degrees of binding activity recovery.

FIG. 34: The TMEAbodies with significant decreased binding activity to human CTLA-4 protein also show dramatic decrease of receptor blocking activity.

FIG. 35: TMEAbody with decreased binding activity against CTLA-4 shows impairment of functional efficacy of T cell activation, and protease mediated activation can restore the activity of TMEAbody.

FIG. 36: Ipilimumab TMEAbodies significantly down regulate Treg population in the tumor, with the similar efficacy as WT Ipilimumab.

FIG. 37: TMEAbody shows high stability in mouse serum, without significant degradation after 96 h at 37 °C.

FIG. 38: CTLA-4 TMEAbodies show increased half-life and exposure by conjugation with S47 functional group comparing with WT-Ipilimumab and CTLA-4 probody.

FIG. 39: Ipilimumab TMEAbodies control the tumor size with comparable efficacy as WT Ipilimumab, whereas control human IgG fails to show any efficacy.

FIG. 40: TMEAbody causes very low immune response in the animals, with comparable or lower antibody titer than WT Ipilimumab.

FIG. 41: Protection of an immune system by a conjugate of anti-CTLA4 TMEAbody in combination therapy conjugate could reduce autoimmunity as compared to the primary antibody.

FIG. 42: Niv-se001 shows increased activity after R4-7 conjugation or protease cleavage of 40 kD conjugated TMEAbody, which is 432% of WT Nivolumab.

FIG. 43: After conjugation with R4-7, or after protease cleavage of Niv-se005 conjugated with 40 kD functional group, the binding activity of Niv-se005 is restored with comparable level than WT Nivolumab (125% of WT).

FIG. 44: Pembrolizumab and Nivolumab TMEAbodies control the tumor size with comparable efficacy as WT Pembrolizumab or Nivolumab antibodies, whereas control human IgG failed to show any efficacy.

FIG. 45: Anti-CTLA4 or anti-PD1 TMEAbody in combination therapy could reduce autoimmunity as compared to the primary antibody.

FIG. 46: Tumor volumes after treatment.

FIG. 47: Spectrum of intact molecular weight of SEQ ID No. 106

FIG. 48: Spectrum of intact molecular weight of SEQ ID No. 44

### EMBODIMENTS

It should be understood that, within the scope of the present disclosure, each of the technical features mentioned above and each of those mentioned hereinafter, such as in the Examples, can be combined with each other to constitute preferred technical solutions. Additionally, the present disclosure is not limited to particular embodiments described, as such may, of course, vary. It is also to be understood that, unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” “and,” and “the” include plural referents unless the context clearly dictates otherwise. Thus, for example, reference to “an antibody” includes a plurality of antibodies and reference to “the antibody” includes reference to one or more antibodies and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

One of the purposes of the present disclosure is to provide modified biomolecules, which are only activated in a pathologic microenvironment, such as in a tumor microenvironment or an inflammatory environment, to release a biomolecule (R5) in the pathologic microenvironment which has the same or even improved binding capacity to its ligand, thereby improving the targeting and efficacy of the biomolecule, overcoming drug resistance and reducing toxicity.

In the present disclosure, the modified biomolecule is a conjugate, which comprises a biomolecule and a functional moiety covalently linked to the biomolecule. Biomolecules suitable for use in the present disclosure may be biomolecules having a biological function or activity of interest, including but not limited to various functional proteins. Biological function or activity of interest includes but is not limited to functions or activities in enzymology and immunology. Therefore, biomolecules suitable for use in the present disclosure include but are not limited to antibodies or functional fragments thereof, enzymes, fusion proteins (such as protein-antibody fusions), antibody drug conjugates, cytokines, and any other genetically engineered biological molecules.

As used herein, the term “antibody” is used in the broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al (2003) Jour. of Immunology 170:4854-4861). In the context and in the Figures, an antibody is abbreviated as “Ab”.

The basic antibody structural unit is known to comprise a tetramer composed of two identical pairs of polypeptide chains, each pair having one light and one heavy chain. The N terminal portion of each chain includes a variable region of about 100 to 110 or more amino acids primarily responsible for antigen recognition. The C terminal portion of each chain defines a constant region primarily responsible for effector function. The variable region of heavy chain (VH) and the variable region of light chain (VL) each contain 3 complementarity determining region (CDR), including HCDR1, HCDR2, HCDR3 and LCDR1, LCDR2 and LCDR3. These six CDRs form antigen-binding site of an antibody. The remaining amino acids of the variable region are relatively conservative and are termed as framework region (FR). VH and VL each contain 4 framework regions, called FR1, FR2, FR3 and FR4, respectively.

Antibodies may be murine, human, humanized, chimeric, or derived from other species. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

Antibody fragments comprise a portion of a full length antibody, generally the antigen binding or variable region thereof. Preferably, fragment of an antibody is a functional fragment, i.e., retaining the antigen-binding capability of the intact antibody. Examples of antibody fragments or functional fragments include Fab, Fab′, F(ab′)2, and Fv fragments; diabodies; linear antibodies; and single chain antibody molecules (scFv); etc.

Fusion protein used herein may contain an antigen binding domain of an antibody and optionally a cytokine. The antigen binding domain of an antibody contained in fusion protein may be an antigen binding domain to an antigen selected from the group consisting of HER2, CD19, EGFR, CD22, CD3, TROP2, Glycoprotein NMB, Guanylyl cyclase C, CEA, CD79b, PSMA, ENPP3, Mesothelin, CD138, NaPi2b, CD56, CD74, FOLR1, DLL3, CEACAM5, CD142, SLAMF7, CD25, SLTRK6, CD37, CD70, AGS-22, C4.4A, FGFR2, Ly6E, MUC16, BCMA, pCadherin, Ephrin-A, LAMP1, MUC1, CD19, PDL1, HER2, NY-ESO-1, BCMA, WT1, MUC1, CD20, CD23, ROR1, CD123, CD33, CD44v6, CD174, CD30, CD133, cMet, EGFR, FAP, EphA2, GD2, GPC3, IL-13Ra2, LewisY, Mesothelin, SS1, CEA, CD171, EGFR, EGFRvIII, VEGFR2, NY-ESO-1, MUC-1 and MAGE-A3, or may be an antigen binding domain of any antibodies as described herein. In some embodiments, the fusion protein is a bispecific antibody, which contain an antigen binding domain to an antigen selected from the group consisting of HER2, CD19, EGFR, CD22, CD3, TROP2, Glycoprotein NMB, Guanylyl cyclase C, CEA, CD79b, PSMA, ENPP3, Mesothelin, CD138, NaPi2b, CD56, CD74, FOLR1, DLL3, CEACAM5, CD142, SLAMF7, CD25, SLTRK6, CD37, CD70, AGS-22, C4.4A, FGFR2, Ly6E, MUC16, BCMA, pCadherin, Ephrin-A, LAMP1, MUC1, CD19, PDL1, HER2, NY-ESO-1, BCMA, WT1, MUC1, CD20, CD23, ROR1, CD123, CD33, CD44v6, CD174, CD30, CD133, cMet, EGFR, FAP, EphA2, GD2, GPC3, IL-13Ra2, LewisY, Mesothelin, SS1, CEA, CD171, EGFR, EGFRvIII, VEGFR2, NY-ESO-1, MUC-1 and MAGE-A3, or which contain an antigen binding domain from any antibodies as described herein. Preferably, the bispecific antibody is a single chain bispecific antibody, which contains two scFv from the same or different antibodies.

In some embodiments, the fusion protein is an antibody-cytokine fusion protein, which contains an antibody or functional fragment thereof and a cytokine selected from the group consisting of IL-2, IL-7, IL-10, IL-11, IL-12, IL-15, IL-21, IFN-α, IFN-β, IFN-γ, G-CSF, GM-CSF, TNF-α, TRAP, and TRAIL. An example of such fusion protein is a fusion protein of anti-PD-1 antibody or anti-CD3 antibody or antigen binding domain thereof with IL2.

Antibody used herein may be any antibodies known in the art and functional fragments thereof. For example, antibody used herein may be an antibody or functional fragment thereof selected from the group consisting of anti-Her2 antibody, anti-EGFR antibody, anti-VEGFR antibody, anti-CD20 antibody, anti-CD33 antibody, anti-PD-L1 antibody, anti-PD-1 antibody, anti-CTLA-4 antibody, anti-TNFα antibody, anti-CD28 antibody, anti-4-1BB antibody, anti-OX40 antibody, anti-GITR antibody, anti-CD27 antibody, antib-CD40 antibody, or anti-ICOS antibody, anti-CD25 antibody, anti-CD30 antibody, anti-CD3 antibody, anti-CD22 antibody, anti-CCR4 antibody, anti-CD38 antibody, anti-CD52 antibody, anti-Complement C₅ antibody, anti-F protein of RSV, anti-GD2 antibody, anti-GITR antibody, anti-Glycoprotein receptor lib/Illa antibody, anti-ICOS antibody, anti-IL2R antibody, anti-LAG3 antibody, anti-Integrinα4 antibody, anti-lgE antibody, anti-PDGFRa antibody, anti-RANKL antibody, anti-SLAMF7 antibody, anti-LTIGIT antibody, anti-TIM-3 antibody, anti-VEGFR2 antibody, anti-VISTA antibody.

In preferred embodiments, antibody used herein may be an antibody or functional fragment thereof selected from the group consisting of utomilumab, urelumab, ADG106, Poteligeo® (mogamulizumab), Bexxar™ (tositumomab), Zevalin™ (ibritumomab tiuxetan), Rituxan® (rituximab), Arzerra® (ofatumumab), Gazyva® (obinutuzumab), Besponsa® (inotuzumab ozogamicin), Zenapax® (daclizumab), varlilumab, theralizumab, Adcetris® (brentuximab vedotin), Myelotarg™ (gemtuzumab), Darzalex® (daratumumab), CDX-1140, SEA-CD40, R07009789, JNJ-64457107, APX-005M, Chi Lob 7/4, Campath™ (alemtuzumab), Raptiva™ (efalizumab), Soliris® (eculizumab), Yervoy® (ipilimumab), tremelimumab, Erbitux® (cetuximab), Vectibix® (panitumumab), Portrazza™ (Necitumumab), TheraCIM® (nimotuzumab), Synagis® (palivizumab), Unituxin® (dinutuximab), TRX-518, MK-4166, MK-1248, GWN-323, INCAGN0186, BMS-986156, AMG-228, ReoPro® (abiciximab), Herceptin® (trastuzumab), Perjeta® (pertuzumab), Kadcyla® (ado-trastuzumab emtansine), GSK-3359609, JTX-2011, Simulect™ (basiliximab), Tysabri® (natalizumab), BMS-986016, REGN3767, LAG525, Xolair® (omalizumab), tavolimab, PF-04518600, BMS-986178, MOXR-0916, GSK-3174998, INCAGN01949, IBI-101, Keytruda® (pembrolizumab), Opdivo® (nivolumab), Lartruvo™ (olaratumab), Tencentriq® (atezolizumab), BMS-936559, Bavencio® (avelumab), Imfinzi® (duralumab), Prolia® (denosumab), Empliciti® (elotuzumab), MTIG7192A, TSR-022, MBG-453, Remicade® (infliximab), Humira® (adalimumab), Avastin® (bevacizumab), Lucentis® (ranibizumab), Cyramza® (ramucirumab), and JNJ-61610588.

In the present disclosure, cytokine may have a meaning and structure commonly used in the art. It generally refers to a kind of small proteins having wide biological activities, which are synthesized and secreted by stimulating immunocytes, such as monocytes, macrophages, T cells, B cells, NK cells, etc., or nonimmune cells, such as endothelial cells, epidermal cells, fibroblasts, etc. Cytokine may regulate cell growth, differentiation and effect, and immune response through binding to a corresponding receptor. Suitable cytokines include interleukins, interferons, tumor necrosis factor superfamily, colony stimulating factors, chemotactic factors and growth factors, etc.

Exemplary cytokines include but are not limited to IL-2, IL-7, IL-10, IL-11, IL-12, IL-15, IL-21, IFN-α, IFN-β, IFN-γ, G-CSF, GM-CSF, TNF-α, TRAP, and TRAIL.

In the present disclosure, one or more (such as 5 or less or 3 or less) amino acids in suitable position(s) of the amino acid sequence of the biomolecule are mutated to cysteine and the biomolecule is covalently linked to the functional moiety of the present disclosure (R1-R2-R3-R4) via the thiol group of the cysteine. For example, one or two amino acids of interest in the biomolecule of interest are mutated to cysteine for conjugation to the functional moiety. For an antibody, the mutation position may be present in a complementarity determining region or a non-complementarity determining region of a variable region. Preferably, the mutation is a mutation by substitution. More preferably, the mutation occurs in a variable region of the light chain of antibody. Generally, a mutant can be prepared and then its binding activity to a corresponding antigen is tested. If a mutant retains 70% or more, preferably 80% or more, more preferably 90% or more of binding activity as compared to the wild-type antibody, it is believed that the amino acid residue at the mutation position may be mutated to cysteine to covalently link to a functional moiety. Alternatively, in certain embodiments, if a conjugate produced by linking a mutant to R4 retains 80% or more, preferably 90% or more, preferably 95% or more of binding activity, it is believed that the amino acid residue at the mutation position may be mutated to cysteine to covalently link to a functional moiety.

In general, one or more of G, A, S, T, L, I, F, E, K, D and Y, etc., more preferably one or more of G, A, S, T, L, I, K and Y, more preferably one or more of G, A, T, L and S, in a CDR of a variable region of a light chain of an antibody may be mutated to cysteine. In some embodiments, one or more of G, A, S, T, L, I, F, E, K, D and Y, etc., more preferably one or more of G, A, S, T, L, I, K and Y, more preferably one or more of G, A, T, L, Y and S, in a CDR of a variable region of a heavy chain of an antibody may be mutated to cysteine. If mutation occurs in a non-CDR of a light chain of an antibody, one or more of G, A, S, T, L, I, F, E, K, D and Y, etc., preferably G, A, S, T, K, I, Y and L, more preferably one or more of G, A, T, Y and S, in the non-CDR in the variable region of a light chain or a heavy chain may be mutated to cysteine. In some embodiments, one or more of S, T, L, I, F, E, K, D, N, Q, R and Y residues, etc., in non-complementarity determining region (such as in FR1, FR2 or FR3) of a variable region of a light chain or a heavy chain may be mutated to cysteine. In some embodiments, substitution mutation may be introduced into one or more the following conservative sites: Gln3, Ser7, Ser26, Glu46, Thr68 and Asp72 in non-complementarity determining region (FR1, FR2 or FR3) of VH, and Thr5, Tyr49, Arg61, Ser63, Ser65, Ser67, Thr72, Thr74, Ser76 and Asp82 in non-complementarity determining region (FR1, FR2 or FR3) of VL.

For example, in some embodiments of the present disclosure, the mutation position in a heavy chain of anti-PD-1 antibody (such as Pembrolizumab) may be selected from the group consisting of: Ser7, Gly8, Gly15, Ala16, Ser17, Ala24, Ser25, Gly26, Tyr27, Thr28, Thr30, Asn31, Tyr32, Tyr33, Tyr35, Ala40, Gly42, Gly44, Leu45, Gly49, Gly50, Ile51, Asn52, Ser54, Asn55, Gly56, Gly57, Thr58, Asn59, Lys63, Lys65, Thr69, Leu70, Thr71, Thr72, Asp73, Ser74, Ser75, Thr76, Thr77, Thr78, Ala79, Leu83, Ser85, Leu86, Thr91, Ala92, Arg99, Asp100, Tyr101, Arg102, Asp104, Gly106, Gly111, Gly113, Thr114, 115Thr, 117Thr, Ser119, Ser120, Ala121, Ser122, Thr123, Lys124, Gly125 and Ser127; and the mutation position in a light chain may be selected from the group consisting of: Ile2, Thr5, Ser7, Ala9, Thr10, Leu11, Ser12, Leu13, Ser14, Gly16, Ala19, Thr20, Ala25, Ser26, Lys27, Gly28, Ser30, Thr31, Ser32, Gly33, Tyr34, Ser35, Tyr36, Leu37, Gly45, Ala47, Leu50, Leu51, Ile52, Tyr53, Leu54, Ala55, Ser56, Tyr57, Leu58, Ser60, Gly61, Ala64, Ser67, Gly68, Ser69, Gly70, Ser71, Gly72, Thr73, Ala76, Thr78, Ser80, Ser81, Ser95, Arg96, Asp97, Leu98, Leu100, Thr101, Phe102, Gly104, Ile110, Lys111 and K130.

Alternatively, the mutation position in a heavy chain of anti-PD-1 antibody (such as Nivolumab) may be selected from the group consisting of: Gln3, Ser7, Gly8, Gly9, Gly10, Gly15, Ser17, Lys23, Ala24, Ser25, Gly26, Ile27, Asn31, Thr28, Ser30, Ser32, Gly33, Ala40, Gly42, Gly44, Leu45, Ala49, Ile51, Tyr53, Asp54, Gly55, Ser56, Lys57, Tyr59, Tyr60, Ala61, Asp62, Ser63, Lys65, Gly66, Thr69, Ile70, Ser71, Arg72, Asp73, Asn74, Ser75, Lys76, Asn77, Thr78, Leu79, Leu81, Ser85, Leu86, Ala88, Thr91, Ala92, Thr98, Asn99, Asp100, Asp101, Tyr102, Gly104, Gly106, Thr107, Leu108, Thr110, Ser112, Ser113, Ala114, Ser115, Thr116, Lys117, Gly118 and Ser120; and the mutation position in a light chain may be selected from the group consisting of: Ile2, Leu4, Thr5, Ser7, Ala9, Thr10, Leu11, Ser12, Leu13, Ser14, Gly16, Ala19, Thr20, Leu21, Ala25, Ser26, Ser28, Ser30, Ser31, Tyr32, Leu33, Ala34, Tyr36, Gly41, Ala43, Leu46, Leu47, Ile48, Tyr49, Asp50, Ala51, Ser52, Asn53, Arg54, Ala55, Thr56, Gly57, Ile58, Ala60, Arg61, Ser63, Gly64, Ser65, Gly66, Ser67, Gly68, Thr69, Thr72, Leu73, Thr74, Ile75, Ser76, Ser77, Leu78, Ala84, Ser91, Ser92, Asn93, Arg96, Thr97, Phe98, Gly99, Gly101, Thr102, Ile106, Lys107, Thr109, Ala111, Ala112, Ser114, Ile117 and Ser121.

The mutation position in a heavy chain of anti-CTLA-4 antibody may be selected from the group consisting of: Ser7, Gly8, Gly9, Gly10, Gly15, Ser17, Leu18, Leu20, Ala24, Ser25, Gly26, Phe27, Thr28, Phe29, Ser30, Ser31, Tyr32, Thr33, Ala40, Gly42, Lys43, Gly44, Leu45, Glu46, Thr49, Phe50, Ile51, Ser52, Tyr53, Asp54, Gly55, Lys58, Tyr59, Tyr60, Ala61, Asp62, Ser63, Lys65, Gly66, Thr69, Ile70, Ser71, Ser75, Lys76, Thr78, Leu79, Leu81, Ser85, Leu86, Ala88, Gly89, Asp90, Thr91, Ala92, Tyr94, Tyr95, Ala97, Phe98, Thr99, Gly100, Leu102, Gly103, Asp106, Tyr107, Gly109, Gly111, Thr112, Leu113, Thr115, Ser117, Ser118, Ala119, Ser120, Thr121 and Lys122; and the mutation position in a light chain may be selected from the group consisting of Ile2, Leu4, Thr5, Ser7, Gly9, Thr10, Leu11, Ser12, Leu13, Ser14, Gly16, Ala19, Thr20, Leu21, Ala25, Ser26, Ser28, Gly30, Ser31, Ser32, Tyr33, Leu34, Ala35, Tyr37, Lys40, Gly42, Ala44, Leu47, Leu48, Ile49, Tyr50, Gly51, Ala52, Phe53, Ser54, Ala56, Thr57, Gly58, Ile59, Ser64, Gly65, Ser66, Gly67, Ser68, Gly69, Thr70, Asp71, Thr73, Leu74, Thr75, Ile76, Ser77, Leu79, Ala85, Tyr92, Gly93, Ser94, Ser95, Thr98, Phe99, Gly100, Gly102, Thr103, Lys104, Ile107, Lys108, Thr110, Ala112, Ala113, Ser115, Ser128, Gly129 and Thr130.

The mutation position in a heavy chain of the anti-CTLA-4 antibody Ipilimumab may be selected from the group consisting of: Gln3, Arg19, Leu20, Ser25, Gly26, Phe27, Thr28, Phe29, Ser30, Ser31, Tyr32, Thr33, Met34, His35, Gly44, Phe50, Ile51, Ser52, Tyr53, Asp54, Gly55, Asn56, Asn57, Lys58, Tyr59, Tyr60, Thr69, Ser71, Arg72, Asp73, Asn74, Ser75, Lys76, Asn77, Thr99, Gly100, Trp101, Leu102, Gly103 and Pro104; the mutation position in a light chain is selected from the group consisting of: Gln6, Arg24, Ala25, Ser26, Gln27, Ser28, Val29, Gly30, Ser31, Ser32, Tyr33, Ile49, Tyr50, Gly51, Ala52, Phe53, Ser54, Arg55, Ala56, Phe53, Ser54, Arg55, Ala56, Thr57, Gly58, Ile59, Pro60, Asp61, Arg62, Ser68, Gly69, Thr70, Gln90, Gln91, Tyr92, Gly93, Ser94, Ser95, Pro96 and Trp 97.

The mutation position in a heavy chain of anti-TNFα antibody may be selected from the group consisting of: Ser7, Gly8, Gly9, Gly10, Leu11, Gly15, Ser17, Leu18, Leu20, Ala24, Ser25, Gly26, Thr28, Asp30, Asp31, Tyr32, Ala33, Ala40, Gly42, Gly44, Leu45, Ser49, Ala50, Ile51, Thr52, Asn54, Ser55, Gly56, Ile58, Asp59, Tyr60, Ala61, Asp62, Ser63, Glu65, Gly66, Phe68, Thr69, Ile70, Ser71, Asp73, Asn74, Ala75, Lys76, Ser78, Leu79, Tyr80, Leu81, Ser85, Leu86, Ala88, Thr91, Ala92, Lys98, Ser100, Tyr101, Leu102, Ser103, Thr104, Ala105, Ser106, Ser107, Leu108, Asp109, Tyr110, Gly112, Gly114, Thr115, Leu116, thr118, Ser120, Ser121, Ala122, Ser123 and Thr124; and the mutation position in a light chain may be selected from the group consisting of: Asp1, Thr5, Ser7, Ser9, Ser10, Leu11, Ser12, Ala13, Ser14, Gly16, Thr20, Ile21, Ala25, Ser26, Gln27, Gly28, Ile29, Arg30, Asn31, Tyr32, Leu33, Ala34, Tyr36, Lys39, Gly41, Lys42, Ala43, Leu48, Leu47, Ile48, Tyr49, Ala50, Ala51, Ser52, Thr53, Leu54, Gln55, Ser56, Gly57, Ser60, Ser63, Gly64, Ser65, Gly66, Ser67, Gly68, Thr69, Asp70, Thr72, Leu73, Thr74, Ile75, Ser76, Ser77, Leu78, Ala84, Thr85, Tyr91, Asn92, Arg93, Ala94, Tyr96, Thr97, Phe98, Gly99, Gly101, Thr102, Ile106, Lys107, Thr109 and Ala111.

The mutation position in a heavy chain of anti-CD28 antibody may be selected from the group consisting of: Ser7, Gly8, Gly15, Ala16, Ser17, Ser21, Ala24, Ser25, Gly26, Tyr27, Thr28, Thr30, Ser31, Tyr32, Ala40, Gly42, Gly44, Gly49, Tyr52, Gly54, Thr58, Ala68, Thr69, Thr71, Thr74, Ser75, Ser77, Thr78, Ala79, Ser84, Leu86, Ser88, Thr91, Ala92, Thr97, Ser99, Tyr101, Gly102, Leu103, Gly113, Thr114, Thr115, Thr117, Ser119, Ser120, Ala121, Ser122 and Thr123; and the mutation position in a light chain may be selected from the group consisting of: Thr5, Ser7, Ser9, Ser10, Ser11, Ser12, Ala13, Ser14, Gly16, Thr20, Thr22, Ala25, Ser26, Ser27, Ile29, Tyr30, Ala43, Leu46, Leu47, Tyr49, Lys50, Ala51, Ser52, Leu54, Thr56, Gly57, Ser60, Ser63, Gly64, Ser65, Gly66, Ser67, Gly68, Thr69, Asp70, Thr72, Thr74, Ser76, Ser77, Ala84, Thr85, Gly91, Thr93, Tyr94, Tyr96, Thr97, Phe98, Gly99, Gly100, Gly101, Thr102, Thr109 and Ala111.

The mutation position in a heavy chain of the anti-4-1BB antibody may be selected from the group consisting of: Thr31, Tyr32, Ser35, Lys50, Tyr52, Asp55, Ser56, Tyr57, Thr58, Asn59, Tyr60, Ser61, Gln65, Gly66, Gly99, Tyr100, Gly101, Asp104 and Tyr105; the mutation position in a light chain may be selected from the group consisting of: Ser23, Gly24, Asp25, Asn26, Gly28, Asp29, Gln30, Tyr31, Gln49, Asp50, Lys51, Asn52, Arg53, Ser55, Gly56, Thr89, Tyr90, Thr91, Gly92, Gly94 and Ser95.

The mutation position in a heavy chain of the anti-Her2 antibody (such as Trastuzumab) may be selected from the group consisting of: Arg19, Lys30, Asp31, Tyr33, Arg50, Tyr62, Asn55, Tyr57, Arg59, Tyr60, Asp62, Lys65, Asp102 and Tyr105; the mutation position in a light chain is selected from the group consisting of: Asp1, Gln3, Gln27, Asp28, Asn30, Tyr49, Tyr55, Arg66, Asp70 and Tyr92.

The mutation position in a heavy chain of the anti-PD-L1 antibody (such as Atezolizumab) is selected from the group consisting of: Gln3, Asp31, Tyr54, Tyr59, Tyr60, Asp62, Lys65, Asp73, Lys76, Asn77 and Arg99; the mutation position in a light chain is selected from the group consisting of: Arg24, Gln27, Asp28, Tyr49, Tyr55, Asp70, Gln89, Gln90, Tyr91 and Tyr93.

In some preferred embodiments, the mutation positions of various antibodies are preferably selected from the positions listed in Tables 5 to 19 which retain 70% or more, preferably 80% or more, preferably 90% or more of binding activity after mutation or retain 80% or more, preferably 90% or more of binding activity after conjugating to R4. These mutation positions include those in CDR and non-CDR. For example, preferred mutation positions of a light chain of anti-PD-1 antibody 1 may include Ala25, Ser26, Gly28, Ser30, Thr31, Ser32, Gly33, Ser35, Tyr36, Leu37, Leu54, Ala55, Ser56, Tyr57, Ser60, Gly61, Ser95, Thr101 and Gly104; more preferably, the mutation positions include Ser26, Gly28, Ser30, Ser32, Gly33, Ser35, Ala55, Ser56, Ser60, Gly61 and Ser95. Preferred mutation positions of a light chain of anti-PD-1 antibody 2 may include Ala25, Ser26, Ser28, Ser30, Ser31, Ala34, Ala51, Ser52, Ser54, Ala55, Thr56, Gly57, Ile58, Ala60, Ser91, Ser92, Thr97 and Gly99; more preferably, the mutation positions include Ser26, Ser28, Ser30, Ser31, Ala34, Ser52, Ser54, Thr56, Gly57, Ser91, Ser92, Thr97 and Gly99. Preferred mutation positions of a light chain of anti-CTLA-4 antibody may include Ala25, Ser26, Ser28, Gly30, Ser31, Ser32, Leu34, Ala35, Gly51, Ala52, Ser54, Ala56, Thr57, Gly58, Gly93, Ser94, Ser95, Thr98 and Gly100; more preferably, the mutation positions include Ala25, Ser26, Ser28, Gly30, Ser31, Ser32, Gly51, Ser54, Thr57, Gly58, Gly93, Ser94, Ser95 and Gly100. Preferred mutation positions of a light chain of anti-CD28 antibody may include Ser26, Ile29, Tyr30, Lys50, Ala51, Ser52, Gly91, Thr93, Tyr94, Tyr96, Thr97 and Gly99; more preferably, the mutation positions include Tyr30, Ala51, Tyr96, Thr97 and Gly99. Preferred mutation positions of a light chain of anti-TNFα antibody may include Ala25, Ser26, Gly28, Ile29, Tyr32, Leu33, Ala34, Ala50, Ala51, Ser52, Thr53, Leu54, Ser56, Gly57, Tyr91, Ala94 and Thr97; more preferably, the mutation positions include Ser26, Gly28, Ala51, Ser56, Gly57, Tyr91 and Ala94.

Preferred mutation positions of a heavy chain of anti-PD-1 antibody 1 may include Thr30, Tyr32, Tyr35, Gly50, Ile51, Ser54, Gly56, Gly57, Thr58, Lys63, Tyr101 and Gly106; more preferably, the mutation positions include Thr30, Gly50, Ser54, Gly56, Gly57, Thr58, Lys63 and Gly106. Preferred mutation positions of a heavy chain of anti-PD-1 antibody 2 may include Ser30, Ser32, Gly33, Ile51, Tyr53, Asp54, Gly55, Ser56, Lys57, Ala61, Ser63, Lys65, Gly66, Asp101, Gly104, Gly106, Thr107 and Leu108; more preferably, the mutation positions include Ser30, Ser32, Gly33, Tyr53, Gly55, Ser56, Ser63, Lys65, Gly66, Gly104, Gly106 and Thr107. Preferred mutation positions of a heavy chain of anti-CTLA-4 antibody may include Ser30, Ser31, Tyr32, Thr33, Ile51, Asp54, Gly55, Lys58, Tyr59, Tyr60, Ala61, Asp62, Ser63, Lys65, Gly66, Gly100, Leu102, Gly103, Asp106 and Tyr107; more preferably, the mutation positions include Ser30, Ser31, Thr33, Gly55, Tyr60, Ala61, Ser63, Lys65, Gly66, Gly100, and Gly103. Preferred mutation positions of a heavy chain of anti-CD28 antibody may include Ser25, Gly26, Tyr27, Thr28, Thr30, Ser31, Tyr32, Tyr33, Tyr52, Gly54, Thr58, Ser99, Tyr101, Gly102 and Leu103; more preferably, the mutation positions include Gly26, Tyr27, Thr28, Thr30, Ser31, Tyr52, Gly54 and Leu103. Preferred mutation positions of a heavy chain of anti-TNFα antibody may include Tyr32, Ala33, Ile51, Thr52, Ser55, Gly56, Ile58, Tyr60, Ala61, Ser63, Gly66, Ser100, Tyr101, Leu102, Ser103, Thr104, Ala105, Ser106, Ser107, Leu108 and Tyr110; more preferably, the mutation positions include Ala33, Ile51, Ala61, Ser63, Gly66, Ser100, Thr104 and Ser106.

Similarly, when the mutation occurs in a non-CDR, i.e., in a framework region of a variable region of a light chain, mutation positions of various antibodies may be preferably selected from those listed in Tables 7 and 8 which retain 80% or more, preferably 90% or more of binding activity after mutation. For example, for the light chain of the anti-PD-1 antibody 1 as disclosed herein, the preferred mutation positions in the framework region include Thr5, Ser7, Ala9, Thr10, Leu11, Ser12, Leu13, Ser14, Gly16, Ala19, Thr20, Gly45, Ala47, Leu50, Leu51, Ile52, Ala64, Ser67, Gly68, Ser69, Gly70, Ser71, Gly72, Thr73, Ala76, Thr78, Ser80, Ser81, Ile110 and Lys111; more preferably, the mutation positions include Thr5, Ser7, Ala9, Leu11, Leu13, Ser14, Ala47, Leu50, Ala64, Gly68, Ser69, Ser71, Gly72, Thr73, Ser80 and Ile110. For the heavy chain of anti-PD-1 antibody 2 as disclosed herein, the preferred mutation positions of the framework region include Ser7, Gly8, Gly9, Gly10, Gly15, Ser17, Ala24, Ser25, Gly26, Thr28, Ser30, Ala40, Gly42, Gly44, Thr68, Ser71, Ser75, Thr78, Thr85, Ala88, Thr91, Ala92, Thr98, Thr110, Ser112, Ser113, Ala114, Ser115, Thr116, Lys117, Gly118 and Ser120; more preferably, the mutation positions include Ser7, Gly8, Gly9, Ala24, Ser25, Gly26, Gly44, Thr68, Ser75, Thr78, Thr85, Thr110, Ser112, Ser113, Ser115, Thr116, Lys117, Gly118 and Ser120.

With respect to other functional proteins, such as cytokines, if its mutant retains 70% or more, preferably 80% or more, more preferably 90% or more of binding activity (e.g., binding capacity to its nature ligand) of a wild-type protein, it is believed that the amino acid residue at the position may be mutated to cysteine to link to a functional moiety. For example, the mutation position of IL2 may be selected from the group consisting of Lys32, Lys35, Thr37, Met39, Thr41, Lys43, Lys48, Lys49, Lys64, Leu72, Ala73, Ser75, Lys76, Leu94, Thr101, Thr102, Tyr107, Ala108, Thr11 and Ala112, so as to block an alpha receptor; or is selected from the group consisting of Leu12, His16, Leu19, Met23, Gly27, Ser75, Arg81, Leu85, Ser87, Leu94, Gly98, Ser99, Thr101 and Thr133, so as to block a beta receptor; or is selected from the group consisting of Leu36, Ala50, Thr51, Thr123, Ser126, Ser127 and Ser130, so as to block a gamma receptor. In some embodiments, the mutation position of the IL2 may be selected from the group consisting of Lys32, Lys35, Thr37, Thr41, Lys43, Lys48, Lys49, Ala50, Leu72, Ala73, Ser75, Lys76, Leu94, Thr101, Thr102, Ala108, Thr111 and Alal 12, so as to block alpha receptor of IL2; or may be selected from the group consisting of Leu19, Gly27, Ser75, Leu80, Ser87, Leu94, Gly98, Ser99, Thr101 and Thr133, so as to block beta receptor of IL2; or is selected from the group consisting of Leu36, Ala50, Thr51, Thr123, Ser126, Ser127 and Ser130, so as to block gamma receptor of IL2.

One or more of Ala, Gly, Ser, Thr, Leu, Lys, Tyr, Phe, Asp, Glu and Ile of cytokines, such as IL2, IL10 and IL15, may be mutated to cysteine. Preferably, any of Thr, Leu, Ala, Gly and Ser in the amino acid sequence of cytokines is mutated to cysteine.

In some embodiments, the mutation position in a Human IL10 may be selected from the group consisting of Thr6, Ser8, Ser11, Thr13, Gly17, Arg24, Ser31, Arg32, Lys34, Thr35, Lys40, Leu46, Lys49, Ser51, Lys57, Gly58, Ser66, Tyr72, Lys88, His90, Ser93, Lys99, Thr100, Arg104, Lys117, Ser118, Lys119, Lys125, Lys130, Lys134, Gly135, Tyr137, Tyr149, Thr155, Lys157 and Arg159.

For the fusion protein of the present disclosure, one or more cysteines may be introduced into any protein of the fusion protein. For example, for a bispecific antibody, the mutation(s) may be introduced into the CDR or non-CDR of either or both of the scFv, or into the either or both of the antigen binding domain. For a fusion protein of an antibody and a cytokine, either or both of the antibody and cytokine may contain the mutation(s).

Exemplified sequences may be referred to SEQ ID NO: 13-83.

Mutation, transfection, expression and purification of a biomolecule may be performed by the methods known in the art. For example, a nucleic acid of a biomolecule having a mutation position may be directly synthesized, then nucleic acid molecules of different fragments obtained by enzyme digestion may be ligated into an expression vector and the expression vector is transformed into bacteria or eukaryotic host cells. The biomolecule containing the cysteine mutation may be obtained through recombination in the host cells.

Bacteria or eukaryotic host cells suitable for use in the present disclosure may be host cells commonly used in the art, including but not limited to bacteria, yeast and mammal cells. Useful eukaryotic host cells include CHO cells, HEK293T cells, or Pichia pastoris.

Expression vectors suitable for use in the present disclosure may be virus-based expression vectors known in the art, including but not limited to baculovirus, simian virus (SV40), retrovirus or vaccinia based viral vectors. Expression vectors containing suitable regulatory elements and selecting markers may be used to prepare mammal cell lines which stably express a mutant. For example, GS Eukaryotic Expression System (Lonza), DHFR Eukaryotic Expression System (Invitrogen) and Pichia pastoris Expression System (Invitrogen) may be used in the expression and preparation.

Biomolecules of the present disclosure may be purified by the isolation methods known in the art. These methods include but are not limited to DEAE ionic exchange, gel filtration and hydroxyapatite chromatography. For example, protein G column may be used to isolate biomolecules in the supernatant of cell cultures or in extracts of cytoplasm. In some embodiments, the biomolecules may be subjected to “engineering modification” to make them to contain an amino acid sequence that allows the biomolecules to be captured to an affinity matrix. For example, a tag may be used to facilitate purification of a polypeptide. The tag includes but is not limited to c-myc, hemagonium, poly-His (e.g., 6His) or Flag™ (Kodak). Such kind of tags may be inserted to any position within a polypeptide, including a carboxyl terminus or an amino terminus. Biomolecules of the present disclosure may also be purified by immunoaffinity chromatography.

The functional moiety suitable for use in the present disclosure may be represented by formula R1-R2-R3-R4. In the functional moiety, R1, R2, R3 and R4 are linked together via any suitable linkage manner, including but not limited to amide, ester, carbamate, urea or hydrazone bond. In the present disclosure, amide bond may be represented by “—CO—NH—”, ester bond may be represented by “—C(O)—O—”, carbamate bond may be represented by “—NH—C(O)—O—”, urea bond may be represented by “—NH—CO—NH—”, and hydrazone bond may be represented by “—CH═N—NH—”.

R1 is a protective group for the biomolecule R5. It may be selected from any group that can prevent the biomolecule from binding to its ligand or receptor, so as to prevent it from being interfered by other molecules, for example, those that prevent it from binding to its ligand or receptor, before it reaches a pathologic microenvironment, such as tumor or inflammatory microenvironment. Suitable R1 may be selected from the group consisting of polyethylene glycol-C₁₋₅ alkylcarbonyl, naphthylcarbonyl, quinolylcarbonyl, fluorenylcarbonyl, adamantylcarbonyl,

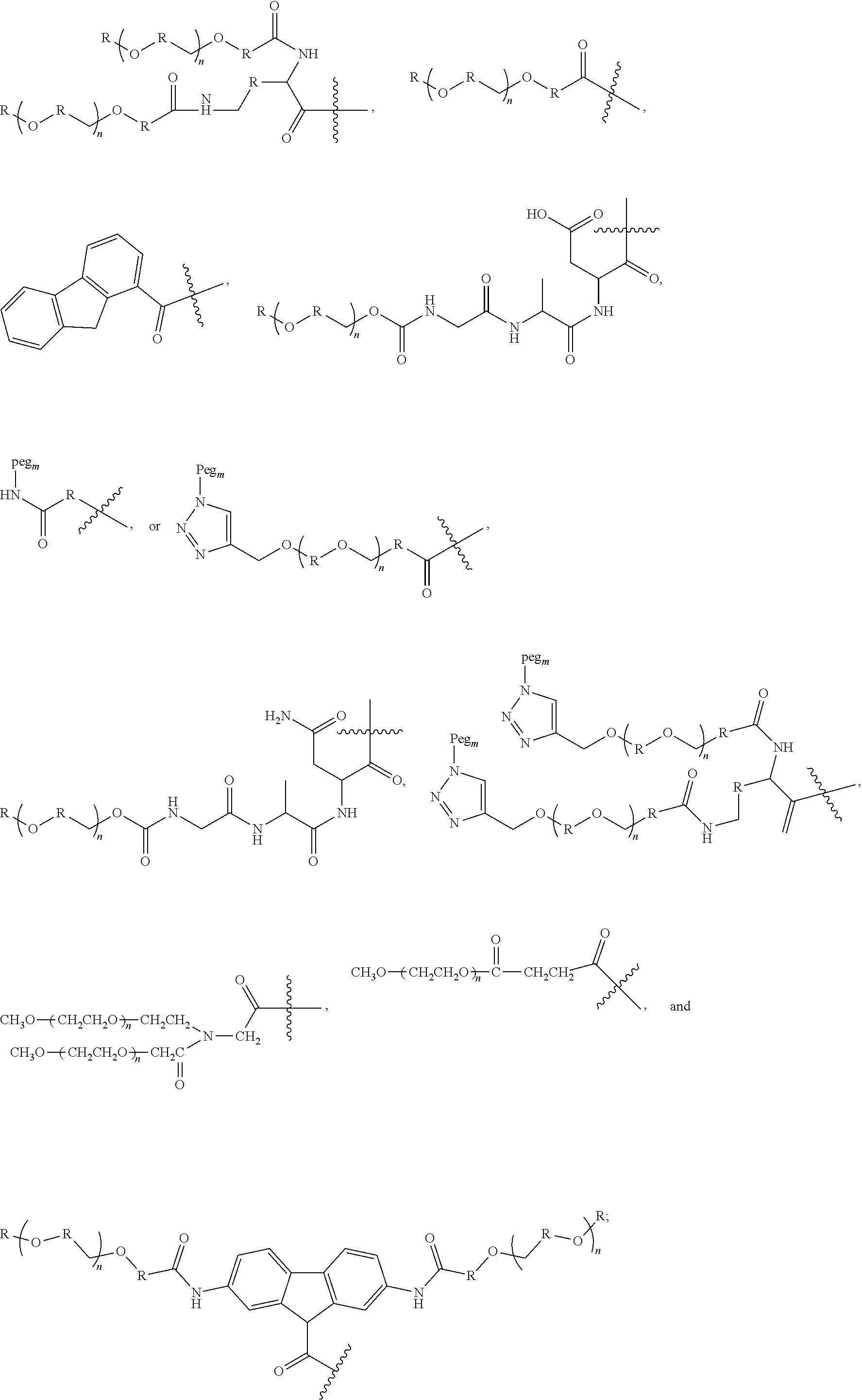

wherein each R is independently a C₁₋₄alkyl; each n is independently an integer in a range of 1 to 30000, such as an integer in a range of 1 to 15000, 1 to 5000, 1 to 2000, 1 to 300, 1 to 150, 1 to 50, 1 to 20 or 3 to 12; polyethylene glycol or pegₘ represents a polyethylene glycol having a molecular weight in a range of 44 to 132000, such as that in a range of 1000 to 50000 or 10000 to 30000; m represents the molecular weight of the polyethylene glycol; and the wave line indicates the position of R1 linking to R2.

In some embodiments, R1 is selected from the group consisting of:

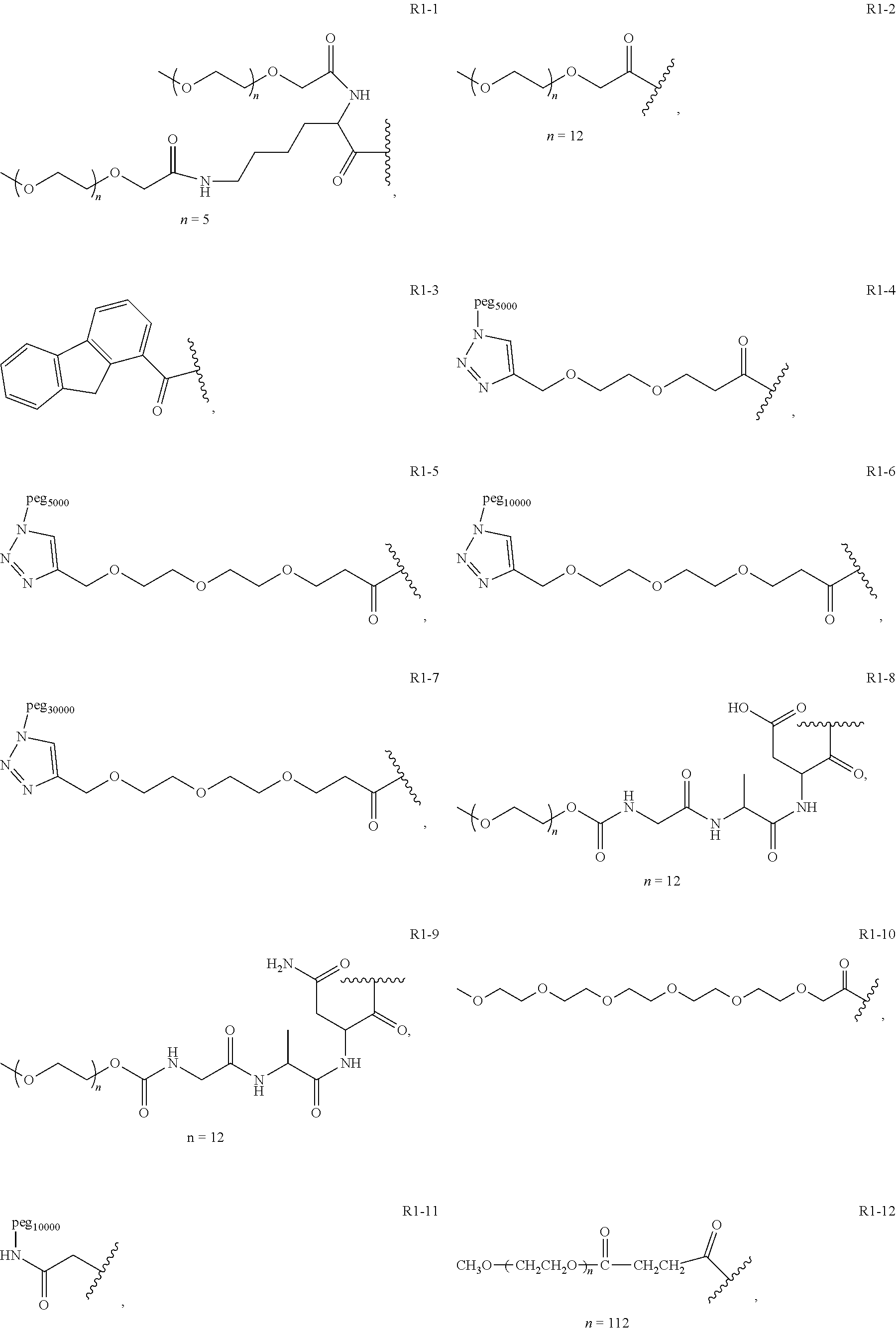

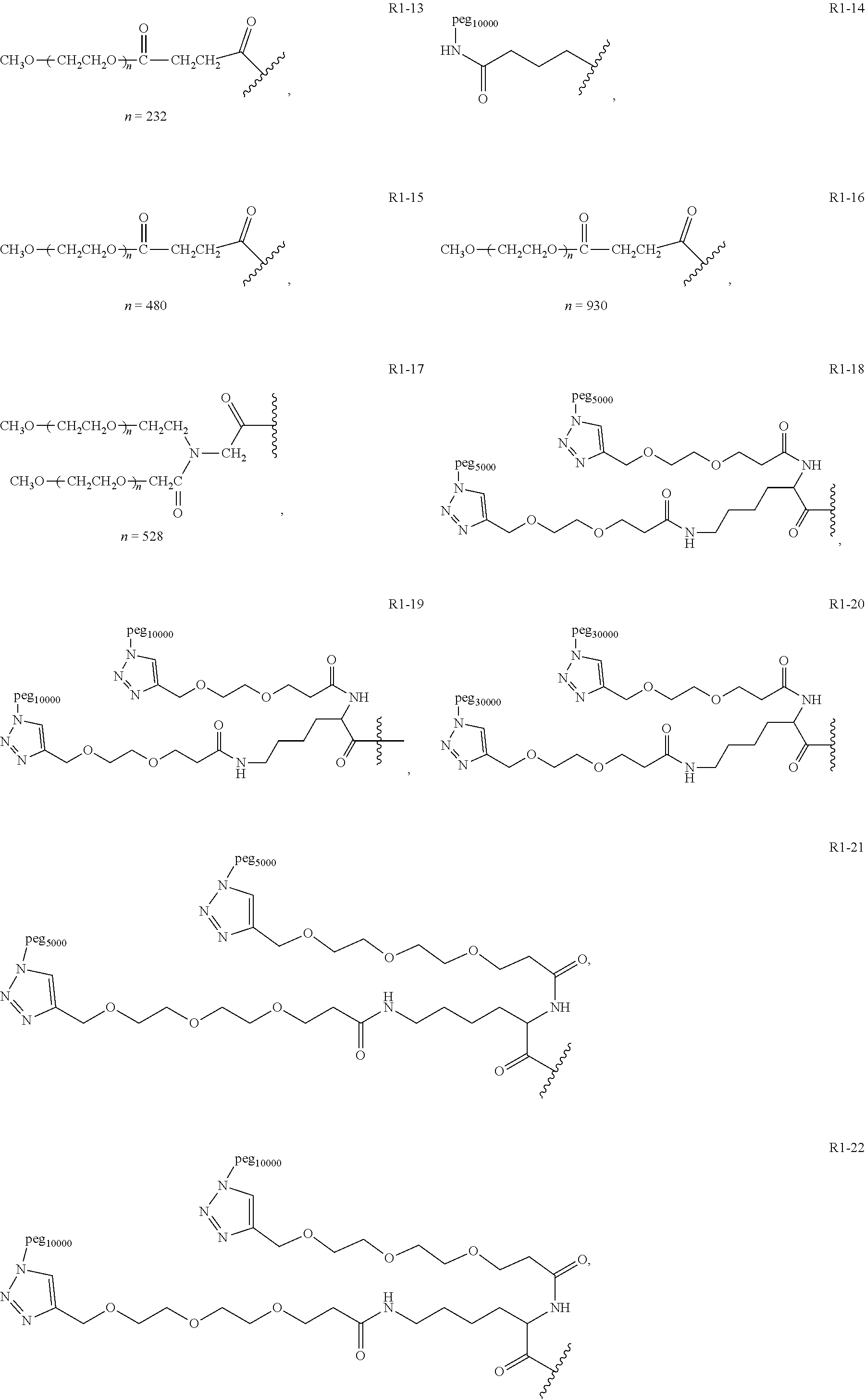

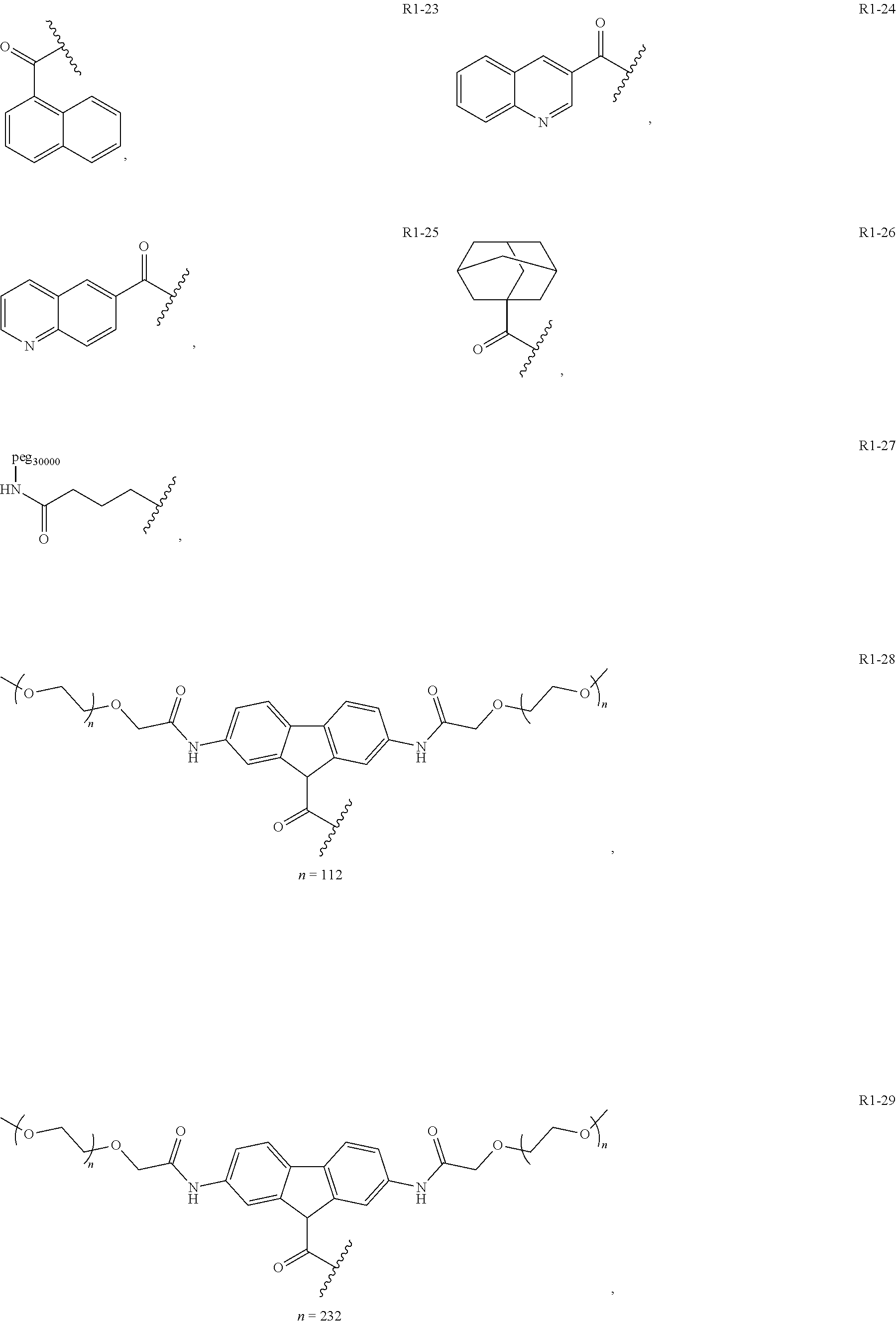

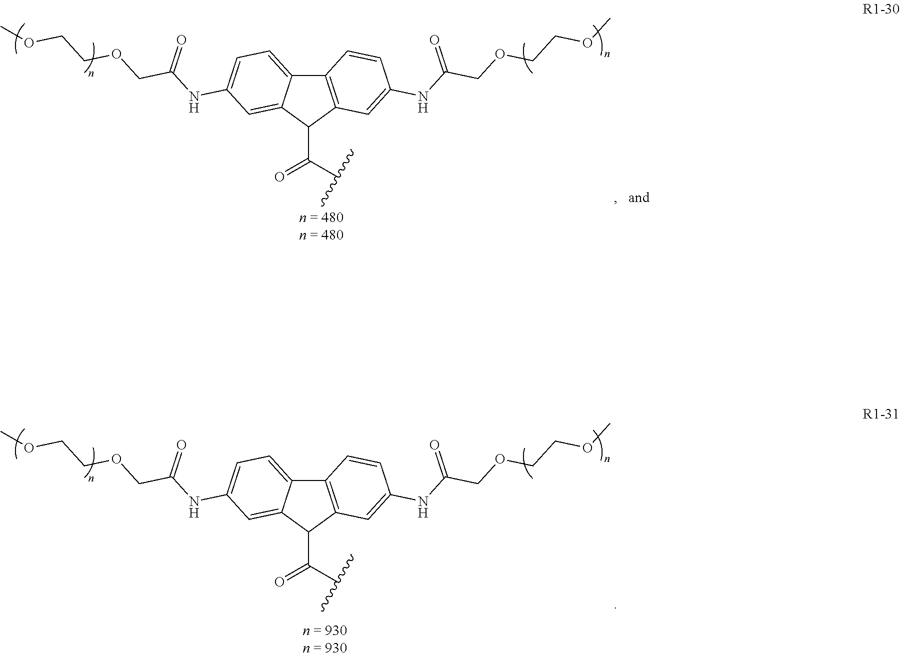

Generally, if a mutation position is present in the functional domain of a biomolecule, for example, in a CDR of an antibody, there is no specific limitation on the molecular weight of R1 and R1 may have a relatively low molecular weight. If a mutation position is present outside the functional domain of a biomolecule, for example, in a non-CDR of an antibody, R1 is preferably selected to make the molecular weight of R1-R2-R3-R4 to be higher than 200, preferably higher than 500, more preferably to be higher than 1000, so as to make the molecular weight of the conjugate of biomolecule to be 5000 or more, preferably 8000 or more, more preferably 10000 or more, thereby better preventing the biomolecule from binding to its ligand or receptor before arriving at a pathologic microenvironment.

In the present disclosure, R2 is a cleavable linker arm. It may be a peptide capable of being activated by a proteolytic enzyme, protease or peptidase or a chemical bond capable of being acidically activated in a pathologic microenvironment. In the present disclosure, the proteolytic enzyme, protease or peptidase may be various proteolytic enzymes, proteases or peptidases present in a pathologic microenvironment. For example, protease may be cysteine protease, aspartate protease, glutamic acid protease, threonine protease, gelatinase, metallopro-teinase, or asparagine peptide lyase. In some embodiments, R2 may be cleaved by at least one of a Legumain, Cathepsin B, Cathepsin C, Cathepsin D, Cathepsin E, Cathepsin K, Cathepsin L, kallikrein, hKl, hKlO, hK15, plasmin, collagenase, Type IV collagenase, astromelysin, Factor Xa, chymotrypsin-like protease, trypsin-like protease, elastase-like protease, subtilisin-like protease, actinidain, bromelain, calpain, caspase, caspase-3, Mirl-CP, papain, HIV-1 protease, HSV protease, CMV protease, chymosm, pepsm, matriptase, legumain, plasmepsm, nepenthesin, metalloexopeptidase, metalloendopeptidase, matrix metalloprotease (MMP), MMPl, MMP2, MMP3, MMP8, MMP9, MMPlO, MMP11, MMP12, MMP13, MMP14, ADAMlO, ADAM12, urokinase plasminogen activator (uPA), nenterokinase, prostate-specific antigen (PSA, hK3), interleukin-113 converting enzyme, thrombin, FAP (FAP-a), meprin, granzyme, dipeptidyl peptidase, and dipeptidyl peptidase IV (DPPIV/CD26). In preferred embodiments, the present disclosure specifically relates to Legumain, which is largely expressed and secreted by tumor cells in a tumor microenvironment. Tumor-associated macrophage (M2 type) is also different from monocyte and inflammatory macrophage (M1 type) by the expression of Legumain. In the present disclosure, the peptide is a substrate of the proteolytic enzyme. It can be recognized and cleaved by the proteolytic enzyme.

R2 of the present disclosure may be represented by —R2a-, —R2b-, —R2a-N—, —R2a-D-, —R2a-AAN—, —R2a-AAD-, or —R2a-R2b-; wherein R2a is a peptide capable of being cleaved at amide bond by one or more proteolytic enzymes; R2b is a kind of peptide with its nitrogen in side chain forming a carbamate with R3 and the carbamate can be cleaved by one or more proteolytic enzymes; A is alanine; N is asparagine with its nitrogen in side chain forming a carbamate with R3 and the carbamate can be cleaved by Legumain; D is aspartic acid with its nitrogen in side chain forming a carbamate with R3 and the carbamate can be cleaved by Granzyme B. R2a and R2b can be linked by forming an amide bond. After legumain, granzyme B cleaving the bonds (such as carbamate) between R2 and R3, R3 can quickly undergo auto-releasing. Then the R4 moiety may be retained. The other enzymes may cleave at amide bond of R2, which may cause some amino acid residues remaining in the linker and thus auto-releasing of R3 will not occur. Examples of such R2 include but are limited to LTPRLGPAAN (SEQ ID NO:84), GPAAN (SEQ ID NO:85) and LSGRSDN (SEQ ID NO:86).

In some embodiments, suitable peptide capable of being activated by a proteolytic enzyme may be a tripeptide. Any substrate peptide capable of being recognized and cleaved (activated) by a proteolytic enzyme in a pathologic microenvironment known in the art may be used as R2 as disclosed herein. Such peptides may have structures disclosed in WO 2016/026458, the entity of which is incorporated in the present disclosure by reference. In some embodiments, in the tripeptide structure suitable for use in the present disclosure, the amino acid residue linked to R1 may be selected from the group consisting of Ala, Thr, Val and Ile, the middle amino acid residue may be selected from the group consisting of Ala, Thr, Val and Asn, and the amino acid residue linked to R3 may be selected from the group consisting of Asn and Asp. Generally, R2 links to R1 via an amino group of its amino acid residue in a linkage manner of amide, ester, carbamate, urea or hydrazone bond, and links to R3 via a carboxyl group of its amino acid residue in a linkage manner of amide, ester, carbamate, urea or hydrazone bond. In some preferred embodiments of the present disclosure, the tripeptide is selected from the group consisting of Ala-Ala-Asn and Ala-Ala-Asp. Ala-Ala-Asn may be recognized and cleaved by Legumain, and Ala-Ala-Asp may be recognized and cleaved by granzyme.

In some embodiments, R2 may be a chemical bond capable of being acidically activated in a pathologic microenvironment. Such bond includes but is not limited to amide bond, ester bond, carbamate bond, urea bond or hydrazone bond. When R2 is a chemical bond, the functional moiety as disclosed herein may be represented by formula R1-R2-R3-R4, wherein R1 links to R3 by the chemical bond capable of being acidically activated in a pathologic microenvironment. For example, in some embodiments, the structure of R1-R2-R3-R4 may be represented as follows:

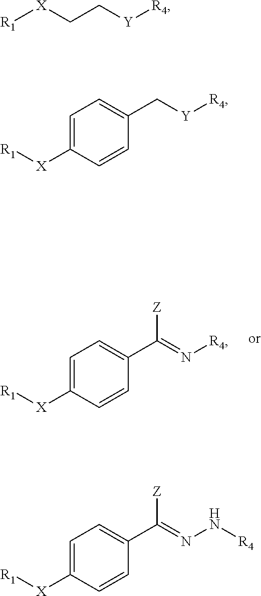

wherein X and Y are each independently NR′ or O, Z is H or C₁₋₁₀alkyl, preferably C₁₋₄alkyl, and R′ is H or C₁₋₄alkyl; R3 links to R1 and R4, respectively, via X and Y in a linkage manner of amide, ester, carbamate, urea or hydrazone bond.

In some embodiments, the structure of R1-R2-R3-R4 may be represented as follows:

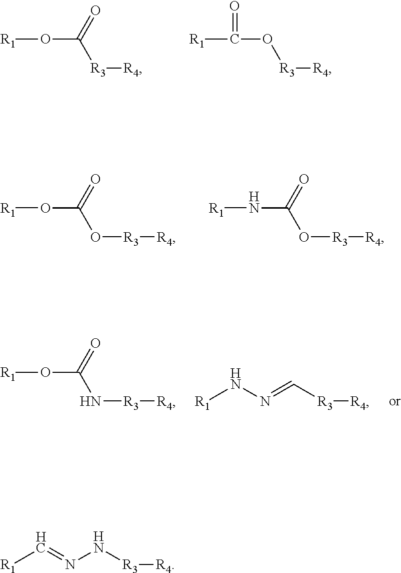

In the present disclosure, R3 may be an automatically cleavable linker arm capable of automatically shedding after cleavage of R2 to release R4-S-cys-R5. For example, such linker arm includes but is not limited to:

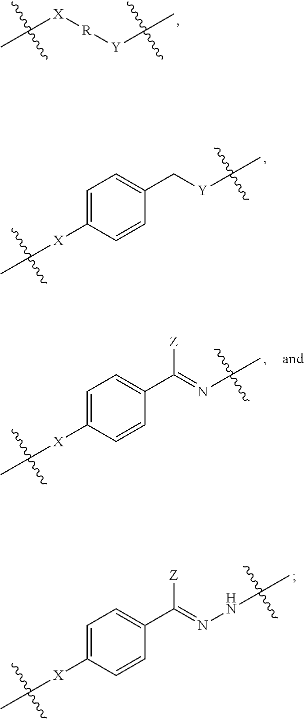

wherein X and Y are each independently NR′ or O, Z is H or C₁₋₁₀alkyl, preferably C₁₋₄alkyl; R is C₁₋₄alkyl; and R′ is H or C₁₋₄alkyl; wherein R4 links to R3 via Y or N in above formula in a manner of, such as amide bond, ester bond, carbamate bond, urea bond and hydrazone bond. In some embodiments, R3 is selected from the group consisting of —NH-phenyl-CH₂O—, —NH— phenyl-CH═N—, —NH-phenyl-C(CH₃)═N—, —O-phenyl-CH═N— and —O-phenyl-C(CH₃)═N—.

In some embodiments, R3 is a chemical bond capable of being acidically activated in a pathologic microenvironment. The chemical bond may be selected from the group consisting of amide bond, ester bond, carbamate bond, urea bond and hydrazone bond.

When R3 is a chemical bond capable of being acidically activated, R2 may be absent, such that the R1-R3-R4-S-cys-R5 can merely be acidically activated. On the other hand, when R2 is absent, R3 must be a chemical bond capable of being acidically activated.

In some embodiments, R3 is represented by any of the following structures:

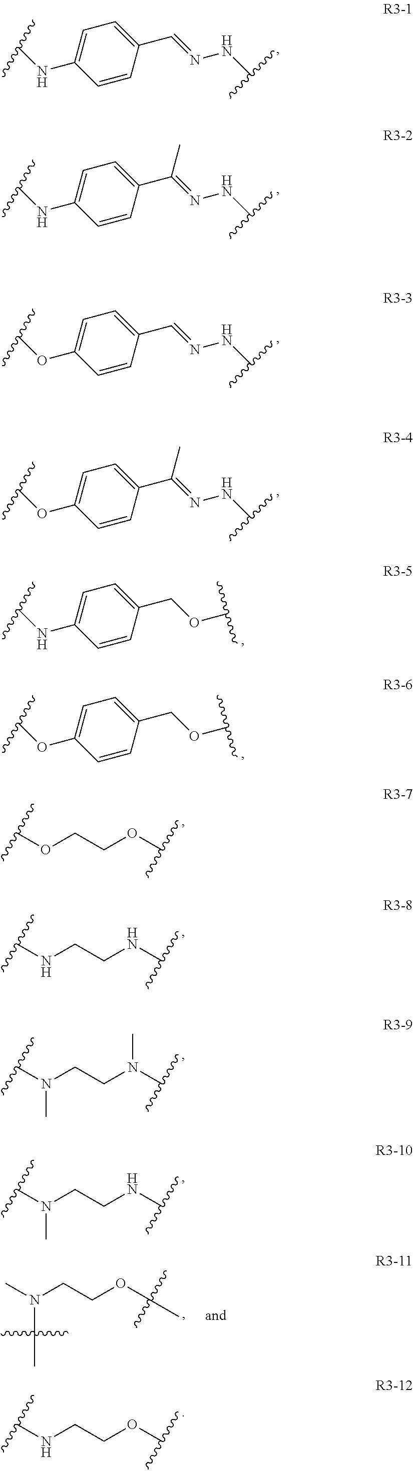

In the above formulae R3-1 to R3-12, R2 or R1 if R2 is absent may link to either end of the formulae as long as they form amide bond, ester bond, carbamate bond, urea bond and hydrazone bond.

In the present disclosure, R4 is a binding group capable of recovering, maintaining, reducing or promoting the binding capacity of a biomolecule to its antigen, ligand or receptor after cleavage of R2 and R3. In some embodiments, the resultant R4-s-Cys-R5 exhibits >60% affinity of native R5 to its antigen, ligand or receptor after cleavage of R2 and R3.

Suitable R4 may be represented by —R₄₋ₐ-R_{4-b}-R_{4-c}—, wherein R₄₋ₐ is selected from the group consisting of:

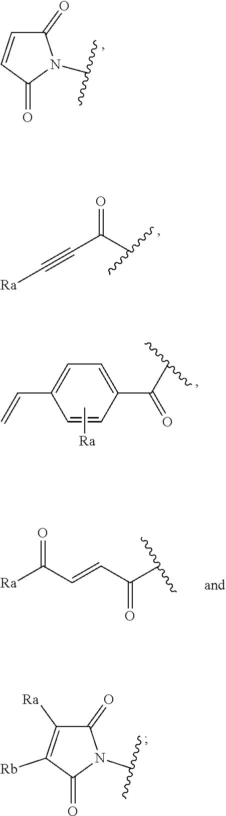

wherein Ra and Rb are each independently selected from the group consisting of H and C₁₋₆ alkyl or C₁₋₆ alkoxyl;
R_{4-b} is selected from the group consisting of:

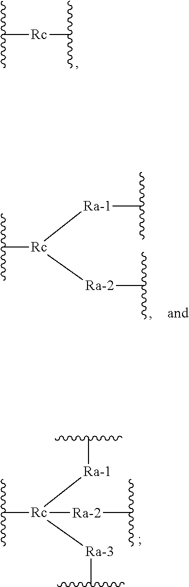

wherein in formula R4-b1, Rc is absent, or is selected from the group consisting of C₁₋₁₂ alkyl, C₁₋₁₂alkoxy-C₁₋₁₂alkyl, C₁₋₁₂alkyl-C₃₋₈cycloalkyl, (C₁₋₄alkyl-O)ₚ—C₁₋₁₂alkyl, C₁₋₁₂alkylcarbonylamino-(C₁₋₄alkyl-O)ₚ—C₁₋₁₂alkyl, phenyl-C₁₋₁₂alkyl, C₃₋₈cycloalkyl, C₁₋₁₂alkyl-C₃₋₈cycloalkyl-C₁₋₁₂alkyl, and C₁₋₁₂alkyl-phenyl-C₁₋₁₂alkyl; in formula R4-b2, Rc is a C₁₋₁₂alkylamino with Ra-1 and Ra-2 substituted at N atom of the amino group, and in formula 4-b3, Rc is a C₁₋₁₂alkyl with the last C atom at the end of the alkyl being substituted by Ra-1, Ra-2 and R2-3, wherein Ra-1, Ra-2 and Ra-3 are each independently selected from the group consisting of C₁₋₁₂alkyl, C₁₋₁₂alkyl-OH, and C₁₋₁₂alkyl-NR″R′″, wherein R″ and R′″ are each independently selected from the group consisting of H and C₁₋₁₂alkyl; wherein in formulae R4-b2 and R4-b3, R4-b links to R4-c via at least one of the Ra-1, Ra-2 and Ra-3;
R_{4-c} is selected from the group consisting of:

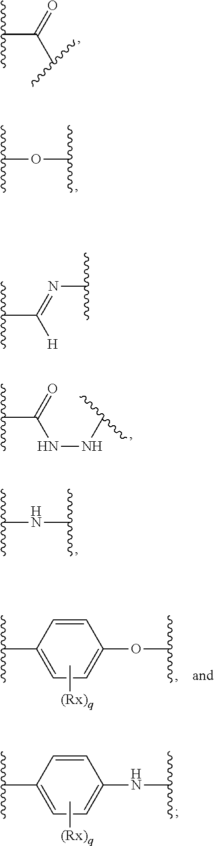

wherein Rx is selected from the group consisting of H, halo and C₁₋₄alkyl; p is an integer in a range of 1 to 10, such as an integer in a range of 1 to 5; and q is an integer in a range of 1 to 4, such as an integer in a range of 1 to 2; with the proviso that R4-c is absent when R4-a is selected from the group consisting of formulae R4-a2, R4-a3 and R4-a4;
wherein R3 links to R4 via the R_{4-c} of R4, and the wave line shown in each formula of R4-a indicates the position at which R4-a links to R4-b. Preferably, in formula R4c-III, Rc4-IV, R4c-VI and R4c-VII, R3 links to the carbon atom of these groups.

Generally, R4 links to the S atom of the cysteine of R5 via maleimide (R4-a1), acetylene (R4-a2), vinyl (R4-a3), mono-substituted butenedioic acid (R4-a4), or di-substituted maleimide (R4-a4).

In some embodiments, R4 is selected from the group consisting of:

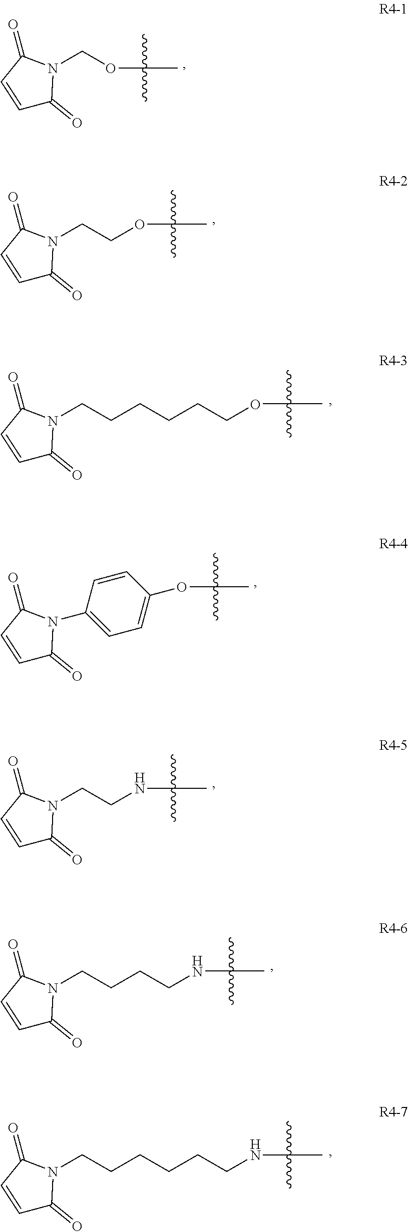

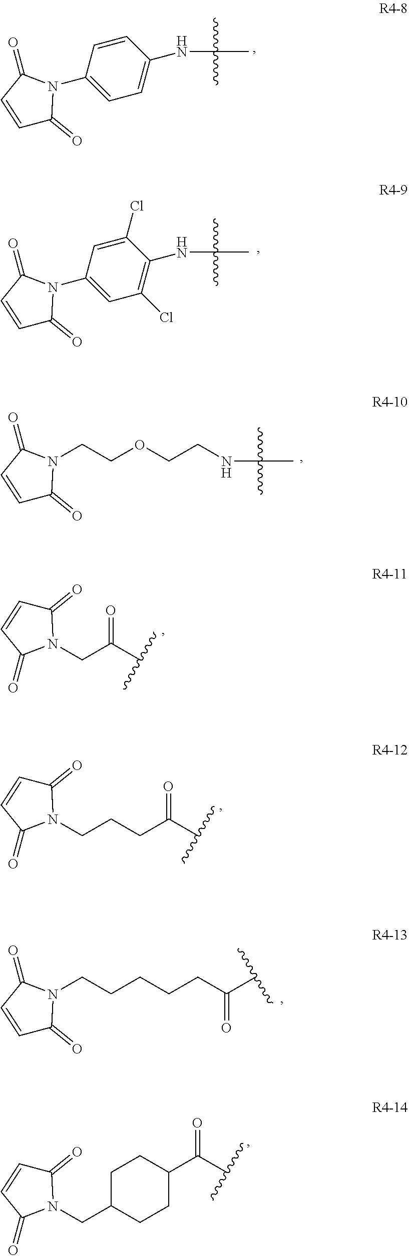

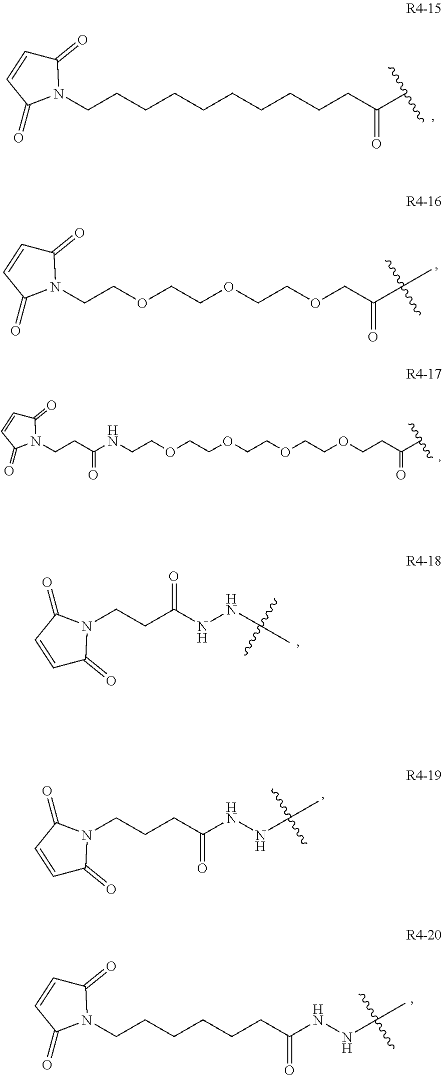

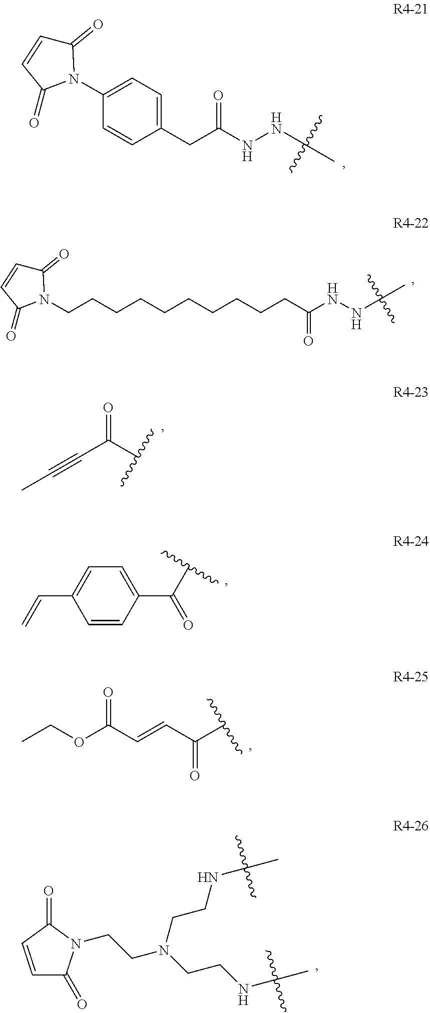

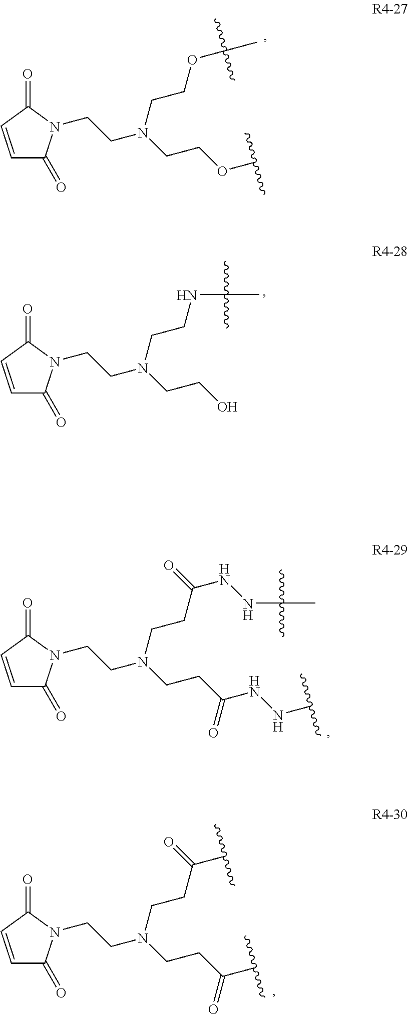

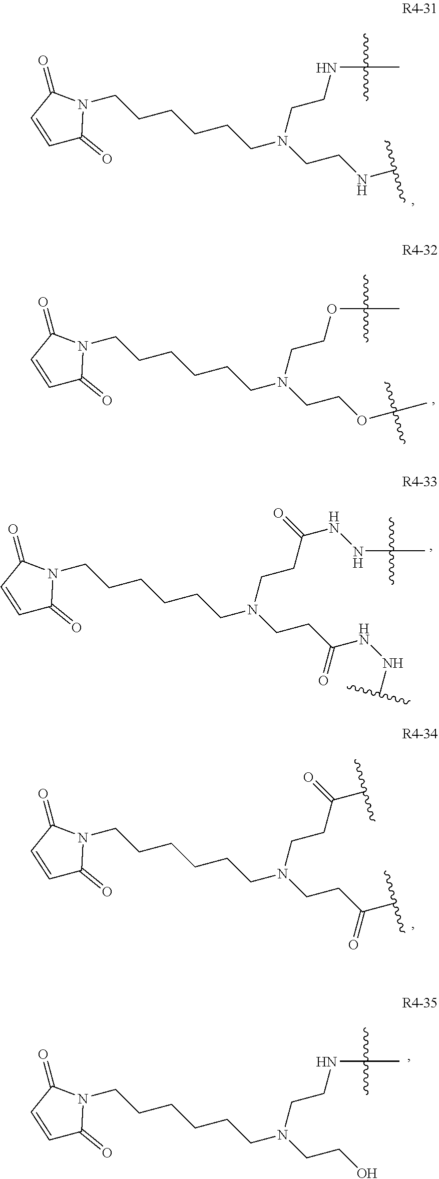

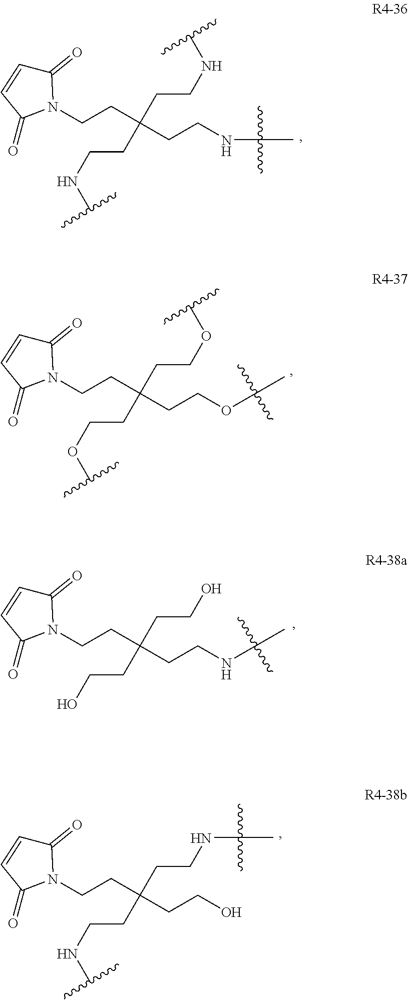

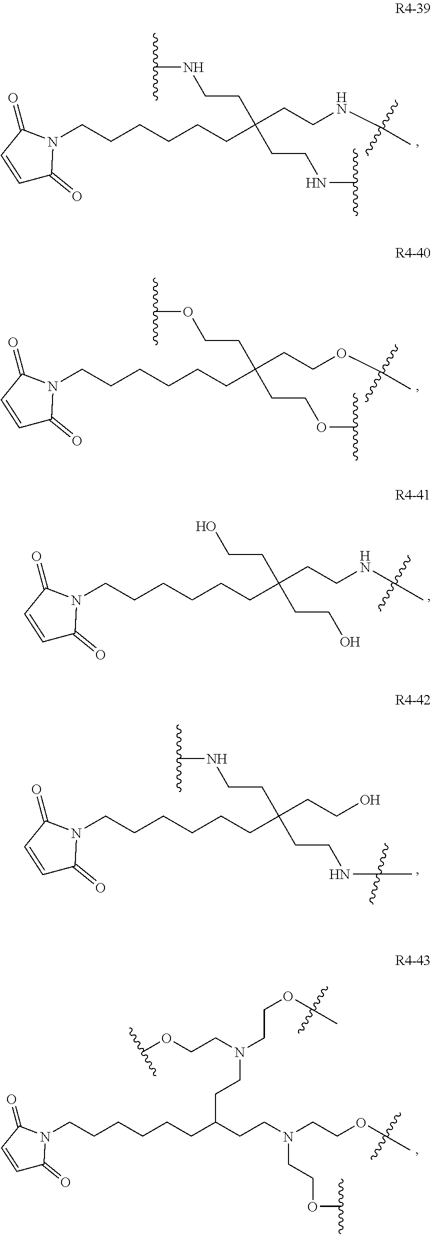

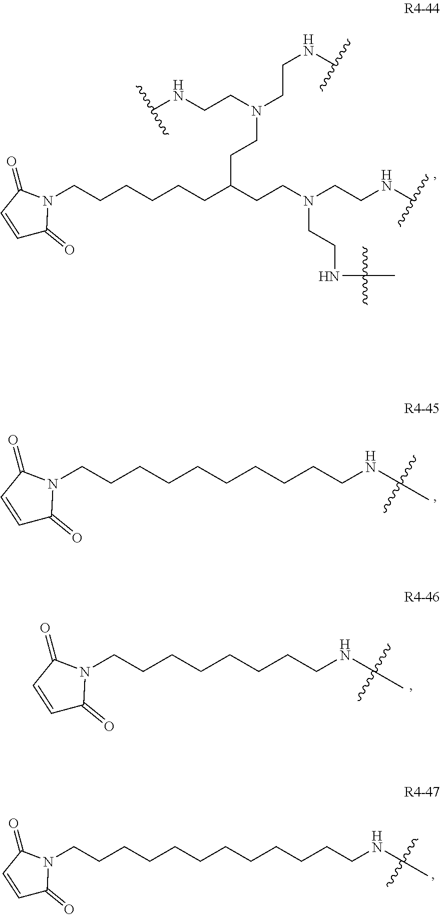

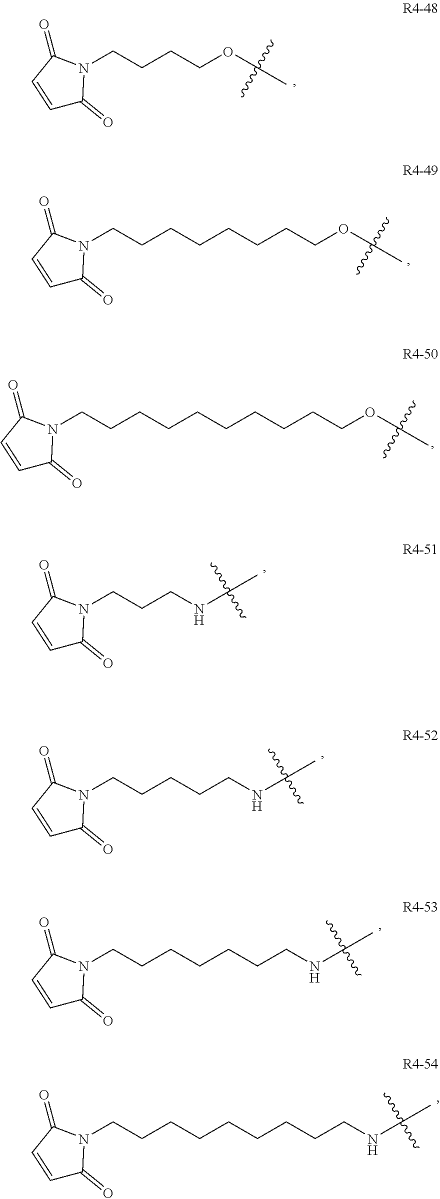

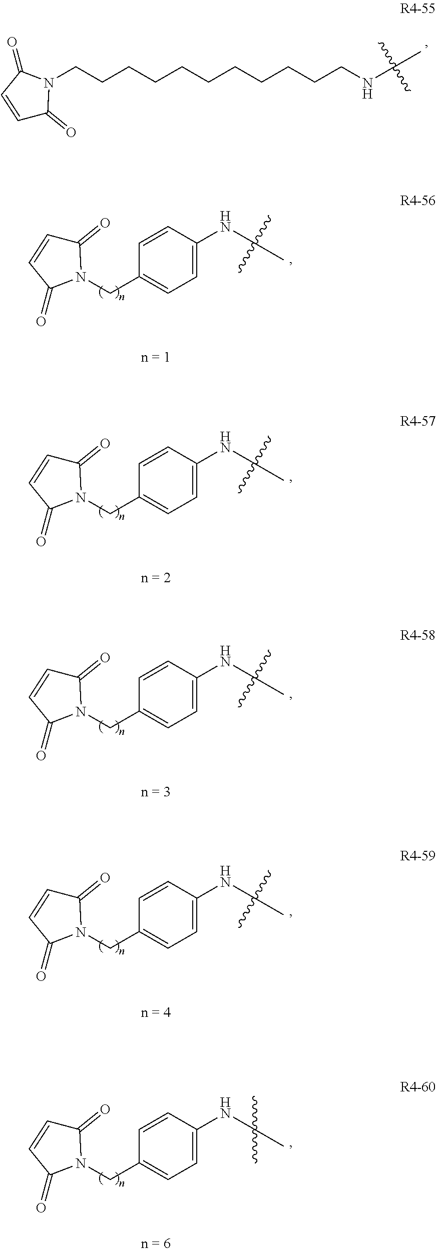

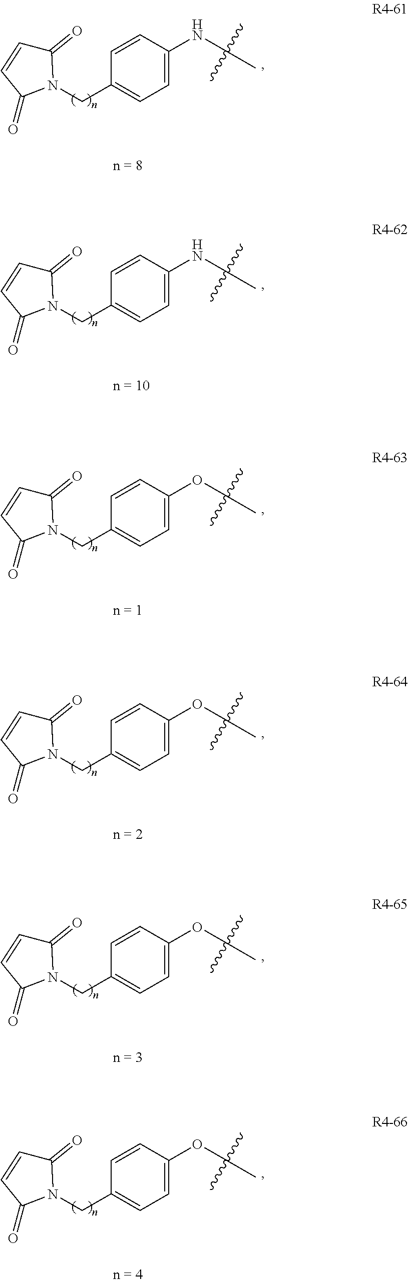

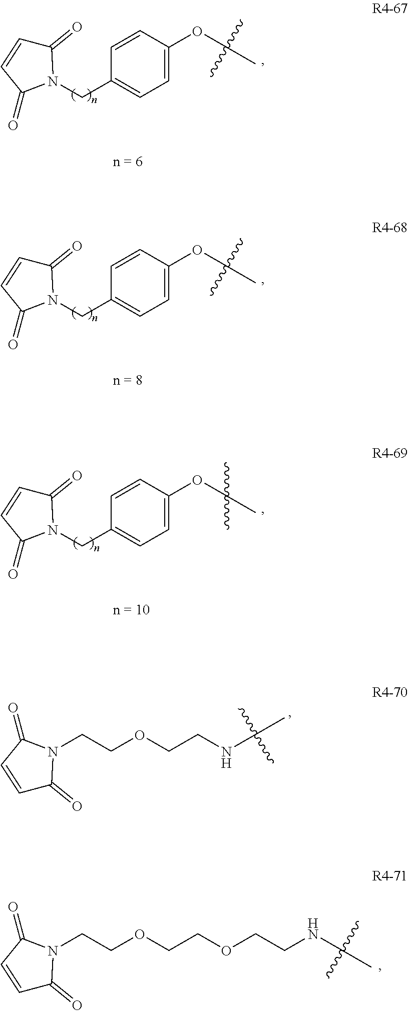

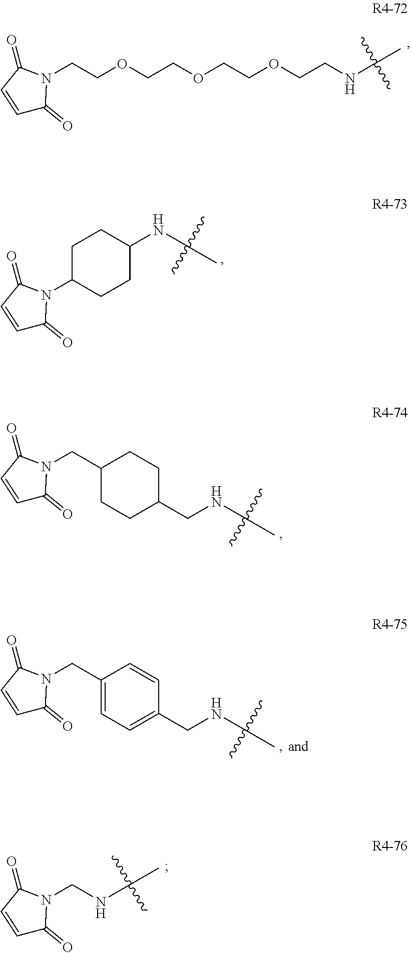

wherein the wave line indicates the position of R4 linking to R3.

In some embodiments, R4 is represented by:

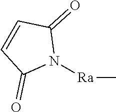

wherein:
Ra is selected from the group consisting of C₁₋₁₂ alkyl, C₁₋₁₂ alkoxy, C₁₋₁₂alkylcarbonyl, phenoxy or phenyl amino optionally substituted by one or two halogens, C₁₋₁₂alkylamino, C₁₋₁₂alkoxy-C₁₋₁₂alkylamino, C₁₋₁₂alkylcarbonyloxy, C₁₋₁₂alkyl-C₃₋₈cycloalkylcarbonyloxy, (C₁₋₄alkyl-O)ₚ—C₁₋₁₂alkylcarbonyloxy, C₁₋₁₂alkylcarbonylamino-(C₁₋₄alkyl-O)ₚ—C₁₋₁₂alkylcarbonyloxy, C₁₋₁₂alkylcarbonylamino and phenyl-C₁₋₁₂alkylcarbonylamino;
p is an integer in a range of 1 to 10, such as an integer in a range of 1 to 5;
wherein R3 links to R4 via the Ra of R4, and to the thiol group of the cysteine of R5 via the maleimide group of R4.

In the conjugate of the present disclosure, R4 covalently links to R5 via the S of cysteine contained in R5. R1-R2 is cleaved from R3-R4-S-cys-R5 by a proteolytic enzyme or under an acidic condition of a pathologic microenvironment, and R3-R4-S-cys-R5 is released. Then R3 automatically sheds and R4-S-cys-R5 is released. The R4-S-cys-R5 can recover or promote the binding capacity of R5 to its ligand or receptor.

It should be understood that in the present disclosure, the wave line(s) used in each of the indicated formulae indicate the linking position of the group containing the wave line(s) to other groups, and all position numbers of the amino acid mentioned for amino acid residue of an antibody is based on Kabat numbering.

R5, as described above, represents a biomolecule with one or more amino acid residues mutated to cysteine. R5 in fact is a moiety of the biomolecule without the hydrogen atom of the thiol group of the introduced cysteine. Absence of the hydrogen atom of the thiol group allows R5 being regarded as a group to link to R4 of the present disclosure.

Conjugates of the present disclosure may be prepared by a method comprising reducing the mutant biomolecule by DTT, TCEP or other reducing agent; oxidizing by Cu₂SO₄, dehydroascorbic acid or other oxidizing agent; and then conjugating the oxidized biomolecule (R5) to R1-R2-R3-R4 in a liquid phase or solid phase condition. The final product may be collected in a liquid phase.

Therefore, in addition to the conjugate, the present disclosure also comprises the functional moiety, i.e., RT-R2-R3-R4; R2-R3-R4; R3-R4-S-cys-R5; R4-S-cys-R5; and the mutated biomolecule; wherein R1, R2, R3, R3, R5 and their linkage manner and the mutated biomolecule are defined as in any part or any embodiments of the present disclosure. In some embodiments, the functional moieties are shown by S1-S64. In the present disclosure, the —S-cys- indicates that R4 covalently links to R5 via the thiol group of cysteine introduced by mutation in R5. The R3-R4-S-cys-R5 is a conjugate produced by cleavage of R1-R2 by a proteolytic enzyme or under an acidic condition of a pathologic microenvironment. Generally, after separation of R3 from R2, the group of R3 previously linked to R2 forms a hydroxyl (—OH) or an amino group (—NH₂). R4-S-cys-R5 is a conjugate formed after automatic shedding of R3. Generally, after automatic shedding of R3, the group of R4 previously linked to R3 forms a hydroxyl (—OH) or an amino group (—NH₂).

The conjugate, the functional moiety, R2-R3-R4, R3-R4-S-cys-R5 and R4-S-cys-R5 as described herein may be synthesized by the methods known in the art. For example, they may be prepared according to the method described in Example 1 of the present application.

The present disclosure also includes a pharmaceutical composition which comprises the conjugate as described herein. The pharmaceutical composition may further comprise a pharmaceutically acceptable carrier. The carrier may be any pharmaceutically acceptable carrier or excipient, which may be varied according to the dosage form and administration mode. The pharmaceutically acceptable carrier is generally safe and non-toxic, and may comprise any known substance used in formulating a pharmaceutical composition in the pharmaceutical industry, including filler, diluent, coagulant, adhesive, lubricant, glidant, stabilizer, colorant, wetting agent, and disintegrant, etc. Suitable pharmaceutically acceptable carrier include sugars, such as lactose or sucrose, mannitol or sorbitol; cellulose formulation and/or calcium phosphate, such as tricalcium phosphate or calcium hydrogen phosphate; amylum, including corn starch, wheat starch, rice starch, potato starch, gelatin, tragacanth, methyl cellulose, hydroxylpropylmethyl cellulose, sodium carboxyl methyl cellulose and/or polyvinylpyrrolidone; silica, talc, stearic acid or salt thereof, such as magnesium stearate or calcium stearate; and/or polyethylene glycol; and the like. When selecting a pharmaceutically acceptable carrier, the main consideration is the administration mode of the pharmaceutical composition. This is well known in the art.

The pharmaceutical composition may comprise a conjugate in a therapeutically or in a prophylactically effective amount. The “effective amount” indicates that the amount of an ingredient is sufficient to produce a desired reaction. The specific effective amount will depend on various factors, such as the specific disease to be treated, the physical condition of the patient, such as body weight, age and sex, the duration time of treatment, the therapy co-administered (if any), and the specific formulation used. Generally, the “effective amount” as described herein is a conventional amount of the biomolecule. However, in some embodiments, the therapeutically or prophylactically effective amount of the conjugate contained in the present pharmaceutical composition may be lower than the conventional amount of the biomolecule but may produce better treatment or prevention effect, because the biomolecule is protected by a protective group from binding to its ligand or receptor before arriving at a pathologic microenvironment.

The pharmaceutical composition of the present disclosure may be formulated into various suitable dosage forms, including but not limited to tablet, capsule, injection, etc., and it can be administered via any suitable route to achieve the expected purpose. For example, it can be administered parenterally, subcutaneously, intravenously, muscularly, intraperitoneally, transdermally, orally, intrathecally, intracranially, nasally or externally. The dose of a drug may depend on age, health status and body weight of a patient, treatment carried out in parallel, and frequency of treatment, etc. The pharmaceutical composition of the present disclosure may be administered to any subject in need thereof, such as a mammal, especially a human being.

In a tumor patient, tumor cells or antigen-presenting cells (APC) bearing a tumor antigen partially or fully inhibit immunological killing of the tumor by a host via binding to T cells. However, the conjugate of the present disclosure is activated and released by a proteolytic enzyme, especially Legumain or granzyme, or under an acidic condition, in a pathologic microenvironment. For example, the conjugate of the present disclosure in which the biomolecule is IL2, anti-CD28 antibody or anti-PD-1 antibody and the like can selectively stimulate proliferation of T cell or enhance its function to secrete anti-tumor cytokines. Therefore, the conjugate of the present disclosure can effectively break through the immune barrier of an individual, arrive at a pathologic microenvironment and then be activated and released in the pathologic microenvironment. As a result, it can selectively promote proliferation or killing effect of T cells, etc., in a tumor or inflammatory microenvironment, thereby realizing low autoimmunity and high efficacy.

Therefore, each of the conjugates, R4-S-cys-R5 or mutated biomolecules disclosed in the present disclosure may be used for treating tumor or inflammation or can be used as an active ingredient for preparing a medicament for treating tumor or inflammation. The tumor or inflammation described herein can be any tumor or inflammation which is known to be treated by the biomolecule as described herein, including but not limited to a cancer in bladder, brain, breast, cervix, colon-rectum, esophagus, kidney, liver, lung, nasopharynx, pancreas, prostate, skin, stomach, uterus, ovary, testiculus and blood, etc. Specifically, the cancer includes bladder cancer, brain cancer, breast cancer, cervical cancer, colorectal cancer, esophageal cancer, renal cancer, liver cancer, lung cancer, nasopharyngeal cancer, pancreatic cancer, prostate cancer, skin cancer, gastric cancer, uterus cancer, ovarian cancer, testicular cancer and blood cancer.

Further included is a method for treating or preventing tumor or inflammation, comprising administering a subject in need thereof a therapeutically or prophylactically effective amount of a conjugate as described herein or a pharmaceutical composition thereof. The method may be used in combination with any known radiotherapy or immunotherapy.

It should be understood that the term “comprising” and “including” or similar expressions used in the present disclosure also means “consisting of” or the like. The sum of all weight percentages or volume percentages should be equal to 100%. Unless otherwise specified, various reagents and products used in the Examples are commercial products. Unless otherwise specified, the methods mentioned in the Examples were implemented according to the conventional technique. The following examples are not intended to limit the scope of the present disclosure.

Information of sequence is summarized below:

| SEQ | |
|---|---|
| ID NO: | Description |
| 1 | heavy chain of anti-PD-1 antibody |
| 2 | light chain of anti-PD-1 antibody |
| 3 | heavy chain of anti-PD-1 antibody |
| 4 | light chain anti-PD-1 antibody |
| 5 | heavy chain of anti-CTLA-4 antibody |
| 6 | light chain of anti-CTLA-4 antibody |
| 7 | heavy chain of anti-TNFα antibody |
| 8 | light chain of anti-TNFα antibody |
| 9 | heavy chain of anti-CD28 antibody |
| 10 | light chain of anti-CD28 antibody |
| 11 | amino acid sequence of wild type IL2 |
| 12 | amino acid sequence of wild type IL2 |
| 13 | Ipilimumab heavy chain |
| 14 | Ipilimumab light chain |
| 15 | Pembrolizumab heavy chain |
| 16 | Pembrolizumab light chain |
| 17 | Nivolumab heavy chain |
| 18 | Nivolumab light chain |
| 19 | Anti-human PD-1 antibody (WO 2017/124050 A1) heavy chain |
| 20 | Anti-human PD-1 antibody (WO 2017/124050 A1) light chain |
| 21 | Anti-mouse PD-1 antibody J43v2 heavy chain |
| 22 | Anti-mouse PD-1 antibody J43v2 light chain |
| 23 | Anti-mouse CTLA-4 9D9 antibody heavy chain |
| 24 | Anti-mouse CTLA-4 9D9 antibody light chain |
| 25 | Anti-human 4-1BB antibody heavy chain |
| 26 | Anti-human 4-1BB antibody light chain |
| 27 | Trastuzumab heavy chain |
| 28 | Trastuzumab light chain |
| 29 | Adalimumab heavy chain |
| 30 | Adalimumab light chain |
| 31 | Atezolizumab heavy chain |
| 32 | Atezolizumab light chain |
| 33 | Anti-human CD28 antibody heavy chain |
| 34 | Anti-human CD28 antibody light chain |
| 35 | Ipi-se008 light chain |
| 36 | Ipi-se010 light chain |
| 37 | Ipi-se009 light chain |
| 38 | Pem-se010 light chain |
| 39 | Pem-se009 light chain |
| 40 | Pem-se007 light chain |
| 41 | Niv-se001 heavy chain variable domain |
| 42 | Niv-se005 light chain |
| 43 | Niv-se007 light chain |
| 44 | Interleukin-2 with mutation |
| 45 | Interleukin-2 with mutation |
| 46 | Interleukin-2 with mutation |
| 47 | Interleukin-2 with mutation |
| 48 | Interleukin-2 with mutation |
| 49 | Interleukin-2 with mutation |
| 50 | Interleukin-2 with mutation |
| 51 | Interleukin-2 with mutation |
| 52 | Interleukin-2 with mutation |
| 53 | Interleukin-2 with mutation |
| 54 | Interleukin-10 with mutation |
| 55 | Interleukin-10 with mutation |
| 56 | Interleukin-10 with mutation |
| 57 | Interleukin-10 with mutation |
| 58 | Interleukin-10 with mutation |
| 59 | Interleukin-10 with mutation |
| 60 | Anti-human VEGF antibody heavy chain |
| 61 | Anti-human VEGF antibody light chain |
| 62 | Anti-human CD20 antibody heavy chain |
| 63 | Anti-human CD20 antibody light chain |
| 64 | VH of Atezolizumab |
| 65 | VL of Atezolizumab |
| 66 | VH of Cetuximab with mutation |
| 67 | VL of Cetuximab with mutation |
| 68 | VH of Ramucirumab with mutation |
| 69 | VL of Ramucirumab with mutation |
| 70 | Fusion protein of scFv of anti-Her2 antibody with CD3, with |
| | mutation |
| 71 | VH of anti Her2 antibody with mutation |
| 72 | VL of anti Her2 antibody with mutation |
| 73 | Fusion protein of scFvs of anti-Her2 antibody with anti-CD3 |
| | antibody |
| 74 | Fusion protein of scFvs of anti-Her2 antibody with anti-CD3 |
| | antibody |
| 75 | anti CD3 antibody |
| 76 | VH of Cetuximab |
| 77 | VL of Cetuximab |
| 78 | VH of Ramucirumab |
| 79 | VL of Ramucirumab |
| 80 | VH of anti Her2 antibody |
| 81 | VL of Her2 antibody |
| 82 | Fusion protein of scFvs of anti-Her2 antibody with anti-CD3 |
| | antibody, with mutation |
| 83 | Fusion protein of scFvs of anti-Her2 antibody with anti-CD3 |
| | antibody, with mutation |
| 84 | Sequence of R2 |
| 85 | Sequence of R2 |
| 86 | Sequence of R2 |
| 87 | forward primer |
| 88 | reverse primer |
| 89 | Light chain of Cetuximab |
| 90 | Light chain of Pertuzumab |
| 91 | Heavy chain of Pertuzumab |
| 92 | VH of Opdivo ® (nivolumab) |
| 93 | VL of Opdivo ® (nivolumab) |
| 94 | VH of Keytruda ® (pembrolizumab) |
| 95 | VL of Keytruda ® (pembrolizumab) |
| 96 | VL of Ipilimumab |
| 97 | VH of Ipilimumab |
| 98 | VL of Anti-human 4-1BB antibody |
| 99 | VH of Anti-human 4-1BB antibody |
| 100 | VL of Adalimumab |
| 101 | VH of Adalimumab |
| 102 | VH of Pembrolizumab |
| 103 | VL of Pembrolizumab |
| 104 | VL of Trastuzumab |
| 105 | VH of Trastuzumab |
| 106 | Interleukin-2 with mutation |

### Example 1: Synthesis of Chemical Structure of Activatable and Binding Arms

When R2 has an amino acid sequence of Ala-Ala-Asn and R3 is PABC (R3-5), the synthetic scheme is shown below:

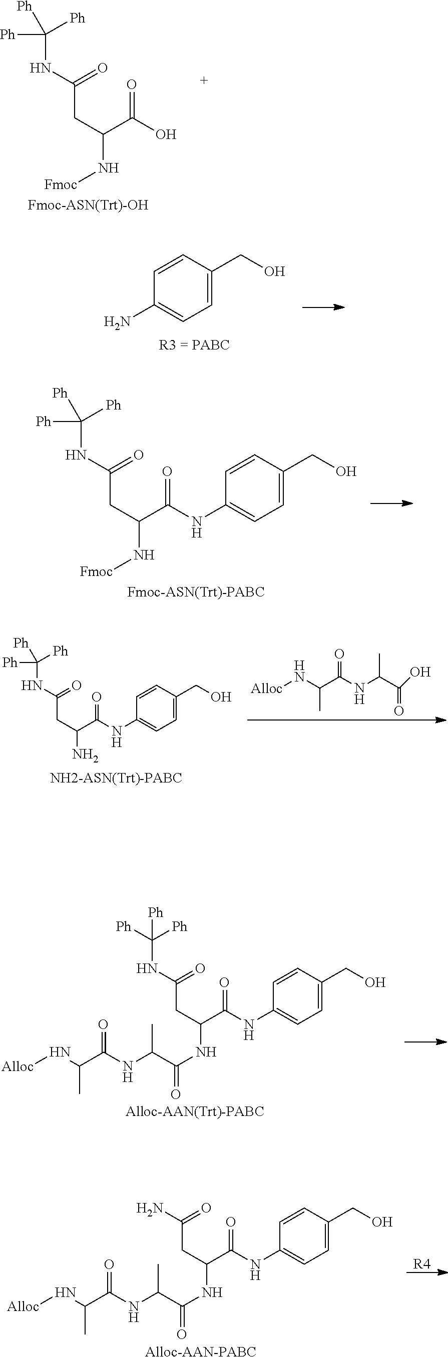

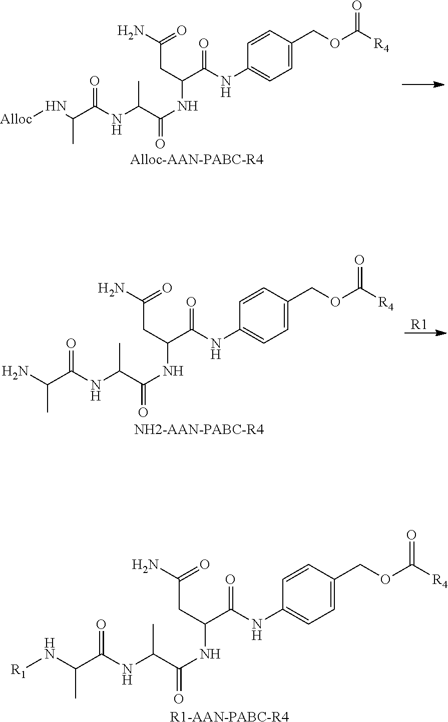

When R1 and R4 are different substituents, the following compounds shown in Table 1 were obtained.

TABLE 1
R1

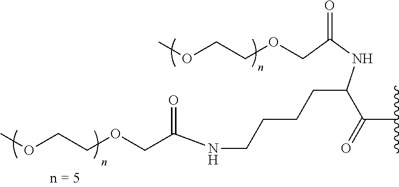

R1-1

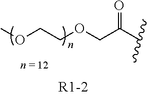

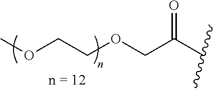

R1-2

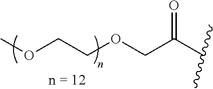

R1-2

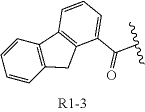 [SMILES: *C(=O)c1cccc2c1Cc1ccccc1-2]

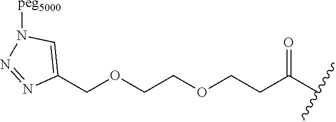

R1-4

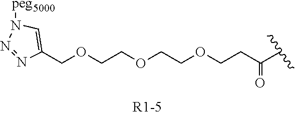

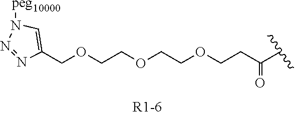

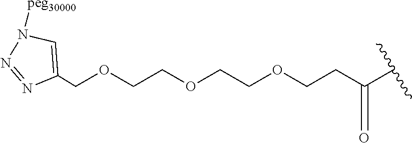

R1-7

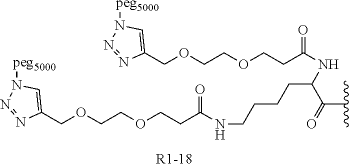

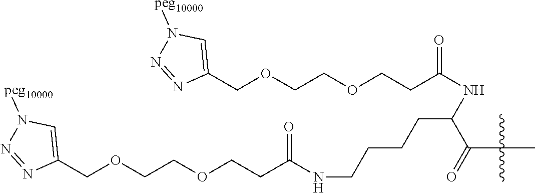

R1-19

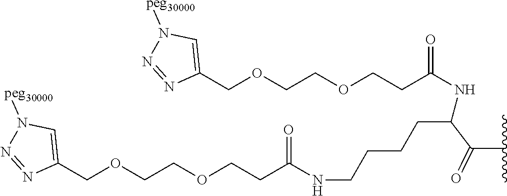

R1-20

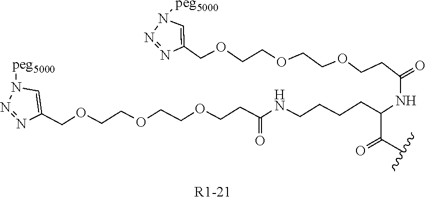

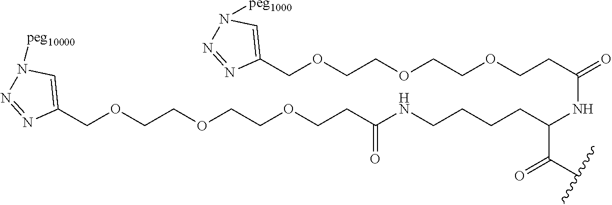

R1-22

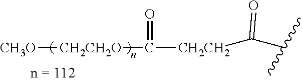

R1-12

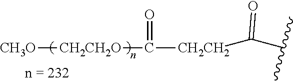

R1-13

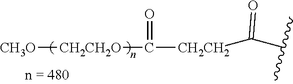

R1-15

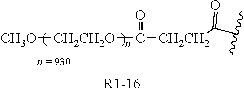

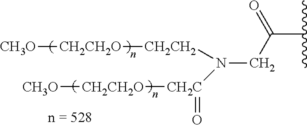

R1-17

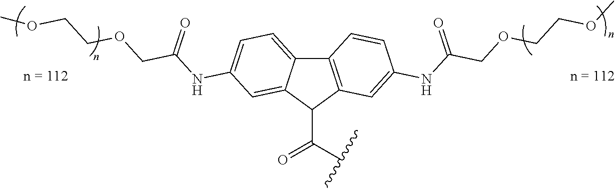

R1-28

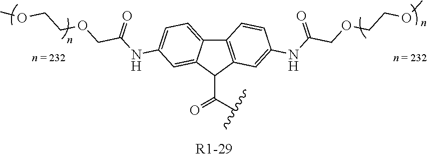

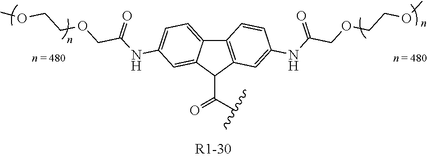

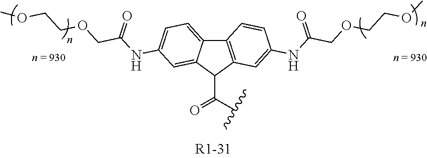

R4

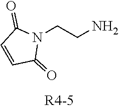

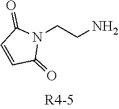

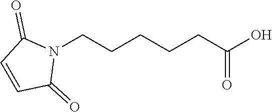 [SMILES: O=C(O)CCCCCN1C(=O)C=CC1=O]

R4-13

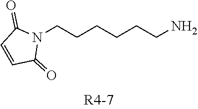 [SMILES: NCCCCCCN1C(=O)C=CC1=O]

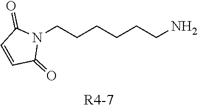 [SMILES: NCCCCCCN1C(=O)C=CC1=O]

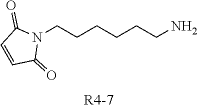 [SMILES: NCCCCCCN1C(=O)C=CC1=O]

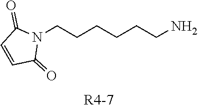 [SMILES: NCCCCCCN1C(=O)C=CC1=O]

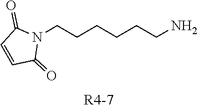 [SMILES: NCCCCCCN1C(=O)C=CC1=O]

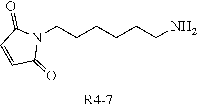 [SMILES: NCCCCCCN1C(=O)C=CC1=O]

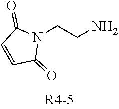

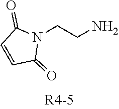

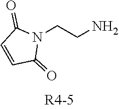

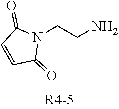

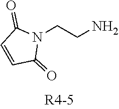

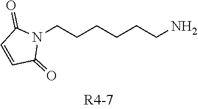 [SMILES: NCCCCCCN1C(=O)C=CC1=O]

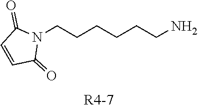 [SMILES: NCCCCCCN1C(=O)C=CC1=O]

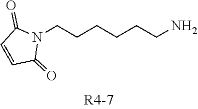 [SMILES: NCCCCCCN1C(=O)C=CC1=O]

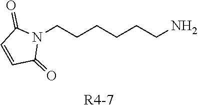 [SMILES: NCCCCCCN1C(=O)C=CC1=O]

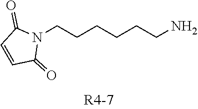 [SMILES: NCCCCCCN1C(=O)C=CC1=O]

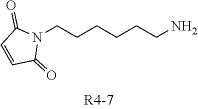 [SMILES: NCCCCCCN1C(=O)C=CC1=O]

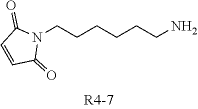 [SMILES: NCCCCCCN1C(=O)C=CC1=O]

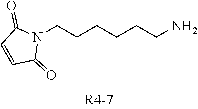 [SMILES: NCCCCCCN1C(=O)C=CC1=O]

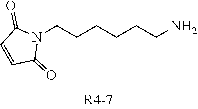 [SMILES: NCCCCCCN1C(=O)C=CC1=O]

Compound

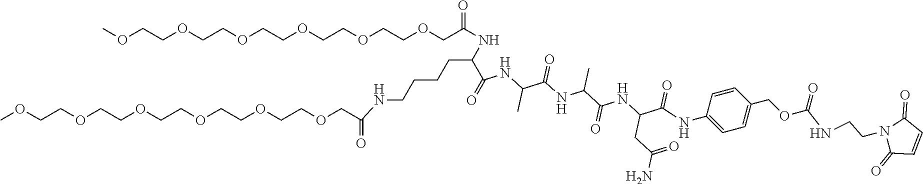 [SMILES: COCCOCCOCCOCCOCCOCC(=O)NCCCCC(NC(=O)COCCOCCOCCOCCOCCOC)C(=O)NC(C)C(=O)NC(C)C(=O)NC(CC(N)=O)C(=O)Nc1ccc(COC(=O)NCCN2C(=O)C=CC2=O)cc1]

S1

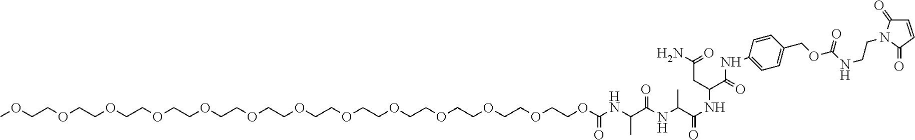 [SMILES: COCCOCCOCCOCCOCCOCCOCCOCCOCCOCCOCCOCCOC(=O)NC(C)C(=O)NC(C)C(=O)NC(CC(N)=O)C(=O)Nc1ccc(COC(=O)NCCN2C(=O)C=CC2=O)cc1]

S3

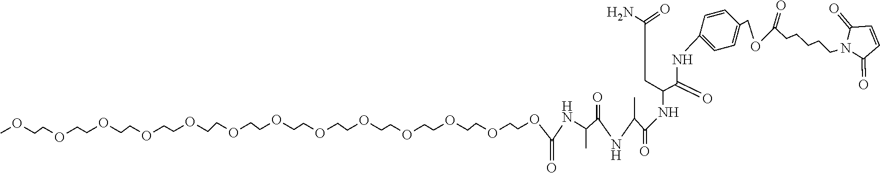 [SMILES: COCCOCCOCCOCCOCCOCCOCCOCCOCCOCCOCCOCCOC(=O)NC(C)C(=O)NC(C)C(=O)NC(CC(N)=O)C(=O)Nc1ccc(COC(=O)CCCCCN2C(=O)C=CC2=O)cc1]

S7

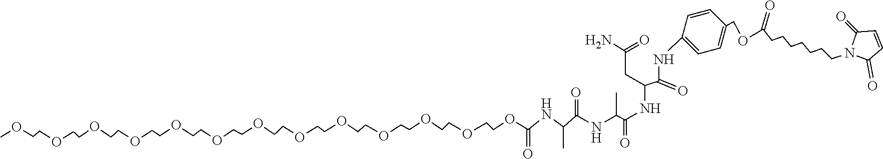 [SMILES: COCCOCCOCCOCCOCCOCCOCCOCCOCCOCCOCCOCCOC(=O)NC(C)C(=O)NC(C)C(=O)NC(CC(N)=O)C(=O)Nc1ccc(COC(=O)CCCCCCCN2C(=O)C=CC2=O)cc1]

S-9

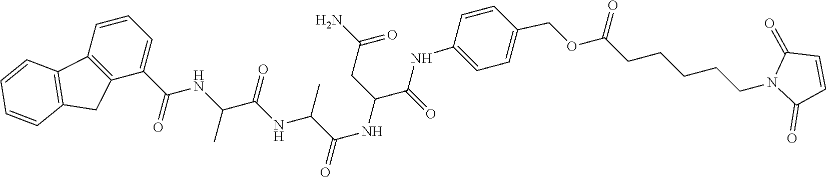 [SMILES: CC(NC(=O)c1cccc2c1Cc1ccccc1-2)C(=O)NC(C)C(=O)NC(CC(N)=O)C(=O)Nc1ccc(COC(=O)CCCCCN2C(=O)C=CC2=O)cc1]

S11

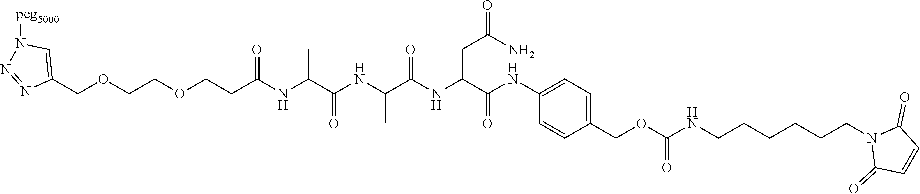

S13

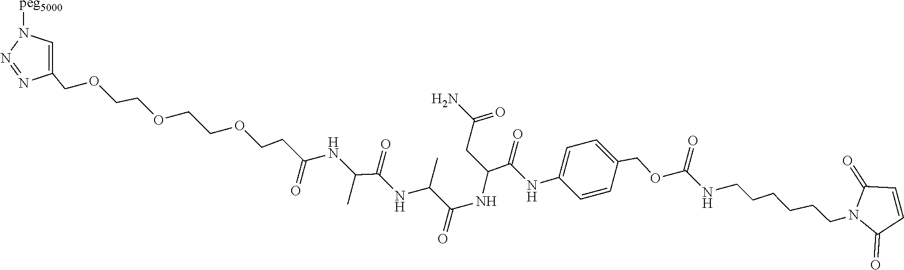

S15

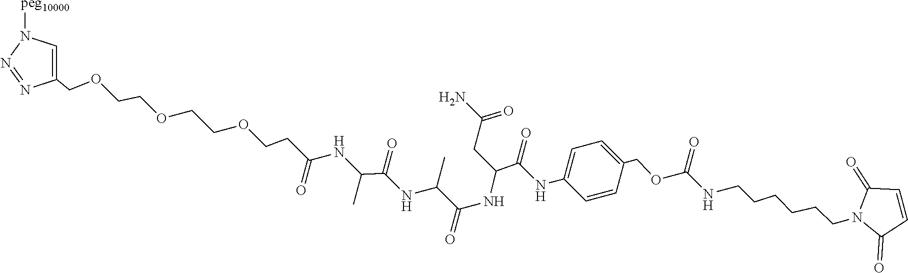

S17

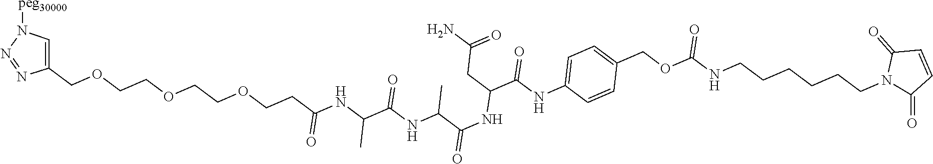

S25

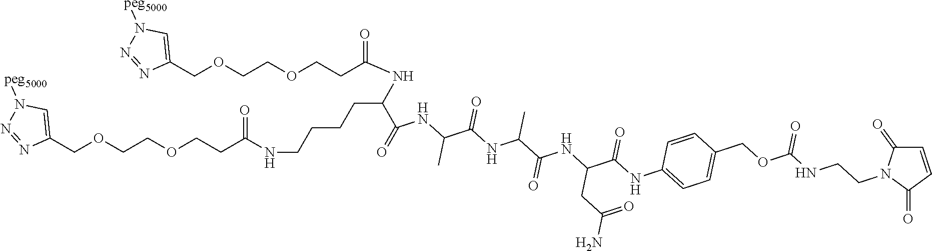

S29

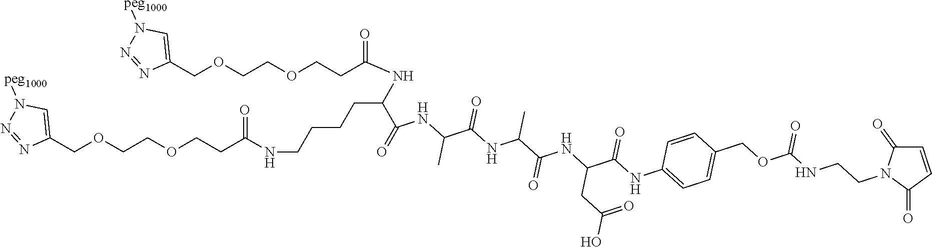

S31

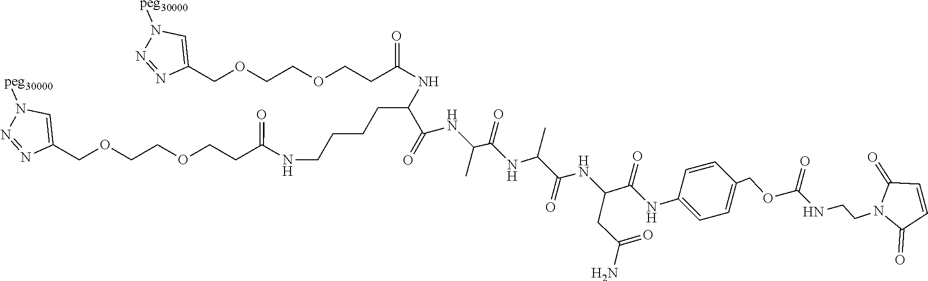

S33

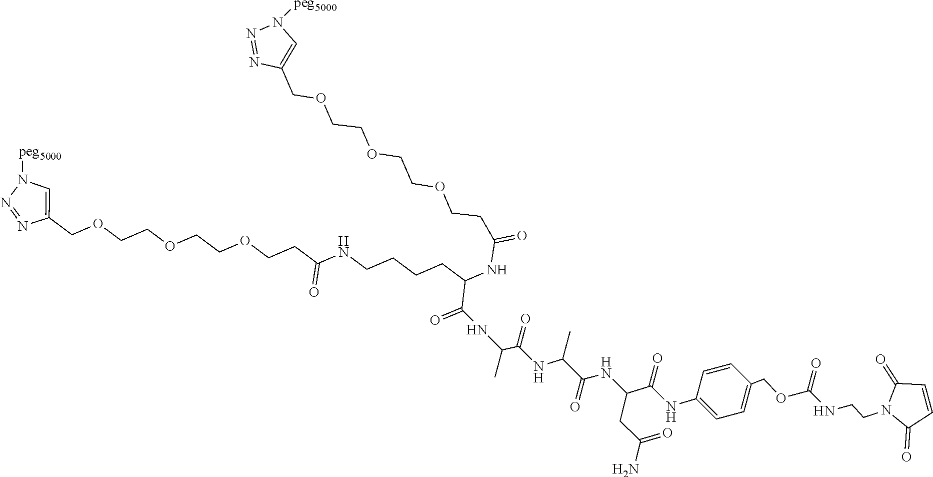

S35

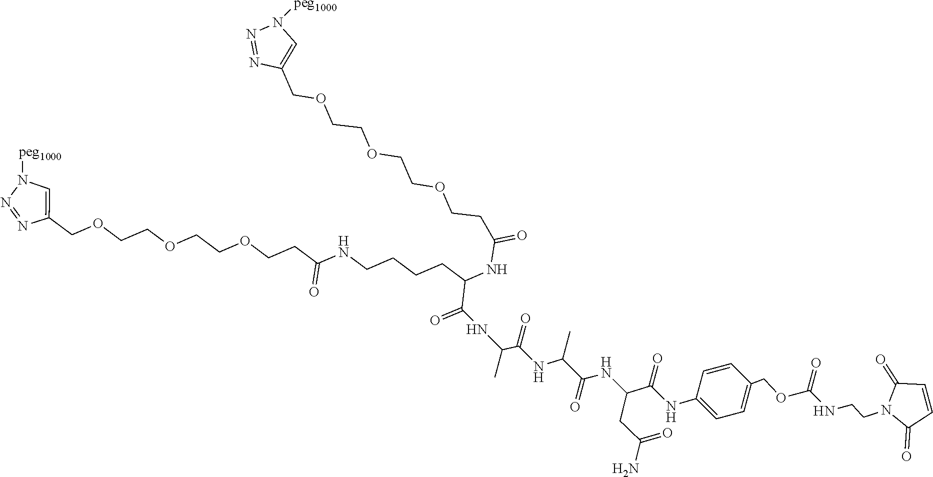

S37

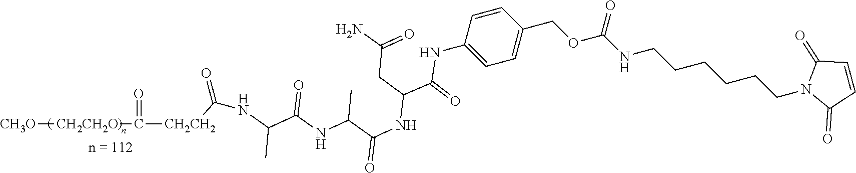

S39

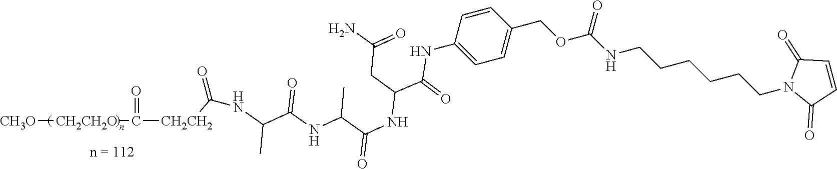

S41

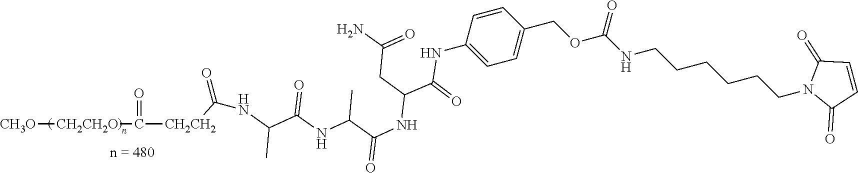

S43

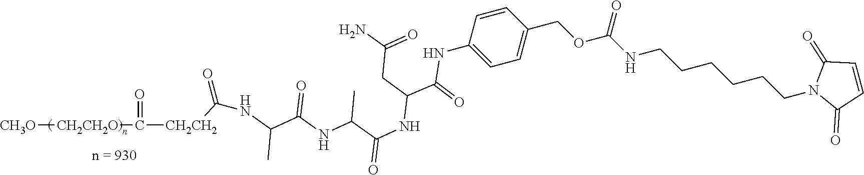

S45

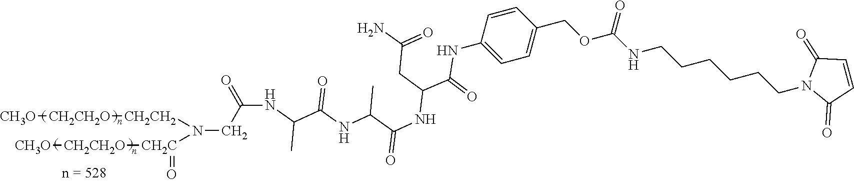

S47

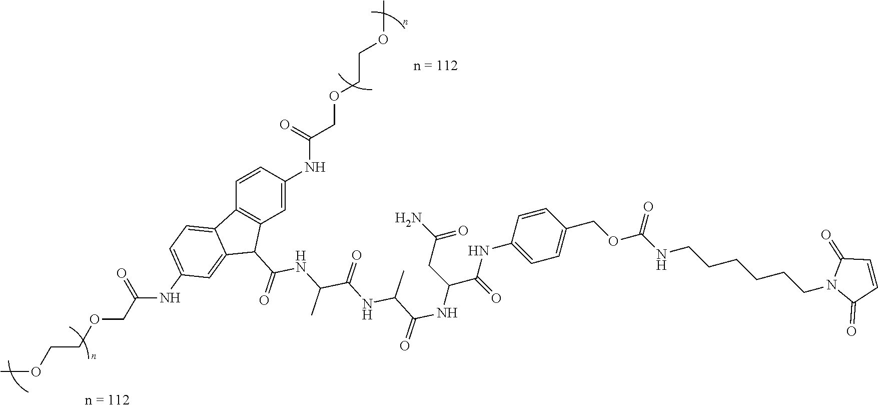

S57

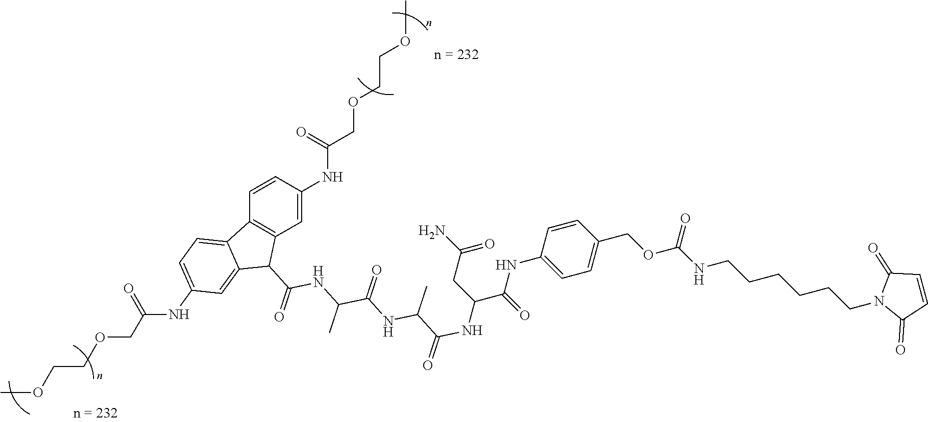

S59

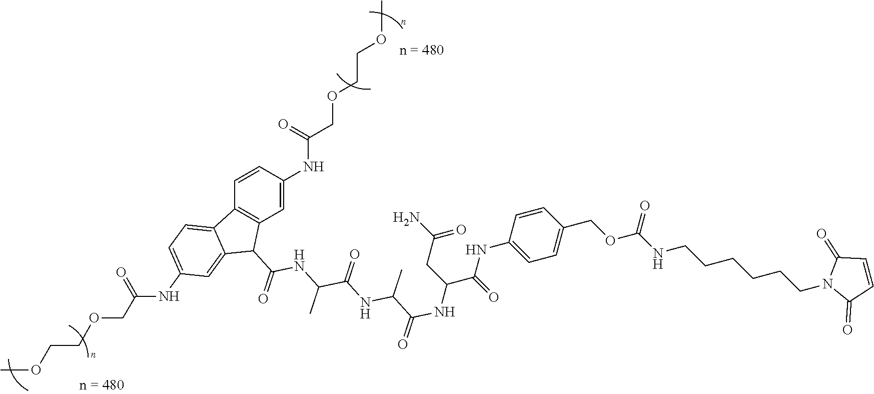

S61

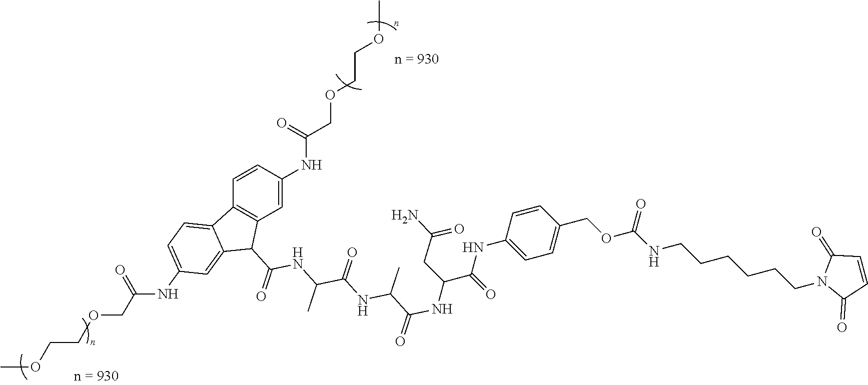

S63

As exemplified by S15, the specific synthesis process was shown below:

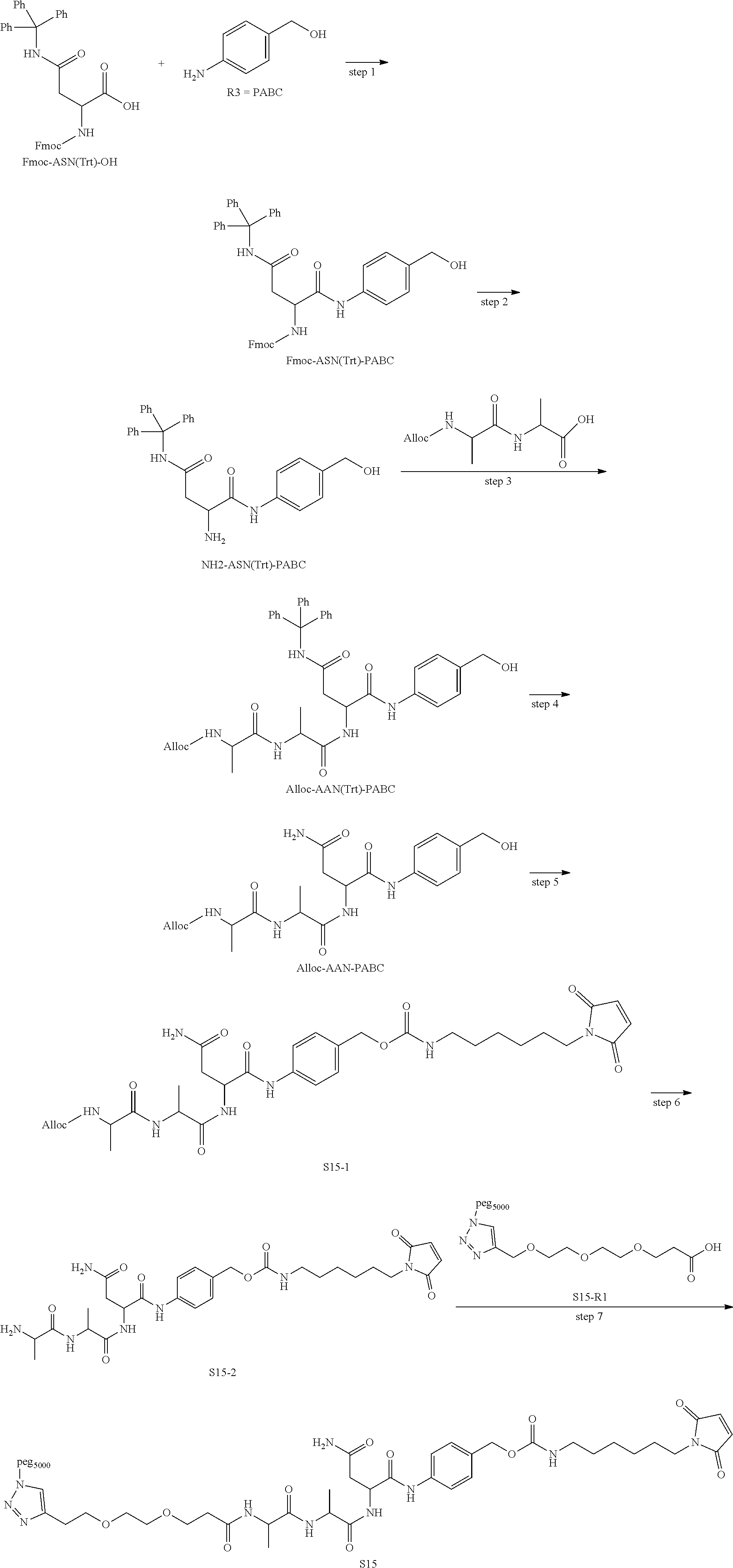

1) Fmoc-Asn(Trt)-OH (20 g, 0.03 mol), 2-(7-azabenzotriazole)-N,N,N′,N′-tetramethyluronium hexafluophosphate (HATU) (15 g, 0.04 mol), and DMF (200 mL) were added to a three-necked flask and stirred for 30 min. p-Aminobenzyl alcohol (4.1 g, 0.03 mol) and N,N-diisopropyl ethylamine (8.7 g, 0.06 mol) were added at 0 °C, respectively and then stirred at room temperature for 3 hours. Most of DMF were removed by rotary evaporation. The residue was dissolved in acetic acetate (200 mL), washed with saturated ammonia chloride solution and saturated sodium chloride solution subsequently, dried over anhydrous sodium sulfate followed by filtration. The solvents were removed by evaporation. The crude product was beaten to obtain a white solid Fmoc-Asn(Trt)-PABC (21.3 g; Yield: 90%).

2) Fmoc-Asn(Trt)-PABC (16.0 g, 22 mmol) was dissolved in N,N-dimethyl formamide (80 mL). Piperidine (30 mL) were added and then stirred at room temperature for 2 hours. The solvents were removed by evaporation under reduced pressure. The residue was dried under high vacuum in a vacuum oven to remove a small amount of piperidine to produce 9.8 g pale yellow solid NH₂-Asn(Trt)-PABC which could be used in the next step without purification.

3) Alloc-Ala-Ala-OH (5.0 g, 20.4 mmol), benzotriazole-N,N,N′,N′-tetramethyluronium hexafluophosphate (HBTU) (11.6 g, 30.6 mmol) and DMF (50 mL) were added into a three-necked flask and stirred for 30 min in an ice bath. NH₂-Asn(Trt)-PABC (9.8 g, 20.4 mmol) and N,N-diisopropyl ethylamine (7.89 g, 61.2 mmol) were added respectively at 0 °C and then stirred at room temperature overnight. The solvents were removed by evaporation under reduced pressure. The residue was dissolved in acetic acetate (200 mL), washed with saturated ammonia chloride solution and saturated sodium chloride solution subsequently, dried over anhydrous sodium sulfate followed by filtration. The solvents were removed by evaporation. The resulting crude product was subjected to recrystallization to obtain a white solid Alloc-AAN(Trt)-PABC (13.0 g; Yield: 90%).

4) Alloc-AAN(Trt)-PABC (10.0 g, 14.2 mmol) was dissolved in dichloromethane (100 mL). Triluoroacetic acid (20 mL) were added and then stirred at room temperature for 4 hours. After washing with water and fraction, the organic phase was dried over anhydrous sodium sulfate. The solvents were removed by evaporation under reduced pressure and the residual trifluoroacetic acid was removed by evaporation under high vacuum. The crude product was isolated by column chromatography to obtain Alloc-AAN-PABC (5.9 g; Yield: 89%).

5) Alloc-AAN-PABC (467 mg, 1.01 mmol) dissolved in dichloromethane (10 mL) were added to a three-necked flask. 4-Nitrophenyl chloroformate (406 mg, 2.02 mmol) in dichloromethane and pyridine (160 mg, 2.03 mmol) in dichloromethane were dropped into the flask, respectively, in an ice bath and under nitrogen gas protection and then stirred at room temperature overnight. 1-(6-Aminohexyl)-1H-pyrolo-2,5-dione (235 mg, 1.2 mmol) were added in batches into the above solution and was allowed to react at room temperature for 4 hours. The reaction solution was dried by rotary evaporation. The resulting crude product was purified by silica gel column chromatography to obtain a white solid S15-1 (540 mg; Yield: 80%).

6) DMF (10 ml), S15-1 (208 mg, 0.31 mmol), acetic acid (274 mg, 4.65 mmol), triphenylphosphine palladium (72 mg, 0.062 mmol) and tributyltin hydride (1.17 g, 4.03 mmol) were added successively into a one-neck flask. After replacing the air in the flask with nitrogen gas, the mixture was stirred at room temperature until S15-1 is reacted completely. After the reaction was completed, DMF was removed by evaporation under reduced pressure. The crude product was isolated and purified by silica gel column chromatography to obtain S15-2 (white solid, 116 mg, Yield: 62%).

7) S15-R1 (940 mg, 0.18 mmol), benzotriazole-N,N,N′,N′-tetramethyluronium hexafluophosphate (HBTU) (95 mg, 0.25 mmol) and DMF (10 mL) were added to a three-necked flask, and then stirred in an ice bath for 30 min. Then compound S15-2 (110 mg, 0.18 mmol) and N,N-diisopropyl ethylamine (70 mg, 0.54 mmol) were added respectively at 0 °C and then stirred at room temperature overnight. The solvents were removed by evaporation under reduced pressure. The crude product was isolated and purified by silica gel column chromatography to obtain a white solid (418 mg; Yield: 40%), which was Compound S15.

When R2 has an amino acid sequence of Ala-Ala-Asp and R3 is PABC, the synthetic scheme is shown below:

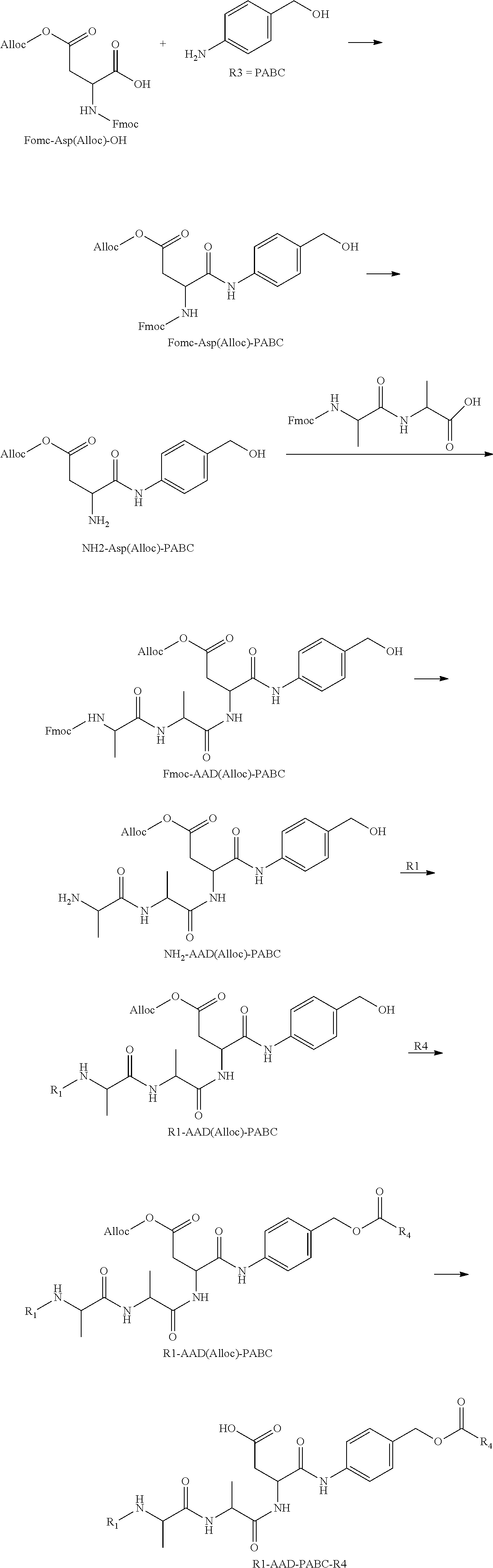

When R1 and R4 are different substituents, the following compounds in Table 2 were obtained.

TABLE 2
R1

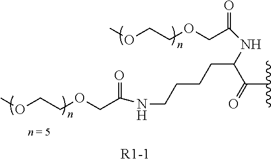

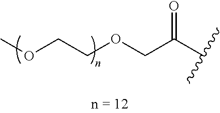

R1-2

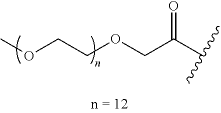

R1-2

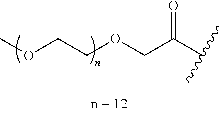

R1-2

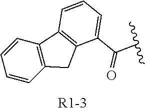 [SMILES: *C(=O)c1cccc2c1Cc1ccccc1-2]

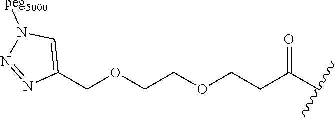

R1-4

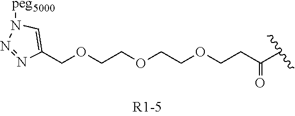

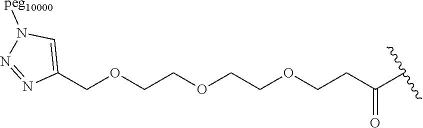

R1-6

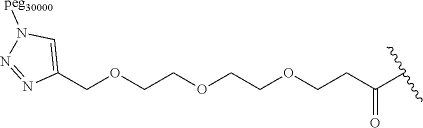

R1-7

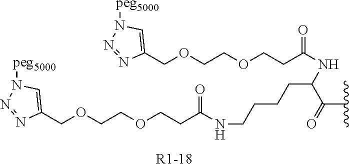

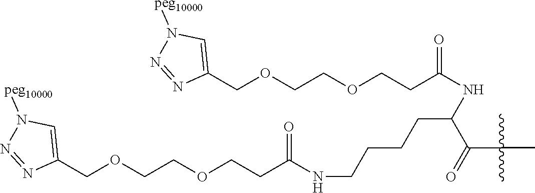

R1-19

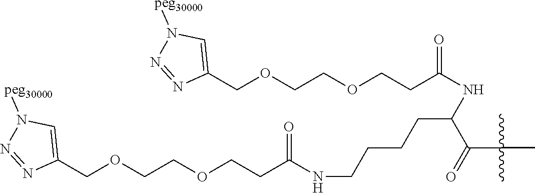

R1-20

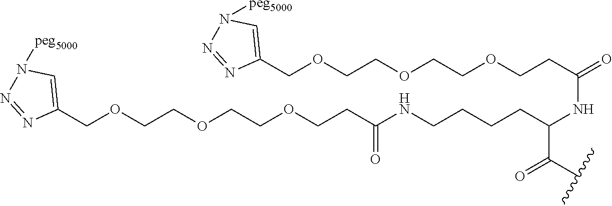

R1-21

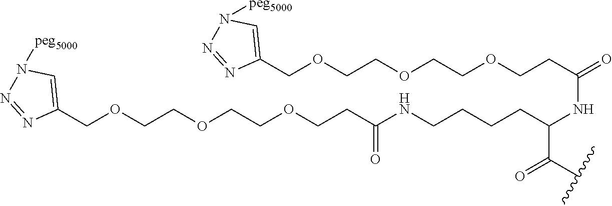

R1-22

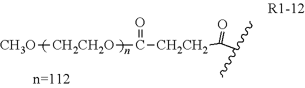

R1-12

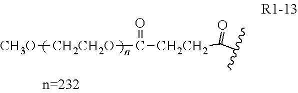

R1-13

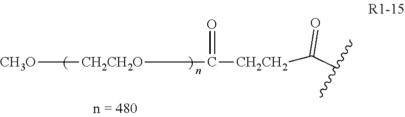

R1-15

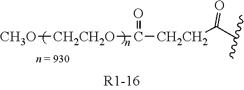

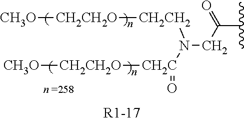

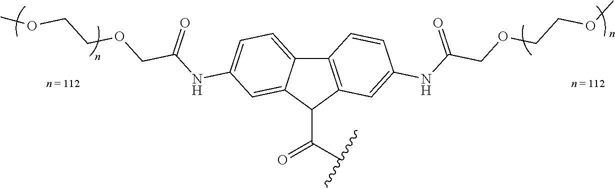

R1-28

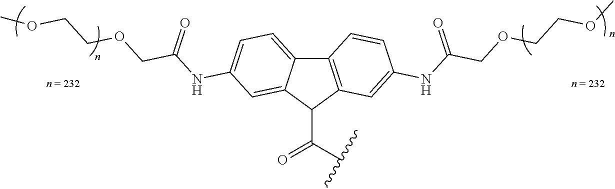

R1-29

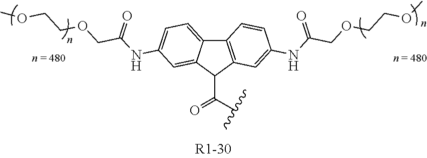

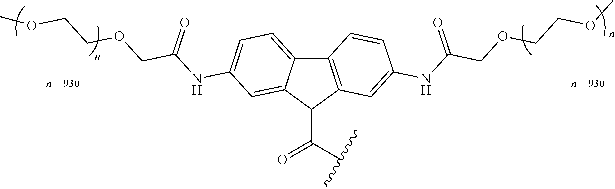

R1-31
R4

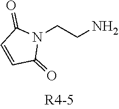

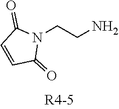

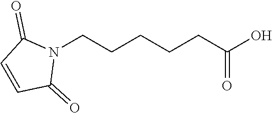 [SMILES: O=C(O)CCCCCN1C(=O)C=CC1=O]

R4-13

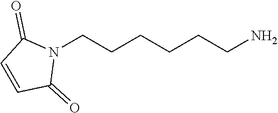 [SMILES: NCCCCCCN1C(=O)C=CC1=O]

R4-7

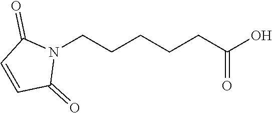 [SMILES: O=C(O)CCCCCN1C(=O)C=CC1=O]

R4-7

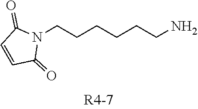 [SMILES: NCCCCCCN1C(=O)C=CC1=O]

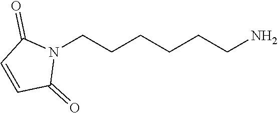 [SMILES: NCCCCCCN1C(=O)C=CC1=O]

R4-7

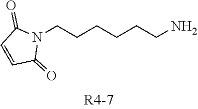 [SMILES: NCCCCCCN1C(=O)C=CC1=O]

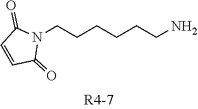 [SMILES: NCCCCCCN1C(=O)C=CC1=O]

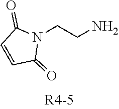

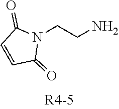

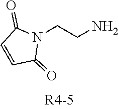

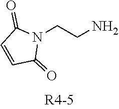

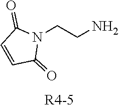

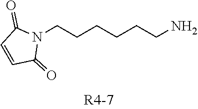 [SMILES: NCCCCCCN1C(=O)C=CC1=O]

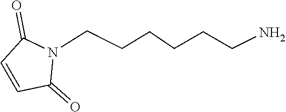 [SMILES: NCCCCCCN1C(=O)C=CC1=O]

R4-7

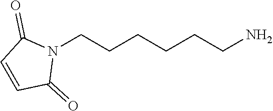 [SMILES: NCCCCCCN1C(=O)C=CC1=O]

R4-7

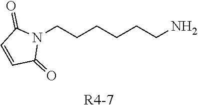 [SMILES: NCCCCCCN1C(=O)C=CC1=O]

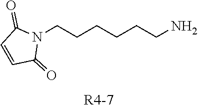 [SMILES: NCCCCCCN1C(=O)C=CC1=O]

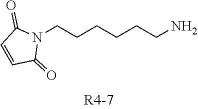 [SMILES: NCCCCCCN1C(=O)C=CC1=O]

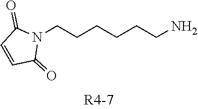 [SMILES: NCCCCCCN1C(=O)C=CC1=O]

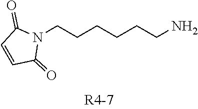 [SMILES: NCCCCCCN1C(=O)C=CC1=O]

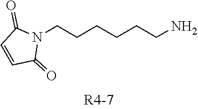 [SMILES: NCCCCCCN1C(=O)C=CC1=O]

Compound

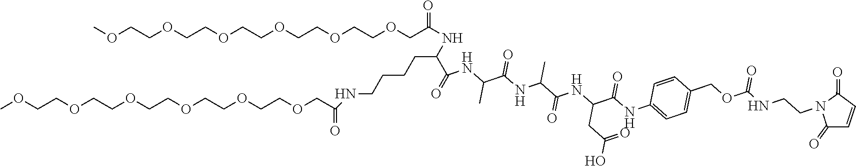 [SMILES: COCCOCCOCCOCCOCCOCC(=O)NCCCCC(NC(=O)COCCOCCOCCOCCOCCOC)C(=O)NC(C)C(=O)NC(C)C(=O)NC(CC(=O)O)C(=O)Nc1ccc(COC(=O)NCCN2C(=O)C=CC2=O)cc1]

S2

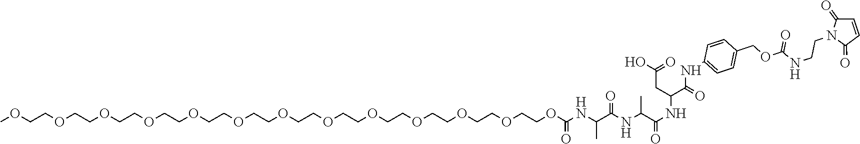 [SMILES: COCCOCCOCCOCCOCCOCCOCCOCCOCCOCCOCCOCCOC(=O)NC(C)C(=O)NC(C)C(=O)NC(CC(=O)O)C(=O)Nc1ccc(COC(=O)NCCN2C(=O)C=CC2=O)cc1]

S4

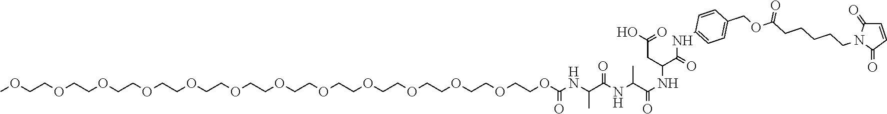 [SMILES: COCCOCCOCCOCCOCCOCCOCCOCCOCCOCCOCCOCCOC(=O)NC(C)C(=O)NC(C)C(=O)NC(CC(=O)O)C(=O)Nc1ccc(COC(=O)CCCCCN2C(=O)C=CC2=O)cc1]

S8

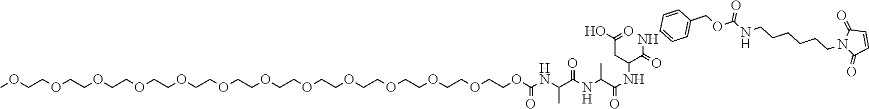 [SMILES: COCCOCCOCCOCCOCCOCCOCCOCCOCCOCCOCCOCCOC(=O)NC(C)C(=O)NC(C)C(=O)NC(CC(=O)O)C(=O)Nc1ccc(COC(=O)NCCCCCCN2C(=O)C=CC2=O)cc1]

S10

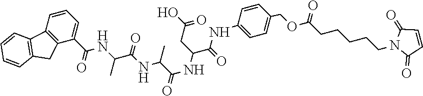 [SMILES: CC(NC(=O)c1cccc2c1Cc1ccccc1-2)C(=O)NC(C)C(=O)NC(CC(=O)O)C(=O)Nc1ccc(COC(=O)CCCCCN2C(=O)C=CC2=O)cc1]

S12

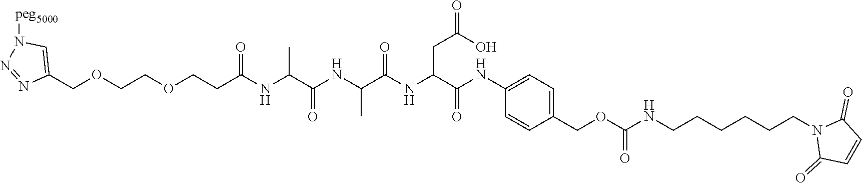

S14

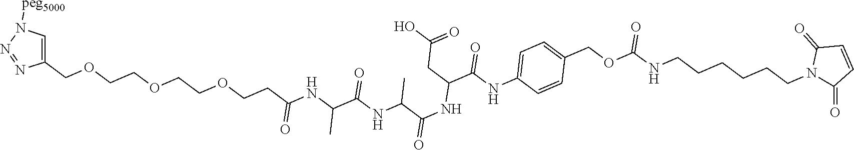

S16

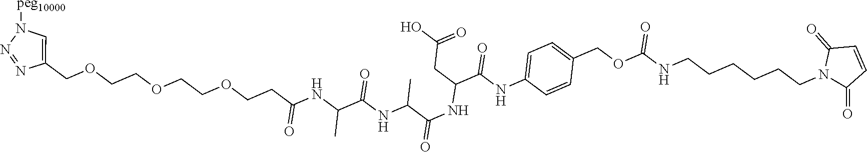

S18

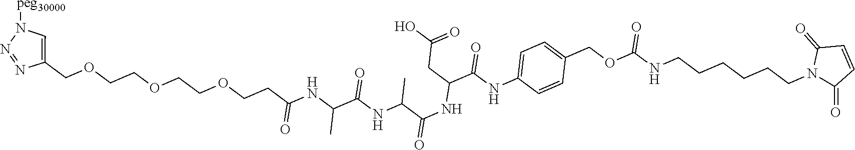

S26

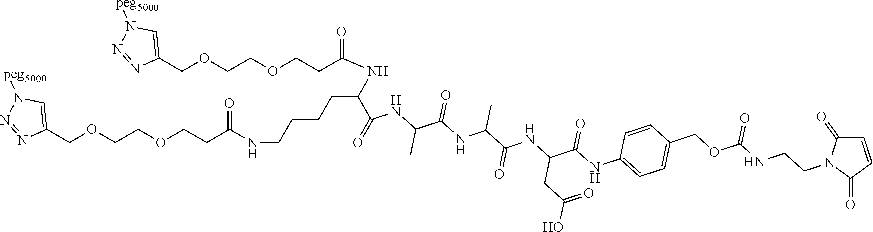

S30

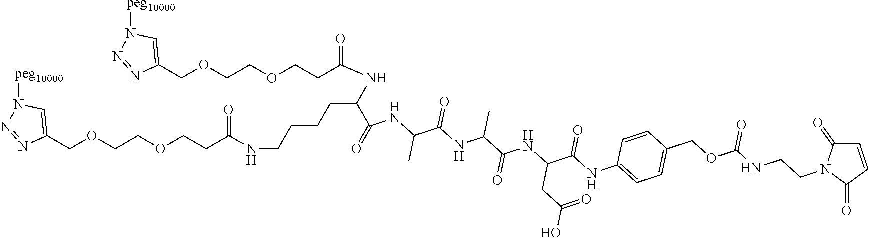

S32

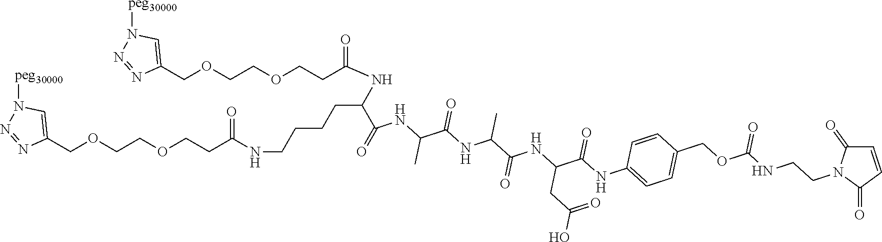

S34

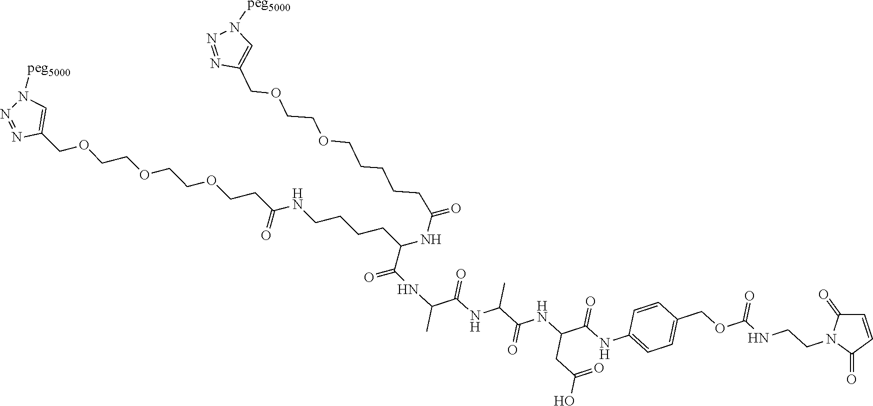

S36

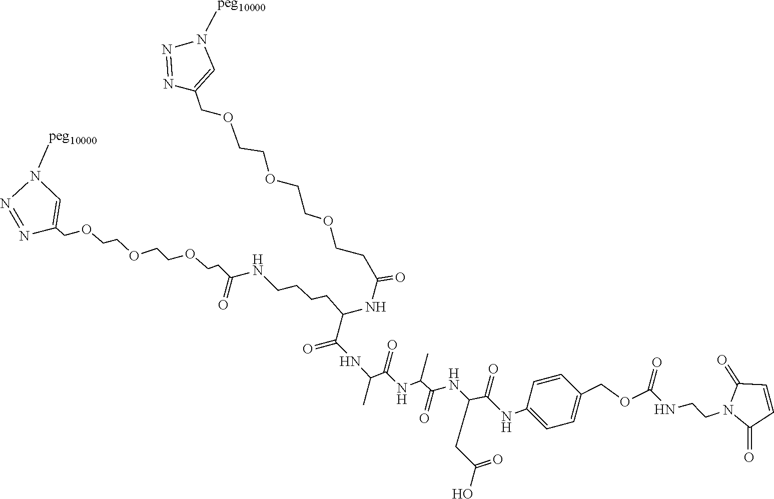

S38

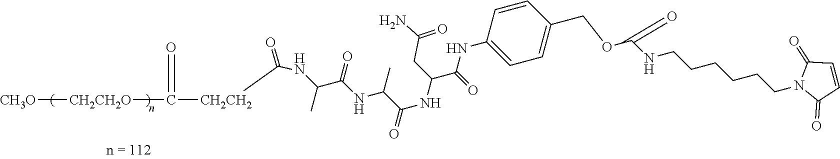

S40

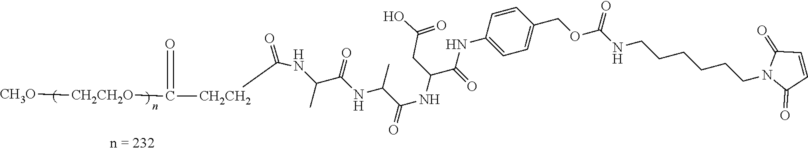

S42

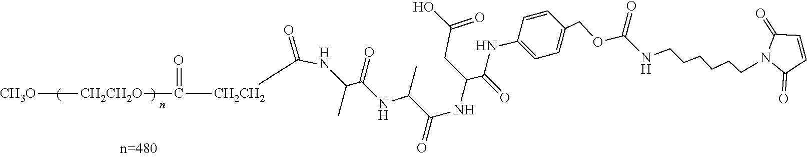

S44

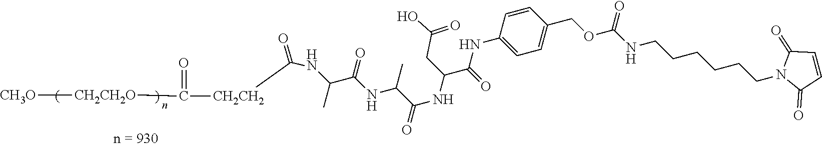

S46

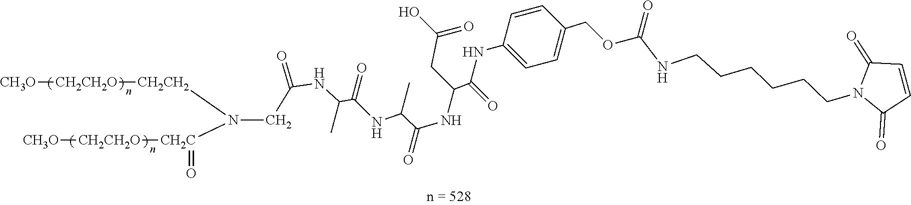

S48

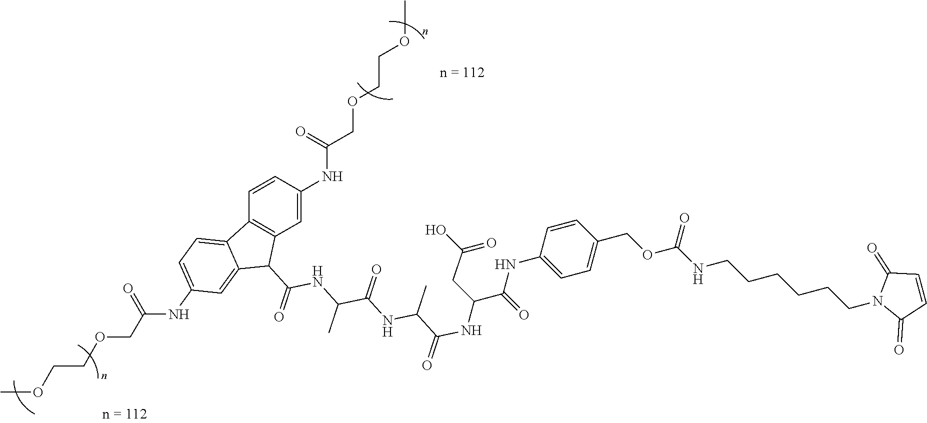

S58

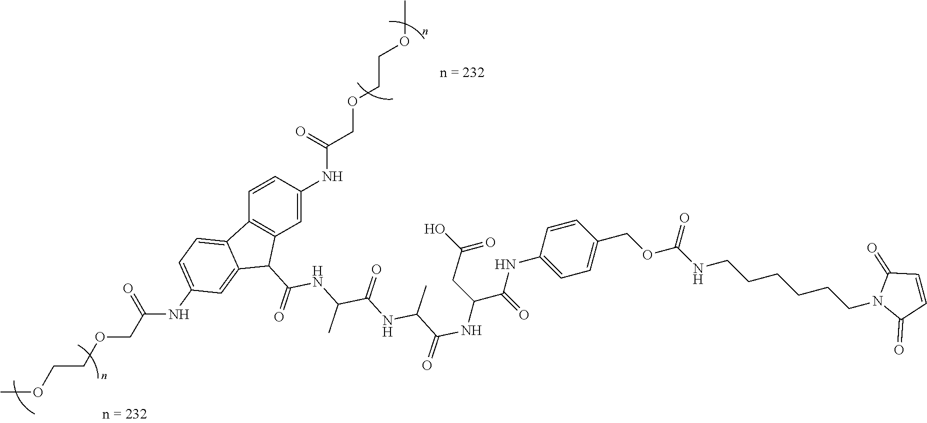

S60

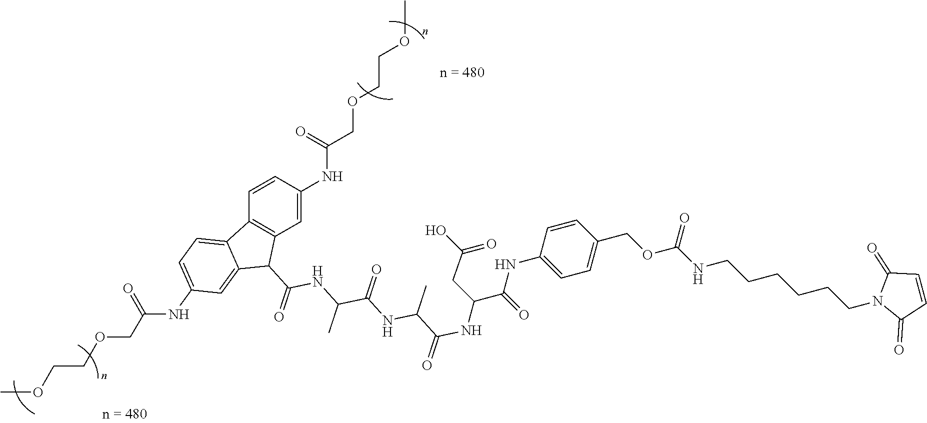

S62

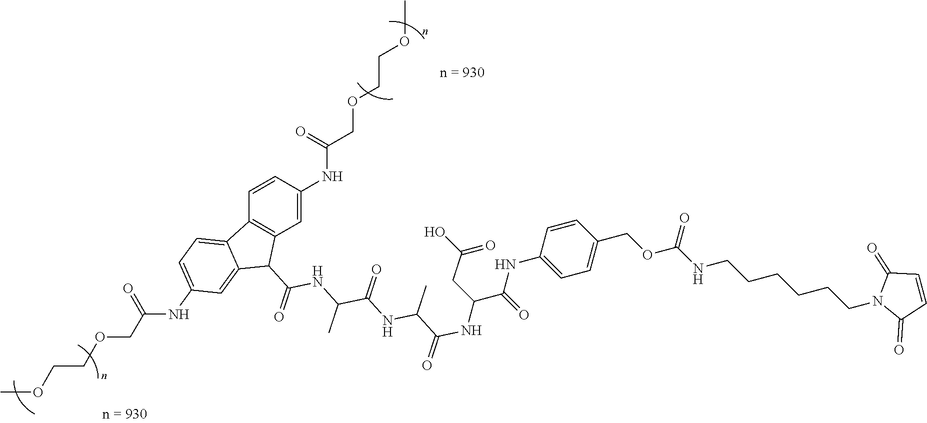

S64

As exemplified by S16, the specific synthesis process was shown below:

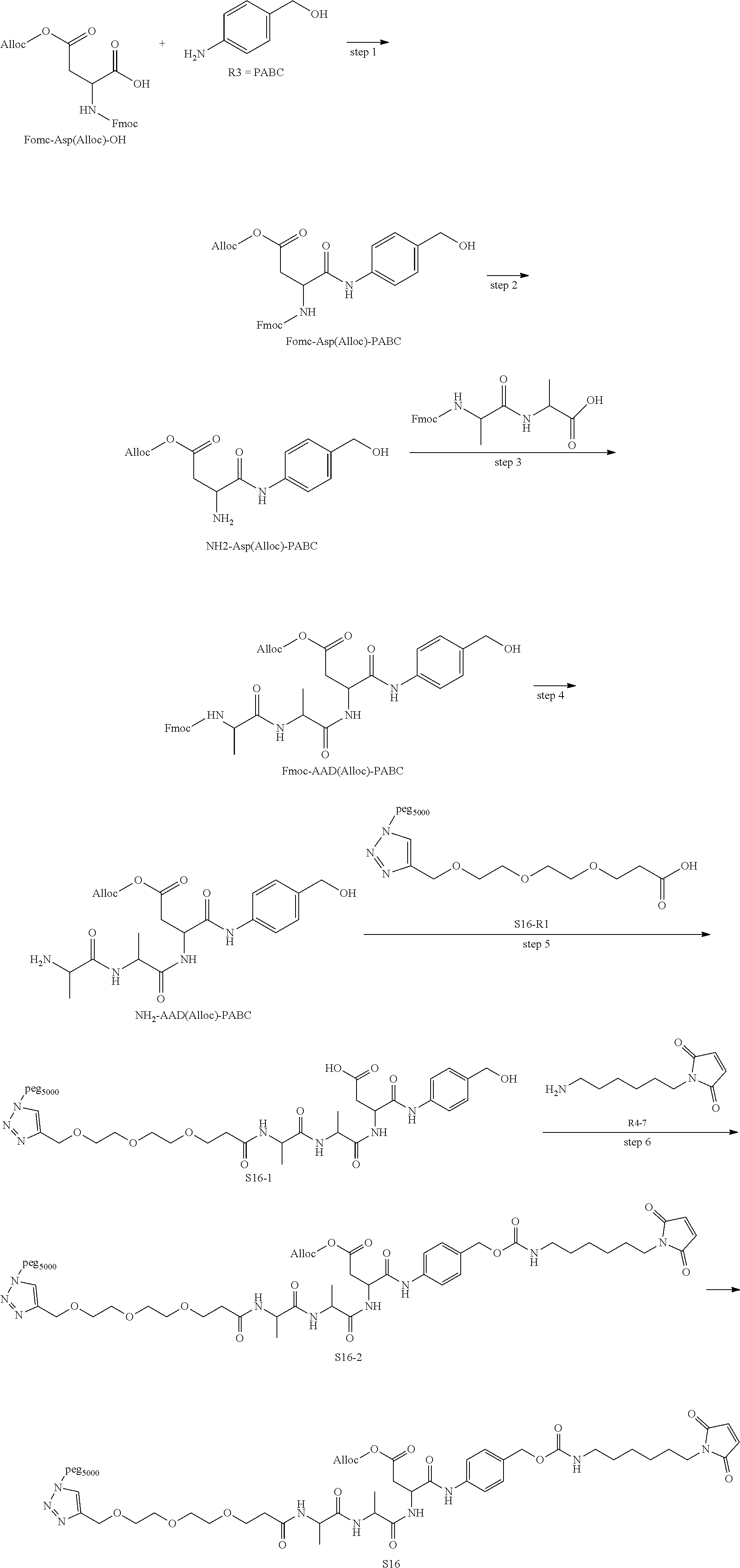

1) Fmoc-Asp(Alloc)-OH (13.2 g, 0.03 mol), 2-(7-azabenzotriazole)-N,N,N′,N′-tetramethyluronium hexafluophosphate (HATU) (15 g, 0.04 mol), and DMF (200 mL) were added to a three-necked flask and stirred for 30 min. p-Aminobenzyl alcohol (4.1 g, 0.03 mol) and N,N-diisopropyl ethylamine (8.7 g, 0.06 mol) were added at 0 °C, respectively and then stirred at room temperature for 3 hours. DMF was removed by rotary evaporation. The residue was purified by column chromatography to obtain a white solid Fmoc-Asp(Alloc)-PABC (14.7 g; Yield: 89%).

2) Fmoc-Asp(Alloc)-PABC (14.0 g, 25 mmol) was dissolved in N,N-dimethyl formamide (80 mL). Piperidine (30 mL) were added and then stirred at room temperature for 2 hours. The solvents were removed by evaporation under reduced pressure. The residue was dried under high vacuum in a vacuum oven to produce 8.0 g pale yellow solid NH₂-Asp(Alloc)-PABC which can be used in the next step without purification.

3) Fmoc-Ala-Ala-OH (7.8 g, 20.4 mmol), benzotriazole-N,N,N′,N′-tetramethyluronium hexafluophosphate (HBTU) (11.6 g, 30.6 mmol) and DMF (50 mL) were added into a three-necked flask and stirred for 30 min in an ice bath. NH₂-Asp(Alloc)-PABC (6.6 g, 20.4 mmol) and N,N-diisopropyl ethylamine (7.89 g, 61.2 mmol) were added respectively at 0 °C and then stirred at room temperature overnight. The solvents were removed by evaporation under reduced pressure. The residue was purified by silica gel column chromatography to obtain a white solid Fmoc-AAD(Alloc)-PABC (12.6 g; Yield: 90%).

4) Fmoc-AAD(Alloc)-PABC (12 g, 17.5 mmol) was dissolved in dichloromethane (80 mL). Piperidine (30 mL) were added and then stirred at room temperature for 2 hours. The solvents were removed by evaporation under reduced pressure. The residue was dried under high vacuum in a vacuum oven to obtain 7.0 g pale yellow solid, which was directly used in the next step.

5) Intermediate S16-R1 (522 mg, 0.1 mmol), 2-(7-azabenzotriazole)-N,N,N′,N′-tetramethyluronium hexafluophosphate (46 mg, 0.12 mmol), and DMF (20 mL) were added into a three-necked flask and stirred for 30 min. NH₂-AAD(Alloc)-PABC (46 mg, 0.1 mmol) and N,N-diisopropyl ethylamine (38.7 mg, 0.3 mmol) were added, respectively, at 0 °C and then stirred at room temperature for 3 hours. DMF was removed by rotary evaporation. The residue was purified by column chromatography to obtain S16-1 (white solid, 251 mg, Yield: 45%).

6) S16-1 (240 mg, 0.046 mmol) dissolved in dichloromethane (10 mL) were added into a three-necked flask. 4-Nitrophenyl chloroformate (18 mg, 0.093 mmol) in dichloromethane and pyridine (7.3 mg, 0.093 mmol) in dichloromethane were dropped into the flask, respectively, in an ice bath and under nitrogen gas protection and then stirred at room temperature overnight. R4-7 (11 mg, 0.055 mmol) were added into the above solution and allowed to react at room temperature for 4 hours. The reaction solution was dried by rotary evaporation. The resultant crude product was purified by silica gel column chromatography to obtain a white solid S16-2 (96 mg; Yield: 38%).

7) DMF (10 ml), compound S16-2 (96 mg, 0.016 mmol), acetic acid (127 mg, 2.15 mmol), triphenylphosphine palladium (33 mg, 0.029 mmol) and tributyltin hydride (0.54 g, 1.86 mmol) were added successively into a one-necked flask. After replacing the air in the flask with nitrogen gas, the mixture was stirred at room temperature until compound S16-2 was reacted completely. After the reaction was completed, DMF was removed by evaporation under reduced pressure. The crude product was isolated and purified by silica gel column chromatography to obtain a white solid (54 mg; Yield: 62%), which was compound S16.

When an acidically activatable group was contained, the synthetic scheme was shown as follows:

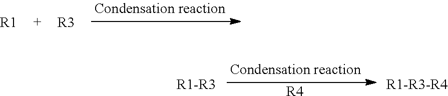

As exemplified by S20, the specific synthesis process was shown below:

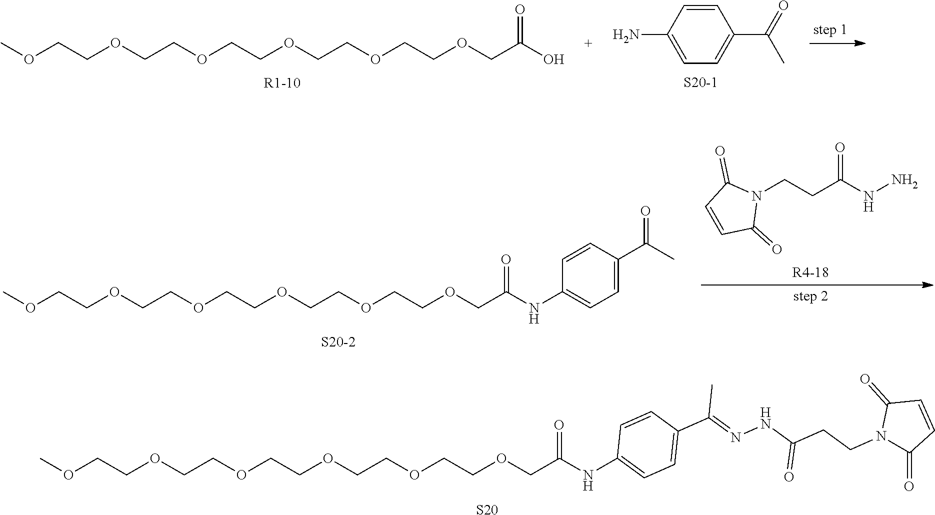

1) R1-10 (3.1 g, 0.01 mol), 2-(7-azabenzotriazole)-N,N,N′,N′-tetramethyluronium hexafluophosphate (HATU) (4.56 g, 0.012 mol), and DMF (20 mL) were added into a three-necked flask and stirred for 30 min. S20-1 (1.35 g, 0.01 mol) and N,N-diisopropyl ethylamine (3.87 g, 0.03 mol) were added respectively at 0 °C and then stirred at room temperature for 3 hours. DMF was removed by rotary evaporation. The residue was purified by silica gel column chromatography to obtain a pale yellow oily substance S20-2 (2.5 g; Yield: 59%).

2) S20-2 (98 mg, 0.23 mmol) and R4-18 (42 mg, 0.23 mmol) were weighed and successively added into a 50 ml one-necked flask. Dichloromethane (5 mL) was added to dissolve S20-2 and R4-18 followed by addition of 4 A molecular sieves (81 mg). After replacing the air in the flask with nitrogen gas, the mixture was allowed to react at room temperature overnight. The reaction solution was dried by rotary evaporation. The crude product was purified by silica gel column chromatography to obtain a white solid (81 mg; Yield: 60%), which was compound S20.

When R1, R3 and R4 were different substituents, the following compounds in Table 3 were obtained.

TABLE 3
R1

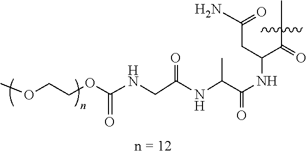

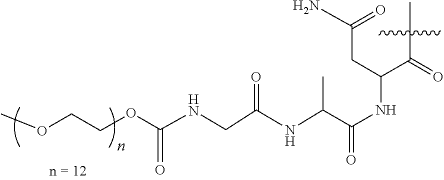

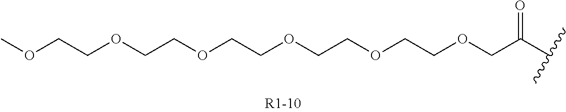 [SMILES: *C(=O)COCCOCCOCCOCCOCCOC]

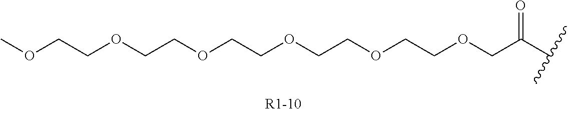 [SMILES: *C(=O)COCCOCCOCCOCCOCCOC]

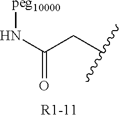

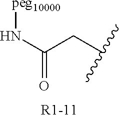

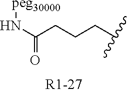

R1-14

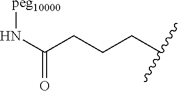

R1-14

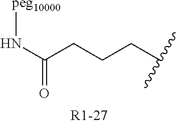

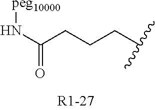

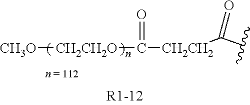

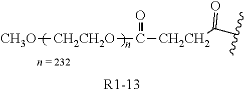

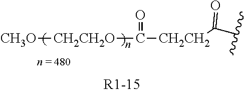

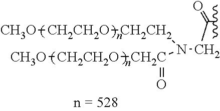

R1-17

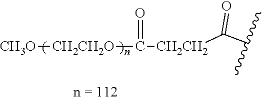

R1-12

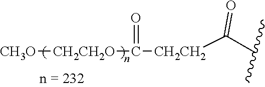

R1-13

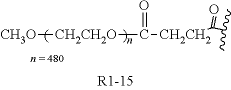

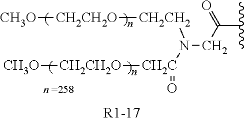

| R3 | R4 |
|---|---|
| 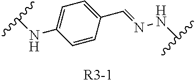 [SMILES: *N/N=C/c1ccc(N*)cc1] | 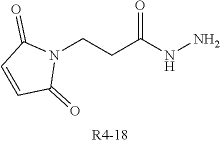 |
| 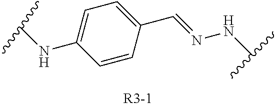 [SMILES: *N/N=C/c1ccc(N*)cc1] | 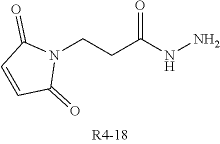 |
| 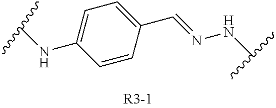 [SMILES: *N/N=C/c1ccc(N*)cc1] | 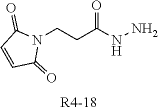 |
| 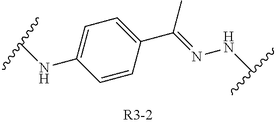 [SMILES: *N/N=C(\C)c1ccc(N*)cc1] | 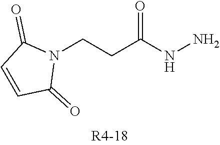 |
| 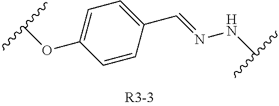 [SMILES: *N/N=C/c1ccc(O*)cc1] | 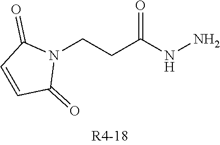 |
| 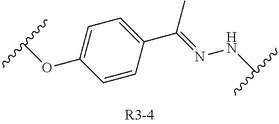 [SMILES: *N/N=C(\C)c1ccc(O*)cc1] | 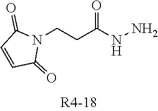 |
| 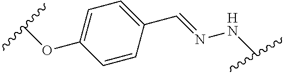 | 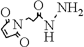 |
| R3-3 | R4-18 |
| 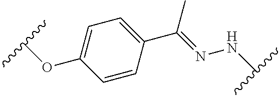 [SMILES: *N/N=C(\C)c1ccc(O*)cc1] | 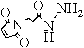 |
| R3-4 | R4-18 |
| 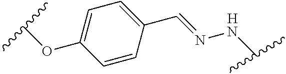 R3-3 | 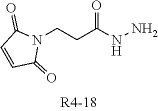 |
| | R4-18 |
| 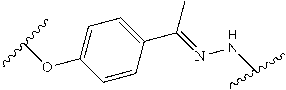 | 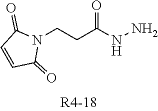 |
| 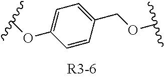 [SMILES: *OCc1ccc(O*)cc1] | 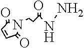 |
| | R4-18 |
| 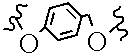 [SMILES: *OCc1ccc(O*)cc1] R5-6 | 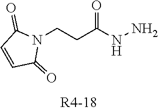 |
| 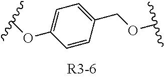 [SMILES: *OCc1ccc(O*)cc1] | 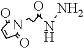 R4-18 |
| 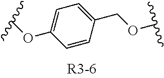 [SMILES: *OCc1ccc(O*)cc1] | 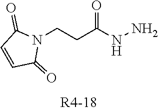 |
| 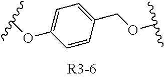 [SMILES: *OCc1ccc(O*)cc1] | 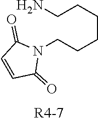 |
| 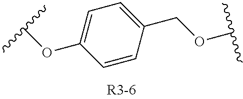 [SMILES: *OCc1ccc(O*)cc1] | 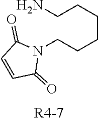 |
| 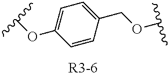 [SMILES: *OCc1ccc(O*)cc1] | 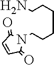 |
| | R4-7 |
| 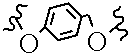 [SMILES: *OCc1ccc(O*)cc1] R3-6 | 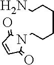 |
| | R4-7 |
| 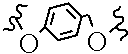 [SMILES: *OCc1ccc(O*)cc1] R3-6 | 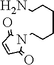 |
| | R4-7 |

Compound

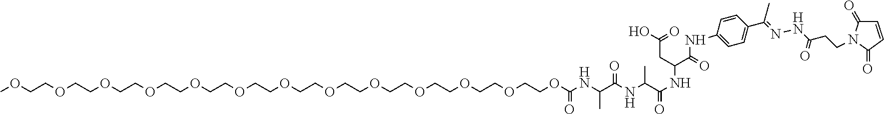 [SMILES: COCCOCCOCCOCCOCCOCCOCCOCCOCCOCCOCCOCCOC(=O)NC(C)C(=O)NC(C)C(=O)NC(CC(=O)O)C(=O)Nc1ccc(/C(C)=N/NC(=O)CCN2C(=O)C=CC2=O)cc1]

S5

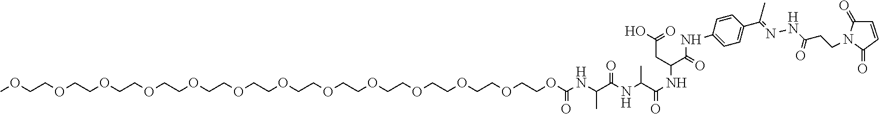 [SMILES: COCCOCCOCCOCCOCCOCCOCCOCCOCCOCCOCCOCCOC(=O)NC(C)C(=O)NC(C)C(=O)NC(CC(=O)O)C(=O)Nc1ccc(/C(C)=N/NC(=O)CCN2C(=O)C=CC2=O)cc1]

S6

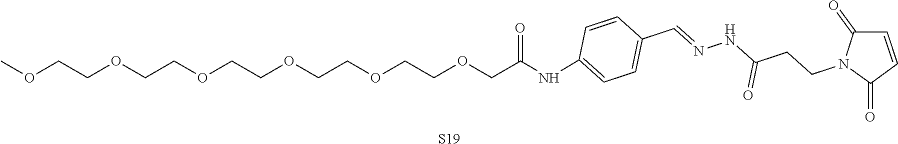 [SMILES: COCCOCCOCCOCCOCCOCC(=O)Nc1ccc(/C=N/NC(=O)CCN2C(=O)C=CC2=O)cc1 ; "S19"]

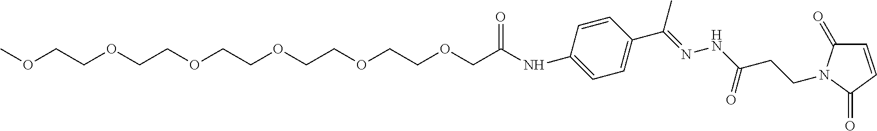 [SMILES: COCCOCCOCCOCCOCCOCC(=O)Nc1ccc(/C(C)=N/NC(=O)CCN2C(=O)C=CC2=O)cc1]

S20

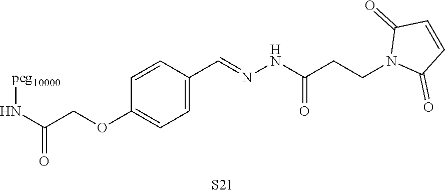

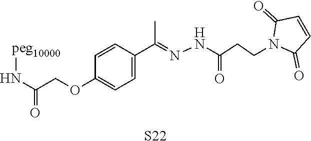

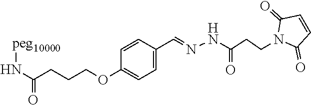

S23

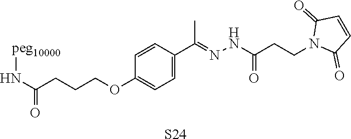

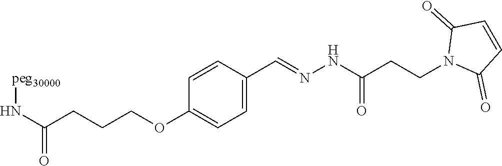

S27

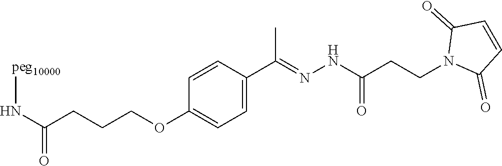

S28

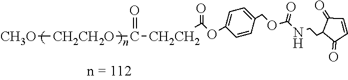

S49

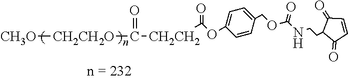

S50

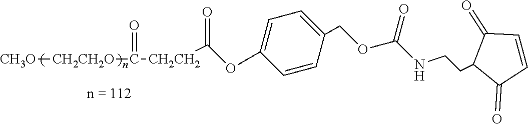

S51

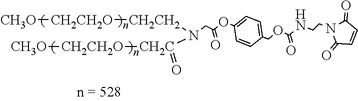

S52

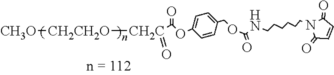

S53

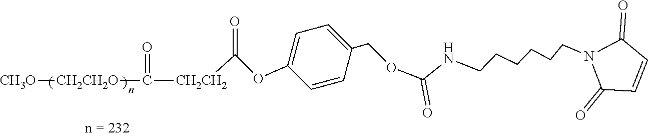

S54

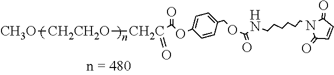

S55

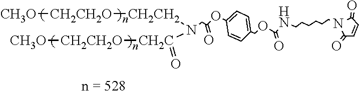

S56

Compounds S1-S64 were verified by mass spectrum (MS) and their molecular weights were shown in Table 4, which were consistent to the calculated molecular weights based on their structures.

TABLE 4

| No. | Detection by MS | Molecular Weight | Character | Yield | |
|---|---|---|---|---|---|
| S1 | 1258 | 1258.37 | white solid | 71 | mg |
| S2 | 1258 | 1258.37 | white solid | 49 | mg |
| S3 | 1132 | 1132.21 | white solid | 236 | mg |
| S4 | 1133 | 1133.20 | white solid | 93 | mg |
| S5 | 1129 | 1129.21 | white solid | 37 | mg |
| S6 | 1130 | 1130.20 | white solid | 46 | mg |
| S7 | 1159 | 1159.28 | white solid | 158 | mg |
| S8 | 1160 | 1160.26 | white solid | 102 | mg |
| S9 | 1188 | 1188.32 | white solid | 34 | mg |
| S10 | 1189 | 1189.30 | white solid | 28 | mg |
| S11 | 765 | 764.82 | white solid | 18 | mg |
| S12 | 766 | 765.81 | white solid | 31 | mg |
| S13 | 5756 | 5755.81 | white solid | 364 | mg |
| S14 | 5757 | 5756.80 | white solid | 270 | mg |
| S15 | 5800 | 5799.87 | white solid | 418 | mg |
| S16 | 5801 | 5800.85 | white solid | 54 | mg |
| S17 | 10800 | 10799.87 | white solid | 189 | mg |
| S18 | 10801 | 10800.85 | white solid | 167 | mg |
| S19 | 579 | 578.61 | white solid | 102 | mg |
| S20 | 593 | 592.64 | white solid | 81 | mg |
| S21 | 10326 | 10326.32 | white solid | 106 | mg |
| S22 | 10340 | 10340.35 | white solid | 97 | mg |
| S23 | 10354 | 10354.38 | white solid | 139 | mg |
| S24 | 10368 | 10368.40 | white solid | 76 | mg |
| S25 | 30800 | 30799.87 | white solid | 143 | mg |
| S26 | 30801 | 30800.85 | white solid | 125 | mg |
| S27 | 30354 | 30354.38 | white solid | 136 | mg |
| S28 | 30368 | 30368.40 | white solid | 121 | mg |
| S29 | 10982 | 10982.37 | white solid | 223 | mg |
| S30 | 10983 | 10982.46 | white solid | 184 | mg |
| S31 | 20982 | 20982.28 | white solid | 274 | mg |
| S32 | 20983 | 20982.32 | white solid | 231 | mg |
| S33 | 60982 | 60982.19 | white solid | 362 | mg |
| S34 | 60983 | 60983.26 | white solid | 284 | mg |
| S35 | 11070 | 11070.34 | white solid | 164 | mg |
| S36 | 11071 | 11071.41 | white solid | 182 | mg |
| S37 | 21070 | 21070.18 | white solid | 155 | mg |
| S38 | 21071 | 21071.27 | white solid | 169 | mg |
| S39 | 5655 | 5655.37 | white solid | 156 | mg |
| S40 | 5656 | 5656.22 | white solid | 231 | mg |
| S41 | 10947 | 10947.42 | white solid | 143 | mg |
| S42 | 10948 | 10948.36 | white solid | 157 | mg |
| S43 | 21883 | 21883.84 | white solid | 241 | mg |
| S44 | 21884 | 21884.72 | white solid | 185 | mg |
| S45 | 41728 | 41728.86 | white solid | 174 | mg |
| S46 | 41729 | 41729.73 | white solid | 169 | mg |
| S47 | 47358 | 47358.46 | white solid | 248 | mg |
| S48 | 47359 | 47359.39 | white solid | 312 | mg |
| S49 | 5353 | 5343.61 | white solid | 254 | mg |
| S50 | 10635 | 10635.68 | white solid | 216 | mg |
| S51 | 21572 | 21572.47 | white solid | 198 | mg |
| S52 | 47047 | 47047.18 | white solid | 183 | mg |
| S53 | 5400 | 5399.76 | white solid | 175 | mg |
| S54 | 10692 | 10691.74 | white solid | 168 | mg |
| S55 | 21628 | 21628.51 | white solid | 156 | mg |
| S56 | 47103 | 47103.24 | white solid | 141 | mg |
| S57 | 10835 | 10835.22 | white solid | 139 | mg |
| S58 | 10836 | 10836.09 | white solid | 145 | mg |
| S59 | 21407 | 21407.28 | white solid | 182 | mg |
| S60 | 21408 | 21408.17 | white solid | 163 | mg |
| S61 | 43256 | 43256.02 | white solid | 196 | mg |
| S62 | 43257 | 43257.10 | white solid | 175 | mg |
| S63 | 81901 | 81901.07 | white solid | 154 | mg |
| S64 | 81902 | 81902.14 | white solid | 139 | mg |

### Example 2: Analysis on the Binding Activity after Mutation in CDR of a Variable Region of an Antibody and Screening for R4

The amino acid sequence of anti-PD-1 antibody 1 was disclosed in WO200815712A1 and its DNA sequence was optimized for expression in a host and synthesized (GENEWIZ, Inc., Suzhou, China). The amino acid sequence of anti-PD-1 antibody 2 was disclosed in WO2006121168A2 and its DNA sequence was optimized for expression in a host and synthesized (GENEWIZ, Inc., Suzhou, China). The amino acid sequence of anti-CTLA-4 antibody was disclosed in US2015/0283234 and its DNA sequence was optimized and synthesized (GENEWIZ, Inc., Suzhou, China). The amino acid sequence of anti-TNFα antibody was disclosed in US009534046 and its DNA sequence was optimized for expression in a host and synthesized (GENEWIZ, Inc., Suzhou, China). The amino acid sequence of anti-CD28 antibody was disclosed in US007939638, and its DNA sequence was optimized for expression in a host and synthesized (GENEWIZ, Inc., Suzhou, China). The synthesized DNAs were digested and ligated to a modified pTT5 vector (Biovector) to produce pTT5-anti-PD-1-1, pTT5-anti-PD-1-2, pTT5-anti-CTLA-4, pTT5-anti-TNFα and pTT5-anti-CD28. Cysteine mutation was introduced by using pTT5-anti-PD-1-1, pTT5-anti-CTLA-4, pTT5-anti-TNFα and pTT5-anti-CD28 as templates, designing primer to replace the codon at the mutation position with that of cysteine, PCR and digesting and constructing the mutated fragment into the modified pTT5 vector.

The expression host was HEK293T cell (Life Technologies). Before transfection, HEK293T cells were cultured in a complete medium containing 10% FBS (Gibco) at 37 °C and 5% CO₂. One day before transfection, the cells were inoculated onto a 15 cm culture dish in an appropriate density and the culture medium was changed into FBS with low IgG (Gibco). 6 hours after transfection or on day 2, the culture medium was changed into Freestyle293 (Gibco).

On the day of transfection, when the cells reached a certain confluence, lipofectamine 2000 (Life Technologies) and PEI (sigma) were used to co-transfect the plasmids expressing the target protein to 293T cells. Antibody-expressing plasmids included heavy chain and light chain of the antibody. Culture supernatants were recovered on day 4 and day 6 after transfection, respectively. Expression and activity of the protein or antibody were detected, and the protein or antibody was purified.

The hypervariable regions of a variable region within heavy chain and light chain of an antibody (Ab) constitute an antigen (Ag) binding site of the antibody. Because the antigen binding site is complementary to a structure of an antigen epitope, the hypervariable region is also called as complementarity-determining region (CDR) of an antibody. The sequences of variable regions in light chain of anti-PD-1 antibody 1, anti-PD-1 antibody 2 and CTAL4 antibody were aligned and their CDRs were shown in FIG. 1.

In the present disclosure, the activity of each mutant having point mutation as compared to its initial antibody was detected by ELISA. Specifically, a 96-well plate was coated by an antigen of an antibody overnight and then blocked with 1% BSA blocker (ThermoFisher) for 2 hours at 37 °C and washed by PBST three times. Corresponding antibody or corresponding mutant was added and allowed to bind at 37 °C for 1 hour, then washed with PBST three times. HRP enzyme-conjugated anti-human IgG was added and allowed to bind at 37 °C for 1 hour and then washed with PBST three times. TMB substrate (Solarbio., Inc.) was used to detect absorbance at 450 nm. Effect on the binding strength of mutant was calculated by OD_{after mutation}/OD_{wild type}.

The binding activity of mutants of three antibodies, which had mutation at different positions as shown in Tables 5 and 6, were tested according to the above methods. In Tables 5 and 6, A represents a binding activity of 90-110%, B represents a binding activity of 70-90% and the symbol ↓ indicates a binding activity of less than 70%.

TABLE 5
Effect of mutation position in CDR of a variable region in a light chain of an antibody
on binding activity

| | | PD-1 | Binding to | anti-PD-1 | Binding to | anti-CTLA-4 | Binding |
|---|---|---|---|---|---|---|---|
| CDR | Position | Antibody 1 | PD-1 | antibody 2 | PD-1 | antibody | to CTLA-4 |
| CDR1 | 25 | A | B | A | B | A | A |
| CDR1 | 26 | S | A | S | A | S | A |
| CDR1 | 27 | K | ↓ | | | | |
| CDR1 | 28 | G | A | S | A | S | A |
| CDR1 | 29 | | | | | | |
| CDR1 | 30 | S | A | S | A | G | A |
| CDR1 | 31 | T | B | S | A | S | A |
| CDR1 | 32 | S | A | Y | ↓ | S | A |
| CDR1 | 33 | G | A | L | ↓ | Y | ↓ |
| CDR1 | 34 | Y | ↓ | A | A | L | B |
| CDR1 | 35 | S | A | | | A | B |
| CDR1 | 36 | Y | ↓ | Y | ↓ | | |
| CDR1 | 37 | L | B | | | Y | ↓ |
| CDR2 | 51 | | | A | B | G | A |
| CDR2 | 52 | | | S | A | A | ↓ |
| CDR2 | 53 | Y | ↓ | | | | |
| CDR2 | 54 | L | B | S | A | S | A |
| CDR2 | 55 | A | B | A | B | | |
| CDR2 | 56 | S | A | T | A | A | B |
| CDR2 | 57 | | | G | A | T | A |
| CDR2 | 58 | | | I | B | G | A |
| CDR2 | 60 | S | A | A | B | | |
| CDR2 | 61 | G | A | | | | |
| CDR3 | 91 | | | S | A | | |
| CDR3 | 92 | | | S | A | Y | ↓ |
| CDR3 | 93 | | | | | G | A |
| CDR3 | 94 | | | | | S | A |
| CDR3 | 95 | S | A | | | S | A |
| CDR3 | 96 | | ↓ | | | | |
| CDR3 | 97 | D | ↓ | T | A | | |
| CDR3 | 98 | L | ↓ | F | ↓ | T | B |
| CDR3 | 99 | | ↓ | G | A | F | ↓ |
| CDR3 | 100 | L | ↓ | | | G | A |
| CDR3 | 101 | T | B | G | ↓ | | |
| CDR3 | 102 | F | ↓ | | | | |

TABLE 6
Effect of mutation position in CDR of a variable region in a heavy chain of an
antibody on binding activity

| | | PD-1 | Binding to | anti-PD-1 | Binding to | anti-CTLA-4 | Binding |
|---|---|---|---|---|---|---|---|
| CDR | Position | Antibody 1 | PD-1 | antibody 2 | PD-1 | antibody | to CTLA-4 |
| CDR1 | 30 | T | A | S | A | S | A |
| CDR1 | 31 | | | | | S | A |
| CDR1 | 32 | Y | ↓ | S | A | Y | B |
| CDR1 | 33 | Y | ↓ | G | A | T | A |
| CDR1 | 35 | Y | B | | | | |
| CDR2 | 50 | G | A | | | | |
| CDR2 | 51 | I | B | I | B | I | B |
| CDR2 | 53 | | | Y | ↓ | Y | ↓ |
| CDR2 | 54 | S | A | D | B | D | B |
| CDR2 | 55 | | | G | A | G | A |
| CDR2 | 56 | G | A | S | A | S | A |
| CDR2 | 57 | G | A | K | ↓ | | |
| CDR2 | 58 | T | A | | | K | ↓ |
| CDR2 | 59 | | | Y | ↓ | Y | ↓ |
| CDR2 | 60 | | | Y | ↓ | Y | ↓ |
| CDR2 | 61 | | | A | B | A | A |
| CDR2 | 62 | | | D | ↓ | D | B |
| CDR2 | 63 | K | B | S | A | S | A |
| CDR2 | 65 | K | ↓ | K | ↓ | K | ↓ |
| CDR2 | 66 | | | G | A | G | A |
| CDR3 | 100 | D | ↓ | D | ↓ | | |
| CDR3 | 101 | Y | ↓ | D | B | G | A |
| CDR3 | 102 | | | Y | ↓ | L | A |
| CDR3 | 104 | | | G | A | T | A |
| CDR3 | 105 | | | | | L | B |
| CDR3 | 106 | G | A | G | A | D | ↓ |
| CDR3 | 107 | | | T | A | Y | B |

The results showed that in variable region of different antibodies, mutating G, S and a portion of T to C had less effect on the binding activity as compared to the wild type antibody, which was 90% or more of the original binding activity. Mutating A, I and a portion of T to C produced a binding activity which was 70-90% of the binding activity of the wild type antibody.

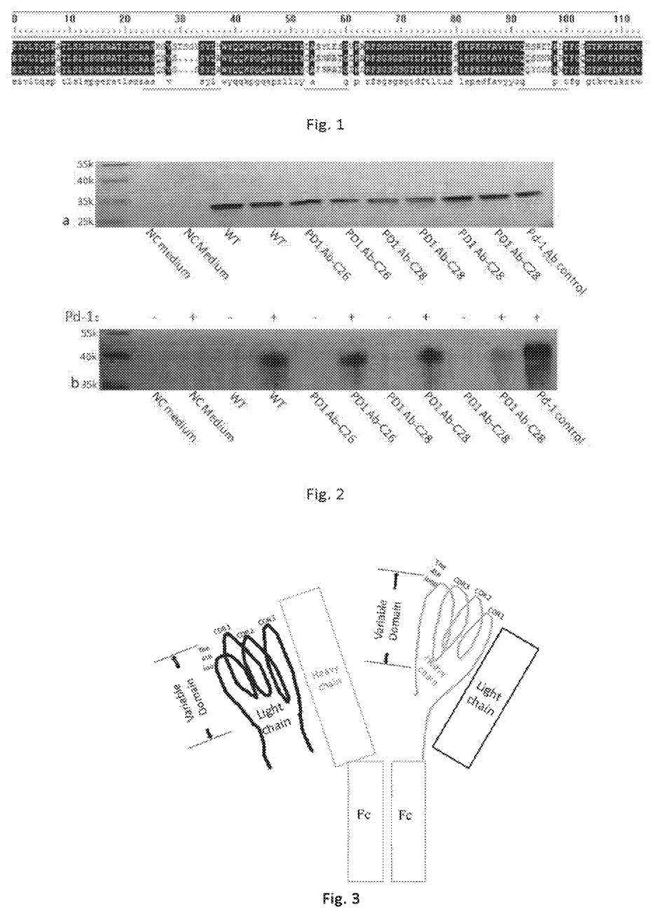

Results obtained from Co-IP screening for point mutation on light chain of anti-PD1 antibody 1 are shown in FIG. 2, wherein panel a shows the effect of mutation of a wild-type antibody on expression detected by WB, and panel b shows the binding of the mutated antibody to PD1 detected by WB.

Analysis on the Binding Activity of Mutant of Antibody Having Mutation in CDR after Binding to R4

As shown in Tables 7-16, antibodies having mutation in CDR of a light chain or a heavy chain were conjugated to R4 in a library of small molecules for conjugation, and their binding activities were compared to wild type antibodies (binding activity of a conjugate to antibody/binding activity of wild type antibody*100%) to obtain a conjugation manner which would provide intermolecular force and enhance the binding activity.

TABLE 7
Binding effect of mutants of anti-PD-1 antibody 1 having mutation
in CDR of a light chain after conjugating to R4-1 or R4-5

| | | anti-PD-1 | Conjugating | Conjugating |
|---|---|---|---|---|
| CDR | Position | antibody 1 | to R4-1 | to R4-5 |
| CDR 1 | 26 | S | 93.2% | |
| CDR 1 | 30 | S | 86.9% | |
| CDR 1 | 31 | T | 84.4% | |
| CDR 1 | 28 | G | 104.6% | 107.6% |
| CDR 1 | 35 | S | 88.3% | |
| CDR 1 | 36 | Y | 85.4% | |
| CDR 1 | 55 | A | 92.1% | |
| CDR 2 | 57 | Y | 87.7% | |
| CDR 2 | 60 | S | 89.4% | |
| CDR 3 | 104 | G | 83.6% | |

TABLE 8
Binding effect of mutants of anti-PD-1 antibody 1 having
mutation in CDR of a heavy chain after conjugating to R4

| | | anti-PD-1 | Conjugating | Conjugating | Conjugating |
|---|---|---|---|---|---|
| CDR | Position | antibody 1 | to R4-1 | to R4-4 | to R4-7 |
| CDR 1 | 30 | T | 91.2% | | |
| CDR 1 | 32 | Y | | 89.5% | |
| CDR 2 | 50 | G | 84.4% | | |
| CDR 2 | 54 | S | 103.6% | | |
| CDR 2 | 58 | T | 93.9% | | |
| CDR 2 | 63 | K | | | 95.2% |
| CDR 3 | 101 | Y | | 86.1% | |
| CDR 3 | 106 | G | 95.1% | | |

According to Tables 7 and 8, after mutating A, G, S, Y, T or K in the CDRs of anti-PD-1 antibody 1 to C and binding to R4, the mutants could retain a binding efficiency of >80%.

TABLE 9
Binding effect of mutants of anti-PD-1 antibody 2 having mutation
in CDR of a light chain after conjugating to R4-1 or R4-5

| | | anti-PD-1 | Conjugating | Conjugating |
|---|---|---|---|---|
| CDR | Position | antibody 2 | to R4-1 | to R4-5 |
| CDR 1 | 25 | A | 26.5% | 87.2% |
| CDR 1 | 26 | S | 86.8% | |
| CDR 1 | 28 | S | 88.9% | |
| CDR 1 | 30 | S | 34.4% | |
| CDR 1 | 31 | S | 95.7% | |
| CDR 1 | 32 | Y | 25.6% | |
| CDR 1 | 33 | L | 1.6% | |
| CDR 1 | 34 | A | 1.7% | 83.9% |
| CDR 2 | 51 | A | 95.6% | |
| CDR 2 | 52 | S | 34.6% | |
| CDR 2 | 55 | A | 99.5% | |
| CDR 2 | 56 | T | | 89.4% |
| CDR 3 | 57 | G | 89.8% | |
| CDR 3 | 91 | S | 93.5% | |
| CDR 3 | 92 | S | 45.5% | |
| CDR 3 | 97 | T | 93.7% | |

TABLE 10
Binding effect of mutants of anti-PD-1 antibody 2 having mutation in CDR of a
heavy chain after conjugating to R4

| | | anti-PD-1 | Conjugating | Conjugating | Conjugating | Conjugating |
|---|---|---|---|---|---|---|
| CDR | Position | antibody 2 | to R4-1 | to R4-3 | toR4-4 | to R4-7 |
| CDR 1 | 30 | S | 107.2% | | | |
| CDR 1 | 32 | S | 86.8% | | | |
| CDR 1 | 33 | G | 88.9% | | | |
| CDR 2 | 53 | Y | | | 91.6% | |
| CDR 2 | 55 | G | 95.7% | | | |
| CDR 2 | 56 | S | 105.6% | | | |
| CDR 2 | 57 | K | | | | 89.7% |
| CDR 2 | 61 | A | 87.0% | | 83.9% | |
| CDR 2 | 63 | S | 95.6% | | | |
| CDR 2 | 65 | K | | | | 92.1% |
| CDR 2 | 66 | G | 99.5% | | | |
| CDR 3 | 104 | G | 89.4% | | | |
| CDR 3 | 106 | G | 91.8% | | | |
| CDR 3 | 107 | T | 95.7% | | | |
| CDR 3 | 108 | L | | 86.4% | | |

According to Tables 9 and 10, after mutating G, S, A, Y, K, L or T in the CDRs of anti-PD-1 antibody 2 to C and binding to R4, the mutants could retain a binding efficiency of >80%.

TABLE 11
Binding effect of mutants of anti-CTLA-4 antibody having mutation
in CDR of a light chain after conjugating to R4-1

| CDR | Position | Anti-CTLA-4 Antibody | Conjugating to R4-1 |
|---|---|---|---|
| CDR 1 | 25 | A | 95.40% |
| CDR 1 | 26 | S | 91.00% |
| CDR 1 | 28 | S | 82.00% |
| CDR 1 | 30 | G | 95.30% |
| CDR 1 | 31 | S | 80.90% |
| CDR 1 | 32 | S | 99.00% |
| CDR 1 | 34 | L | 94.90% |
| CDR 1 | 35 | A | 91.50% |
| CDR 2 | 51 | G | 97.00% |
| CDR 2 | 52 | A | 92% |
| CDR 2 | 54 | S | 97.70% |
| CDR 2 | 56 | A | 102.30% |
| CDR 2 | 57 | T | 92.50% |
| CDR 2 | 58 | G | 88.10% |
| CDR 3 | 93 | G | 99.10% |
| CDR 3 | 94 | S | 92.40% |
| CDR 3 | 95 | S | 95.40% |
| CDR 3 | 98 | T | 91.00% |

TABLE 12
Binding effect of mutants of anti-CTLA-4 antibody having mutation in CDR of a
heavy chain after conjugating to R4

| | | Anti-CTLA-4 | Conjugating | Conjugating | Conjugating | Conjugating |
|---|---|---|---|---|---|---|
| CDR | Position | Antibody | to R4-1 | to R4-3 | to R4-4 | to R4-7 |
| CDR 1 | 30 | S | 93.4% | | | |
| CDR 1 | 31 | S | 109.5% | | | |
| CDR 1 | 33 | T | 102.3% | | | |
| CDR 2 | 55 | G | 95.2% | | | |
| CDR 2 | 58 | K | | | | 87.6% |
| CDR 2 | 59 | Y | | | 89.1% | |
| CDR 2 | 60 | Y | | | 91.8% | |
| CDR 2 | 65 | K | | | | 96.2% |
| CDR 2 | 66 | G | 92.7% | | | |
| CDR 3 | 100 | G | 98.3% | | | |
| CDR 3 | 102 | L | | 83.9% | | |
| CDR 3 | 103 | G | 93.8% | | | |
| CDR 3 | 106 | D | | 87.6% | | |

According to Tables 11 and 12, after mutating A, G, S, L, T, K, Y or D in the CDRs of anti-CTLA-4 antibody to C and binding to R4, the mutants could retain a binding efficiency of >80%.

TABLE 13
Binding effect of mutants of anti-CD28 antibody having mutation in CDR of a light
chain after conjugating to R4

| | | anti-CD28 | Conjugating | Conjugating | Conjugating | Conjugating |
|---|---|---|---|---|---|---|
| CDR | Position | antibody | to R4-1 | to R4-3 | to R4-4 | to R4-7 |
| CDR 1 | 26 | S | 87.0% | | | |
| CDR 1 | 29 | I | | 88.5% | | |
| CDR 1 | 30 | Y | 19.5% | | 93.6% | |
| CDR 2 | 50 | K | 26.6% | | | 88.9% |
| CDR 2 | 51 | A | 97.7% | | | |
| CDR 2 | 52 | S | 84.0% | | | |
| CDR 3 | 91 | G | 88.3% | | | |
| CDR 3 | 93 | T | 87.0% | | | |
| CDR 3 | 94 | Y | 20.1% | | 83.7% | |
| CDR 3 | 96 | Y | 14.9% | | 91.4% | |
| CDR 3 | 97 | T | 95.5% | | | |
| CDR 3 | 99 | G | 96.6% | | | |

TABLE 14
Binding effect of mutants of anti-CD28 antibody having mutation
in CDR of a heavy chain after conjugating to R4

| | | anti-CD28 | Conjugating | Conjugating | Conjugating |
|---|---|---|---|---|---|
| CDR | Position | antibody | to R4-1 | to R4-3 | to R4-4 |
| CDR 1 | 25 | S | 87.0% | | |
| CDR 1 | 26 | G | 89.5% | | |
| CDR 1 | 27 | Y | | | 94.2% |
| CDR1 | 28 | T | 108.6% | | |
| CDR1 | 30 | T | 87.3% | | |
| CDR1 | 31 | S | 97.5% | | |
| CDR1 | 32 | Y | | | 81.8% |
| CDR 2 | 52 | Y | | | 91.6% |
| CDR 2 | 54 | G | 97.9% | | |
| CDR 2 | 58 | T | 89.3% | | |
| CDR 3 | 99 | S | 87.6% | | |
| CDR 3 | 101 | Y | | | 89.3% |
| CDR 3 | 102 | G | 80.1% | | |
| CDR3 | 103 | L | 14.9% | 94.6% | |

According to Tables 13 and 14, by binding mutation position of G, S, A, I, L, K, Y or T in the CDRs of anti-CD28 antibody to R4, the mutants could retain a binding efficiency of >80%.

TABLE 15
Binding effect of mutants of anti-TNFα antibody having
mutation in CDR of a light chain after conjugating to R4

| | | anti-TNFα | Conjugating | Conjugating | Conjugating |
|---|---|---|---|---|---|
| CDR | Position | antibody | to R4-1 | to R4-3 | to R4-4 |
| CDR 1 | 25 | A | 88.6% | | |
| CDR 1 | 26 | S | 97.1% | | |
| CDR 1 | 28 | G | 92.3% | | |
| CDR 1 | 29 | I | | 87.8% | |
| CDR 1 | 32 | Y | | | 89.1% |
| CDR 1 | 33 | L | | 89.4% | |
| CDR 1 | 34 | A | 85.7% | | |
| CDR 2 | 50 | A | 85.5% | | |
| CDR 2 | 51 | A | 95.1% | | |
| CDR 2 | 52 | S | 86.8% | | |
| CDR 2 | 53 | T | 89.4% | | |
| CDR 2 | 54 | L | | 82.2% | |
| CDR 2 | 56 | S | 95.4% | | |
| CDR 2 | 57 | G | 95.5% | | |
| CDR 3 | 91 | Y | 34.5% | | 90.1% |
| CDR 3 | 94 | A | 97.5% | | |
| CDR 3 | 97 | T | 82.5% | | |

TABLE 16
Binding effect of mutants of anti-TNFα antibody having
mutation in CDR of a heavy chain after conjugating to R4

| | | anti-TNFα | Conjugating | Conjugating | Conjugating |
|---|---|---|---|---|---|
| CDR | Position | antibody | to R4-1 | to R4-3 | to R4-4 |
| CDR 1 | 32 | Y | | | 88.6% |
| CDR 1 | 33 | A | 95.1% | | |
| CDR 2 | 51 | I | | 102.6% | |
| CDR 2 | 52 | T | 82.8% | | |
| CDR 2 | 55 | S | 86.9% | | |
| CDR 2 | 56 | G | 88.7% | | |
| CDR 2 | 58 | I | | 86.7% | |
| CDR 2 | 60 | Y | | | 85.2% |
| CDR 2 | 61 | A | 95.7% | | |
| CDR 2 | 63 | S | 96.8% | | |
| CDR 2 | 66 | G | 99.4% | | |
| CDR 3 | 100 | S | 92.2% | | |
| CDR 3 | 101 | Y | | | 85.4% |
| CDR 3 | 102 | L | | 85.5% | |
| CDR 3 | 103 | S | 84.5% | | |
| CDR 3 | 104 | T | 97.3% | | |
| CDR 3 | 105 | A | 86.5% | | |
| CDR 3 | 106 | S | 98.3% | | |
| CDR 3 | 107 | S | 87.4% | | |
| CDR 3 | 108 | L | | 81.4% | |
| CDR 3 | 110 | Y | | | 82.5% |

According to Tables 13 and 14, after mutating A, G, S, L, I, Y or T in the CDRs of anti-TNFα antibody to C and binding to R4, the mutants could retain a binding efficiency of >80%.

### Example 3: Analysis on the Binding Activity of Mutants Having a Mutation in a Sequence of High Homology in the Non-CDR of a Variable Region and Screening for R1

An antibody consists of 4 peptide chains, including two identical light chains (LC) and two identical heavy chains (HC). The chains form a monomer by disulfide bond(s) and non-covalent bonds. There are two types of light chains, i.e., κ and λ, and five types of heavy chains, i.e., μ, δ, γ, ε and α. An antibody, as a whole, is divided into a constant region and a variable region. The variable region is located at the terminus of the two arms of the Y-shaped structure. Humanized or human antibodies have a certain generality, that is, they all contain 4 loops in heavy chain or light chain at the terminus of the two arms of the Y-shaped structure (FIG. 3). Three loops are highly variable and directly anticipate in binding to an antigen. The regions in these loops are termed CDRs, wherein CDR1, CDR2 and CDR3 are present in these three loops, respectively. Another loop is also present at the same side where antibody binds to antigen from the four-dimensional space. Some loops not only have a conservative structure but also have a conservative sequence.

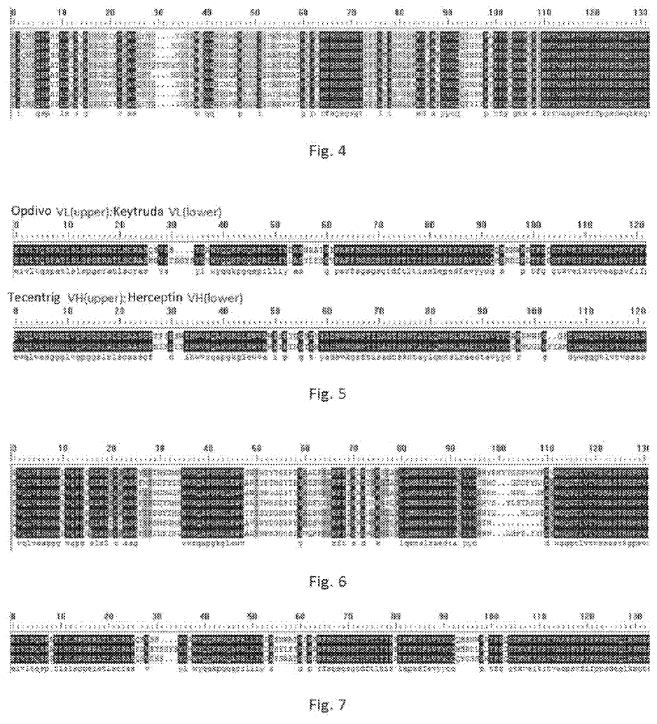

Light chains from 8 commercial antibodies (Tecentriq® (atezolizumab), Yervoy® (ipilimumab), Humira® (adalimumab), Keytruda® (pembrolizumab), Opdivo® (nivolumab), Erbitux® (cetuximab), Rituxan® (rituximab) and Perjeta® (pertuzumab), respectively, shown in the first to the last antibody of FIG. 4) were aligned and the results were shown in FIG. 4. The fourth loop in the variable region of each light chain (GSGSGST) was conservative.

There were 4 loops at the same side of the CDR of the light chain of anti-PD-1 antibody, wherein 3 loops were CDRs, including CDR1, CDR2 and CDR3. The remaining loop had a structure and a sequence which was conservative compared to many drugs approved by FDA. The sequence of this loop was RFSGSGSGT, located at positions 64-72 (FIG. 4). Each of the amino acid residues in loop 4 of anti-PD-1 antibody 1 was mutated to Cys and conjugated to S9 or S13. Effect of conjugation to R1-R4 of different length on binding activity of the mutant to PD-1 was detected by ELISA. Results were shown in Table 17. It could be found that 70% or more of an activity of the mutants may be inhibited by varying the steric structure or length of a conjugating arm.

TABLE 17
Binding activity of conjugates of anti-PD-1 antibody 1 having
a mutation in loop 4 of a variable region of a light chain

| | | | Binding | Conjugating | Conjugating |
|---|---|---|---|---|---|
| | Position | Residue | Activity | to S9 | to S13 |
| Loop4 | 67 | S | 87.8% | 27.2% | 17.8% |
| Loop 4 | 68 | G | 93.5% | 89.9% | 13.2% |
| Loop 4 | 69 | S | 96.1% | 90.6% | 16.2% |
| Loop 4 | 70 | G | 89.3% | 65.2% | 17.9% |
| Loop 4 | 71 | S | 99.5% | 89.0% | 16.0% |
| Loop 4 | 72 | G | 98.3% | 92.9% | 10.9% |
| Loop 4 | 73 | T | 93.5% | 90.2% | 10.6% |

Antibody was produced by recombination of genes of immunoglobulin superfamily in vivo. Some framework regions of antibodies against different antigens may be derived from a gene or an amino acid sequence of a same germline antibody. For example, the heavy chain of an Her2 antibody, Herceptin® (trastuzumab) is different from that of Tecentriq® (atezolizumab), an anti-PD-1 antibody in the three CDRs. They have the same non-CDR framework sequence, which is derived from the same germline antibody (FIG. 5). Similar to the heavy chains of Herceptin® (trastuzumab) and Tecentriq®, the light chains of Opdivo® (nivolumab) and Keytruda® (pembrolizumab), which are PD-1 antibodies, are different in the 3 CDRs in the variable region. Their non-CDR framework regions are derived from a same germline antibody (FIG. 5).

Similar situation was found in many antibodies. As exemplified by reshaping antibodies (also called CDR-implanted antibodies) approved by FDA, to retain their specificity to an antigen and reduce heterology, the CDRs from murine derived antibody was directly replaced by CDRs from human derived antibody. As maturation of humanization technology and genetic engineering technology, the humanized antibodies later developed were mainly the humanized antibodies or human antibodies. However, even so, the heavy chains of many commercial antibodies still exhibit very high similarity. For example, FIG. 6 shows the comparison results for similarity of heavy chains of 7 commercial antibodies Avastin® (bevacizumab), Herceptin® (trastuzumab), Tecentriq® (atezolizumab), Humira® (adalimumab), Yervoy® (ipilimumab), Opdivo® (nivolumab) and Perjeta® (pertuzumab), shown in the first to the last antibody in FIG. 6), which is 85% or more. They are different from each other mainly in the CDRs and their framework regions in the variable regions have similar sequences. Even more, the framework structures of Tecentriq (Atezolizumab) and Herceptin® (trastuzumab) are identical (FIG. 5). After comparing the light chain variable regions of immune checkpoint antibodies, Opdivo® (nivolumab), Keytruda® (pembrolizumab) and Yervoy® (ipilimumab) (the first to the last antibody shown in FIG. 7, respectively), it could be found that their homology is 92%. The main difference is in the CDRs and the sequences in their framework regions are close to each other (FIG. 7).

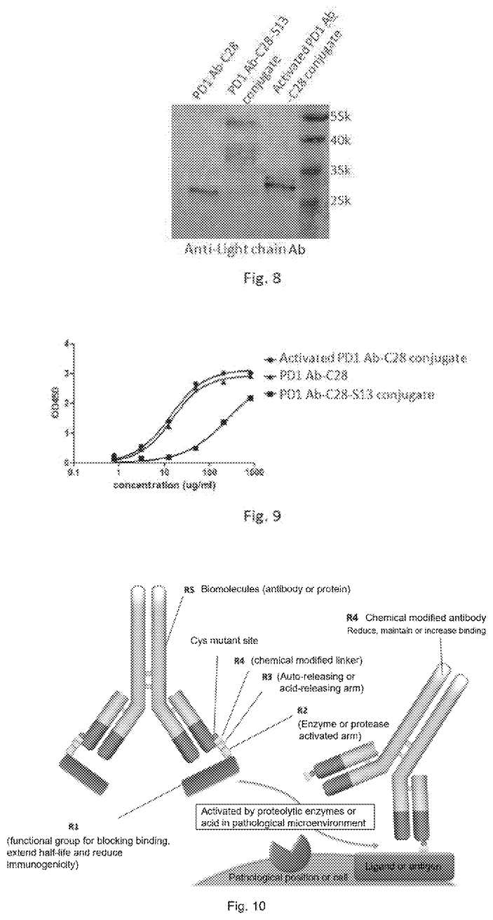

For antibodies having a conservative framework region, activities of their mutants having a mutation in a framework region (non-CDR) of heavy or light chain variable region and conjugates thereof were tested. Each of the amino acid positions shown in tables 18 and 19 was mutated to cysteine and each of the resulted mutants was conjugated to S9 or S13. Effect of conjugation to a R1-R4 side chain having a different length on its binding activity to PD-1 was tested by ELISA. Results were shown in Tables 18 and 19. FIG. 8 shows conjugation of PD1 Ab-C28 (PD1-Ab-Gly28Cys) to S13, and FIG. 9 shows the binding of anti-PD1-Ab-C28 to PD1 before and after conjugating to S13.

TABLE 18
Binding activities of mutant of anti-PD-1 antibody
1 having a mutation in a framework region of a light
chain variable region and conjugates thereof

| | Amino acid | Binding activity | Conjugating | Conjugating |
|---|---|---|---|---|
| Position | residue | after mutation | to S9 | to S13 |
| 5 | T | 95.3% | 67.4% | 26.30% |
| 7 | S | 98.2% | 59.5% | 34.20% |
| 9 | A | 96.5% | 78.4% | 44.60% |
| 10 | T | 84.3% | 65.7% | 37.40% |
| 11 | L | 96.3% | 32.50% | |
| 12 | S | 85.5% | 13.50% | |
| 13 | L | 97.5% | 80.2% | 42.30% |
| 14 | S | 97.8% | 19.7% | |
| 16 | G | 89.4% | 75.3% | 21.70% |
| 19 | A | 86.4% | 49.9% | 25.00% |
| 20 | T | 89.7% | 69.7% | 32.10% |
| 25 | A | 83.3% | 70.3% | 37.20% |
| 41 | G | 88.5% | 17.4% | |
| 43 | A | 99.3% | 82.1% | 30.80% |
| 46 | L | 95.9% | 80.2% | 37.50% |
| 47 | L | 81.1% | 68.9% | 22.90% |
| 48 | I | 89.5% | 23.8% | |
| 51 | A | 92.1% | 82.1% | 31.2% |
| 55 | A | 87.3% | 72.3% | 29.4% |
| 69 | T | 83.4% | 67.9% | 20.5% |
| 76 | S | 90.2% | 68.7% | 21.9% |
| 77 | S | 83.8% | 69.3% | 32.1% |
| 106 | I | 98.3% | 76.7% | 26.8% |
| 107 | K | 87.1% | 70.1% | 35.6% |

TABLE 19
Binding activities of mutant of anti-PD-1 antibody
2 having a mutation in a framework region of a heavy
chain variable region and conjugates thereof

| | Amino acid | Binding activity | Conjugating | Conjugating |
|---|---|---|---|---|
| Position | residue | after mutation | to S9 | to S13 |
| 7 | S | 95.3% | 65.9% | 26.3% |
| 8 | G | 93.2% | 78.3% | 34.2% |
| 9 | G | 97.8% | 87.4% | 24.6% |
| 10 | G | 84.3% | 69.3% | 37.4% |
| 15 | G | 85.6% | 32.50% | 21.5% |
| 17 | S | 85.0% | 75.3% | 42.4% |
| 24 | A | 99.6% | 45.6% | 32.3% |
| 25 | S | 99.8% | 48.7% | 27.0% |
| 26 | G | 95.4% | 31.8% | 11.7% |
| 28 | T | 86.4% | 29.9% | 15.0% |
| 30 | S | 89.7% | 31.8% | 15.3% |
| 40 | A | 85.3% | 50.3% | 37.2% |
| 42 | G | 83.4% | 63.6% | 29.8% |
| 44 | G | 99.3% | 78.3% | 30.8% |
| 69 | T | 95.9% | 78.6% | 37.5% |
| 71 | S | 81.4% | 56.9% | 22.9% |
| 75 | S | 96.3% | 38.7% | 14.6% |
| 78 | T | 92.2% | 86.3% | 42.4% |
| 85 | S | 98.8% | 69.4% | 32.3% |
| 88 | A | 84.3% | 46.8% | 27.0% |
| 91 | T | 86% | 48.9% | 21.7% |
| 92 | A | 85.0% | 64.6% | 15.0% |
| 98 | T | 89.6% | 59.9% | 32.1% |
| 110 | T | 92.1% | 78.4% | 37.2% |
| 112 | S | 95.4% | 68.6% | 29.8% |
| 113 | S | 96.0% | 70.5% | 30.8% |
| 114 | A | 89.7% | 66.9% | 37.5% |
| 115 | S | 95.3% | 70.4% | 22.9% |
| 116 | T | 93.4% | 69.5% | 14.6% |
| 118 | G | 99.3% | 72.5% | 31.3% |
| 120 | S | 95.9% | 68.4% | 36.8% |

Analysis on the binding activities of mutants having a mutation in a non-CDR region of an antibody variable region showed that there was no significant effect on the binding activity when a mutation was introduced into a non-CDR and when the mutant was conjugated to a small conjugating molecule S9. However, when the mutant was conjugated to S13, the binding activity was reduced. These indicate that amino acid residue in a non-CDR could be mutated to cysteine and then the mutant could be conjugated to a R1-R2-R3-R4 having a large molecule weight or bearing a specific functional group to block the binding activity of the mutant. With this method, the activity of the mutant could be inhibited.

Anti-PD1 antibodies having two or more mutations in light chain were prepared and conjugated to S13. The mutation sites were selected from the mutation sites of the aforementioned anti-PD1 antibody having one mutation and having 95% or more binding activity to PD1 antigen. The activity of such conjugates after activation was verified and the results were shown in Table 20.

TABLE 20
Binding activity of conjugates of anti-PD1 antibody 1 with
two or more mutations to PD1 before and after activation

| | | Binding | Binding efficiency |
|---|---|---|---|
| R1-R2- | | efficiency before | after enzymatic |
| R3-R4 | R5 | activation | or acidic activation |
| S13 | PD1 Ab-Ser7/Ser71 | 18.1% | 109.5% |
| S13 | PD1 Ab-Ser12/Gly72 | 19.6% | 95.3% |
| S13 | PD1 Ab-Ser14/Thr73 | 10.2% | 99.8% |
| S13 | PD1 Ab-Ser7/Ser14/ | 11.9% | 95.1% |
| | Thr73 | | |

The above results demonstrate that conjugates of anti-PD1 antibody with two or more mutations could retain 95% or more binding activity to PD1 after activation.

### Example 4: Screening for Biomolecule R5, Conjugation to R1, R2-R3-R4, and Activation after Conjugation

During activation by a proteolytic enzyme in a pathological microenvironment, the site at which the activated linker arm binds to the biomolecule and its steric conformation has different effects on activation efficiency. Steric hindrance and structure-activity relationship determine an effect of cleavage by activation. To investigate an effect of the biomolecular conformation of conjugates of Example 3 in a pathological environment on activation by an enzyme or an acid in the pathological environment, the following in vitro activation investigations were performed.

In the activation test, 10 micrograms of a proteolytic enzyme for activation were added to 1 mg/ml Peg1000-R2-R3-R4-S-Cys-R5 conjugate for reaction for 1 hour at 37 °C. The concentration of small compound Peg1000 produced after activation was detected by HPLC and activation or cleavage efficiency (%) in relative to a control group was calculated. Cleavage efficiency of each conjugate is shown in the following Table 21.

TABLE 21

| | | | | | Cleavage |
|---|---|---|---|---|---|
| Mutant | R1 | R2 | R3 | R4 | efficiency |
| PD-1-Ab- | Peg1000 | -Ala-Ala-Asn- | | | Control, 100% |
| Gly28Cys | | | | | |
| PD-1-Ab- | Peg1000 | -Ala-Ala-Asn- | R3-1 | R4-3 | 99.5 |
| Gly28Cys | | | | | |
| PD-1-Ab- | Peg1000 | -Ala-Ala-Asn- | R3-2 | R4-3 | 99.8 |
| Gly28Cys | | | | | |
| PD-1-Ab- | Peg1000 | -Ala-Ala-Asn- | R3-3 | R4-3 | 98.6 |
| Gly28Cys | | | | | |

As shown in Tables 22 and 23, the automatically shedding arm R3 substantially had no effect on the activation of Legumain due to its small molecular weight and linear structure, with R3-2 producing the lowest effect.

TABLE 22
anti-PD-1 antibody 1

| | | | | Cleavage |
|---|---|---|---|---|
| R2 | R3 | R4 | R5 | efficiency |
| -Ala-Ala-Asn- | R3-2 | | | Control, |
| | | | | 100% |
| -Ala-Ala-Asn- | R3-2 | Paclitaxel | | 42.5 |
| | | (2-OH) | | |
| -Ala-Ala-Asn- | R3-2 | R4-5 | PD-1-Ab1-Gly28Cys | 88.4 |
| -Ala-Ala-Asn- | R3-2 | R4-6 | PD-1-Ab1-Gly28Cys | 93.5 |
| -Ala-Ala-Asn- | R3-2 | R4-7 | PD-1-Ab1-Gly28Cys | 99.8 |
| -Ala-Ala-Asn- | R3-2 | R4-8 | PD-1-Ab1-Gly28Cys | 63.5 |
| -Ala-Ala-Asn- | R3-2 | R4-9 | PD-1-Ab1-Gly28Cys | 62.4 |
| -Ala-Ala-Asn- | R3-2 | R4-1 | PD-1-Ab1-Gly28Cys | 83.8 |
| -Ala-Ala-Asn- | R3-2 | R4-3 | PD-1-Ab1-Gly28Cys | 94.5 |
| -Ala-Ala-Asn- | R3-2 | R4-1 | PD-1-Ab1-Gly28Cys | 86.7 |
| -Ala-Ala-Asn- | R3-2 | R4-3 | PD-1-Ab1-Gly28Cys | 97.5 |

TABLE 23
anti-CTLA-4 antibody

| | | | | Cleavage |
|---|---|---|---|---|
| Acidic activation | R3 | R4 | R5 | efficiency |
| Amide bond linkage | R3-2 | | | Control, |
| | | | | 100% |
| Amide bond linkage | R3-2 | R4-2 | CTLA-4-Ser26Cys | 99.8 |
| Amide bond linkage | R3-2 | R4-15 | CTLA-4-Ser26Cys | 99.5 |
| Amide bond linkage | R3-2 | R4-16 | CTLA-4-Ser26Cys | 91.7 |
| Amide bond linkage | R3-2 | R4-17 | CTLA-4-Ser26Cys | 94.3 |
| Amide bond linkage | R3-2 | R4-18 | CTLA-4-Ser26Cys | 93.4 |
| Ester bond | R3-2 | | | Control, |
| | | | | 100% |
| Ester bond | R3-2 | R4-4 | CTLA-4-Ser26Cys | 92.8 |
| Ester bond | R3-2 | R4-19 | CTLA-4-Ser26Cys | 97.4 |
| Ester bond | R3-2 | R4-20 | CTLA-4-Ser26Cys | 93.8 |
| Ester bond | R3-2 | R4-21 | CTLA-4-Ser28Cys | 95.4 |
| Ester bond | R3-2 | R4-22 | CTLA-4-Ser28Cys | 96.4 |

In Tables 22 and 23, the activation efficiency produced by molecules without conjugating with the biomolecule was used as a positive control, while the activation efficiency produced by molecules with conjugation to a hydroxyl at position 2 of a heterocyclic compound Paclitaxel was used as a negative control. The length and side chain group of R4 affected the cleavage efficiency of the whole conjugate when conjugating to a mutation site in a variable region or a non-variable region of an antibody. A longer chain structure of R4 produced enhanced cleavage efficiency. As detected, after conjugating to Ala-Ala-Asn or Ala-Ala-Asp and then binding to antibody, R4-1 to R4-25 of the present disclosure all allowed the mutants to retain >60% of the activation efficiency, and >90% of the activation efficiency in an acid-sensitive activation.

### Effect of R1 Selection and Test for Recovery of Binding Capacity

PBS (pH 7.2) was used to dilute ligand molecules PD1, TNFα, CTLA-4, CD28 to 1 ug/ml, respectively. Then each of the diluted solutions was used to fix in a 96-well plate (Nunc) overnight. 1× block BSA (ThermoFisher) was used to block the plate for 2 h. Corresponding conjugate of anti-PD-1 antibody, anti-TNFα antibody, anti-CTLA-4 antibody and anti-CD28 antibody was added, respectively, in an equal concentration, and allowed to bind at 37 °C for one hour. The plate was washed with PBST three times. HRP enzyme-conjugated human antibody used as secondary antibody was allowed to react at 37 °C for 1 hour and then washed with PBST three times. TMB substrate (Solarbio., Inc.) recognized by HRP enzyme was used for reaction at 37 °C for 15 minutes in dark. A 1/2 volume of stopping solution was used to stop the reaction. Absorbance strength (OD450) was read. The relative binding efficiency was calculated by a percentage ratio between the binding efficiency of the conjugate and the binding efficiency of the wild type antibody before or after activation. The results were shown in Tables 24-26.

TABLE 24
Change of the binding activity of anti-PD-1 antibody after conjugating to RI and
after activating the conjugating arm

| | | | | | | Binding |
|---|---|---|---|---|---|---|
| | | | | | | efficiency |
| | | | | | Binding | after |
| | | | | | efficiency | activating |
| | | | | | before | by enzyme |
| R1 | R2 | R3 | R4 | R5 | activation | or acid |
| | -Ala-Ala-Asn- | R3-2 | R4-3 | PD-1-Ab1-Gly28Cys | 67.4% | 143.5% |
| R1 from S1 | -Ala-Ala-Asn- | R3-2 | R4-3 | PD-1-Ab1-Gly28Cys | 24.4% | 145.3% |
| R1 from S3 | -Ala-Ala-Asn- | R3-2 | R4-3 | PD-1-Ab1-Gly28Cys | 22.5% | 143.4% |
| R1 from S11 | -Ala-Ala-Asn- | R3-2 | R4-3 | PD-1-Ab1-Gly28Cys | 15.6% | 137.4% |
| R1 from S17 | -Ala-Ala-Asn- | R3-2 | R4-3 | PD-1-Ab1-Gly28Cys | 3.2% | 156.3% |

The results indicate that conjugating to R1 inhibits the activity of the anti-PD-1 antibody but does not affect the activation of the antibody. The binding efficiency of the conjugate without R1 to its antigen before activation was 67.4% of that of the wild type antibody to the antigen. However, after activation, the binding efficiency of the conjugate was improved to be 143% of that of the wild type antibody. After binding R1 to the same R2-R3-R4 and mutated antibody, it prevented the resultant conjugate from binding to its antigen. And the prevention of binding increased as the molecular weight of R1 increased. S17 could completely prevent the conjugate from binding to its antigen. However, the binding activity was recovered after cleaving by Legumain.

TABLE 25
Change of the binding activity of anti-TNFα antibody after conjugating to R1 and
after activating the conjugating arm

| | | | | | | Binding |
|---|---|---|---|---|---|---|
| | | | | | | efficiency |
| | | | | | Binding | after |
| | | | | | efficiency | activating |
| | | | | | before | by enzyme |
| R1 | R2 | R3 | R4 | R5 | activation | or acid |
| | -Ala-Ala-Asp- | R3-2 | R4-2 | TNFα-Ab-Ala50Cys | 57.9% | 125.6% |
| R1 from S2 | -Ala-Ala-Asp- | R3-2 | R4-2 | TNFα-Ab-Ala50Cys | 28.3% | 128.9% |
| R1 from S4 | -Ala-Ala-Asp- | R3-2 | R4-2 | TNFα-Ab-Ala50Cys | 21.9% | 127.5% |
| R1 from S12 | -Ala-Ala-Asp- | R3-2 | R4-2 | TNFα-Ab-Ala50Cys | 11.6% | 127.4% |
| R1 from S18 | -Ala-Ala-Asp- | R3-2 | R4-2 | TNFα-Ab-Ala50Cys | 0.9% | 125.3% |

The results indicate that conjugating to R1 inhibits the activity of the anti-TNFα antibody but does not affect the activation of the antibody. The binding efficiency of the conjugate without R1 to its antigen before activation was 57.9% of that of the wild type antibody to the antigen. However, after activation, the binding efficiency of the conjugate was 25.6% of that of the wild type antibody. After binding R1 to the same R2-R3-R4 and mutated antibody, it prevented the resultant conjugate from binding to its antigen. And the prevention of binding increased as the molecular weight of R1 increased. S18 could completely prevent the conjugate from binding to its antigen. However, the binding activity was recovered after cleaving by Legumain.

TABLE 26
Change of the binding activities of anti-CTLA-4 antibody
and anti-CD28 antibody after conjugating to the conjugate
and after activating the conjugating arm

| | | Binding | Binding |
|---|---|---|---|
| | | efficiency | efficiency after |
| conjugating | | before | activating by |
| arm | R5 | activation | enzyme or acid |
| S7 | CD28-Gly44Cys | 22.5% | 143.4% |
| S8 | CD28-Ala40Cys | 15.6% | 137.4% |
| S9 | CD28-Thr97Cys | 3.2% | 156.3% |
| S10 | CTLA-4-Ser26Cys | 67.4% | 143.5% |
| S13 | CTLA-4-Ser28Cys | 24.4% | 145.3% |
| S14 | CTLA-4-Tyr92Cys | 22.5% | 143.4% |
| S15 | CTLA-4-Leu34Cys | 15.6% | 137.4% |
| S16 | CTLA-4-Gly51Cys | 3.2% | 156.3% |

The results show that conjugating to R1 inhibits the activity of anti-CD28 antibody and anti-CTLA-4 antibody but does not affect the activation of the antibody.

From Example 4, it could be found that the activation efficiency of a conjugate and its binding capacity to antigen after recovery were influenced by the mutation position in the antibody to some extent. However, with the use of R4 expected activation efficiency by enzymatic cleavage could be produced. As for the binding between a biomolecule and its ligand, the exposed group of R4 produced after cleavage by activation could regulate the binding capacity of an antibody mutant. Different exposed groups had different effects on different mutation positions. By screening for R1 and D4, it could be found that the hindering function of R1 led to complete loss of binding capacity of a conjugate to its antigen. However, the binding capacity was recovered or even enhanced after target cleavage by an enzyme. Such conjugate-type antibodies are only activated in a region that a target enzyme is highly expressed or secreted in a pathological microenvironment to release the antibody or protein. Thus, such microenvironment-activated antibodies are new target-activated antibodies as shown in FIG. 10.

### Example 5: Promotion or Inhibition of Antibody on Immune Indexes

### 1. Anti-PD-1 Antibody Promoted T Cells to Secrete IFN-γ

Human whole blood (from Shang Ruijin Hospital) was diluted and uniformly mixed with PBS in an equal amount. The diluted solution was slowly transferred to another centrifugal tube containing lymphocyte separation solution along the wall of the centrifugal tube to allow the diluted solution to form a separated layer on the lymphocyte separation solution. The interface was kept clear. The tube was centrifuged at 2000 rpm for 20 minutes. After centrifugation, the centrifugal tube was taken out. The solution within the tube was layered. A mononuclear cell layer was extracted to a 50 ml centrifugal tube. Five-time volume of PBS was added and uniformly mixed and the mixture was centrifuged at 2000 rpm for 10 minutes. The supernatant was discarded and the precipitate was resuspended with PBS. Then the resultant mixture was centrifuged at 1500 rpm for 10 min and the resultant human peripheral blood mononuclear cells (PBMC) were resuspended with the RPMI1640 complete medium containing 5% heat-activated human serum. The human PBMC was inoculated in a 96-well plate in a concentration of 1~2×10⁵ cells per well. The cells were stimulated with 0.1 ug/ml SEB for three days. The non-adherent cells (mainly mononuclear cells) were collected and uniformly inoculated to a new 96-well plate. Different concentrations of anti-PD-1 antibody without mutation or PD-1-Ab-Cys28-S-S13 conjugate were added and cultured at 37 °C, 5% CO₂ for 4 days. Supernatant was collected and concentration of cytotoxic factor IFN-γ was detected by ELISA kit. Results showed that the activated anti-PD-1 antibody exhibited a close or even a significantly improved activity of secreting IFN-γ, as shown in FIG. 11.

The results indicate that after activation of the anti-PD-1 antibody conjugate in vitro, not only the stimulation of anti-PD-1 antibody to T cell could be recovered, but also the remaining group produced after cleavage could introduce one or several new functional groups for the antibody. As a result, after activation, PD-1-Ab-Cys28-S-S13 enhanced the activity of such activating T cells. Such kinds of conjugated-type antibodies are merely activated in a region highly expressing or secreting a target enzyme in a pathological microenvironment to release the antibody. Thus, such microenvironment-activated antibodies are new target-activated antibodies.

### 2. Conjugates of Anti-PD-1 Antibody and Anti-CD28 Antibody Reduce Body's Autoimmunity

As is well known in the art, though anti-PD-1 antibody is a drug effective for treating tumor, it was found in the current clinic research that anti-PD-1 antibody exhibited two main issues. One of the issues is that a patient would exhibit a side effect of high fever and false progress after administration of the antibody, but the mechanism is unknown. We presume these side effects may be improved if the antibody is inhibited or blocked by a conjugate and is released after arriving at a local environment of tumor so as to reduce the exposed time or dose of active drug in a non-diseased environment. For this reason, experiments were conducted with mice suffered from type I diabetes mellitus (NOD). Diabetes mellitus of this kind of mice is an autoimmune disease, wherein self-activated T lymphocyte cells destroy pancreatic islet β cells, resulting in insufficient secretion of insulin. First, female NOD of 10 weeks old (Beijing Vital River Laboratory Animal Technology Co., Ltd.) were injected with control IgG, low (1 mpk), medium (5 mpk) or high (15 mpk) dose of antibody or conjugate of antibody, respectively at day 0. Indicators of diabetes mellitus, including glucose in urine and two blood sugar levels were observed every day for 12 days until no new indicator of urine glucose was observed.

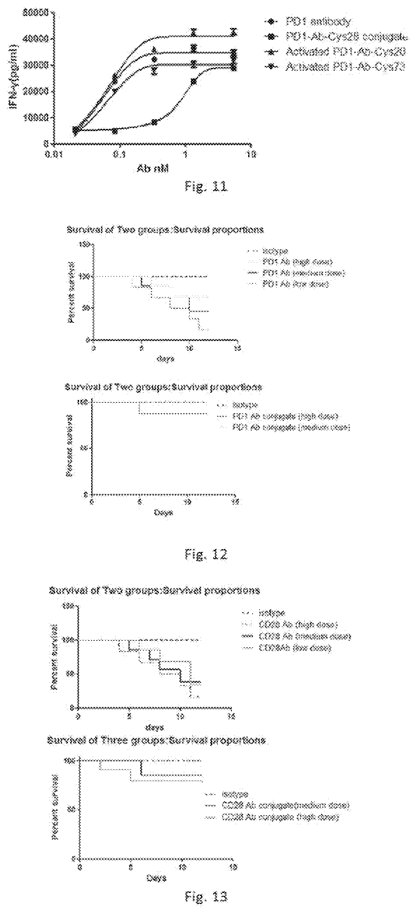

Protection from autoimmunity by the PD-1-Ab-Cys28-S-S13 conjugate was shown in FIG. 12 and protection from autoimmunity by the CD28-Ab-Cys-S-S13 conjugate was shown in FIG. 13.

Results showed that protection of an immune system by a conjugate of anti-PD-1 antibody was increased by about 15 times as compared to anti-PD-1 antibody, and protection of an immune system by a conjugate of anti-CD28 antibody was increased by about 10 times as compared to anti-CD28 antibody. It can thus be seen that, when a linking moiety was used to hinder or reduce the activity of a protein or antibody, the binding of a conjugated protein or antibody to a related ligand in a normal tissue was reduced, because it was difficult to activate anti-PD-1 antibody and anti-CD28 antibody due to hydrolase and physiological environment outside a diseased microenvironment. As a result, the conjugate could reduce autoimmunity as compared to the primary antibody.

### 3. Conjugate of Anti-CD28 Antibody Specifically Activates CD4 or CD8 Cells.

CD8+ T cells and CD4+ T cells were isolated from human peripheral blood mononuclear cells with CD8 or CD4 magnetic beads (Dynabeads) according to the specific steps provided in the specification of a kit, counted and stained according to the CFSE specification. The cells were inoculated in a 96-well plate at a concentration of 1~2×10⁵ cells per well. A suitable amount of control anti-CD3/anti-CD28 antibody, 0.3 ug/ml of anti-CD28 antibody or conjugate of anti-CD28 antibody were added, respectively.

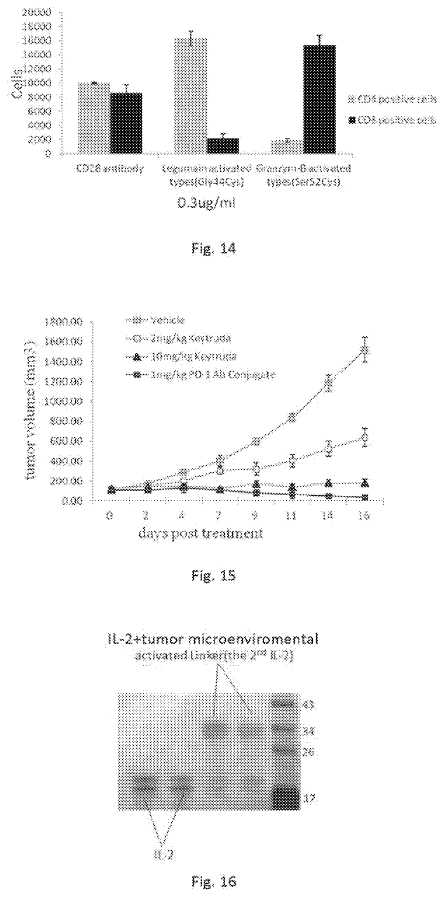

Results were shown in FIG. 14. According to the results, as compared to the original anti-CD28 antibody, the heavy chain conjugate CD28-Ab-Gly44Cys (CD28-Ab-Cys44-S-S13) could selectively activate CD4 T cells, and the light chain conjugate CD28-Ab-Ser52Cys (CD28-Ab-Cys52-S-S13) could selectively activated CD8 T cells. The results indicate that, when a linking moiety was used to hinder or reduce the activity of a protein or an antibody, the binding of the antibody conjugated to the linking moiety to a related antigen in a normal tissue was reduced before the antibody or protein arrives at a target tissue, because the hydrolase is expressed in a low level outside a diseased microenvironment and it is very difficult for the hydrolase to activate the linking moiety conjugated to the antibody or the protein. When the antibody conjugated to the linking moiety arrives at the diseased microenvironment, such as an inflammatory microenvironment, the conjugated linking moiety is activated by Legumain expressed on the surface of Treg, thereby stimulating proliferation of Treg and inhibiting inflammatory reaction. In a tumor region, the conjugated linking moiety is activated by Granzym-B expressed on the surface of CD8 cells, thereby stimulating proliferation of CD8 cells and inhibiting progress of tumor. The designed conjugates are regulated by hydrolase in a microenvironment and the activity of antibody is released only after the conjugated linking moiety is hydrolyzed. As a result, antibodies conjugated to a linking moiety could be greatly enriched in a microenvironment. Eventually, a whole effect, i.e., reduced side effects and improved efficacy could be achieved.

### Example 6: Anti-PD-1 Antibody Activated in a Microenvironment could Cure MC38 Tumor

MC38 cells were subcutaneously inoculated to transgenic C57BL/6 mice (Shanghai Research Center for Model Organisms) transformed with human PD-1 fusion protein in a concentration of 2×10⁶ cells per mouse. One week later, the mice implanted with MC38 tumor were randomly divided into 4 groups. Group 1 was injected with 10 mg/kg anti-PD-1 antibody Keytruda® (pembrolizumab), group 2 was injected with 2 mg/kg anti-PD-1 antibody Keytruda® (pembrolizumab), group 3 was injected with 1 mg/kg PD-1-Ab-G28C-S13 conjugate, and group 4 was injected with a solvent control, twice a week for 2 weeks. The tumors in mice were recorded 3 times each week. Results were shown in FIG. 15.

Mouse transformed with human PD-1 fusion protein is a transgenic mouse. Its endogenous PDCD1 gene is replaced with human PDCD1, thereby expressing human PDCD1 protein. Such mouse could respond to human anti-PD-1 antibody and stimulate downstream immunity. The results show that 10 mg/kg anti-PD-1 antibody could effectively inhibit growth of MC38 tumor, with one mouse being cured and five mice having a significant inhibitory effect, which was 80% or more. 1 mg/kg PD-1-Ab-G47C-S13 conjugate produced better inhibitory effect than 10 mg/kg anti-PD-1 antibody Keytruda® (pembrolizumab). The results demonstrate that the conjugate of anti-PD-1 antibody could enhance the activity of effector T cells and enrichment of the antibody in a tumor microenvironment, as proved in Example 5. Thus, the conjugate exhibits an improved efficacy than the original antibody.

### Example 7a: IL2 Protein Activated in a Microenvironment

The amino acid sequence of IL2 is a native human IL2 protein. Its DNA sequence was optimized for expression in a host and synthesized (GENEWIZ, Inc., Suzhou, China). The synthesized DNA was digested and ligated to a modified pTT5 vector (Biovector) to produce pTT5-IL2. The mutated vector was constructed by using pTT5-IL2 as template, designing primer to replace the codon at the mutation position with that of cysteine, PCR and digesting and constructing the mutated fragment to the modified pTT5 vector. Synthesis, mutation and transfection of IL2 gene were performed according to the methods described in Example 2. Its binding activity to ligand IL2Ra or IL2Rb was analyzed by IP and ELISA in accordance with the mutation position.

To verify that activation of macromolecule in a microenvironment is not limited to antibody but is applicable to various proteins, cytokine IL2 was used as an example herein. Its mutation position, activity, linker arm, and activity and function after activation were screened. Results are shown in the following Tables 27-29.

TABLE 27
Activity of mutated IL2 and screening for linker arm

| | | | Binding | Binding | | |
|---|---|---|---|---|---|---|
| | | Type of | activity to | activity to | | |
| Amino | | inhibitory | IL2Ra after | IL2Rb after | Conjugating | Conjugating |
| acid | Position | ligand | mutation | mutation | to S3 | to S13 |
| Lys | 32 | α | 86.5% | 100% | 26.1% | |
| Lys | 35 | α | 87.1% | 100% | | 32.4% |
| Thr | 37 | α | 98.1% | 100% | | |
| Thr | 41 | α | 99.2% | 100% | 18.7% | |
| Lys | 43 | α | 89.9% | 100% | | 31.3% |
| Lys | 48 | α | 88.4% | 100% | | |
| Lys | 49 | α | 79.8% | 100% | | 39.4% |
| Leu | 72 | α | 98.8% | 100% | | 25.5% |
| Ala | 73 | α | 84.3% | 100% | 27.9% | |
| Ser | 75 | α | 86.2% | 100% | | |
| Lys | 76 | α | 85.0% | 100% | | 32.2% |
| Leu | 94 | α | 99.6% | 100% | | 27.2% |
| Thr | 101 | α | 99.8% | 100% | | 32.0% |
| Thr | 102 | α | 95.3% | 100% | | 29.6% |
| Ala | 108 | α | 93.9% | 100% | | 16.9% |
| Thr | 111 | α | 94.9% | 100% | | 35.3% |
| Ala | 112 | α | 97.3% | 100% | | 41.9% |
| Leu | 19 | β | 100% | 97.5% | 39.5% | |
| Gly | 27 | β | 100% | 42.3% | | |
| Ser | 75 | β | 100% | 93.3% | | 31.1% |
| Leu | 80 | β | 100% | 27.0% | | |
| Ser | 87 | β | 100% | 31.6% | | |
| Leu | 94 | β | 100% | 47.6% | | |
| Gly | 98 | β | 100% | 79.5% | | 16.9% |
| Ser | 99 | β | 100% | 99.2% | | 18.9% |
| Thr | 101 | β | 100% | 21.7% | | |
| Thr | 133 | β | 100% | 96.9% | | 27.8% |
| Thr | 51 | γ | 100% | 100% | 89.70% | 91.60% |

It could be found from the results that, for the mutants retaining 8000 or more of binding activity to its corresponding receptor as compared to the native IL2, their activity of binding receptor could be inhibited or hindered by regulating the length of a linker arm. For example, after mutating Lys32, Thr41, Ala73 or Leu19 to Cys, conjugating each of the resulted mutants to S3 may inhibit 60% of binding activity to their corresponding receptor; whereas after mutating Lys35, Lys43, Leu72, Lys76, Leu94, Thr101, Thr102, Ala108, Thr111, Ala112, Gly98, Ser99, Thr133, or the like to Cys, conjugating each of the resulted mutants to S13 may inhibit 60% or more of binding activity to their corresponding receptor.

TABLE 28
Cleavage efficiency of conjugated IL2

| | | | | Cleavage |
|---|---|---|---|---|
| R2 | R3 | R4 | R5 | efficiency |
| -Ala-Ala-Asp- | R3-2 | | | Control, 100% |
| -Ala-Ala-Asp- | R3-2 | Paclitaxel (2-OH) | | 45.5% |
| -Ala-Ala-Asp- | R3-2 | R4-10 | IL2-Thr37 | 86.5% |
| -Ala-Ala-Asp- | R3-2 | R4-11 | IL2-Thr37 | 92.7% |
| -Ala-Ala-Asp- | R3-2 | R4-12 | IL2-Thr37 | 96.7% |
| -Ala-Ala-Asp- | R3-2 | R4-13 | IL2-Thr37 | 73.9% |
| -Ala-Ala-Asp- | R3-2 | R4-14 | IL2-Thr37 | 62.4% |
| -Ala-Ala-Asp- | R3-2 | R4-1 | IL2-Thr37 | 73.8% |
| -Ala-Ala-Asn- | R3-2 | R4-3 | IL2-Thr37 | 84.7% |
| -Ala-Ala-Asp- | R3-2 | R4-1 | IL2-Thr41 | 86.7% |
| -Ala-Ala-Asp- | R3-2 | R4-3 | IL2-Thr41 | 95.5% |

Results indicate that activation by Legumain is not influenced due to the small molecular linear structure of the automatically shedding arm R3.

TABLE 29
Change of binding efficiency to its receptor after conjugating
IL2 to R1 an after activation of a linking arm

| | | Binding efficiency | Binding efficiency after |
|---|---|---|---|
| R1 | R5 | before activation | enzymatic or acidic activation |
| S3 | IL2-Thr37 | 28.1% | 143.5% |
| S5 | IL2-Ser87 | 31.6% | 145.3% |
| S6 | IL2-Thr-41 | 99.2% | 124.8% |
| S13 | IL2-Leu-19 | 97.5% | 118.7% |

It can thus be seen that the activation efficiency of a conjugate and its binding capacity to receptor after activation were influenced by the mutation position in a protein to some extent. However, with the use of R4, activation efficiency by target enzymatic cleavage could be produced. As for the binding between a biomolecule and its ligand, the exposed group of R4 produced after cleavage by activation could regulate the binding capacity of a mutated antibody. Different exposed groups had different effects on different mutation positions. By screening for R1 and D4, it could be found that the hindering function of R1 led to complete loss of binding capacity of the conjugate to its antigen. However, the binding capacity was recovered or even enhanced after cleavage by a target enzyme. Such conjugated-type proteins are merely activated or released their activity in a region highly expressing or secreting a target enzyme in a pathological microenvironment. Thus, such proteins are new target-activated proteins.

IL2 mutants having two or more mutations were prepared and conjugated to S13. The mutation sites were selected from the mutation sites of the aforementioned IL2 mutants having one mutation and having 95% or more binding activity to its receptor. The activity of such conjugates after activation was verified and the results were shown in Table 30.

TABLE 30
Binding activity of conjugates of IL2 mutants with two or
more mutations to its receptor before and after activation

| | | Binding | Binding efficiency |
|---|---|---|---|
| R1-R2- | | efficiency before | after enzymatic |
| R3-R4 | R5 | activation | or acidic activation |
| S13 | IL2-Thr37/Leu19 | 12.1% | 113.5% |
| S13 | IL2-Ser37/Thr41 | 11.6% | 98.3% |
| S13 | IL2-Thr-41/Ser87 | 19.2% | 102.8% |
| S13 | IL2-Leu-19/Ser87 | 17.5% | 96.7% |
| S13 | IL2-Leu19/Ser37/ | 10.9% | 95.9% |
| | Ser87 | | |

The above results demonstrate that conjugates of IL2 mutants with two or more mutations could retain 95% or more binding activity to corresponding receptor after activation.

### Conjugate of IL2 Protein Specifically Activates CD4 or CD8 Cells.

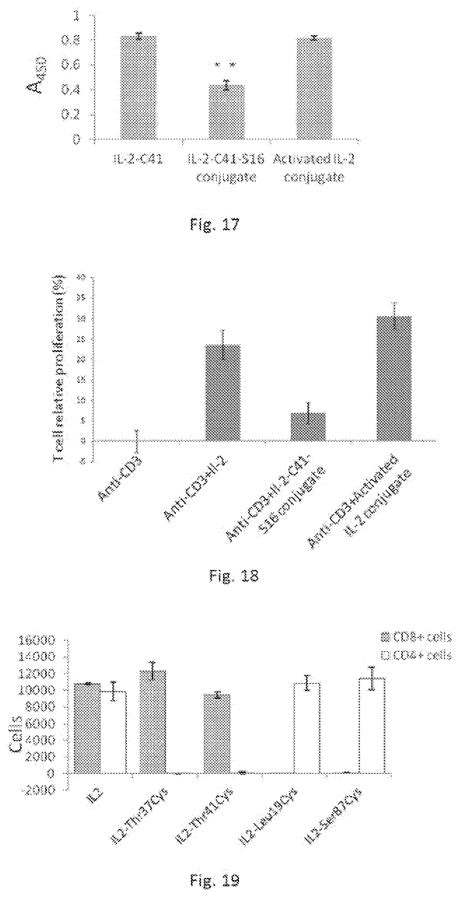

CD8+ T cells and CD4+ T cells were isolated from human peripheral blood mononuclear cells with CD8 or CD4 magnetic beads (Dynabeads) according to the specific steps provided in the specification of a kit, counted and stained according to the CFSE specification. The cells were inoculated in a 96-well plate at a concentration of 1~2×10⁵ cells per well. A suitable amount of control anti-CD3 antibody, 0.05 ug/ml of IL2 protein or conjugate IL2-T41C-S16 conjugated to S16 were added, respectively. FIG. 16 shows conjugation of IL2-T41C (IL2-Thr41Cys) to S16. FIG. 17 shows the binding to IL2 receptor alpha before and after conjugation. FIG. 18 shows effect of the IL2-C41-S16 conjugation on proliferation of T cells before and after activation.

Native IL2 exhibited similar stimulation on CD8 and CD4. However, after conjugating to a functional moiety activated in a microenvironment, IL2-Thr37Cys and IL2-Thr41Cys could specifically activate proliferation of CD8 T cells and reduce proliferation of CD4 cells. The ratio of cell number between CD8 cells and CD4 cells increased from the original 1:1 to 410:1 and 157:1, respectively. IL2-Leu19Cys and IL2-Ser87Cys could specifically activate proliferation of CD4 T cells, with the cell ratio of CD4/CD8 being increased to 435:1 and 126:1, respectively. Results are shown in FIG. 19. The results demonstrate that CD8 or CD4 could be selectively activated by conjugating IL2 to a functional moiety to control its binding activity to each of receptors and to allow it to be activated in a specific microenvironment.

### Conjugates of IL2 Protein Effectively Inhibit Growth of Melanoma B16F10 and Colon Cancer MC38.

0.5×10⁶ B16F10 cells per mouse were subcutaneously inoculated to C57BL/6 mice. One week later, when the tumor's volume reached 100 mm³, the mice implanted with melanoma were randomly divided into 4 groups, with 6 mice per group. Group 1 was injected with 2.5 mg/kg of IL2-T37C-S14 protein conjugate weekly. Group 2 was injected with 0.5 mg/kg of IL2-Thr37Cys protein conjugate weekly. Group 3 was injected with 3 mg/kg of aldesleukin (control) twice a week. Group 4 was injected with solvent (control) twice a week. All groups were continuously administered for 3 weeks. Mice tumors were measured twice a week and mice were weighed twice a week.

MC38 cells were subcutaneously inoculated to transgenic C57BL/6 mice transformed with human PD-1 fusion protein in a concentration of 0.5×10⁶ cells per mouse. One week later, the mice implanted with MC38 tumor were randomly divided into 4 groups. Group 1 was injected with 2 mg/kg of IL2-T37C-S14 protein conjugate. Group 2 was injected with 0.5 mg/kg of IL2-Thr37Cys conjugate and 100 μg per time of anti-PD-1 antibody twice a week. Group 3 was injected with 3 mg/kg of aldesleukin (control) twice a week. Group 4 was injected with solvent (control) twice a week. All groups were continuously administered for 3 weeks. Mice tumors were measured twice a week and mice were weighed twice a week.

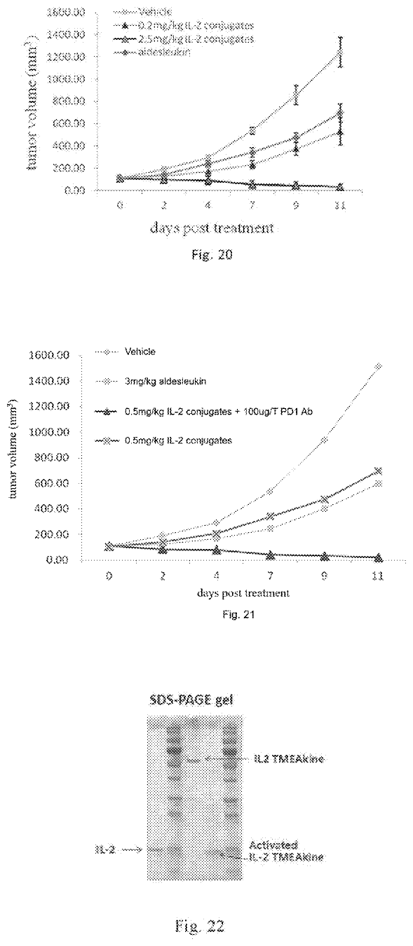

Results are shown in FIGS. 20 and 21, which demonstrate that the IL2-Thr37Cys conjugate could not only be used in a reduced use dose, but also significantly enhance the effect of treating B16F10 tumor, as compared to the existing IL2 product, aldeslekin. And in the combination treatment group with anti-PD-1 antibody, there were MC38 mice being cured.

From the above results, it could be found that, when using a functional moiety to hinder or reduce the activity of a protein or antibody, the binding of a protein to its receptor or ligand in a normal tissue could be reduced before it arrives at a target tissue because it is very difficult to activate the functional moiety conjugated to the protein by a hydrolase and a physiological environment outside a diseased microenvironment. However, in a diseased microenvironment, IL2-Thr37Cys and IL2-Thr41Cys and the like were influenced by the hydrolase in the diseased microenvironment and activated on the surface of Granzym-B highly expressing CD8 cells, thereby binding to a receptor on the surface of CD8 cells and activating CD8 cells. As a result, the protein conjugated with a functional moiety could reduce immune toxicity while enhancing targeted efficacy.

For the same reasons, IL2-Leu19Cys and IL2-Ser87Cys were activated on the surface of Legumain highly expressing Treg cells, allowing the activated IL2 to bind to a receptor on the surface of Treg cells to activate proliferation of Treg cells.

### Example 7b: Tumor Microenvironment Activated IL2 Cytokine (IL2 TMEAkine)

### 1. Expression and Purification of the Mutant IL2 Cytokine

The mutant IL2 DNA sequence ligated to a modified pTT5 vector (Biovector) was optimized for expression in 293T cells and synthesized (GENEWIZ Inc., Suzhou, China). Transfection of the mutant IL2 DNA was performed. After incubation for 4-7 days, the supernatant containing mutant IL2 was collected.

In eukaryotic expression, the expression vector pPICZα A containing the mutant IL2 genes was optimized and prepared (GENEWIZ Inc., Suzhou, China). The amino acid sequence of wild type IL2 was described is shown below:

(SEQ ID NO: 11)
APASSTKKTQLQLEHLLLDLQMILNGINNYKNPKLTRMLTFKF
YMPKKATELKHLQCLEEELKPLEEVLNLAQSKNFHLRPRDLIS
NINVIVLELKGSETTFMCEYADETATIVEFLNRWITFSQSIIS
TLT.

The expression vector pPICZα A was transformed in E. coli (DH5α) for plasmid purification. Then pPICZα A was transformed into GS115 by electroporation. The transformed colony was selected by obtaining the growing colonies after growing on the 100, 300, 500, 1000, 1500, 2000 μg/mL Zeocin™ containing YPD plates. After finally selecting the transformant, the recombinant GS115 strain was grown in BMGY medium at 30 °C, with vigorous shaking in baffled flasks to an OD600 of 2-6. The cells were then pelleted by centrifugation and suspended in BMMY to an OD600 of 1, to which was added 0.5% methanol daily in order to induce the heterologous protein expression. After a four-day induction, supernatant containing the secreted mutant IL2 protein was collected by centrifugation. The total protein in the supernatant was concentrated by ultrafiltration using a 10-kDa molecular mass cutoff membrane. The concentrated protein was dialyzed with buffer A (50 mM HAc/NaAc, pH4.5) for more than 24 h, then loaded onto a cation-exchange column equilibrated with buffer A. Mutant IL2 was eluted from the column with gradient concentration of NaCl and the eluent was collected and concentrated. The condensed sample was further purified on Sephacryl S-100 HR gel filtration column using 20 mM Tris-HCl, 20 mM NaCl, pH7.4, as the elution buffer.

In prokaryotic expression, the expression vector pET22b (+) containing the mutant IL2 genes was optimized and prepared (GENEWIZ Inc., Suzhou, China). The amino acid sequences of wild type IL2 were described in SEQ ID NO: 12. The positive clone was selected and transformed into the E. coli cells (BL21DE3). Standard procedure for induction of the target protein using isopropyl thiogalactoside (IPTG) was followed. The induced E. coli cells were centrifuged and the cell pellet was resuspended in 100 mM Tris-HCl buffer (pH 8.0) containing 5 mM EDTA and 1 mM PMSF. Cells were lysed by sonication and centrifuged to isolate IL2 protein inclusion bodies (IBs). The IB pellet was then washed with 100 mM Tris-HCl buffer (pH 8.0) containing 5 mM EDTA and 2% deoxycholate, and distilled water, respectively. The IBs were solubilized in 6 M guanidine hydrochloride (GuHCl) solution (prepared in 0.1 M Tris buffer, pH 8.0) and incubated for 30 min at room temperature with gentle vortexing, followed by centrifugation. The supernatant was diluted with refolding buffer (0.1 M Tris buffer, pH 8.0 containing 10 mM reduced and 1 mM oxidized glutathione in a ratio of 10:1) so as to obtain a protein concentration and GuHCl of 0.1 mg/mL and 2 M, respectively. Subsequently, the solution was kept for 16 h at room temperature for slow refolding of IL2. The insoluble protein was removed by centrifugation. The supernatant was concentrated and loaded on a gel filtration column Sephacryl S-100 HR, equilibrated with 0.1 M Tris buffer containing 2 M GuHCl.

Wild type IL2 amino acid sequence produced by the transformant is shown below:

(SEQ ID NO: 12)
PTSSTKKTQLQLEHLLLDLQMILNGINNYKNPKLTRMLTFKFY
MPKKATELKHLQCLEEELKPLEEVLNLAQSKNFHLRPRDLISN
INVIVLELKGSETTFMCEYADETATIVEFLNRWITFSQSIIST
LT.

2. Screening the Mutants of their Binding Activity to IL2Rα or IL2Rβ after Mutation

Various IL2 mutants were expressed by 293T cells and secreted to the medium. Supernatant containing IL2 mutants was obtained through centrifugation as described in section 1. Then 1 ug His-tagged IL2Rα or IL2Rβ was added to 1 mL supernatant mentioned above and incubate for 1 h at 4 °C with gentle agitation. 50 uL pre-washed Ni-NTA resin was transferred to the mixed solution of the supernatant and IL2Rα or IL2Rβ, and incubated for 1 h at 4 °C with gentle agitation. The mixture was subjected to centrifugation and the supernatant was discarded. The resultant pellet was washed with 500 μL PBS containing 25 mM imidazole for three times. The amount of IL2 mutants and IL2Rα/Rβ was visualized by western blotting using IL2 antibody and anti His-tag monoclonal antibody.

### 3. Conjugating S47 to the Mutant IL2

Mutant IL2 protein was generated and purified as described above in section 1. Purified mutant IL2 was incubated as the concentration of 0.3 mg/mL in 50 mM phosphate buffer (pH 7.4) containing 5 mM EDTA. TCEP solution was added to mutant IL2 in a molar ratio of 100:1 and the resultant mixture was incubated for 4 h at 4 °C with gentle agitation. Then the mixture was dialyzed with 50 mM phosphate buffer (pH 7.4) containing 150 mM NaCl for 2 h at 4 °C. Afterwards, S47 was immediately added to the mixture in a molar ratio of 20:1 and the resultant mixture was incubated for 16 h at 25 °C with gentle agitation. The reaction was stopped and residual S47 was removed. Before enzyme cleavage, the buffer used for the conjugate of IL2-S47 (IL2 TMEAkine, Tumor Microenvironment Activated IL2 cytokine) was changed to a buffer used for enzyme through dialysis. Then enzyme was added to the IL2 TMEAkine solution and the mixture was incubated at 37 °C for 16 h. FIG. 22 shows the SDS-PAGE results, with a colloidal blue stain for mutant IL2, IL2 TMEAkine and the recovery active IL2 after enzyme cleavage in vitro.

4. Screening IL2 TMEAkine that Blocks the Binding to IL2Rα or IL2Rβ and Recovers the Binding Activity after Enzyme Cleavage In Vitro

60 μl PBS buffer containing 1 ug IL2Rα-Fc/IL2Rβ-Fc solution were dispensed into wells. Sealing tape was applied to the top of the plate and then the plate was incubated at 4 °C overnight. After incubation, the tape was removed to aspirate each well. After washing with PBST for three times, the plate was blocked by dispensing 200 μl of PBS buffer containing 2% BSA into each well and then the plate was incubated at room temperature for 2 h. The plate was washed three times and 60 μl of serial diluted samples were added to the appropriate wells. The plate was incubated at room temperature for 1.5 h. After washing with PBST for three times, 60 μl of 1 μg/mL IL2 biotinylated antibody solution was dispensed to each well and the resultant mixture was incubated at room temperature for 1 hour. The plate was washed three times and then 60 μl of streptavidin solution was dispensed to each well. Then the plate was incubated at room temperature for 30 minutes. After washing three times, 100 μl of the HRP substrate solution was dispensed into each well and the plate was incubated at 37 °C for 15 minutes. After color development, 50 μl of stop solution was dispensed into each well and the absorbance of each well was immediately measured at a wavelength of 450 nm. ELISA results were shown in FIG. 23, indicating the binding activity of IL2 TMEAkine to IL2Rα or RP before and after enzyme cleavage in vitro. Compared with binding activity of wild type IL2 to IL2Rα and IL2Rβ, IL2 TMEAkine binds to IL2Rα with much lower binding affinity but binds to IL2Rβ with almost the same binding affinity. After activation by enzyme, the binding affinity of IL2 TMEAkine to IL2Rα equals to that of wild type IL2 to IL2Rα.

### 5. Summary of Various IL2 Mutation Sites

IL2 receptors may associate on the cell surface to form the following heteromers: intermediate-affinity receptor: IL2Rβγ to IL2 (Kd=1 nM) and low-affinity receptor: IL2Rα to IL2 (Kd=10 nM). Because it is a low-affinity binding (Kd=10 nM), it is easier than CDR region of antibody to screen a position for linking to R4 group for recovering the binding affinity. Because we did not want to increase the binding affinity between R4-S-IL2 and IL2Rα, we selected the conjugated IL2 with the special R4, which can recover the native binding affinity. In some cases, there are some R4 groups which can enhance the binding affinity, but we did not select them as drug candidates. We prefer R-1, R4-7, R4-5, R4-8 and R4-12 for the large-scale synthesis in our CMC development. S47 is cleaved by Legumain. After cleaving, its R4-7 chemical group remained. To select drug candidates, we also performed the screening expression and S47 conjugation reaction with all amino acids of IL2 in the domain that binds IL2Rα and IL2Rβ. We acquired the possible drug candidates and results are shown in the following Tables 31-33.

TABLE 31
Activity of mutated IL2 and screening for linker arm

| | | | | | Decreased fold of |
|---|---|---|---|---|---|
| | | Type of | | Binding | binding to ligand |
| | Amino | inhibitory | | recovery | after Conjugating |
| Position | acid | ligand | R4 | (>60%) | with S47(>3fold) |
| 32 | K | α | R4-7 | 100.2% | 3-fold |
| 35 | K | α | R4-7 | 85.4% | 89-fold |
| 37 | T | α | R4-7 | 98.9% | 51-fold |
| 38 | R | α | R4-18 | 81.2% | 145-fold |
| 41 | T | α | R4-7 | 99.2% | 135-fold |
| 42 | F | α | R4-8 | 82.3% | 120-fold |
| 43 | K | α | R4-7 | 89.3% | 110-fold |
| 43 | K | α | R4-18 | 109.7% | |
| 44 | F | α | R4-8 | 96.7% | 60-fold |
| 45 | Y | α | R4-7 | 96.8% | 125-fold |
| 45 | Y | α | R4-8 | 143.6% | |
| 48 | K | α | R4-7 | 100.2% | 12-fold |
| 49 | K | α | R4-18 | 100.7% | |
| 60 | E | α | R4-12 | 97.1% | 5-fold |
| 61 | E | α | R4-12 | 83.4% | 134-fold |
| 62 | E | α | R4-12 | 86.6% | 120-fold |
| 63 | L | α | R4-5 | 99.3% | 13-fold |
| 64 | K | α | R4-7 | 97.1% | |
| 67 | E | α | R4-12 | 99.6% | 9-fold |
| 68 | E | α | R4-12 | 95.4% | 89-fold |
| 71 | N | α | R4-18 | 98.6% | 5-fold |
| 72 | L | α | R4-5 | 90.1% | 110-fold |
| 74 | Q | α | R4-19 | 100.0% | 3-fold |
| 75 | S | α | R4-7 | 100.0% | 4-fold |
| 76 | K | α | R4-7 | 100.0% | 3-fold |
| 101 | T | α | R4-7 | 100.0% | 5-fold |
| 102 | T | α | R4-7 | 100.0% | 4-fold |
| 102 | T | α | R4-3 | 122.5% | |
| 107 | Y | α | R4-4 | 97.7% | 78-fold |
| 108 | A | α | R4-5 | 100.0% | 10-fold |
| 111 | T | α | R4-7 | 98.9% | 20-fold |
| 112 | A | α | R4-5 | 100.0% | 5-fold |
| 5 | S | β | R4-7 | 99.8% | 3-fold |
| 5 | S | ß | R4-2 | 109.5% | |
| 8 | K | β | R4-7 | 66.7% | 6-fold |
| 9 | K | β | R4-6 | 97.0% | 9-fold |
| 9 | K | β | R4-7 | 124.6% | |
| 12 | L | β | R4-5 | 93.4% | 13-fold |
| 13 | Q | β | R4-19 | 85.1% | 28-fold |
| 15 | E | β | R4-12 | 81.4% | 25-fold |
| 19 | L | β | R4-5 | 92.3% | 27-fold |
| 20 | D | β | R4-11 | 87.3% | 31-fold |
| 27 | G | β | R4-1 | 100.0% | 3-fold |
| 76 | K | β | R4-6 | 100.0% | 3-fold |
| 76 | K | β | R4-7 | 130.4% | |
| 80 | L | β | R4-2 | 100.0% | 5-fold |
| 81 | R | β | R4-18 | 95.2% | 12-fold |
| 84 | D | β | R4-11 | 80.1% | 36-fold |
| 85 | L | β | R4-7 | 98.4% | 9-fold |
| 85 | L | β | R4-5 | 113.6% | |
| 87 | S | β | R4-7 | 99.5% | 30-fold |
| 88 | N | β | R4-18 | 82.3% | 46-fold |
| 91 | V | β | R4-5 | 94.5% | 25-fold |
| 92 | I | β | R4-5 | 89.3% | 19-fold |
| 94 | L | β | R4-5 | 99.5% | 30-fold |
| 95 | E | β | R4-12 | 60.1% | 8-fold |
| 98 | G | β | R4-1 | 98.6% | 4-fold |
| 99 | S | β | R4-7 | 100.0% | 3-fold |
| 101 | T | β | R4-7 | 100.0% | 3-fold |
| 133 | T | β | R4-7 | 100.0% | 3-fold |

TABLE 32
Binding activity of conjugates of IL2 mutants with two or more
mutation sites to its receptors before and after activation

| | | | Decreased fold of | Decreased fold of | |
|---|---|---|---|---|---|
| | Mutation | Mutation | binding to α ligand | binding to β ligand | Binding |
| | position (Rα | position (Rβ | after Conjugating | after Conjugating | recovery |
| TMEAkine | binding) | binding) | with S47 | with S47 | (>60%) |
| IL2-Thr37/Thr3 | IL2-Thr37, | | 51-fold | N.D. | 98.9% |
| | IL2-Thr3 | | | | |
| IL2-Thr3/Thr41 | IL2-Thr3, | | 135-fold | N.D. | 101.8% |
| | IL2-Thr41 | | | | |
| IL2-Thr-41/Ser87 | IL2-Thr-41 | IL2-Ser87 | 135-fold | 30-fold | 96.8% |
| IL2-Thr37/Ser87 | IL2-Thr37 | IL2-Ser87 | 51-fold | 30-fold | 98.4% |
| IL2-Thr3/Ser87 | IL2-Thr3 | IL2-Ser87 | N.D. | 30-fold | 99.3% |

The results demonstrate that conjugates of IL2 mutants with two or more mutation sites could retain 95% or more binding activity to corresponding receptor after activation.

After screening expression, binding activity, conjugation, cleavage, recovery and functional assay, we acquired the drug candidates with one or more stable mutations on the binding domain with Rα and one conjugation on the binding domain with Rβ, as shown in Table 33. Particularly, stable mutation sites on the domain binding with Rα were Arg38 and Glu61, in which Arg38 was mutated to Asp and Glu61 was mutated to Arg, affecting binding activity of IL2 and IL2Rα. Therefore, these conjugations are releasing stable mutants on the binding domain with Rα after cleaving in tumor microenvironment.

TABLE 33
Binding activity of candidates with one or more stable mutations
and one conjugation to its receptors before and after activation

| | | Mutation | Decreased fold | Decreased fold | Binding |
|---|---|---|---|---|---|
| | | position (Rβ | of binding to α | of binding to β | recovery |
| | Stable mutation | binding) for | ligand after | ligand after | of β |
| | for blocking | conjugation | conjugating | conjugating | ligand |
| TMEAkine | Rα binding | with S47 | with S47 | with S47 | (>60%) |
| IL2-Arg38/Ser87 | Arg38Asp | Ser87 | 200-fold | 32-fold | 99.5% |
| IL2-Glu61/Ser87 | Glu61Arg | Ser87 | 110-fold | 30-fold | 99.5% |
| IL2-Arg38/ | Arg38 Asp/ | Ser87 | >200-fold | 32-fold | 96.4% |
| Glu61/Ser87 | Glu61Arg | | | | |

TABLE 34
Binding activity of candidates with two stable mutants and one
conjugation to its receptors before and after activation

| | Mutant of Rβ | Decreased | |
|---|---|---|---|
| Mutation for | binding for | fold of | Binding |
| adjusting | conjugation | binding | recovery |
| Rα binding | with R4-7 | to Rα | to Rβ |
| Arg38Asp/Glu61Arg | | >200-fold | |
| | Lys9 | | 124.6% |
| | Lys76 | | 130.4% |
| Arg38Asp/Glu61Arg | Lys9, Lys76 | >200-fold | 165.7% |

The results demonstrated that, with conjugation of R4 library screening and mutation, we got a new chemically modified IL2 with decreasing 200 folds of binding to Rα and increasing 1.35 folds of binding to Rβ.

### 6. Stability in Human Serum

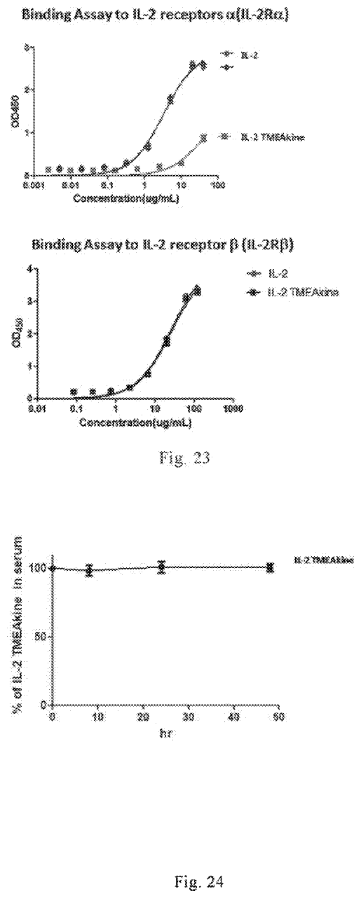

IL2-T41C-S47 solution and human serum were mixed in a ratio of 1:19 (v/v) and the mixture was incubated at 37 °C for 0 h, 8 h, 24 and 48 h, respectively. Then the amount of IL2-T41C-S47 was detected by western blot and the corresponding results were shown in FIG. 24. IL2-T41C-S47 was stable in human serum after 48 hours, indicating that IL2-T41C-S47 may be much more stable than IL2 in human serum in vitro. The stability in human serum of other conjugates of IL2 mutants was shown in Table 35.

TABLE 35
The stability of other conjugates of IL2 mutants in human serum

| R1-R2-R3-R4 | R5 | Stability in Human Serum |
|---|---|---|
| S47 | IL2-Thr37 | 100% |
| S47 | IL2-Thr41 | 99.8% |
| S47 | IL2-Ser87 | 99.2% |
| S47 | IL2-Thr41/Ser87 | 99.5% |
| S47 | IL2-Ser37/Ser87 | 99.5% |

### 7. Pharmacokinetics in Mice

Mice received a single intravenous injection of IL2-T41C-S47 (0.8 mg/kg), n=3 mice per sampling time. Approximately 200 μL blood was collected into K2EDTA-coated tubes. Plasma was separated after centrifugation and frozen at −80 °C until analysis. The IL2-T41C-S47 concentration was then measured using a quantified ELISA. Results were shown in FIG. 25. The pharmacokinetic study in vivo indicates that IL2 TMEAkine has a long half-life and a high exposure in plasma when compared with IL2.

### 8. Toxicity

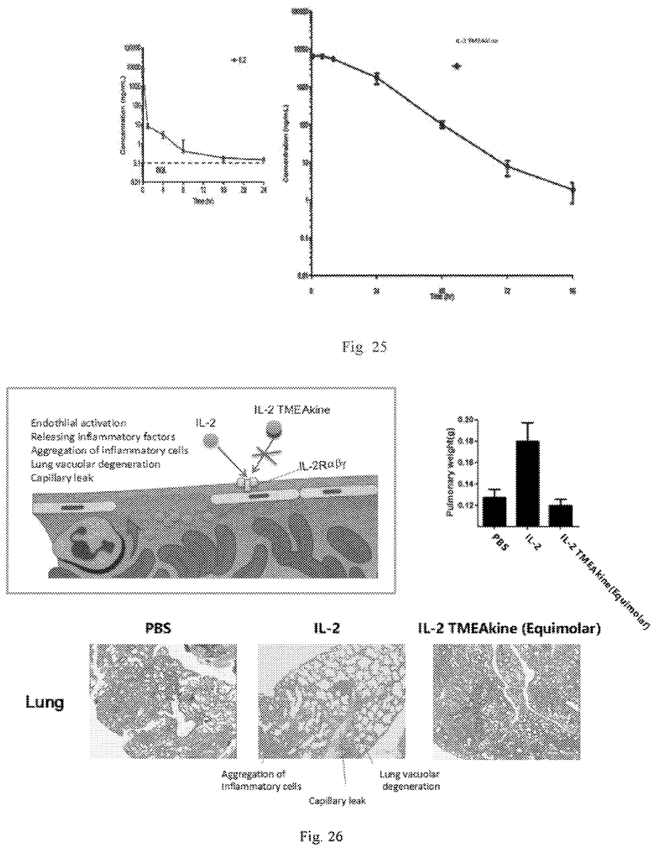

C57BL/6 mice received daily i.p. injection of PBS or 25 ug IL2 for 5 days or i.p. injection of equimolar IL2-T41C-S47 every 5 days for 5 doses, respectively. Mice were sacrificed and lungs were fixed in 10% formalin solution and paraffin-embedded sections were stained with hematoxylin and eosin. The results were shown in FIG. 26. The measurement of pulmonary (wet weight) and the sections stained with hematoxylin and eosin indicate that IL2 TMEAkine induces less toxicity to lung than wild-type IL2.

C57BL/6 mice received i.p. injection of PBS, 25 ug IL2-T41C/S87C-S47 and equimolar IL2-R38D/E61R/S87C-S47 every 5 days for 5 doses, respectively. Mice were sacrificed. The wet weight of lungs was measured and lungs were fixed in 10% formalin solution and paraffin-embedded sections were stained with hematoxylin and eosin. Results were shown in FIG. 27. The measurement of pulmonary indicates that IL2-T41C/S87C-S47 and IL2-R38D/E61R/S87C-S47 induce almost no toxicity to lung.

9. Study on Efficacy of IL2-T41C-S47 and IL2-T41C-S47 in Combination with Anti-PD-1 Antibody on the CT26 Tumor Model in BALB/C Mice Model

Test purpose: to investigate the anti-tumor efficacy of IL2-T41C-S47 and IL2-T41C-S47 in combination with anti-PD-1 antibody in BALB/C mice for treatment of the CT26 tumor model.

Test drug: IL2-T41C-S47, anti-PD-1 antibody and IL2 injection, diluted to corresponding concentrations by PBS when testing.

### Method and Results

1. Animal: BALB/C mice of 5 weeks old, all female.
2. Production of tumor model
1) CT26 cells were purchased from American type culture collection (ATCC) and identified according to the specification provided by ATCC. Cells were cultivated in RPMI 1640 culture solution containing 10% fetal bovine serum at 37 °C and 5% CO₂. The cells were passaged for every three days and cells within the 9th passage were used.
2) Production of tumor model. CT26 cells were subcutaneously injected to the back of the BALB/C mice. Mice were randomly grouped after the tumor grew to about 100-200 mm³ and drug treatment began. Mice were killed after anesthesia on day 31.
3) Course of treatment

There were 5 groups with 6 animals in each group. Included were a control group treated on day 0, 5 and 11, and three single agent groups (treated by anti-PD-1 antibody on day 2, 4, 7, 9, 13 and 15, or by IL2 on day 0, 5, 11, or by IL2-T41C-S47 on day 0, 5 and 11) and one combined immunotherapy group in which IL2-T41C-S47 (given on day 0, 5 and 11) treatment was initiated prior to anti-PD-1 antibody treatment (given on day 2, 4, 7, 9, 13 and 15).
4) Grouping and test results are shown in Table 36.

TABLE 36
Effects of IL2-T41C-S47and IL2-T41C-S47 in combination with anti-
PD-1 antibody on the CT26 tumor model in BALB/C mice model

| Group | Complete Regression |
|---|---|
| Control Group | 0 |
| Anti-PD-1 antibody | 16.79% |
| IL2 | 16.79% |
| IL2-T41C-S47 | 33% |
| IL2-T41C-S47 in combination | 100% |
| with Anti-PD-1 antibody | |

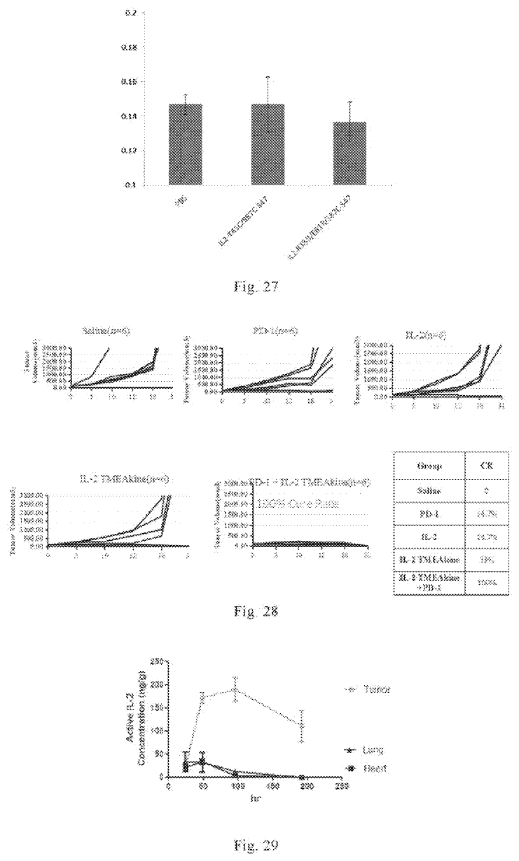

Tumor volumes were monitored 2-3 times a week and were presented in FIG. 28.
5) Results and discussion. As shown in Table 36, the regression on the CT26 tumor of BALB/C mice was greatly improved after injection of IL2-T41C-S47 in combination with anti-PD-1 antibody, indicating that IL2-T41C-S47 in combination with anti-PD-1 antibody exhibits an excellent anti-tumor efficacy on the CT26 tumor model.
   10. Biodistribution of Active IL2 from IL2-T41C-S47 in Tumor, Lung and Heart

BALB/C mice were implanted subcutaneously into the right flank with CT26 cells. Seven days after implantation, when tumors measured 200 mm³, animals were administered with IL2-T41C-S47 (2 mg/kg×1). After 24 h, 48 h, 96 h and 192 h, tumor, lung and heart were harvested (n=2 per observation time), homogenized in ice-cold PBS containing protease inhibitor and 0.25% acetic acid, and centrifuged to obtain supernatant. To quantify IL2-T41C-S47 level, ELISA was performed in which PEG antibody was used as capture antibody and IL2 biotinylated antibody was used as detection antibody. To quantify IL2-T41C-S47 and active IL2 levels, ELISA was performed in which IL2 monoclonal antibody was used as capture antibody and IL2 biotinylated antibody was used as detection antibody. The results were shown in FIG. 29, indicating high exposure of active IL2 in tumor, which is consistent with high efficacy of anti-tumor effect, and low exposure of active IL2 in lung and heart, which is consistent with low toxicity of pulmonary edema.

### Example 8: Section of Chemical Modified Linker to Acquire High Activation Efficiency

R2-R3 is a chemical modified linker showing high activation efficiency as compared to the native peptide sequence linker. The activation of different R2-R3 linkers in which R1 was a 5 kDa PEG and R4 was R4-2 and control linker (C₁-C₄ shown in the Tables) was evaluated in activation assay. The R1-R2-R3-R4 conjugates were dissolved and diluted ten times to a final concentration of 0.1 mM/ml. At 37 °C, test compounds were added into 10 0 μg acidized human breast cancer (MDA-MB435) tumor tissue homogenates (pH6.0) in a concentration of 1 mg/ml. The enzyme in tumor tissue homogenates could release R1. The released R1 was detected by HPLC, thereby comparing the activation efficiency of the linkers. Results were shown in Tables 37-39.

TABLE 37
Activation efficiency (%) of different linkers

| R2-R3 | Activation efficiency (%) |
|---|---|
| C1: AANL | 68.4 |
| C2: AANP | 45.9 |
| C3: SDNP | 22.7 |
| C4: SANP | 32.6 |
| C5: SDNH | 23.9 |
| AAN-R3-5 | 99.6 |
| AAN-R3-6 | 97.5 |
| AAN-R3-7 | 73.3 |
| AAN-R3-8 | 93.3 |
| AAN-R3-9 | 63.3 |
| AAN-R3-10 | 55.1 |
| AAN-R3-11 | 76.3 |
| AAN-R3-12 | 64.5 |
| AAN-R3-1 | 99.7 |
| AAN-R3-2 | 99.5 |
| AAN-R3-3 | 99.1 |
| AAN-R3-4 | 98.9 |

TABLE 38
Activation efficiency (%) of different linkers

| R2-R3 | Activation efficiency (%) |
|---|---|
| C1: AADL | 41.7 |
| C2: AADP | 37.8 |
| C3: SDDP | 46.8 |
| C4: SADP | 31.3 |
| C5: SDNH | 10.7 |
| AAD-R3-5 | 92.3 |
| AAD-R3-6 | 90.5 |
| AAD-R3-7 | 84.6 |
| AAD-R3-8 | 86.7 |
| AAD-R3-9 | 73.4 |
| AAD-R3-10 | 53.6 |
| AAD-R3-11 | 49.6 |
| AAD-R3-12 | 57.7 |
| AAD-R3-1 | 93.7 |
| AAD-R3-2 | 96..5 |
| AAD-R3-3 | 90.1 |
| AAD-R3-4 | 97.9 |

TABLE 39
Activation efficiency (%) of different linkers

| R2-R3 (R2 is absent) | Activation Efficiency (%, pH 6.0) |
|---|---|
| R3-1 | 43.3 |
| R3-2 | 65.1 |
| R3-3 | 46.3 |
| R3-4 | 60.1 |
| R3-5 | 8.8 |
| R3-6 | 79.5 |

MDA-MB435 tumor tissue exhibits high activity of Legumain, Grazym B and MMP2 or other protease. In the assay, it was proved that AAN-R3-5, AAN-R3-6, AAD-R3-5, AAD-R3-6, AAD-3-7 and AAD-R3-8 had relatively higher activation efficiency (>80%). When R2 is absent, R3-5 is stable at pH6.0, and R3-2, R3-4 and R3-6 are an acidically (pH6.0) activated linkers with relatively higher activation efficiency (>60%).

### Example 9: Activation Efficiency of Different Activatable Linker for Targeted Activation in Different Tumor Microenvironment

Six R1-R2-R3-R4 conjugates were detected in different human tumor tissue, wherein R1 was a 5 kDa PEG and R4 was R4-2. The R1-R2-R3-R4 conjugates were each dissolved and diluted ten times to a concentration of 0.1 mM/ml. At 37 °C, test compounds were added into 100 g different acidized human tumor tissue homogenates (pH6.0) in a concentration of 1 mg/ml. The enzyme in tumor tissue homogenates could release R1. The released R1 was detected by HPLC, thereby comparing the activation efficiency of the linkers. Results were shown in Table 40.

TABLE 40
Activation efficiency (%) of R3-3, R3-5, R3-6, AAN-R3-5, AAN-R3-6, and AAD-R3-5 in
homogenates from different tumor tissues

| | Cells | | | | | | |
|---|---|---|---|---|---|---|---|
| | producing | Activation efficiency (%) | | | | | |
| Tumor tissues | tumor | R3-2 | R3-4 | R3-6 | AAN-R3-5 | AAN-R3-6 | AAD-R3-5 |
| Human fibrosarcoma | HT-1080 | 63.7 | 64.7 | 78.9 | 87.2 | 84.5 | 89.5 |
| Human breast cancer | MDA-MB231 | 66.3 | 53.7 | 89.6 | 97.8 | 84.7 | 92.6 |
| Human ovarian cancer | SK-OV-3 | 56.6 | 35.7 | 78.4 | 99.4 | 85.8 | 95.8 |
| Human colon cancer | HT-29 | 63.8 | 56.7 | 58.8 | 93.6 | 83.7 | 91.5 |
| Human chronic leukemia | K562 | 64.8 | 53.8 | 58.3 | 88.2 | 84.6 | 84.8 |
| Human pancreatic cancer | Panc-1 | 46.8 | 73.8 | 74.8 | 96.1 | 94.8 | 79.8 |
| Human non-small cell lung | A549 | 57.8 | 62.7 | 74.7 | 86.2 | 85.8 | 96.4 |
| cancer | | | | | | | |
| Human prostate cancer | PC-3 | 37.8 | 64.8 | 89.6 | 96.5 | 94.7 | 89.6 |
| Human liver cancer | Hepg2 | 46.8 | 64.5 | 45.8 | 87.0 | 83.6 | 85.7 |
| Human renal cancer | OS-RC-2 | 35.7 | 54.6 | 75.7 | 95.7 | 90.4 | 88.4 |
| Human heart | | 8.6 | 3.7 | 7.4 | 0.1 | 0.1 | 6.8 |
| Human Lung | | 6.7 | 6.5 | 4.9 | 0.1 | 0.1 | 1.8 |

### Example 10: Chemical Modified Linker Shows No Steric Hindrance to Different Biomolecules when Activated by Tumor Tissue Protease

R4-1 is the shortest chemical group in the exemplified R4 groups. Different biomolecules were conjugated to R1-R2-R3-R4, in which R was a 5 kDa PEG, R2-R3 were shown in the following table and R4 was R4-1. The conjugates were dissolved and diluted ten times to a concentration of 0.1 mM/ml. At 37 °C, the conjugates were added into 100 μg different acidized human tumor tissue homogenates (pH6.0) in a concentration of 1 mg/ml or were added into a legumain solution (0.1 ug/ml) or a Granzyme B solution (0.1 ug/ml), respectively. The enzyme in tumor tissue homogenates could release R1. The released R1 was detected by HPLC, thereby comparing the activation efficiency of the linkers. Results were shown in Table 41.

TABLE 41
Activation efficiency (%) of linker of different antibody

| | Activation efficiency (%) | | | | Legumain | Granzyme B |
|---|---|---|---|---|---|---|
| Biomolecule | AAN-R3-5 | AAN-R3-6 | AAD-R3-5 | R3-6 | AAN-R3-5 | AAD-R3-5 |
| Human IL2 | 95.6 | 94.4 | 93.4 | 67.8 | 99.5 | 102.8 |
| (SEQ ID NO: 11) | | | | | | |
| PD-1 | 91.4 | 88.6 | 82.8 | 76.4 | 101.6 | 94.7 |
| (SEQ ID NO: 19) | | | | | | |
| Nivolumab | 82.4 | 92.9 | 94.6 | 63.6 | 103.6 | 99.5 |
| (SEQ ID NO: 18) | | | | | | |
| Pembrolizumab | 87.7 | 76.3 | 73.2 | 67.2 | 100.6 | 97.7 |
| (SEQ ID NO: 15) | | | | | | |
| Ip1( CTLA-4 Ab) | 97.8 | 93.8 | 94.5 | 66.1 | 99.4 | 102.7 |
| (SEQ ID NO: 13) | | | | | | |
| Anti-human 4-1BB | 89.5 | 92.4 | 84.4 | 66.2 | 96.8 | 104.4 |
| (SEQ ID NO: 25) | | | | | | |
| Adalimumab | 100.3 | 101.4 | 79.3 | 76.5 | 99.1 | 93.7 |
| (SEQ ID NO: 29) | | | | | | |
| Niv-se001 | 88.7 | 87.6 | 76.5 | 77.0 | 103.7 | 95.7 |
| (SEQ ID NO: 41) | | | | | | |

The results demonstrate that the cleaving site in R2 is distant to the biomolecule (R5). Even with the shortest R4-1, cleavage of R2 is not affected by the biomolecule and the activation efficiency is not affected.

### Example 11: Stability of Chemical Modified Linker in Human Serum

The stability of the chemical modified linker R2-R3 was tested in human serum. R1-R2-R3-R4 conjugates, in which R1 was a 5 kDa PEG (PEG500), R2-R3 was shown in the following Tables and R4 was R4-1, were prepared. The conjugates were dissolved and diluted for ten times to a concentration of 0.1 mM/ml. Conjugates were each added into 100 μg human serum in a concentration of 1 mg/ml at 37 °C for 48 hr. The intact conjugate can be detected by ELISA Assay. By comparing the concentration of remaining conjugates, stability could be calculated. Results were shown in Tables 42 and 43.

TABLE 42
Stability (% of control) of different linkers

| | Stability (% of |
|---|---|
| R2-R3 | control) |
| C1: AANL | 97.4 |
| C2: AANP | 55.9 |
| C3: SDNP | 72.7 |
| C4: SANP | 62.6 |
| C5: SDNH | 73.9 |
| AAN-R3-5 | 99.9 |
| AAN-R3-6 | 99.5 |
| AAN-R3-7 | 99.3 |
| AAN-R3-8 | 98.3 |
| AAN-R3-9 | 97.2 |
| AAN-R3-10 | 98.7 |
| AAN-R3-11 | 96.3 |
| AAN-R3-12 | 97.5 |
| AAN-R3-1 | 89.7 |
| AAN-R3-2 | 89.5 |
| AAN-R3-3 | 79.1 |
| AAN-R3-4 | 88.9 |

TABLE 43
Stability (% of control) of different linkers

| R2-R3 (R2 is absent) | Stability efficiency (%) |
|---|---|
| R3-1 | 88.8 |
| R3-2 | 83.7 |
| R3-3 | 94.6 |
| R3-4 | 91.8 |
| R3-5 | 85.8 |
| R3-6 | 86.4 |

### Example 12: Increased Conjugation Efficiency with Increased Antibody/S13 Linker

For small scale conjugation, 5-10 mg IgG for different variants were buffer exchanged with ultrafiltration tubes (Merck Millipore) into 50 mM Tris-HCl, pH7.5 containing 2 mM EDTA by repeated centrifugation. Then the antibodies were mildly reduced by DTT in a 1:20-1:200 molar ratio at room temperature for 4-16 h. Then the reduced antibodies were dialyzed into 50 mM Tris-HCl, 150 mM NaCl, pH7.5 and re-oxidated by Cu₂SO₄ or Dehydroascorbic acid (DHAA, Sigma) in a 1:50-1:200 molar ratio for 1-3 h at room temperature. Then the re-oxidated antibodies with free sulfydryl were conjugated by S13 chemical linker in a ratio of 1:10, 1:20, 1:50 or 1:100 at room temperature for 4 h. The conjugation efficiency was shown by reduced SDS-PAGE. As shown in Table 44, different conjugation efficiency was obtained.

TABLE 44
Increased conjugation efficiency with increased antibody
(Ipilimumab, SEQ ID NO: 13)/S13 linker ratio

| Antibody/S13 linker molecular ratio | Conjugation efficiency |
|---|---|
| 1:10 | 60% |
| 1:20 | 72% |
| 1:50 | 89% |
| 1:100 | 95% |

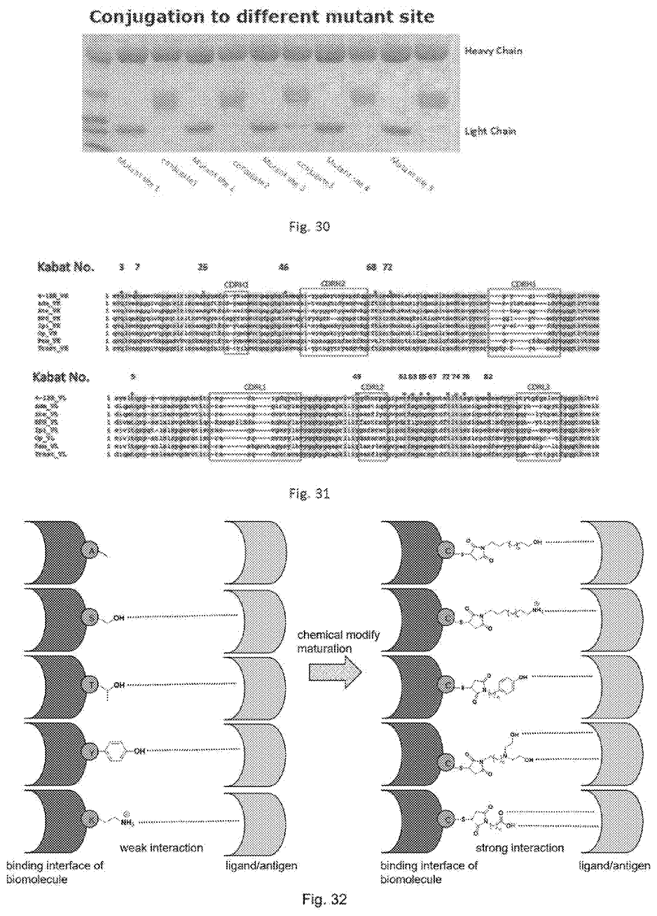

In a conjugation reaction of CTLA4-antibody (Ipilimumab, SEQ ID NO: 14): S13 linker=1:100 condition with Cys mutation in different position of CTLA4-antibody, the mutant sites were shown in Table 45. In the CTLA4-antibody: S13=1:100 condition, all five positions are conjugated with S13 linker in a high efficiency, as shown in reduced SDS-PAGE gel in FIG. 30.

TABLE 45
Different mutation sites for 1:100 molecular ratio conjugation

| Mutation ID | Conjugation efficiency (%) |
|---|---|
| 1# (FR1, L22S) | 96 |
| 2# (FR2, L49Y) | 99 |
| 3# (FR2, L72T) | 95 |
| 4# (CDR1, L24R) | 97 |
| 5# (CDR3, L13G) | 99 |

### Example 13: Conjugation Efficiency and Affinity Change of Framework (Non-CDR) of a Variable Region of Human CTLA-Antibody

We mutated every non-Cysteine amino acid to Cysteine in framework region (FR) of CTLA-4 antibody to make cystine mutant for experiments. Some mutation sites showed nearly 100% conjugation efficiency. The conjugation efficiency for different mutants was high in a CTLA4-antibody:S13=1:100 condition, which was summarized in Table 46 and Table 47. Some mutations show very low conjugation, indicating that Cys may be buried in the interior of antibody.

TABLE 46
Conjugation efficiency for Ipilimumab
light chain framework region

| | | | Conjugation efficiency |
|---|---|---|---|
| Region (Kabat) | Kabat No. | Ipilimumab VL | of S13 (%) |
| FR1 | L1 | E | 35 |
| | L2 | I | 58 |
| | L3 | V | 99 |
| | L4 | L | 29 |
| | L5 | T | 96 |
| | L6 | Q | 95 |
| | L7 | S | 87 |
| | L8 | P | 98 |
| | L9 | G | 96 |
| | L10 | T | 95 |
| | L11 | L | 62 |
| | L12 | S | 88 |
| | L13 | L | 16 |
| | L14 | S | 88 |
| | L15 | P | 95 |
| | L16 | G | 64 |
| | L17 | E | 69 |
| | L18 | R | 72 |
| | L19 | A | 10 |
| | L20 | T | 28 |
| | L21 | L | 13 |
| | L22 | S | 96 |
| | L23 | C | n/d |
| FR2 | L35 | W | 13 |
| | L36 | Y | 88 |
| | L37 | Q | 16 |
| | L38 | Q | 86 |
| | L39 | K | 89 |
| | L40 | P | 19 |
| | L41 | G | 92 |
| | L42 | Q | 85 |
| | L43 | A | 46 |
| | L44 | P | 63 |
| | L45 | R | 25 |
| | L46 | L | 16 |
| | L47 | L | 18 |
| | L48 | I | 85 |
| | L49 | Y | 99 |
| FR3 | L57 | G | 86 |
| | L58 | I | 95 |
| | L59 | P | 94 |
| | L60 | D | 96 |
| | L61 | R | 86 |
| | L62 | F | 75 |
| | L63 | S | 95 |
| | L64 | G | 95 |
| | L65 | S | 94 |
| | L66 | G | 68 |
| | L67 | S | 88 |
| | L68 | G | 88 |
| | L69 | T | 87 |
| | L70 | D | 94 |
| | L71 | F | 28 |
| | L72 | T | 95 |
| | L73 | L | 26 |
| | L74 | T | 95 |
| | L75 | I | 16 |
| | L76 | S | 94 |
| | L77 | R | 96 |
| | L78 | L | 13 |
| | L79 | E | 88 |
| | L80 | P | 96 |
| | L81 | E | 85 |
| | L82 | D | 13 |
| | L83 | F | 18 |
| | L84 | A | 10 |
| | L85 | V | 85 |
| | L86 | Y | 12 |
| | L87 | Y | 19 |
| | L88 | C | n/d |
| FR4 | L98 | F | 87 |
| | L99 | G | 88 |
| | L100 | Q | 19 |
| | L101 | G | 86 |
| | L102 | T | 12 |
| | L103 | K | 95 |
| | L104 | V | 8 |
| | L105 | E | 95 |
| | L106 | I | 75 |
| | L107 | K | 92 |

TABLE 47
Conjugation efficiency for Ipilimumab
heavy chain framework region

| | | | Conjugation efficiency |
|---|---|---|---|
| Region (Kabat) | Kabat No. | Ipilimumab VH | of S13 (%) |
| FR1 | H1 | Q | 68 |
| | H2 | V | 95 |
| | H3 | Q | 86 |
| | H4 | L | 86 |
| | H5 | V | 88 |
| | H6 | E | 18 |
| | H7 | S | 68 |
| | H8 | G | 89 |
| | H9 | G | 85 |
| | H10 | G | 84 |
| | H11 | V | 68 |
| | H12 | V | 18 |
| | H13 | Q | 86 |
| | H14 | P | 85 |
| | H15 | G | 85 |
| | H16 | R | 86 |
| | H17 | S | 78 |
| | H18 | L | 18 |
| | H19 | R | 78 |
| | H20 | L | 8 |
| | H21 | S | 79 |
| | H22 | C | n/d |
| | H23 | A | 89 |
| | H24 | A | 12 |
| | H25 | S | 86 |
| | H26 | G | 89 |
| | H27 | F | 88 |
| | H28 | T | n/d |
| | H29 | F | 89 |
| | H30 | S | 89 |
| FR2 | H36 | W | 13 |
| | H37 | V | 18 |
| | H38 | R | 19 |
| | H39 | Q | 89 |
| | H40 | A | 82 |
| | H41 | P | 98 |
| | H42 | G | 96 |
| | H43 | K | 12 |
| | H44 | G | 96 |
| | H45 | L | 93 |
| | H46 | E | 88 |
| | H47 | W | 18 |
| | H48 | V | 15 |
| | H49 | T | 23 |
| FR3 | H66 | R | 26 |
| | H67 | F | 29 |
| | H68 | T | 95 |
| | H69 | I | 18 |
| | H70 | S | 86 |
| | H71 | R | 28 |
| | H72 | D | 83 |
| | H73 | N | 82 |
| | H74 | S | 85 |
| | H75 | K | 92 |
| | H76 | N | 98 |
| | H77 | T | 25 |
| | H78 | L | 26 |
| | H79 | Y | 95 |
| | H80 | L | 15 |
| | H81 | Q | 95 |
| | H82 | M | 17 |
| FR4 | H103 | W | 68 |
| | H104 | G | 29 |
| | H105 | Q | 92 |
| | H106 | G | 29 |
| | H107 | T | 28 |
| | H108 | L | 95 |
| | H109 | V | 88 |
| | H110 | T | 87 |
| | H111 | V | 27 |
| | H112 | S | 90 |
| | H113 | S | 85 |

The mutant sites with high conjugation efficiency of S13 (>80%) were conjugated with R4-7 and tested for relative binding activity in an ELISA based assay according to EC50 ratio (EC50 of WT antibody: EC50 of mutant antibody-R4-7*100%). Specifically, a 96-well ELISA plate (NUNC) was coated by 1p g/ml His-CTLA-4 protein (Sino Biological) overnight and then blocked with 1% BSA blocker (ThermoFisher) for 2 hours at 37 °C and washed by PBST three times. Corresponding antibody or corresponding mutant with R4-7 conjugation was added and allowed to bind at 37 °C for 1 hour, then washed with PBST three times. HRP enzyme-conjugated anti-human IgG was added and allowed to bind at 37 °C for 1 hour and then washed with PBST three times. TMB substrate (Solarbio., Inc.) was used to detect absorbance at 450 nm. Data analysis was carried out with GraphPad software and EC₅₀ for each antibody or conjugate was calculated.

After comparing the binding affinity R4-7-s-cys-CTLA-4 in FR region and WT antibody of CTLA-4, we found most of position achieved a good effect to maintain the binding affinity (Group A, >60% comparing with WT antibody) while some positions (Group B, <60% comparing with WT antibody) exhibited a lower binding affinity, as shown in Table 48 and Table 49.

TABLE 48
Binding activity of light chain framework
mutants after conjugation with R4-7

| | | | Conjugation | Binding activity |
|---|---|---|---|---|
| | Kabat | Ipilimumab | efficiency of | after conjugation |
| Region | No. | VL | S13 (%) | with R4-7 |
| (Kabat) | L3 | V | 99 | A |
| | L5 | T | 96 | A |
| | L6 | Q | 95 | A |
| | L7 | S | 87 | A |
| | L8 | P | 98 | B |
| | L9 | G | 96 | A |
| | L10 | T | 95 | A |
| | L12 | S | 88 | A |
| | L14 | S | 88 | A |
| | L15 | P | 95 | B |
| | L16 | G | 64 | A |
| | L22 | S | 96 | A |
| | L36 | Y | 88 | A |
| | L38 | Q | 86 | A |
| | L39 | K | 89 | A |
| | L41 | G | 92 | A |
| | L42 | Q | 85 | A |
| | L48 | I | 85 | B |
| | L49 | Y | 99 | A |
| FR3 | L57 | G | 86 | B |
| | L58 | I | 95 | A |
| | L59 | P | 94 | B |
| | L60 | D | 96 | A |
| | L61 | R | 86 | A |
| | L63 | S | 95 | A |
| | L64 | G | 95 | A |
| | L65 | S | 94 | A |
| | L67 | S | 88 | A |
| | L68 | G | 88 | B |
| | L69 | T | 87 | B |
| | L70 | D | 94 | B |
| | L72 | T | 95 | A |
| | L74 | T | 95 | A |
| | L76 | S | 94 | A |
| | L77 | R | 96 | A |
| | L79 | E | 88 | A |
| | L80 | P | 96 | B |
| | L81 | E | 85 | A |
| | L85 | V | 85 | A |
| FR4 | L98 | F | 87 | A |
| | L99 | G | 88 | A |
| | L101 | G | 86 | A |
| | L103 | K | 95 | A |
| | L105 | E | 95 | A |
| | L107 | K | 92 | A |

TABLE 49
Binding activity of heavy chain framework
mutants after conjugation with R4-7

| | Kabat | Ipilimumab | Conjugation efficiency | |
|---|---|---|---|---|
| | No. | VH | of S13 (%) | R4-7 |
| Region (Kabat) | H2 | V | 95 | A |
| | H3 | Q | 86 | A |
| | H4 | L | 86 | A |
| | H5 | V | 88 | A |
| | H8 | G | 89 | A |
| | H9 | G | 85 | A |
| | H10 | G | 84 | A |
| | H13 | Q | 86 | A |
| | H14 | P | 85 | B |
| | H15 | G | 85 | A |
| | H16 | R | 86 | A |
| | H23 | A | 89 | A |
| | H25 | S | 86 | A |
| | H26 | G | 89 | A |
| | H27 | F | 88 | A |
| | H29 | F | 89 | A |
| | H30 | S | 89 | B |
| | H39 | Q | 89 | A |
| | H40 | A | 82 | A |
| | H41 | P | 98 | B |
| | H42 | G | 96 | A |
| | H44 | G | 96 | A |
| | H45 | L | 93 | A |
| | H46 | E | 88 | A |
| | H68 | T | 95 | A |
| | H70 | S | 86 | A |
| | H72 | D | 83 | B |
| | H73 | N | 82 | B |
| | H74 | S | 85 | B |
| | H75 | K | 92 | A |
| | H76 | N | 98 | A |
| | H79 | Y | 95 | A |
| | H81 | Q | 95 | A |
| | H105 | Q | 92 | A |
| | H108 | L | 95 | A |
| | H109 | V | 88 | A |
| | H110 | T | 87 | A |
| | H112 | S | 90 | A |
| | H113 | S | 85 | A |

For these sites with binding activity <60% (class B in the above tables), we conjugated them with different chemical linkers to rescue the binding activity. As shown in Table 50, for some of them, the binding activity can be restored with specific chemical modification of R4. These results indicate that the side chain of these sites might contribute to the antibody/antigen interaction and specific chemical linker can mimic the light chain structure and provide molecular interaction against antigen like the native WT amino acid.

TABLE 50
R4 screening to restore the activity of sites with low activity when conjugated with R4-7

| | Original | | | | | | | | |
|---|---|---|---|---|---|---|---|---|---|
| Kabat | amino | | R4 screening | | | | | | |
| No. | acid | R4-7 | R4-1 | R4-3 | R4-4 | R4-5 | R4-8 | R4-11 | R4-27 |
| L8 | P | B | B | B | B | B | B | A | B |
| L15 | P | B | B | B | B | B | B | B | B |
| L48 | I | B | B | B | B | A | B | B | B |
| L57 | G | B | A | B | B | B | B | B | B |
| L59 | P | B | B | B | B | B | B | B | B |
| L68 | G | B | A | B | B | B | B | B | B |
| L69 | T | B | A | A | B | B | B | B | B |
| L70 | D | B | B | B | B | B | B | A | B |
| L80 | P | B | B | B | B | B | B | B | B |
| H14 | P | B | B | B | B | B | B | B | B |
| H30 | S | B | A | A | B | B | B | B | B |
| H41 | P | B | B | B | B | B | B | B | B |
| H72 | D | B | B | B | B | B | B | A | B |
| H73 | N | B | B | B | B | B | B | A | B |
| H74 | S | B | A | B | B | B | B | B | B |

Blocking Effect of Selected Sites when Conjugated with S13 (5 kD Functional Group) or S47 (40 kD Functional Group) Chemical Linker

We selected the above sites whose activity can be restored after conjugating with R4-7 or other R4 linkers as candidate sites for tumor microenvironment activated antibody (TMEAbody) screening, because after protease cleavage, the conjugated antibody will remain the identical structure with antibody mutant-s-R4 form. Blocking effect after conjugated with S13 (5 kD functional group) was first evaluated with the ELISA assay like Example 1. As shown in Table 51, after conjugation with S13 linker, some of them showed significant blocking effect of the binding activity against the antigen protein. We also categorize these sites with <30% activity as class A and other sites as class B, as shown in the Table 51.

TABLE 51
Blocking efficiency of selected sites
when conjugated with S13 (5kD) linker

| | | Conjugation | |
|---|---|---|---|
| | Original amino | efficiency | Binding Activity after |
| Kabat No. | acid | of S13 (%) | S13 (5kD) blocking |
| L3 | V | 99 | A |
| L5 | T | 96 | B |
| L6 | Q | 95 | B |
| L7 | S | 87 | B |
| L9 | G | 96 | B |
| L10 | T | 95 | B |
| L12 | S | 88 | B |
| L14 | S | 88 | B |
| L22 | S | 96 | A |
| L36 | Y | 88 | A |
| L38 | Q | 86 | B |
| L39 | K | 89 | B |
| L41 | G | 92 | B |
| L42 | Q | 85 | B |
| L49 | Y | 99 | A |
| L58 | I | 95 | A |
| L60 | D | 96 | A |
| L61 | R | 86 | B |
| L63 | S | 95 | B |
| L64 | G | 95 | B |
| L65 | S | 94 | A |
| L67 | S | 88 | A |
| L69 | T | 87 | A |
| L70 | D | 94 | A |
| L72 | T | 95 | B |
| L74 | T | 95 | B |
| L76 | S | 94 | B |
| L77 | R | 96 | B |
| L79 | E | 88 | B |
| L81 | E | 85 | B |
| L85 | V | 85 | B |
| L98 | F | 87 | B |
| L99 | G | 88 | B |
| L101 | G | 86 | B |
| L103 | K | 95 | B |
| L105 | E | 95 | B |
| L107 | K | 92 | B |
| H2 | V | 95 | A |
| H3 | Q | 86 | A |
| H4 | L | 86 | A |
| H5 | V | 88 | A |
| H8 | G | 89 | B |
| H9 | G | 85 | B |
| H10 | G | 84 | B |
| H13 | Q | 86 | B |
| H14 | P | 85 | B |
| H15 | G | 85 | B |
| H16 | R | 86 | B |
| H23 | A | 89 | A |
| H25 | S | 86 | A |
| H26 | G | 89 | A |
| H27 | F | 88 | A |
| H29 | F | 89 | A |
| H30 | S | 89 | A |
| H39 | Q | 89 | B |
| H40 | A | 82 | B |
| H42 | G | 96 | B |
| H44 | G | 96 | B |
| H45 | L | 93 | B |
| H46 | E | 88 | B |
| H68 | T | 95 | A |
| H70 | S | 86 | A |
| H72 | D | 83 | A |
| H73 | N | 82 | A |
| H74 | S | 85 | A |
| H75 | K | 92 | A |
| H76 | N | 98 | A |
| H79 | Y | 95 | A |
| H81 | Q | 95 | B |
| H105 | Q | 92 | B |
| H108 | L | 95 | B |
| H109 | V | 88 | B |
| H110 | T | 87 | B |
| H112 | S | 90 | B |
| H113 | S | 85 | B |

We further investigated if blocking efficiency could be improved with higher molecular weight functional group. S47 (with 40 kD functional group) and S64 (with 80 kD functional group) were used for conjugation and blocking efficiency was measured with the above method in a binding ELISA assay. The results were summarized in Table 52, which showed that the increased molecular weight can significantly improve the blocking efficiency of binding activity. All of the sites showed <30% activity when conjugated with S64 (80 kD) functional group.

TABLE 52
Blocking efficiency of selected sites when conjugated with S13 (5 kD), S37 (20 kD), S47 (40 kD) or
S64 (80 kD) linkers

| | | | Binding | Binding | Binding | Binding |
|---|---|---|---|---|---|---|
| | | Conjugation | Activity | Activity | Activity | Activity |
| | Original | efficiency | after | after | after | after |
| Kabat | amino | of S13 | S13 (5 kD) | S37 (20 kD) | S47 (40 kD) | S64 (80 kD) |
| No. | acid | (%) | blocking | blocking | blocking | blocking |
| L3 | V | 99 | A | A | A | A |
| L5 | T | 96 | B | A | A | A |
| L6 | Q | 95 | B | A | A | A |
| L7 | S | 87 | B | A | A | A |
| L9 | G | 96 | B | B | A | A |
| L10 | T | 95 | B | B | B | A |
| L12 | S | 88 | B | A | A | A |
| L14 | S | 88 | B | A | A | A |
| L22 | S | 96 | A | A | A | A |
| L36 | Y | 88 | A | A | A | A |
| L38 | Q | 86 | B | A | A | A |
| L39 | K | 89 | B | B | A | A |
| L41 | G | 92 | B | B | B | A |
| L42 | Q | 85 | B | A | A | A |
| L49 | Y | 99 | A | A | A | A |
| L58 | I | 95 | A | A | A | A |
| L60 | D | 96 | A | A | A | A |
| L61 | R | 86 | B | A | A | A |
| L63 | S | 95 | B | B | B | A |
| L64 | G | 95 | B | B | B | A |
| L65 | S | 94 | A | A | A | A |
| L67 | S | 88 | A | A | A | A |
| L69 | T | 87 | A | A | A | A |
| L70 | D | 94 | A | A | A | A |
| L72 | T | 95 | B | A | A | A |
| L74 | T | 95 | B | A | A | A |
| L76 | S | 94 | B | B | A | A |
| L77 | R | 96 | B | B | B | A |
| L79 | E | 88 | B | B | B | A |
| L81 | E | 85 | B | B | B | A |
| L85 | V | 85 | B | B | B | A |
| L98 | F | 87 | B | B | B | A |
| L99 | G | 88 | B | B | A | A |
| L101 | G | 86 | B | B | A | A |
| L103 | K | 95 | B | A | A | A |
| L105 | E | 95 | B | A | A | A |
| L107 | K | 92 | B | A | A | A |
| H2 | V | 95 | A | A | A | A |
| H3 | Q | 86 | A | A | A | A |
| H4 | L | 86 | A | A | A | A |
| H5 | V | 88 | A | A | A | A |
| H8 | G | 89 | B | A | A | A |
| H9 | G | 85 | B | A | A | A |
| H10 | G | 84 | B | A | A | A |
| H13 | Q | 86 | B | B | B | A |
| H14 | P | 85 | B | B | B | A |
| H15 | G | 85 | B | A | A | A |
| H16 | R | 86 | B | A | A | A |
| H23 | A | 89 | A | A | A | A |
| H25 | S | 86 | A | A | A | A |
| H26 | G | 89 | A | A | A | A |
| H27 | F | 88 | A | A | A | A |
| H29 | F | 89 | A | A | A | A |
| H30 | S | 89 | A | A | A | A |
| H39 | Q | 89 | B | A | A | A |
| H40 | A | 82 | B | A | A | A |
| H42 | G | 96 | B | B | A | A |
| H44 | G | 96 | B | B | B | A |
| H45 | L | 93 | B | A | A | A |
| H46 | E | 88 | B | A | A | A |
| H68 | T | 95 | A | A | A | A |
| H70 | S | 86 | A | A | A | A |
| H72 | D | 83 | A | A | A | A |
| H73 | N | 82 | A | A | A | A |
| H74 | S | 85 | A | A | A | A |
| H75 | K | 92 | A | A | A | A |
| H76 | N | 98 | A | A | A | A |
| H79 | Y | 95 | A | A | A | A |
| H81 | Q | 95 | B | A | A | A |
| H105 | Q | 92 | B | A | A | A |
| H108 | L | 95 | B | B | A | A |
| H109 | V | 88 | B | B | A | A |
| H110 | T | 87 | B | B | B | A |
| H112 | S | 90 | B | B | B | A |
| H113 | S | 85 | B | B | B | A |

### Improving Binding Affinity of R4 Modified Antibody by R4 Library in Framework (Non-CDR) of a Variable Region of Human CTLA-4 Antibody and PD-1 Antibody

Framework (non-CDR) region is a conservative sequence and has been used to link the CDR region to form different antibody. As shown in Table 51 and Table 52, when the side chain of some amino acid changes, the binding are reduced (class B), indicating that side chain of amino acid may provide an interaction or a space to help antigen binding with CDR. Normally, the interaction between framework and antigen is weaker than that between CDR region and antigen. By conjugation of the different R4 to the selected mutant position of PD-1 antibody (Nivolumab), we have the chance to obtain a higher affinity or to maintain the affinity of the chemical modified antibody, as compared to the wild type antibody.

TABLE 53
Binding activity of R4 conjugated with mutation
biomolecular library in conservative FR sites
(% of WT antibody) for PD-1 antibody (Nivolumab)

| PD-1 | R4-1 | R4-2 | R4-3 | R4-4 | R4-5 | R4-6 | R4-7 | R4-8 | R4-9 |
|---|---|---|---|---|---|---|---|---|---|
| Gly41 | 62.6 | 45.6 | 1.6 | 33.6 | 24.6 | 34.4 | 32.7 | 31.7 | 58.7 |
| Glu46 | 26.4 | 48.9 | 67.8 | 45.1 | 49.6 | 29.7 | 86.4 | 78.4 | 10.4 |
| Tyr49 | 67.8 | 48.6 | 33.4 | 14.5 | 18.4 | 105.4 | 97.4 | 62.4 | 59.8 |
| Arg61 | 67.4 | 108.4 | 48.6 | 78.4 | 64.1 | 89.1 | 91.5 | 57.9 | 48.5 |
| Ser63 | 89.5 | 98.4 | 108.9 | 84.5 | 89.4 | 85.5 | 84.5 | 88.9 | 116.9 |
| Thr72 | 48.9 | 47.6 | 115.9 | 67.4 | 48.5 | 75.8 | 68.7 | 12.4 | 34.9 |

The results showed that in FR region of different antibodies, conjugating different R4 to the conservative site can adjust the binding activity. In a representative Gly41, it also has a chance to recover 62.6% binding affinity by conjugation with R4-1. In conservative Gln3, Ser7, Ser26, Glu46, Thr68, Asp72 in VH, and Thr5, Tyr49, Arg61, Ser63, Ser65, Ser67, Thr72, Thr74, Ser76, Asp82 in framework, it can be screened out higher affinity binding conjugation. Because framework sequences (FR1, FR2 and FR3) are conservative in all kinds of human antibody, by conjugating different R4 to these conservative sites in FR region, any antibody has the chance to maintain or increase the binding affinity.

### Analysis on the Binding Activity of Mutants in a Sequence of High Homology in the Framework (Non-CDR) of a Variable Region of Human Germline Antibody

Human antibody consists of 4 peptide chains, including two identical light chains (LC) and two identical heavy chains (HC). The chains form a monomer by disulfide bond(s) and non-covalent bonds. There are two types of light chains, κ and λ, and five types of heavy chains, i.e., μ, δ, γ, ε and α. An antibody, as a whole, is divided into a constant region and a variable region. The variable region is located at the terminus of the two arms of the Y-shaped structure. Humanized or human antibodies have a certain generality, that is, they all contain 4 loops in heavy chain or light chain at the terminus of the two arms of the Y-shaped structure. Three loops are highly variable and directly anticipate in binding to an antigen. The regions in these loops are termed CDRs, wherein CDR1, CDR2 and CDR3 are present in these three loops, respectively.

Antibody was produced by recombination of genes of immunoglobulin superfamily in vivo. Some framework regions of antibodies against different antigens may be derived from a gene or an amino acid sequence of a same germline antibody. Between the CDR, there are framework sequences (FR1, FR2, and FR3), which are conservative in all kinds of human antibody. All kinds of human antibody for variable region is shown in full from the start codon to the last nucleotide before the variable region gene exon in the case of the leader sequence, from the beginning of the V gene exon (Residue 1 in FR1) to the last nucleotide/amino acid before the heptamer recombination signal sequence in the case of VH, VK and VL. The 8 selected antibodies sequences are shown in FIG. 31.

We mutated other antibodies' heavy chain and light chain DNA sequences corresponding to the same conserved position for expression to compare the binding affinity with other sites in FR region and WT antibody. In the 5 antibody sequences, the conservative sites of Gln3, Ser7, Ser26, Glu46, Thr68 and Asp72 in VH, and Thr5, Tyr49, Arg61, Ser63, Ser65, Ser67, Thr72, Thr74, Ser76 and Asp82 in VL (the position of the amino acid is numbered according to Kabat numbering) were selected to conjugate with R4-7.

TABLE 54
Binding activity of R4-7 conjugated with mutation biomolecular
library in conservative FR sites (% of WT antibody)

| sites | 41BB | Ada | PD-1-k | Ip1 | Trast |
|---|---|---|---|---|---|
| Gln3 | 68.3 | 78.4 | 73.8 | 98.3 | 94.5 |
| Ser7 | 94.5 | 87.4 | 105.6 | 110.4 | 110.6 |
| Ser26 | 87.1 | 68.4 | 97.1 | 74.8 | 104.6 |
| Glu46 | 97.4 | 78.4 | 69.4 | 67.8 | 78.4 |
| Thr68 | 68.7 | 97.8 | 74.7 | 79.8 | 87.7 |
| Thr5 | 86.4 | 97.4 | 84.1 | 67.4 | 69.7 |
| Gly41 | 24.8 | 16.4 | 8.9 | 8.4 | 8.9 |
| Tyr49 | 79.7 | 97.8 | 99.7 | 112.4 | 107.8 |
| Arg61 | 87.9 | 78.9 | 67.8 | 79.8 | 94.1 |
| Ser63 | 89.7 | 69.5 | 69.1 | 78.9 | 97.4 |
| Ser65 | 69.4 | 78.9 | 70.8 | 69.7 | 97.8 |
| Ser67 | 79.7 | 68.7 | 69.2 | 79.0 | 64.9 |
| Thr72 | 68.5 | 66.4 | 72.8 | 70.9 | 79.8 |
| Thr74 | 107.5 | 97.4 | 89.4 | 99.8 | 95.8 |
| Ser76 | 69.4 | 78.9 | 69.1 | 78.4 | 78.4 |
| Asp82 | 98.5 | 74.8 | 68.7 | 79.4 | 67.8 |

The results showed that in FR region of different antibodies, conjugating R4-7 to the conservative sites of Gln3, Ser7, Ser26, Glu46, Thr68 or Asp72 in VH, and Thr5, Tyr49, Arg61, Ser63, Ser65, Ser67, Thr72, Thr74, Ser76 or Asp82 in VL can maintain the binding activity as compared to the wild type antibody or other positions, which was 60% or more of the original binding activity. In a representative negative control Gly41, it lost binding affinity in all antibody (<60% of WT antibody).

### Example 14 Mutated Amino Acid in CDR Region of CTLA-4 Antibody to Screen Different R4 Group for Maintaining or Increasing the Binding Affinity

CTLA-4 is on the T cell surface in tumor microenvironment. We mutated every amino acid in CDR region of its antibody to screen by different R4 group for maintaining or increasing the binding affinity. In some case, there are some R4 groups which could enhance the binding affinity, but we did not select them as drug candidates in our development. We prefer R-1, R4-7, R4-5, R4-8 and R4-12 for the large scale synthesis and stability in our drug development. The conjugated CTLA-4 antibody with R4 can recover the binding >60% in some positions by chemical modified maturation of R4 library screening.

TABLE 55
Binding effect of mutants of anti-CTLA-4 antibody having mutation
in CDR regions after conjugating to different R4 molecules

| | | Original amino | | Binding |
|---|---|---|---|---|
| Region (Kabat) | Kabat No. | acid | R4 | activity |
| CDRL1 | L24 | R | R4-26 | 89% |
| | L25 | A | R4-1 | 95% |
| | L26 | S | R4-1 | 91% |
| | L27 | Q | R4-11 | 88% |
| | L28 | V | R4-7 | 78% |
| | L29 | G | R4-1 | 95% |
| | L30 | S | R4-1 | 81% |
| | L30 | S | R4-2 | 104% |
| | L30 | S | R4-3 | 96% |
| | L31 | S | R4-1 | 99% |
| | L32 | Y | R4-7 | 98% |
| | L32 | Y | R4-8 | 107% |
| | L33 | L | R4-1 | 95% |
| | L34 | A | R4-1 | 92% |
| CDRL2 | L50 | G | R4-1 | 97% |
| | L50 | G | R4-3 | 107% |
| | L51 | A | R4-1 | 92% |
| | L52 | F | R4-1 | 72% |
| | L53 | S | R4-1 | 98% |
| | L54 | R | R4-26 | 163% |
| | L55 | A | R4-1 | 102% |
| | L55 | A | R4-3 | 106% |
| | L56 | T | R4-1 | 93% |
| CDRL3 | L89 | Q | R4-7 | 96% |
| | L90 | Q | R4-7 | 75% |
| | L91 | Y | R4-5 | 95% |
| | L91 | Y | R4-7 | 105% |
| | L92 | G | R4-1 | 99% |
| | L92 | G | R4-3 | 104% |
| | L93 | S | R4-1 | 92% |
| | L94 | S | R4-1 | 95% |
| | L94 | S | R4-11 | 142% |
| | L95 | P | R4-1 | 62% |
| | L96 | W | R4-4 | 72% |
| | L97 | T | R4-1 | 91% |
| CDRH1 | H31 | S | R4-1 | 110% |
| | H32 | Y | R4-7 | 92% |
| | H32 | Y | R4-18 | 208% |
| | H33 | T | R4-1 | 102% |
| | H34 | M | R4-1 | 65% |
| | H35 | H | R4-5 | 72% |
| CDRH2 | H50 | F | R4-4 | 73% |
| | H51 | I | R4-4 | 72% |
| | H52 | S | R4-1 | 95% |
| | H52 | S | R4-3 | 107% |
| | H52A | Y | R4-7 | 93% |
| | H53 | D | R4-11 | 91% |
| | H54 | G | R4-1 | 95% |
| | H55 | N | R4-11 | 96% |
| | H56 | N | R4-11 | 91% |
| | H57 | K | R4-7 | 88% |
| | H58 | Y | R4-7 | 89% |
| | H59 | Y | R4-4 | 92% |
| | H59 | Y | R4-10 | 105% |
| | H60 | A | R4-4 | 65% |
| | H61 | D | R4-11 | 92% |
| | H62 | S | R4-3 | 91% |
| | H63 | V | R4-1 | 68% |
| | H64 | K | R4-7 | 96% |
| | H64 | K | R4-18 | 137% |
| | H65 | G | R4-1 | 93% |
| CDRH3 | H95 | T | R4-3 | 92% |
| | H95 | T | R4-1 | 152% |
| | H96 | G | R4-1 | 65% |
| | H96 | G | R4-2 | 85% |
| | H97 | W | R4-8 | 92% |
| | H98 | L | R4-3 | 84% |
| | H99 | G | R4-1 | 94% |
| | H100 | P | R4-1 | 69% |
| | H100A | F | R4-4 | 84% |
| | H101 | D | R4-3 | 88% |
| | H102 | Y | R4-7 | 95% |
| | H102 | Y | R4-5 | 242% |

According to the results, after mutating G, A, S, L, T, I, F, E, K, D, N, Q, R or Y in the CDRs of anti-CTLA-4 antibody to C and binding to different R4, the mutants could retain a binding efficiency of >60%.

In some cases, there are some R4 groups which could enhance the binding affinity. We selected the binding affinity between 60~100% of R4-s-R5 as drug candidates in our drug development. We prefer R-1, R4-7, R4-5, R4-8 and R4-12 for the large scale synthesis and stability in our drug development. S47 is cleaved by Legumain, and after cleaving the R4-7 chemical group is retained. After S47 conjugating to the amino acid of CDR, all these conjugates can block CTLA-4 binding with decreased affinity (activity <30% than WT CTLA-4). So the positions in CDR regions or mutant can become a primary drug candidate for tumor microenvironment activated antibody.

TABLE 56
Blocking efficiency of CDR regions
conjugated with S47 (40kD) linkers

| | | Original | | | Activity after |
|---|---|---|---|---|---|
| Region | Kabat | amino | | Binding | S47(40kD) |
| (Kabat) | No. | acid | R4 | activity | blocking |
| CDRL1 | L24 | R | R4-26 | 89% | 28% |
| | L25 | A | R4-1 | 95% | 29% |
| | L26 | S | R4-1 | 91% | 18% |
| | L27 | Q | R4-11 | 88% | 8% |
| | L27A | S | R4-1 | 82% | 24% |
| | L28 | V | R4-7 | 78% | 25% |
| | L29 | G | R4-1 | 95% | 19% |
| | L30 | S | R4-1 | 81% | 19% |
| | L31 | S | R4-1 | 99% | 12% |
| | L32 | Y | R4-7 | 95% | 13% |
| | L33 | L | R4-1 | 95% | 6% |
| | L34 | A | R4-1 | 92% | 11% |
| CDRL2 | L50 | G | R4-1 | 97% | 19% |
| | L51 | A | R4-1 | 92% | 25% |
| | L52 | F | R4-1 | 72% | 16% |
| | L53 | S | R4-1 | 98% | 5% |
| | L54 | R | R4-26 | 113% | 17% |
| | L55 | A | R4-1 | 102% | 20% |
| | L56 | T | R4-1 | 93% | 5% |
| CDRL3 | L89 | Q | R4-7 | 96% | 15% |
| | L90 | Q | R4-7 | 75% | 7% |
| | L91 | Y | R4-7 | 95% | 8% |
| | L92 | G | R4-1 | 99% | 16% |
| | L93 | S | R4-1 | 92% | 9% |
| | L94 | S | R4-1 | 95% | 12% |
| | L95 | P | R4-1 | 62% | 13% |
| | L96 | W | R4-4 | 72% | 12% |
| | L97 | T | R4-1 | 91% | 16% |
| CDRH1 | H31 | S | R4-1 | 110% | 25% |
| | H32 | Y | R4-7 | 92% | 23% |
| | H33 | T | R4-1 | 102% | 26% |
| | H34 | M | R4-1 | 65% | 29% |
| | H35 | H | R4-5 | 72% | 28% |
| CDRH2 | H50 | F | R4-4 | 73% | 25% |
| | H51 | I | R4-4 | 72% | 27% |
| | H52 | S | R4-1 | 95% | 5% |
| | H52A | Y | R4-7 | 93% | 18% |
| | H53 | D | R4-11 | 91% | 11% |
| | H54 | G | R4-1 | 95% | 15% |
| | H55 | N | R4-11 | 96% | 22% |
| | H56 | N | R4-11 | 91% | 8% |
| | H57 | K | R4-7 | 88% | 11% |
| | H58 | Y | R4-7 | 89% | 7% |
| | H59 | Y | R4-4 | 92% | 15% |
| | H60 | A | R4-4 | 65% | 22% |
| | H61 | D | R4-11 | 92% | 19% |
| | H62 | S | R4-3 | 91% | 23% |
| | H63 | V | R4-1 | 68% | 22% |
| | H64 | K | R4-7 | 96% | 26% |
| | H65 | G | R4-1 | 93% | 28% |
| CDRH3 | H95 | T | R4-3 | 92% | 18% |
| | H96 | G | R4-1 | 65% | 11% |
| | H97 | W | R4-8 | 92% | 9% |
| | H98 | L | R4-3 | 84% | 6% |
| | H99 | G | R4-1 | 94% | 12% |
| | H100 | P | R4-1 | 69% | 3% |
| | H100A | F | R4-4 | 84% | 16% |
| | H101 | D | R4-3 | 88% | 23% |
| | H102 | Y | R4-7 | 95% | 16% |

According to the results, after mutating native amino acid in the CDRs of anti-CTLA-4 antibody to cystine and chemically conjugating to different R4 (R4 library screening), the mutants could retain a binding efficiency of >60%. After S47 conjugation reaction with amino acid of CDR, all these positions can block CTLA-4 binding with decrease affinity (<30% comparing with WT CTLA-4). Therefore, all S47 conjugates can become primary drug candidates for tumor microenvironment activated antibody.

To improve affinity of antibody, amino acid on CDR loop was mutated during affinity maturation. In fact, optimization of antibody affinity also can be achieved by conjugating to a suitable R4 (termed herein as chemical maturation of antibody). As shown in FIG. 32, by site-specially conjugating different R4 groups to the selected mutant site of antibody, we can selectively change the R4 group for screening. (1) Native amino acid has no H-bond or charge interaction with ligand. Chemical maturation can induce new interaction. (2) Native amino acid has weak H-bond or charge interaction with ligand for the distance. Chemical maturation can adjust distance (by R4-b) to screen the best distance. (3) Native amino acid has weak H-bond or charge interaction. Chemical maturation can change interaction group to screen the best R4-c group. (4) Chemical maturation also can increase charge by increasing NH₂⁺ or O⁻. As a result, there are chances to increase the interaction of H-bond or charge interaction as a kind of new affinity maturation.

We performed chemical maturation of antibody by conjugating R4 to three mutants having one or two mutations at a same CTLA-4 antibody. The results were shown in Table 57.

TABLE 57
Binding activity of conjugating R4 to three mutants

| | Mutant 1 | Mutant 2 | Binding activity |
|---|---|---|---|
| R4-18 | H32 | | 208% |
| R4-18 | | H64 | 137% |
| R4-18 | H32 | H64 | 356% |

Optimization of antibody affinity can also be achieved by chemical maturation in CDR loop of an antibody by conjugating to a suitable R4 group.

### Example 15

### Binding ELISA Characterization of Constructed CTLA-4 TMEAbodies

In drug development, we collected the sites with best blocking efficiency and restored activity for further development. After conjugation with S47, the human antibody become a tumor microenvironment activated antibody, and is named as TMEAbody.

To assess the recovery capability of TMEAbodies in binding against human CTLA-4 protein in a tumor microenvironment, the conjugated TMEAbodies were in vitro digested by Legumain and the digested product was used for evaluating recovered binding activity to human CTLA-4. To characterize the binding property of the constructed TMEAbodies to the human CTLA-4, 0.5 μg/ml CTLA-4 Fc fusion protein (R&D systems) was coated on the Maxisorp ELISA plate (Nunc) by incubation at 4 °C overnight. Then the plate was washed three times with PBST and blocked by 2% BSA at room temperature for 2 h. After washing by PBST for three times, the plate was incubated with serial concentration of conjugated TMEAbodies, TMEAbodies before conjugation (Cysteine mutant form), and control wild type (WT) Ipilimumab antibody at room temperature for 1 h. The plate was then washed three times by PBST and incubated by goat anti-human IgG Fab fragment conjugated with HRP (Sigma) with 1:5000 dilution at room temperature for 1 h. After washing by PBST three times, the plates were developed with tetramethylbenzidine (TMB, Solarbio) and ELISA stop buffer (Solarbio). Absorbance at 450 nm was then measured by ELISA plate reader (Biotek™). Data was then analyzed by GraphPad Prism 5 software.

As shown in table 58, conjugation of chemical linker to different mutant sites can give rise to different degrees of blocking efficiency. Blocking efficiency can be calculated by the fold change the EC₅₀ value of binding curve. The conjugated TMEAbodies with blocking efficiency bigger than 10 fold and Restored (EC₅₀ value <2 fold of WT) were considered as good candidates for further development.

TABLE 58
Ipilimumab TMEAbody candidates selected by blocking efficiency
and restored activity after protease cleavage

| | | | | | | | Blocking | | Restored |
|---|---|---|---|---|---|---|---|---|---|
| Selected | | | | | | Conjugation | efficiency after | | activity after |
| Mutation | Heavy or | Mutation | | Kabat | | efficiency | conjugation | | protease |
| ID | chain | Site | Position | No. | R4 | of S47 | with S47 | | cleavage |
| Ipi-se001 | HC | Y | 53 | H52A | R4-7 | 95% | 20 | fold | Restored |
| Ipi-se002 | HC | D | 54 | H53 | R4-11 | 95% | 9 | fold | Restored |
| Ipi-se003 | HC | Y | 59 | H58 | R4-4 | 94% | 15 | fold | Restored |
| Ipi-se004 | HC | D | 73 | H72 | R4-11 | 94% | 6 | fold | Restored |
| Ipi-se005 | HC | K | 76 | H75 | R4-7 | 94% | 8 | fold | Restored |
| Ipi-se006 | HC | P | 104 | H100 | R4-1 | 96% | 30 | fold | Restored |
| Ipi-se007 | LC | Q | 27 | L27 | R4-11 | 95% | 13 | fold | Resorted |
| Ipi-se009 | LC | S | 54 | L53 | R4-1 | 95% | 22 | fold | Restored |
| Ipi-se053 | LC | Y | 50 | L49 | R4-7 | 98% | 163 | fold | Resorted |
| Ipi-se066 | LC | S | 68 | L67 | R4-7 | 97% | 3 | fold | Resorted |

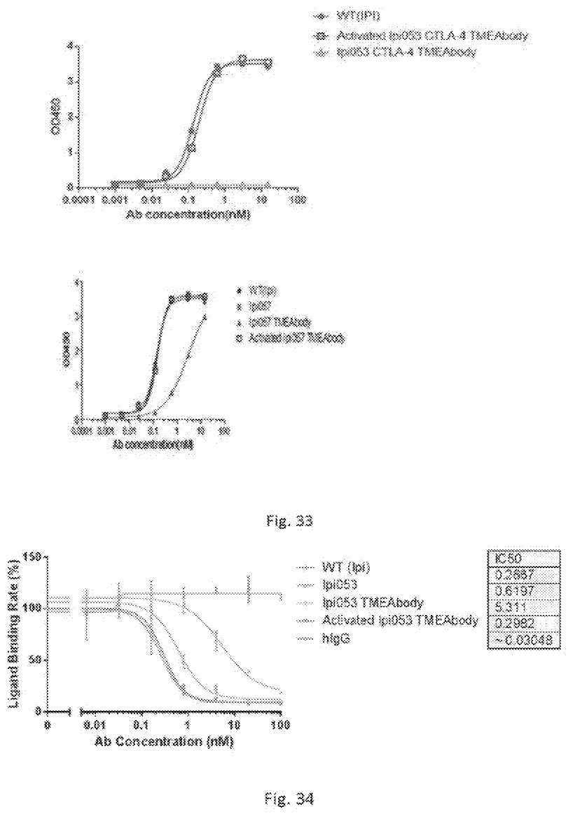

The same ELISA method was performed as described above. As shown in FIG. 33, conjugating to different sites showed different degree of binding activity recovery. Some mutant sites showed comparable binding activity to the WT Ipilimumab after digestion (EC₅₀ change fold <2, or in other words, the activity >50% of WT).

### Blocking of Antigen Binding of TMEAbodies Resulted in Decreased Receptor Blocking Activity of Ipilimumab

Receptor blocking activity (RBA) assay was then employed to prove that the decreased binding of TMEAbodies to the CTLA-4 protein would also decrease the blocking efficacy of Ipilimumab for B7-1 CTLA-4 interaction. 0.5 μg/ml human CTLA-4 Fc fusion protein (R&D systems) was absorbed on the Maxisorp ELISA plate (Nunc) and then the plate was blocked by 2% BSA. 0.02 μg/ml biotinylated human B7-1 or B7-2 protein with different concentration of TMEAbodies or WT Ipilimumab were completely incubated with the plate and then the receptor blocking activity was measured with Streptavidin-HRP (ThermoFisher Scientific) incubation followed by TMB reaction like the procedure of standard ELISA.

As shown in FIG. 34, the TMEAbodies with significant decreased binding activity to human CTLA-4 protein also showed dramatic decrease of receptor blocking activity. The fold change of IC₅₀ could be used as quantitative parameter for evaluating the decrease of receptor blocking activity.

Data of other candidates on RBA assay was summarized in Table 59.

TABLE 59
RBA assay data summary of Ipilimumab TMEAbody candidates

| | Blocking | | Restored RBA activity |
|---|---|---|---|
| Selected Mutation ID | efficiency (RBA) | | after protease cleavage |
| Ipi-se001 | 15 | fold | Restored |
| Ipi-se002 | 5 | fold | Restored |
| Ipi-se003 | 8 | fold | Resorted |
| Ipi-se004 | 7 | fold | Resorted |
| Ipi-se005 | 10 | fold | Resorted |
| Ipi-se006 | 25 | fold | 2.5 fold decreased |
| Ipi-se007 | 16 | fold | Resorted |
| Ipi-se053 | 20 | fold | Resorted |
| Ipi-se009 | 18 | fold | Resorted |
| Ipi-se066 | 9 | fold | 3.5 fold decreased |

### Blocking of Antigen Binding of TMEAbodies Resulted in Decreased Functional Efficacy in SEB Induced T Cell Activation Assay

Next, to evaluate whether the decreased antigen binding activity and receptor blocking activity observed in TMEAbodies can contribute to decrease of functional efficacy of Ipilimumab, staphylococcal enterotoxin (SEB) induced T cell activation assay was performed. SEB is a superantigen which can strongly activate T lymphocyte and induce cytokine secretion. Whole PBMC cell from healthy donors (Allcells) were cultured as 1E5 cells per well in 1640 medium (GIBCO) with 10% FBS (GIBCO), 100 ng/ml SEB (Toxin Technology) and different concentration of TMEAbodies, WT Ipilimumab, or isotype control human IgG, respectively. After 96 h of activation, supernatant were collected by centrifugation and IL2 release was measured by IL2 detection kit with ELISA method (R&D systems).

As shown in FIG. 35, TMEAbody with decreased binding activity against CTLA-4 showed impairment of functional efficacy of T cell activation, and protease mediated activation can restore the activity of TMEAbody.

This assay was also carried out for other TMEAbody at single concentration (10 μg/ml) and blocking efficiency was calculated ((WT−TMEAbody)/(WT−hIgG)*100%)). Results were shown in Table 60.

TABLE 60
TMEAbody blocking efficiency SEB induced T cell activation

| | Blocking efficiency | Restored RBA activity |
|---|---|---|
| Selected Mutation ID | (SEB) | after protease cleavage |
| Ipi-se001 | 63% | Restored |
| Ipi-se002 | 45% | Restored |
| Ipi-se003 | 40% | Restored |
| Ipi-se004 | 43% | Restored |
| Ipi-se005 | 64% | Restored |
| Ipi-se006 | 90% | 3 fold decreased |
| Ipi-se007 | 68% | Restored |
| Ipi-se053 | 70% | Restored |
| Ipi-se009 | 72% | Restored |
| Ipi-se066 | 56% | 4 fold decreased |

### Ipilimumab TMEAbodies Regulated Treg in Tumor but not in Periphery

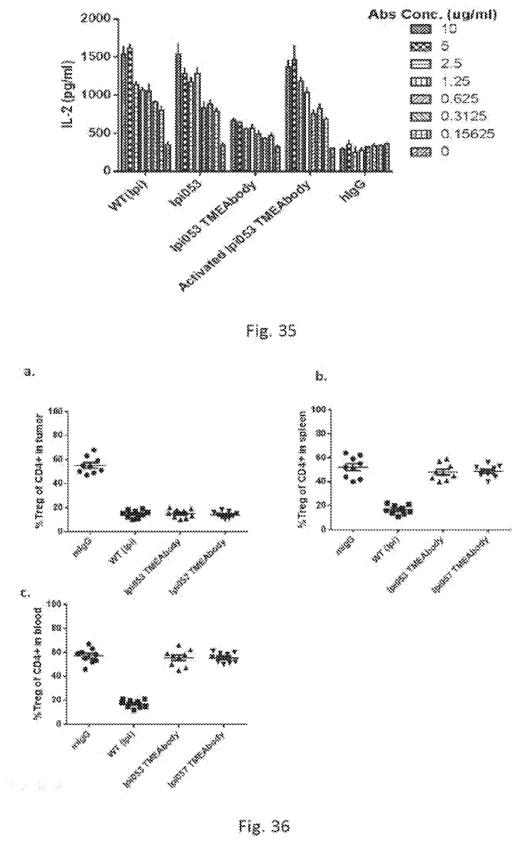

Mechanism study was performed to see whether TMEAbodies were specifically activated in tumor microenvironment but not in the periphery lymph organs. One of the proposed mechanisms for ipilimumab therapy is that it can down-regulate the population of Treg cells through antibody dependent cell mediated cytotoxicity (ADCC) effect, thus to activate the immune response against tumors. Treg population was analyzed with flow cytometry with CD4, CD25, and Foxp3 markers, respectively. As shown in FIG. 36, Ipilimumab TMEAbodies significantly down regulated Treg population in the tumor, with the similar efficacy as WT Ipilimumab. Nevertheless, in the spleen or periphery blood, Ipilimumab TMEAbodies showed very weak or no modulation of Treg population. These results demonstrated that the Ipilimumab TMEAbodies showed specific activity in the tumor microenvironment but not in periphery lymph organs or blood.

### TMEAbody are Stable in Human Plasma

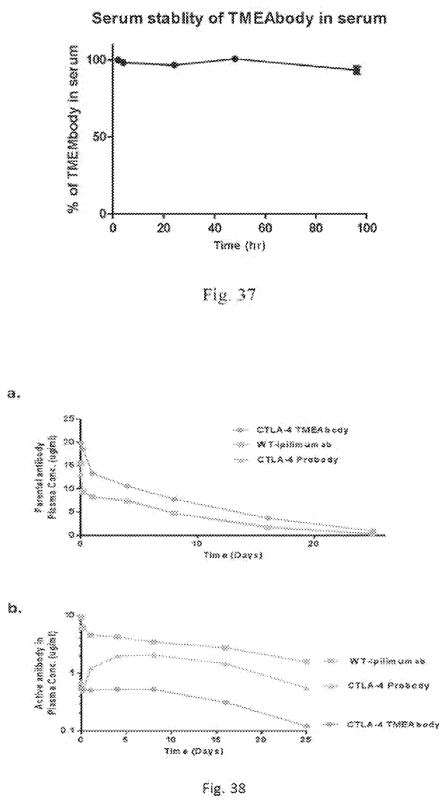

To evaluate the stability of TMEAbody in serum, 1 ug CTLA-4 TEMAbody (Ipi053 with conjugation of S13) was put into 20 ul mouse serum and kept in 37 °C 0 for 2 h, 4 h, 24 h, 48 h, and 96 h, respectively. Then the sample was prepared for Western blot with anti-human Fab HRβ antibody (Sigma). Gel intensity was analyzed with ImageJ software and the relative intensity was analyzed by GraphPad. As shown in the following FIG. 37, TMEAbody showed high stability in mouse serum, without significant degradation after 96 h at 37 °C.

CTLA-4 TMEAbodies Showed Increased Half-Life and Exposure by Conjugation with S47 Functional Group Comparing with WT-Ipilimumab and CTLA-4 Probody

To evaluate the potential effect of chemical conjugation in modulating the pharmacokinetics property of TMEAbody, single IV dose of 1 mg/kg WT Ipilimumab or ipilimumab TMEAbody was injected into Balb/c mice. After 0.5 h, 2 h, 4 h, 8 h, 1 d, 2 d, 5 d, 10 d, 15 d, 20 d, plasma was collected for ELISA test of total antibody and active antibody concentration determination. For total antibody concentration determination, anti-human Fc antibody (Invitrogen) was coated on the ELISA plate (NUNC) and injected antibody was detected by anti-human Fab HRP secondary antibody (Invitrogen). For active antibody concentration determination, human CTLA-4 protein (Sino Biological) was coated on the ELISA plate. Active antibody was then detected by anti-human Fc HRP secondary antibody (Invitrogen). Standard curve was drawn by serial dilution of WT Ipilimumab or Ipilimumab TEMAbody and the standard binding curve was established through four-parameter fitting. The concentration of total antibody or active antibody was calculated through interpolating the Y value to the standard curve. As shown in FIG. 38(a), the half-life of TMEAbody was increased after conjugation with 40 kd functional group, comparing with the WT Ipilimumab antibody or Ipilimumab probody (WO 2018/085555 A1 with MY11 as masking peptide and 2011 as cleavage moiety). Moreover, CTLA-4TMEAbody showed less activation in plasma than Ipilimumab probody with the time, as shown in FIG. 38(b).

### In Vivo Characterization of TMEAbodies in Mouse Tumor Model

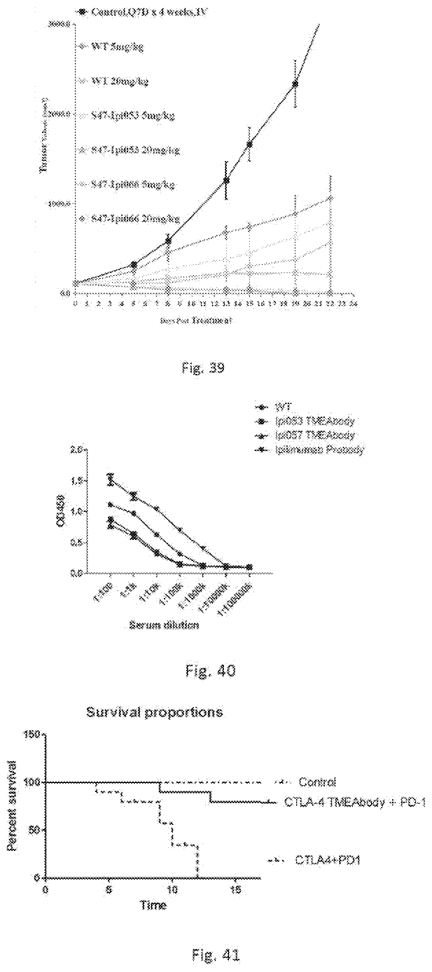

To further characterize the in vivo efficacy of TMEAbodies in treating tumor in animal model, Ipilimumab TMEAbodies, as well as WT Ipilimumab and control human IgG were administrated into MC38 colon adenocarcinoma tumor model in human CTLA-4 knock-in C57BL/6 mice. Human CTLA-4 knock-in C57BL/6 mice were subcutaneously injected with 2E6 MC38 cells into their left lower abdominal quadrant. After 7 days for tumor growth, animals were grouped to have similar mean tumor volume. Animals were administrated with indicated single dose of control human IgG, WT Ipilimumab or equimolar TMEAbodies (the concentration of antibody, n=6), respectively, and tumor volumes were monitored for each animal. As shown in FIG. 39, Ipilimumab TMEAbodies control the tumor size with comparable efficacy as WT Ipilimumab, whereas control human IgG failed to show any efficacy. The tumor volume inhibition rate was summarized in Table 61. This result implied that Ipilimumab TMEAbodies could be activated in the tumor microenvironment and inspired anti-tumor immune response.

TABLE 61
Tumor growth inhibition rate at day 17 after administration

| Mutation ID | Dose | | Cure rate | Dead |
|---|---|---|---|---|
| human IgG | 5 | mg/kg | 0 | 2 |
| WT (Ipi) | 5 | mg/kg | 0 | 0 |
| WT (Ipi) | 20 | mg/kg | 33.3 | 1 |
| S47- Ipi-se053 | 5 | mg/kg | 16.6% | 0 |
| S47- Ipi-se053 | 20 | mg/kg | 83.3% | 0 |
| S47- Ipi-se066 | 5 | mg/kg | 33.3% | 0 |
| S47- Ipi-se066 | 20 | mg/kg | 83.3% | 0 |

As shown in Table 61, inhibition on tumor growth and cure rate by S47-Ipi053 and 547-Ipi066 were greatly improved as compared with the groups treated by WT (Ipi) using the same molar concentration.

### CTLA-4 TMEAbodies Showed Low Immunogenicity in Animals

To evaluate the immunogenicity of TMEAbody, three groups of Balb/c mice (each n=5) were immunized with 50 μg Ipilimumab TMEAbody, WT Ipilimumab, or Ipilimumab Probody (WO 2018/085555 A1 with MY11 as masking peptide and 2011 as cleavage moiety) with complete Freund's adjuvant (CFA). After 14 days of primary immunization, animals were boosted with 25 μg Ipilimumab TMEAbody, WT Ipilimumab, or Ipilimumab Probody with incomplete Freund's adjuvant (IFA). Serum was obtained on the 7^{th} day after boosting, and tested for antibody titer against Ipilimumab TMEAbody, WT Ipilimumab, or Ipilimumab Probody, respectively. 1% human serum was used in the serum dilution buffer to block any antibodies against constant region of human IgG. As shown in FIG. 40, TMEAbody caused very low immune response in the animals, with comparable or lower antibody titer than WT Ipilimumab. Nevertheless, Ipilimumab Probody caused dramatic increase of immunogenicity, which might be due to the foreign sequences included in the N terminal of light chain.

### CTLA-4 TMEAbodies Showed Low Toxicity In Vivo

It is well known in the art, though combination of anti-PD-1 and anti-CTLA-4 antibody are effective_(ORR) for treating melanoma, it was found in the current clinic research that combination exhibited 55% TRAEs grade 3-4 and 30% patient have to discontinue the therapy. We presume these TRAEs may be improved if the antibody is inhibited or blocked by a conjugate and is released after arriving at a local environment of tumor so as to reduce the exposed time or dose of active drug in a non-diseased environment. For this reason, experiments were conducted with mice suffering from type I diabetes mellitus (NOD). Diabetes mellitus of this kind of mice is an autoimmune disease, wherein self-activated T lymphocyte cells destroy pancreatic islet β cells, resulting in insufficient secretion of insulin. First, female NOD of 10 weeks old (Beijing Vital River Laboratory Animal Technology Co., Ltd.) were injected with control IgG, high (15 mpk) dose of anti-PD-1 and anti-CTLA-4 antibody or 15 mpk dose of anti-PD-1 and anti-CTLA-4 TMEAbody, respectively at day 0. Indicators of diabetes mellitus, including glucose in urine and two blood sugar levels were observed every day for 12 days until no new indicator of urine glucose was observed.

Protection from autoimmunity by the conjugate was shown in FIG. 41. Results showed that protection of an immune system by a conjugate of anti-CTLA4 TMEAbody in combination therapy conjugate could reduce autoimmunity as compared to the primary antibody.

### Example 16: Generation and Characterization of PD-1 TMEAbody (Pembrolizumab)

As shown in example of Ipilimumab TMEAbody screening above, multiple sites of anti-PD-1 antibody (Pembrolizumab) were mutated into Cysteine for site specific conjugation. The mutation position in a heavy chain of the anti-PD-1 antibody (Pembrolizumab) is selected from the group consisting of (Numbered sequentially from N terminal to C terminal without using the Kabat or other antibody numbering systems): Ser7, Gly8, Gly15, Ala16, Ser17, Ala24, Ser25, Gly26, Tyr27, Thr28, Thr30, Asn31, Tyr32, Tyr33, Tyr35, Ala40, Gly42, Gly44, Leu45, Gly49, Gly50, Ile51, Asn52, Ser54, Asn55, Gly56, Gly57, Thr58, Asn59, Lys63, Lys65, Thr69, Leu70, Thr71, Thr72, Asp73, Ser74, Ser75, Thr76, Thr77, Thr78, Ala79, Leu83, Ser85, Leu86, Thr91, Ala92, Arg99, Asp100, Tyr101, Arg102, Asp104, Gly106, Gly111, Gly113, Thr114, 115Thr, 117Thr, Ser119, Ser120, Ala121, Ser122, Thr123, Lys124, Gly125 and Ser127; the mutation position in a light chain is selected from the group consisting of: Ile2, Thr5, Ser7, Ala9, Thr10, Leu11, Ser12, Leu13, Ser14, Gly16, Ala19, Thr20, Ala25, Ser26, Lys27, Gly28, Ser30, Thr31, Ser32, Gly33, Tyr34, Ser35, Tyr36, Leu37, Gly45, Ala47, Leu50, Leu51, Ile52, Tyr53, Leu54, Ala55, Ser56, Tyr57, Leu58, Ser60, Gly61, Ala64, Ser67, Gly68, Ser69, Gly70, Ser71, Gly72, Thr73, Ala76, Thr78, Ser80, Ser81, Ser95, Arg96, Asp97, Leu98, Leu100, Thr101, Phe102, Gly104, Ile110, Lys111 and K130. ELISA characterization with human Fc tagged PD-1 protein (Sino Biological) was carried out to identify the candidate sites with good blocking efficiency and recovery efficiency. Sites with blocking efficiency >5 (in other words, the activity <20%) fold and restored activity after enzyme digestion (EC50 change <2 fold, or in other words, the activity >50%) were selected for further development, as shown in Table 62.

TABLE 62
Selected anti-PD-1 TMEAbody (Pembrolizumab) candidates
based on blocking and recovery efficiency

| | | | | | Blocking | | Restored |
|---|---|---|---|---|---|---|---|
| Selected | Heavy or | Mutation | | Conjugation | efficiency | | activity after |
| Mutation ID | light chain | site | R4 | efficiency | of S47 | | R4 conjugation |
| Pem-se001 | HC | Tyr27 | R4-7 | 89% | 5 | fold | Restored |
| Pem-se002 | HC | Tyr32 | R4-7 | 91% | 6 | fold | Restored |
| Pem-se003 | HC | Asn55 | R4-11 | 92% | 5 | fold | Restored |
| Pem-se004 | HC | Lys65 | R4-7 | 88% | 7 | fold | Restored |
| Pem-se005 | HC | Arg102 | R4-26 | 87% | 6 | fold | Restored |
| Pem-se006 | LC | Lys27 | R4-7 | 91% | 6 | fold | Restored |
| Pem-se007 | LC | Gly28 | R4-1 | 94% | 8 | fold | Restored |
| Pem-se008 | LC | Tyr34 | R4-7 | 90% | 10 | fold | Restored |
| Pem-se009 | LC | Tyr36 | R4-7 | 93% | 18 | fold | Restored |
| Pem-se010 | LC | Tyr57 | R4-7 | 95% | 25 | fold | Restored |
| Pem-se011 | LC | Arg96 | R4-26 | 90% | 7 | fold | Restored |

### Example 17: Functional Characterization of Anti-PD-1 TMEAbody (Pembrolizumab)

The human PBMC (Allcells) was inoculated in a 96-well plate in a concentration of 1×10⁵ cells per well. The cells were stimulated with 0.1 ug/ml SEB for three days. Different concentrations of WT anti-PD-1 antibody, TMEAbody, or activated TMEAbody were added and cultured at 37 °C, 5% CO₂ for 4 days. Supernatant was collected and concentration of cytotoxic factor IFN-γ was detected by ELISA kit (R&D). The functional blocking efficiency and recovery rate was summarized in Table 63.

TABLE 63
Functional characterization of anti-PD-1
TMEAbody (Pembrolizumab) candidates

| | | | Blocking | | |
|---|---|---|---|---|---|
| | Heavy or | | efficiency | | Restored |
| Selected | light | Mutation | of S47 in | | activity after |
| Mutation ID | chain | site | IFN-γ assay | | R4 conjugation |
| Pem-se001 | HC | Tyr27 | 3 | fold | Restored |
| Pem-se002 | HC | Tyr32 | 4 | fold | Restored |
| Pem-se003 | HC | Asn55 | 6 | fold | Restored |
| Pem-se004 | HC | Lys65 | 7 | fold | Restored |
| Pem-se005 | HC | Arg102 | 5 | fold | Restored |
| Pem-se006 | LC | Lys27 | 7 | fold | Restored |
| Pem-se007 | LC | Gly28 | 10 | fold | Restored |
| Pem-se008 | LC | Tyr34 | 9 | fold | Restored |
| Pem-se009 | LC | Tyr36 | 19 | fold | Restored |
| Pem-se010 | LC | Tyr57 | 24 | fold | Restored |
| Pem-se011 | LC | Arg96 | 10 | fold | Restored |

### Example 18: Generation and Characterization of PD-1 TMEAbody (Nivolumab)

As shown in example of Ipilimumab and Pembrolizumab TMEAbody screening above, multiple sites of anti-PD-1 antibody (Nivolumab) were mutated into Cysteine for site specific conjugation, the mutation position in a heavy chain of the anti-PD-1 antibody (Nivolumab) is selected from the group consisting of: Gln3, Ser7, Gly8, Gly9, Gly10, Gly15, Ser17, Lys23, Ala24, Ser25, Gly26, Ile27, Asn31, Thr28, Ser30, Ser32, Gly33, Ala40, Gly42, Gly44, Leu45, Ala49, Ile51, Tyr53, Asp54, Gly55, Ser56, Lys57, Tyr59, Tyr60, Ala61, Asp62, Ser63, Lys65, Gly66, Thr69, Ile70, Ser71, Arg72, Asp73, Asn74, Ser75, Lys76, Asn77, Thr78, Leu79, Leu81, Ser85, Leu86, Ala88, Thr91, Ala92, Thr98, Asn99, Asp100, Asp101, Tyr102, Gly104, Gly106, Thr107, Leu108, Thr110, Ser112, Ser113, Ala114, Ser115, Thr116, Lys117, Gly118 and Ser120; the mutation position in a light chain is selected from the group consisting of: Ile2, Leu4, Thr5, Ser7, Ala9, Thr10, Leu11, Ser12, Leu13, Ser14, Gly16, Ala19, Thr20, Leu21, Ala25, Ser26, Ser28, Ser30, Ser31, Tyr32, Leu33, Ala34, Tyr36, Gly41, Ala43, Leu46, Leu47, Ile48, Tyr49, Asp50, Ala51, Ser52, Asn53, Arg54, Ala55, Thr56, Gly57, Ile58, Ala60, Arg61, Ser63, Gly64, Ser65, Gly66, Ser67, Gly68, Thr69, Thr72, Leu73, Thr74, Ile75, Ser76, Ser77, Leu78, Ala84, Ser91, Ser92, Asn93, Arg96, Thr97, Phe98, Gly99, Gly101, Thr102, Ile106, Lys107, Thr109, Ala11, Ala112, Ser114, Ile117 and Ser121Sites with blocking efficiency >5 fold and restored activity after enzyme digestion (EC50 change <2 fold) were selected for further development, as shown in Table 64.

TABLE 64
Selected anti-PD-1 TMEAbody (Nivolumab) candidates
based on blocking and recovery efficiency

| | | | | | Blocking | | Restored |
|---|---|---|---|---|---|---|---|
| ID of selected | Heavy or | Mutation | | Conjugation | efficiency | | activity after |
| Mutation | light chain | site | R4 | efficiency | of S47 | | R4 conjugation |
| Niv-se001 | HC | Lys23 | R4-7 | 95% | 15 | fold | Increased, 432% of |
| | | | | | | | WT |
| Niv-se002 | HC | Tyr53 | R4-7 | 89% | 5 | fold | Restored |
| Niv-se003 | HC | Lys57 | R4-7 | 88% | 6 | fold | Restored |
| Niv-se004 | LC | Tyr32 | R4-7 | 92% | 8 | fold | Restored |
| Niv-se005 | LC | Tyr49 | R4-7 | 94% | 85 | fold | Increased 125% of |
| | | | | | | | WT |
| Niv-se006 | LC | Arg54 | R4-26 | 93% | 7 | fold | Restored |
| Niv-se007 | LC | Ser91 | R4-1 | 95% | 25 | fold | Restored |
| Niv-se008 | LC | Arg96 | R4-26 | 93% | 23 | fold | Restored |

As shown in FIG. 42, Niv-se001 showed increased activity after R4-7 conjugation or protease cleavage of 40 kD conjugated TMEAbody, which is 432% of WT Nivolumab. This might be due to the R4-7 provided increased the binding activity than native Lysine residue.

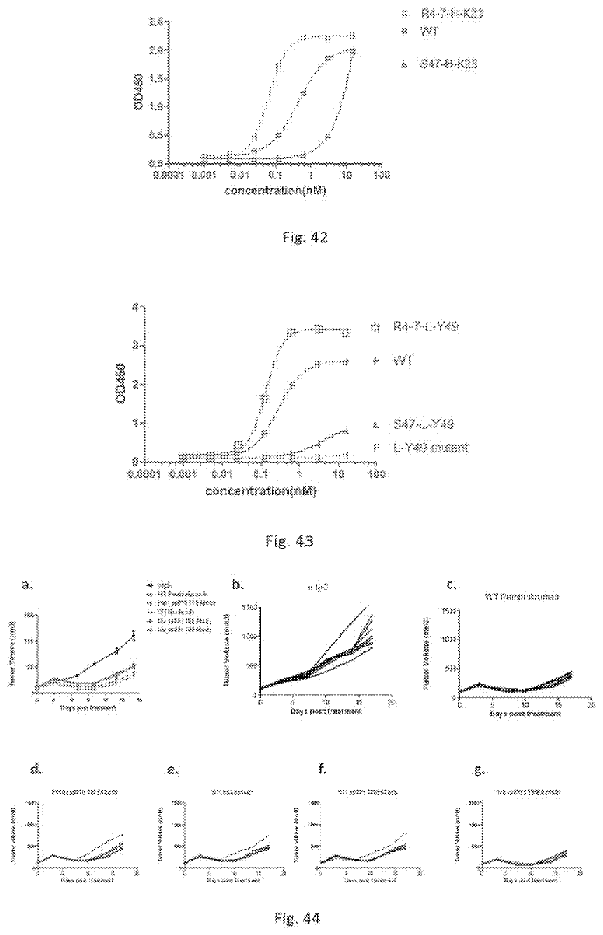

Niv-se005 showed lost binding activity when the Try49 is mutated into Cysteine. However, after conjugation with R4-7, or after protease cleavage of Niv-se005 conjugated with 40 kD functional group, the binding activity is restored with comparable level than WT Nivolumab (125% of WT), as shown in FIG. 43.

### Example 19: Functional Characterization of Anti-PD-1 TMEAbody (Nivolumab)

The human PBMC (Allcells) was inoculated in a 96-well plate in a concentration of 1×10⁵ cells per well. The cells were stimulated with 0.1 ug/ml SEB for three days. Different concentrations of WT anti-PD-1 antibody (Nivolumab), TMEAbody (Nivolumab), or activated TMEAbody (Nivolumab) were added and cultured at 37 °C, 5% CO₂ for 4 days. Supernatant was collected and concentration of cytotoxic factor IFN-γ was detected by ELISA kit (R&D). The functional blocking efficiency and recovery rate was summarized in Table 65.

TABLE 65
Functional characterization of anti-
PD-1 TMEAbody (Nivolumab) candidates

| | | | Blocking | | Restored |
|---|---|---|---|---|---|
| | Heavy | | efficiency of | | activity |
| Selected | chain or | Mutation | S47 in IFN-γ | | after R4 |
| Mutation ID | light chain | site | assay | | conjugation |
| Niv-se001 | HC | Lys23 | 18 | fold | Increased, 332% |
| | | | | | of WT |
| Niv-se002 | HC | Tyr53 | 3 | fold | Restored |
| Niv-se003 | HC | Lys57 | 7 | fold | Restored |
| Niv-se004 | LC | Tyr32 | 8 | fold | Restored |
| Niv-se005 | LC | Tyr49 | 25 | fold | Restored. 122% |
| | | | | | of WT |
| Niv-se006 | LC | Arg54 | 9 | fold | Restored |
| Niv-se007 | LC | Ser91 | 24 | fold | Restored |
| Niv-se008 | LC | Arg96 | 21 | fold | Restored |

### Example 20: In Vivo Characterization of Anti-PD-1 TMEAbody (Pembrolizumab and Nivolumab) in Treating Mouse Tumor

To further characterize the in vivo efficacy of anti-PD-1 TMEAbodies in treating tumor in animal model, anti-PD-1 TMEAbodies (Pem-se010 TMEAbody based on Pembrolizumab and Niv-se007 TMEAbody based on Nivolumab), as well as WT PD-1 antibodies (Pembrolizumab and Nivolumab) and control human IgG were administrated into MC38 colon adenocarcinoma tumor model in human PD-1 knock-in C57BL/6 mice. Human PD-1 knock-in C57BL/6 mice were subcutaneously injected with 2E6 MC38 cells into their left lower abdominal quadrant. After 7 days for tumor growth, animals were grouped to have similar mean tumor volume. Animals were administrated 10 mg/kg single dose of PD-1 TMEAbodies (the concentration of antibody without PEG linker), WT PD-1 antibodies, or control human IgG and tumor volumes were monitored for each animal.

As shown in FIG. 44, Pembrolizumab and Nivolumab TMEAbodies control the tumor size with comparable efficacy as WT Pembrolizumab or Nivolumab antibodies, whereas control human IgG failed to show any efficacy. Interestingly, Niv-se001 TMEAbody showed improved efficacy than WT Nivolumab in treating tumor, which might be due to its enhanced binding activity after protease cleavage. The results implied that these anti-PD-1 TMEAbodies could be activated in the tumor microenvironment and inspire anti-tumor immune response.

### Example 21: Generation of Mouse Anti-PD-1 Antibody (J43v2) for Efficacy Study in Mouse Tumor Model

Hamster anti-mouse PD-1 antibody sequences were disclosed in US 20170044259A1. This heavy chain of this antibody was re-formatted into mouse IgG2a to reduce the immunogenicity in mouse. As the screening method above, multiple sites were designed for screening of TMEAbody with high blocking efficiency. Finally Ser95 on LC was selected for TMEAbody generation due to its high efficiency blocking (35 fold in ELISA assay with mouse PD-1 protein). MC38 mouse tumor model was carried out with 10 mg/kg single dose of WT J43v2 antibody or J43v2 TMEAbody (for each group n=8). At the days of 17 after administration, J43v2 TMEAbody showed 75% tumor inhibition, with comparable inhibition efficacy than WT J43v2 antibody (83%). This result indicated that the PD-1 antibody can be activated and played its anti-tumor activity in vivo.

### Example 22: Generation of Anti-Mouse CTLA-4 Antibody (9D9) for Efficacy Study in Mouse Tumor Model

To generate mouse CTLA-4 surrogate TMEAbody for further functional and toxicity studies, we produced and purified anti-mouse CTLA-4 antibody and its mutant variants (9D9 clone, mIgG2b isotype, sequences shown in WO 2007/123737 A2). As the screening method above, multiple sites were designed for screening of TMEAbody with high blocking efficiency. Finally Tyr54 on LC was selected for TMEAbody generation due to its high efficiency blocking (26 fold in ELISA assay with mouse CTLA-4 protein). CT26 mouse tumor model was carried out with 10 mg/kg single dose of WT 9D9 antibody or 9D9 TMEAbody (for each group n=8). At the days of 17 after administration, 9D9 TMEAbody showed 69% tumor inhibition, with comparable inhibition efficacy than WT 9D9 antibody (74%). This result indicated that the 9D9 TMEAbody can be activated and played its anti-tumor activity in vivo.

### Example 23: Mouse PD-1 and CTLA-4 TMEAbody Showed Decreased Toxicity than WT Antibodies

It is well known in the art, though combination of anti-PD-1 and anti-CTLA-4 antibody are effective (ORR) for treating melanoma, it was found in the current clinic research that combination exhibited 55% TRAEs grade 3-4 and 30% patient have to discontinue the therapy. We presume these TRAEs may be improved if the antibody is inhibited or blocked by a conjugate and is released after arriving at a local environment of tumor so as to reduce the exposed time or dose of active drug in a non-diseased environment. For this reason, experiments were conducted with mice suffering from type I diabetes mellitus (NOD). Diabetes mellitus of this kind of mice is an autoimmune disease, wherein self-activated T lymphocyte cells destroy pancreatic islet β cells, resulting in insufficient secretion of insulin. First, female NOD of 10 weeks old (Beijing Vital River Laboratory Animal Technology Co., Ltd.) were injected with control IgG, high (15 mpk) dose of anti-mouse PD-1 (J43v2) and anti-mouse CTLA-4 antibody (9D9), or one or both of these two antibodies were replaced with its TMEAbody form at day 0. Indicators of diabetes mellitus, including glucose in urine and two blood sugar levels were observed every day for 12 days until no new indicator of urine glucose was observed.

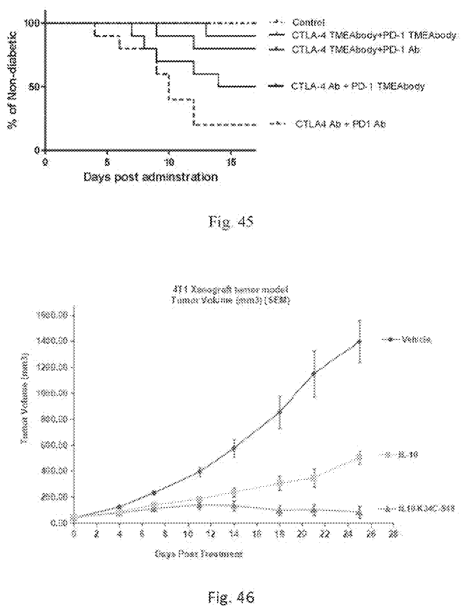

Protection from autoimmunity by the conjugate shown in FIG. 45. Results showed that anti-CTLA4 or anti-PD1 TMEAbody in combination therapy could reduce autoimmunity as compared to the primary antibody. Moreover, combination of these two TMEAbodies resulted in very low toxicity with comparable level than control group.

### Example 24: Generation and Characterization of Anti-PD-1 TMEAbody (Patent WO 2017/124050 A1)

As the method above, anti-PD-1 antibody sequence was downloaded from patent WO 2017/124050 A1 and sites screening was performed to identify TMEAbody with good blocking efficiency. The mutation position in a heavy chain of the anti-PD-1 antibody is selected from the group consisting of Ser28, Ser31, tyr33, Asn36, Gly50, Tyr51, Ser53, Tyr54, Asp55, Ser57, Lys58, Asn59, Tyr60, Asn61, Lys65, Asn66, Thr69, Thr74, Gly100, Asp105, Tyr106; the mutation position in a light chain is selected from the group consisting of Lys24, Gln27, Ser28, Asp31, Asp32, Asn33, Asn34, Gln35, Lys36, Asn37, Tyr38, Ser58, Arg60, Glu61, Ser62, Gly63, Gly70, Ser73, Thr75, Gln95, Gln96, Tyr98, Thr100, Tyr102. Binding ELISA was performed with Fc tagged human PD-1 protein (Sino Biological) and selected sites with good blocking efficiency (EC50 change >5 fold) and recovery (EC50 change <2 fold) was summarized in Table 66.

TABLE 66
Selected anti-PD-1 TMEAbody (WO 2017/124050 A1) candidates
based on blocking and recovery efficiency

| | | | | | Blocking | | Restored |
|---|---|---|---|---|---|---|---|
| ID of selected | Heavy chain | Mutation | | Conjugation | efficiency | | activity after |
| Mutation | or light chain | site | R4 | efficiency | of S47 | | R4 conjugation |
| PD1-se001 | HC | Tyr33 | R4-7 | 99% | 198 | fold | Restored |
| PD1-se002 | HC | Ser53 | R4-1 | 95% | 9 | fold | Restored |
| PD1-se003 | HC | Tyr60 | R4-7 | 88% | 6 | fold | Restored |
| PD1-se004 | LC | Tyr38 | R4-7 | 94% | 15 | fold | Restored |
| PD1-se005 | LC | Ser58 | R4-1 | 93% | 16 | fold | Restored |
| PD1-se006 | LC | Thr100 | R4-3 | 91% | 6 | fold | Restored |

### Example 25: Generation and Characterization of Anti-4-11BB TMEAbody

4-1BB antibody sequence was downloaded from US 2018/0194851 A1 (clone MOR 7480.1). The mutation position in a heavy chain of the anti-4-1BB antibody is selected from the group consisting of: Thr31, Tyr32, Ser35, Lys, Tyr52, Asp55, Ser56, Tyr57, Thr58, Asn59, Tyr6B, Ser61, Gln65, Gly66, Gly99, Tyr100, Gly101, Asp104, Tyr105; the mutation position in a light chain is selected from the group consisting of: Ser23, Gly24, Asp25, Asn26, Gly28, Asp29, Gln3, Tyr31, Gln49, Asp50, Lys51, Asn52, Arg53, Ser55, Gly56, Thr89, Tyr9, Thr91, Gly92, Gly94, Ser95. Human 4-1BB protein was used for ELISA characterization to identify mutant sites with good blocking efficiency (EC50 change >5 fold) as well as good recovery (EC50 change <2 fold) after protease digestion. The selected sites were summarized in Tables 67 and 68.

TABLE 67
Selected anti-4-1BB TMEAbody candidates based on blocking and recovery efficiency

| | | | | | Blocking | | Restored |
|---|---|---|---|---|---|---|---|
| ID of selected | Heavy chain | Mutation | | Conjugation | efficiency | | activity after |
| Mutation | or light chain | site | R4 | efficiency | of S47 | | R4 conjugation |
| 4-1BB-se001 | HC | Tyr32 | R4-7 | 95% | 23 | fold | Restored |
| 4-1BB -se002 | HC | Lys50 | R4-7 | 93% | 16 | fold | Restored |
| 4-1BB -se003 | HC | Tyr105 | R4-7 | 94% | 9 | fold | Restored |
| 4-1BB -se004 | LC | Tyr31 | R4-7 | 94% | 18 | fold | Restored |
| 4-1BB -se005 | LC | Lys51 | R4-7 | 96% | 6 | fold | Restored |
| 4-1BB -se006 | LC | Tyr90 | R4-7 | 95% | 7 | fold | Restored |
| 4-1BB -se007 | LC | Thr91 | R4-1 | 91% | 11 | fold | Restored |

TABLE 68
Selected anti-4-1BB TMEAbody candidates based on blocking and recovery efficiency

| ID of | | | | Blocking | | Blocking | | S48 Restored | S27 Restored |
|---|---|---|---|---|---|---|---|---|---|
| selected | Heavy chain | Mutation | | efficiency of | | efficiency of | | activity by | activity by |
| Mutation | or light chain | site | R4 | S27 | | S48 | | Granzyme B | pH 6.0 |
| 4-1BB- | HC | Tyr32 | R4-7 | 23 | fold | 20 | fold | Restored | Restored |
| se001 | | | | | | | | | |
| 4-1BB - | HC | Lys50 | R4-7 | 16 | fold | 12 | fold | Restored | Restored |
| se002 | | | | | | | | | |
| 4-1BB - | HC | Tyr105 | R4-7 | 9 | fold | 7 | fold | Restored | Restored |
| se003 | | | | | | | | | |
| 4-1BB - | LC | Tyr31 | R4-7 | 18 | fold | 18 | fold | Restored | Restored |
| se004 | | | | | | | | | |
| 4-1BB - | LC | Lys51 | R4-7 | 6 | fold | 5 | fold | Restored | Restored |
| se005 | | | | | | | | | |
| 4-1BB - | LC | Tyr90 | R4-7 | 7 | fold | 6 | fold | Restored | Restored |
| se006 | | | | | | | | | |
| 4-1BB - | LC | Thr91 | R4-1 | 11 | fold | 10 | fold | Restored | Restored |

se007

### Example 26: Generation and Characterization of an B-Her2 TMEAbody (Trastuzumab)

The heavy chain and light chain sequences of Trastuzumab was downloaded from Drug Bank (https://www.drugbank.ca/drugs/DB00072) and sites screening was performed to identify anti-Her2 TMEAbody with good blocking efficiency. The mutation position in a heavy chain of the anti-Her2 antibody (Trastuzumab) is selected from the group consisting of: Arg19, Lys30, Asp31, Tyr33, Arg50, Tyr62, Asn55, Tyr57, Arg59, Tyr60, Asp62, Lys65, Asp102, Tyr105; the mutation position in a light chain is selected from the group consisting of: Asp1, Gln3, Gln27, Asp28, Asn30, Tyr49, Tyr55, Arg66, Asp70, Tyr92. His tagged Her2 protein (Sino Biological) was used for ELISA. The selected sites with good blocking efficiency and good recovery were summarized in Table 69.

TABLE 69
Selected anti-Her2 TMEAbody candidates based on blocking and recovery efficiency

| | | | | | Blocking | | Restored |
|---|---|---|---|---|---|---|---|
| ID of selected | Heavy chain | Mutation | | Conjugation | efficiency | | activity after |
| Mutation | or light chain | site | R4 | efficiency | of S47 | | R4 conjugation |
| Trast-se001 | HC | Tyr33 | R4-7 | 97% | 13 | fold | Restored |
| Trast-se002 | HC | Lys65 | R4-7 | 95% | 8 | fold | Restored |
| Trast-se003 | HC | Tyr105 | R4-7 | 90% | 25 | fold | Restored |
| Trast -se004 | LC | Gln30 | R4-11 | 89% | 16 | fold | Restored |
| Trast -se005 | LC | Tyr49 | R4-7 | 91% | 20 | fold | Restored |
| Trast -se006 | LC | Asp70 | R4-11 | 96% | 7 | fold | Restored |

### Example 27: Generation and Characterization of Anti-TNFa TMEAbody (Adalimumab)

The heavy chain and light chain sequences of Adalimumab was downloaded from Drug Bank (https://www.drugbank.ca/drugs/DB00051) and sites screening was performed to identify anti-TNFa TMEAbody with good blocking efficiency. The mutation position in a heavy chain of the anti-TNFα antibody is selected from the group consisting of: Ser7, Gly8, Gly9, Gly10, Leu11, Gly15, Ser17, Leu18, Leu20, Ala24, Ser25, Gly26, Thr28, Asp30, Asp31, Tyr32, Ala33, Ala40, Gly42, Gly44, Leu45, Ser49, Ala50, Ile51, Thr52, Asn54, Ser55, Gly56, Ile58, Asp59, Tyr60, Ala61, Asp62, Ser63, Glu65, Gly66, Phe68, Thr69, Ile70, Ser71, Asp73, Asn74, Ala75, Lys76, Ser78, Leu79, Tyr80, Leu81, Ser85, Leu86, Ala88, Thr91, Ala92, Lys98, Ser100, Tyr101, Leu102, Ser103, Thr104, Ala105, Ser106, Ser107, Leu108, Asp109, Tyr110, Gly112, Gly114, Thr115, Leu116, thr118, Ser120, Ser121, Ala122, Ser123 and Thr124; the mutation position in a light chain is selected from the group consisting of: Asp1, Thr5, Ser7, Ser9, Ser10, Leu11, Ser12, Ala13, Ser14, Gly16, Thr20, Ile21, Ala25, Ser26, Gln27, Gly28, Ile29, Arg30, Asn31, Tyr32, Leu33, Ala34, Tyr36, Lys39, Gly41, Lys42, Ala43, Leu48, Leu47, Ile48, Tyr49, Ala50, Ala51, Ser52, Thr53, Leu54, Gln55, Ser56, Gly57, Ser60, Ser63, Gly64, Ser65, Gly66, Ser67, Gly68, Thr69, Asp70, Thr72, Leu73, Thr74, Ile75, Ser76, Ser77, Leu78, Ala84, Thr85, Tyr91, Asn92, Arg93, Ala94, Tyr96, Thr97, Phe98, Gly99, Gly101, Thr102, Ile106, Lys107, Thr109 and Ala111. TNFa protein (Sino Biological) was used for ELISA characterization and selected sites with good blocking efficiency (EC50 change >5 fold) and good recovery (EC50 change <2 fold) were summarized in the Table 70.

TABLE 70
Selected anti-TNFa TMEAbody candidates based on blocking and recovery efficiency

| | | | | | Blocking | | Restored |
|---|---|---|---|---|---|---|---|
| ID of selected | Heavy chain | Mutation | | Conjugation | efficiency | | activity after |
| Mutation | or light chain | site | R4 | efficiency | of S47 | | R4 conjugation |
| Ada-se001 | HC | Tyr32 | R4-7 | 95% | 11 | fold | Restored |
| Ada-se002 | HC | Asp59 | R4-11 | 92% | 7 | fold | Restored |
| Ada-se003 | HC | Tyr101 | R4-7 | 93% | 6 | fold | Restored |
| Ada-se004 | LC | Arg30 | R4-26 | 96% | 25 | fold | Restored |
| Ada-se005 | LC | Tyr32 | R4-7 | 94% | 28 | fold | Restored |
| Ada-se006 | LC | Gln55 | R4-11 | 91% | 32 | fold | Restored |
| Ada-se007 | LC | Tyr96 | R4-7 | 92% | 9 | fold | Restored |

### Example 28: Generation and Characterization of Anti-PD-L1 TMEAbody (Atezolizumab)

The heavy chain and light chain sequences of anti-PD-L1 antibody (Atezolizumab) was downloaded from Drug Bank (https://www.drugbank.ca/drugs/DB11595) and sites screening was performed to identify anti-TNFa TMEAbody with good blocking efficiency. The mutation position in a heavy chain of the anti-PD-L1 antibody (Atezolizumab) is selected from the group consisting of: Gln3, Asp31, Tyr54, Tyr59, Tyr60, Asp62, Lys65, Asp73, Lys76, Asn77, Arg99; the mutation position in a light chain is selected from the group consisting of: Arg24, Gln27, Asp28, Tyr49, Tyr55, Asp70, Gln89, Gln90, Tyr91, Tyr93. Fc tagged human PD-L1 protein (Sino Biological) was used for ELISA characterization and selected sites with good blocking efficiency (EC50 change >5 fold) and good recovery (EC50 change <2 fold) were summarized in Table 71.

TABLE 71
Selected anti-PD-L1 TMEAbody candidates based on blocking and recovery efficiency

| | | | | | Blocking | | Restored |
|---|---|---|---|---|---|---|---|
| ID of selected | Heavy chain | Mutation | | Conjugation | efficiency | | activity after |
| Mutation | or light chain | site | R4 | efficiency | of S47 | | R4 conjugation |
| Ate-se001 | HC | Tyr54 | R4-7 | 98% | 15 | fold | Restored |
| Ate-se002 | HC | Lys76 | R4-7 | 93% | 7 | fold | Restored |
| Ate-se003 | LC | Asp28 | R4-11 | 91% | 10 | fold | Restored |
| Ate-se004 | LC | Tyr49 | R4-7 | 96% | 13 | fold | Restored |
| Ate-se005 | LC | Tyr91 | R4-7 | 92% | 23 | fold | Restored |

### Example 29: Generation and Characterization of Anti-CD28 TMEAbody

Anti-human CD28 antibody heavy chain and light chain sequences were downloaded from patent US008709414B2 and sites screening was performed to identify anti-CD28 TMEAbody with good blocking efficiency. The mutation position in a heavy chain of the anti-CD28 antibody is selected from the group consisting of Ser7, Gly8, Gly15, Ala16, Ser17, Ser21, Ala24, Ser25, Gly26, Tyr27, Thr28, Thr30, Ser31, Tyr32, Ala40, Gly42, Gly44, Gly49, Tyr52, Gly54, Thr58, Ala68, Thr69, Thr71, Thr74, Ser75, Ser77, Thr78, Ala79, Ser84, Leu86, Ser88, Thr91, Ala92, Thr97, Ser99, Tyr101, Gly102, Leu103, Gly113, Thr114, Thr115, Thr117, Ser119, Ser120, Ala121, Ser122 and Thr123; the mutation position in a light chain is selected from the group consisting of Thr5, Ser7, Ser9, Ser10, Ser11, Ser12, Ala13, Ser14, Gly16, Thr20, Thr22, A1a25, Ser26, Ser27, Ile29, Tyr30, Ala43, Leu46, Leu47, Tyr49, Lys50, Ala51, Ser52, Leu54, Thr56, Gly57, Ser60, Ser63, Gly64, Ser65, Gly66, Ser67, Gly68, Thr69, Asp70, Thr72, Thr74, Ser76, Ser77, Ala84, Thr85, Gly91, Thr93, Tyr94, Tyr96, Thr97, Phe98, Gly99, Gly100, Gly101, Thr102, Thr109 and Ala111. Fc tagged human CD28 protein (Sino Biological) was used for ELISA characterization and selected sites with good blocking efficiency (EC50 change >5 fold) and good recovery (EC50 change <2 fold) were summarized in the Table 72.

TABLE 72
Selected anti-CD28 TMEAbody candidates based on blocking and recovery efficiency

| | | | | | Blocking | | Restored |
|---|---|---|---|---|---|---|---|
| ID of selected | Heavy chain | Mutation | | Conjugation | efficiency | | activity after |
| Mutation | or light chain | site | R4 | efficiency | of S47 | | R4 conjugation |
| CD28-se001 | HC | Tyr27 | R4-7 | 95% | 6 | fold | Restored |
| CD28-se002 | HC | Thr58 | R4-1 | 93% | 7 | fold | Restored |
| CD28-se003 | HC | Tyr101 | R4-7 | 91% | 6 | fold | Restored |
| CD28-se004 | LC | Tyr30 | R4-7 | 91% | 16 | fold | Restored |
| CD28-se005 | LC | Tyr49 | R4-7 | 98% | 32 | fold | Restored |
| CD28-se006 | LC | Lys50 | R4-7 | 92% | 27 | fold | Restored |
| CD28-se007 | LC | Tyr96 | R4-7 | 90% | 13 | fold | Restored |

### Example 30: Tumor Microenvironment Activated IL-10 Cytokine (IL-10 TMEAkine)

### 1. Expression and Purification of the Mutant IL-10 Cytokine

The mutant IL-10 DNA sequence ligated to a modified pTT5 vector (Biovector) was

optimized for expression in 293T cells and synthesized (GENEWIZ, Inc., Suzhou, China). Transfection of the mutant IL-10 DNA was performed and after incubation for 4-7 days, the supernatant containing mutant IL-10 was collected.

In Pichia Pastoris expression, the expression vector pPICZαA containing the mutant IL-10 genes was optimized and prepared (GENEWIZ, Inc., Suzhou, China). The amino acid sequence of the mutant IL-10 was described in SEQ ID NO.1. The expression vector pPICZα A was transformed in E. coli (DH5α) for plasmid purification. Then pPICZα A was transformed into GS115 by electroporation. The transformed colony was selected by obtaining the growing colonies after growing on the 100, 300, 500, 1000, 1500, 2000 μg/mL Zeocin™ containing YPD plates. After finally selecting the transformant, the recombinant GS115 strain was grown in BMGY medium at 30 °C, with vigorous shaking in baffled flasks to an OD600 of 2-6. The cells were then pelleted by centrifugation and suspended in BMMY to an OD600 of 1, to which was added 0.5% methanol daily in order to induce the heterologous protein expression. After a four-day induction, supernatant containing the secreted mutant IL-10 protein was collected by centrifugation. The total protein in the supernatant was concentrated by ultrafiltration using a 10-kDa molecular mass cutoff membrane. The concentrated protein was dialyzed with buffer A (20 mMBIS-TRIS, 0.065M NaCl, pH 6.5) for more than 24 h, then loaded onto a cation-exchange column equilibrated with buffer A. Mutant IL-10 was eluted from the column with gradient concentration of NaCl in the range of 0.065-0.4M and the eluent was collected and concentrated. The condensed sample was further purified on Sephacryl 5-100 HRgel filtration column using 10 mMTris-HCl, pH7.4, as the elution buffer.

### 2. Conjugate S48 to the Mutant IL-10 and it was Called as the Name of IL-10 TMEAkine (Tumor Microenvironment Activated IL-1-Cytokine).

Mutant IL-10 protein was generated and purified as described above. Purified mutant IL-10 was performed as the concentration of 0.5 mg/mL in 20 mMTris buffer (pH 7.4) containing 2 mM EDTA. Add TCEP solution to mutant IL-10 as a ratio of 100:1 and incubate for 4 h at 4 °C with gentle agitation. Then the mutant IL-10 solution was dialyzed with 20 mMTris buffer (pH 7.4) containing 150 mMNaCl for 4 h at 4 °C. Afterwards, immediately add the S48 to mutant IL-10 solution as a ratio of 20:1 and incubate for 16 h at 25 °C with gentle agitation. Terminate the reaction by removal of residual S48. Before the enzyme cleavage, change the IL-10TMEAkine buffer to enzyme buffer through dialysis. For activation, enzyme was added to IL-10TMEAkine solution and incubated for 16 h at 37 °C.

3. Screening IL-10TMEAkine that Blocks the Binding to IL-10R1 or R2, and Recovers the Binding Activity after Enzyme Cleavage In Vitro.

Dispense 60 μl PBS buffer containing 1 ug IL-TORT-Fc/IL-10R2-Fc/His solution into the wells. Apply sealing tape to the top of the plate and incubate the plate overnight at 4 °C. After incubation, remove the tape and aspirate each well. After three-time wash with PBST, block the plate by dispensing 200 μl of PBS buffer containing 2% BSA into each well and incubate the plate for 2 h at room temperature. Wash the plate three times and add 60 μl of serial diluted samples to the appropriate wells. Incubate the plate for 1.5 h at room temperature. After three-time wash with PBST, dispense 60 μl of 2 ug/mL IL-10 biotinylated antibody solution to each well and incubate for 1 hour at room temperature. Wash the plate three times and dispense 60 μl of streptavidin solution to each well. Incubate for 30 minutes at room temperature. After washing three times, dispense 100 μl of the HRP substrate solution into each well and incubate for 15 minutes at 3700 After color development, dispense 50 μl of stop solution into each well and immediately measure the absorbance of each well at a wavelength of 450 nm.

### 4. Summary of Various IL-1 Mutation Sites

IL-10 receptors on the cell surface have two different forms: high-affinity receptor: IL-10R1 to IL-10 (Kd=50-200 pM) and low-affinity receptor: IL-10R2 to IL10. The conjugated IL-1 with R4 can recover the binding >80% in some positions by chemical modified maturation of R4 library screening. To select drug candidates, we also performed the screening expression and S48 conjugation reaction with all amino acids of IL-10 in the domain of binding IL-10R1 and IL-10R2. We acquired the possible drug candidates and results are shown in the following Table 73.

TABLE 73
Activity of mutated IL-10 and screening for linker arm

| | | | Type of | | Binding | Decreased fold of |
|---|---|---|---|---|---|---|
| | Amino | Amino | inhibitory | | recovery | binding to ligand after |
| Position | acid | acid | ligand | R4 | (>60%) | Conjugating with S48 (>3 fold) |
| 6 | T | Thr | 1 | R4-7 | 101.2% | 3-fold |
| 8 | S | Ser | 1 | R4-5 | 100.0% | 3-fold |
| 11 | S | Ser | 1 | R4-7 | 132.9% | 9-fold |
| 13 | T | Thr | 1 | R4-6 | 99.1% | 7-fold |
| 17 | G | Gly | 1 | R4-1 | 90.2% | 15-fold |
| 18 | N | Asn | 1 | R4-18 | 92.3% | 12-fold |
| 19 | L | Leu | 1 | R4-7 | 95.3% | 8-fold |
| 21 | N | Asn | 1 | R4-18 | 99.7% | 7-fold |
| 22 | M | Met | 1 | R4-1 | 96.8% | 5-fold |
| 23 | L | Leu | 1 | R4-6 | 101.2% | 12-fold |
| 24 | R | Arg | 1 | R4-20 | 90.0% | 33-fold |
| 25 | D | Asp | 1 | R4-12 | 97.1% | 3-fold |
| 26 | L | Leu | 1 | R4-6 | 93.4% | 3-fold |
| 27 | R | Arg | 1 | R4-20 | 86.6% | 12-fold |
| 28 | D | Asp | 1 | R4-11 | 92.3% | 30-fold |
| 30 | F | Phe | 1 | R4-8 | 97.1% | 6-fold |
| 31 | S | Ser | 1 | R4-2 | 124.6% | 39-fold |
| 32 | R | Arg | 1 | R4-7 | 90.4% | 13-fold |
| 34 | K | Lys | 1 | R4-7 | 99.6% | 50-fold |
| 35 | T | Thr | 1 | R4-6 | 90.1% | 36-fold |
| 38 | Q | Gln | 1 | R4-19 | 87.0% | 52-fold |
| 39 | M | Met | 1 | R4-1 | 88.0% | 62-fold |
| 41 | D | Asp | 1 | R4-11 | 95.2% | 13-fold |
| 42 | Q | Gln | 1 | R4-19 | 90.0% | 18-fold |
| 44 | D | Asp | 1 | R4-11 | 92.1% | 43-fold |
| 45 | N | Asn | 1 | R4-18 | 97.7% | 7-fold |
| 46 | L | Leu | 1 | R4-6 | 89.6% | 22-fold |
| 48 | L | Leu | 1 | R4-7 | 98.9% | 3-fold |
| 49 | K | Lys | 1 | R4-5 | 114.0% | 7-fold |
| 50 | E | Glu | 1 | R4-12 | 99.8% | 4-fold |
| 51 | S | Ser | 1 | R4-7 | 96.7% | 3-fold |
| 53 | L | Leu | 1 | R4-6 | 99.4% | 3-fold |
| 54 | L | Leu | 1 | R4-6 | 98.4% | 3-fold |
| 56 | Q | Gln | 1 | R4-19 | 95.1% | 3-fold |
| 57 | E | Glu | 1 | R4-12 | 95.4% | 10-fold |
| 65 | L | Leu | 1 | R4-6 | 92.3% | 3-fold |
| 69 | I | Ile | 1 | R4-6 | 97.3% | 3-fold |
| 72 | Y | Tyr | 1 | R4-4 | 109.0% | 3-fold |
| 75 | V | Val | 1 | R4-5 | 98.6% | 4-fold |
| 90 | H | His | 1 | R4-19 | 86.5% | 8-fold |
| 91 | V | Val | 1 | R4-5 | 97.2% | 3-fold |
| 93 | S | Asp | 1 | R4-2 | 100.1% | 3-fold |
| 94 | L | Leu | 1 | R4-6 | 88.4% | 5-fold |
| 97 | N | Asn | 1 | R4-18 | 94.5% | 5-fold |
| 100 | T | Thr | 1 | R4-7 | 99.3% | 4-fold |
| 104 | R | Arg | 1 | R4-20 | 98.5% | 3-fold |
| 105 | L | Leu | 1 | R4-6 | 92.3% | 5-fold |
| 107 | R | Arg | 1 | R4-20 | 99.5% | 3-fold |
| 111 | F | Phe | 1 | R4-8 | 90.1% | 12-fold |
| 117 | K | Lys | 1 | R4-7 | 116.6% | 3-fold |
| 118 | S | Ser | 1 | R4-5 | 100.0% | 3-fold |
| 119 | K | Lys | 1 | R4-7 | 99.0% | 3-fold |
| 130 | K | Lys | 1 | R4-7 | 100.0% | 3-fold |
| 131 | L | Leu | 1 | R4-6 | 98.5% | 5-fold |
| 134 | K | Lys | 1 | R4-7 | 128.3% | 5-fold |
| 135 | G | Gly | 1 | R4-1 | 99.5% | 3-fold |
| 137 | Y | Tyr | 1 | R4-4 | 99.1% | 5-fold |
| 138 | K | Lys | 1 | R4-7 | 97.6% | 23-fold |
| 140 | M | Met | 1 | R4-1 | 89.0% | 3-fold |
| 141 | S | Ser | 1 | R4-7 | 112.5% | 19-fold |
| 142 | E | Glu | 1 | R4-12 | 78.0% | 13-fold |
| 143 | F | Phe | 1 | R4-8 | 69.0% | 7-fold |
| 144 | D | Asp | 1 | R4-11 | 90.5% | 15-fold |
| 148 | N | Asn | 1 | R418 | 84.3% | 10-fold |
| 149 | Y | Tyr | 1 | R4-4 | 115.3% | 5-fold |
| 151 | E | Glu | 1 | R4-12 | 82.5% | 13-fold |
| 155 | T | Thr | 1 | R4-7 | 98.1% | 3-fold |
| 157 | K | Lys | 1 | R4-7 | 98.6% | 3-fold |
| 158 | I | Ile | 1 | R4-6 | 81.7% | 9-fold |
| 159 | R | Arg | 1 | R4-20 | 99.0% | 3-fold |
| 15 | F | Phe | 2 | R4-8 | 90.0% | 3-fold |
| 18 | N | Asn | 2 | R4-18 | 99.8% | 4-fold |
| 21 | N | Asn | 2 | R4-18 | 85.3% | 6-fold |
| 22 | M | Met | 2 | R4-1 | 80.0% | 6-fold |
| 24 | R | Arg | 2 | R4-20 | 98.1% | 9-fold |
| 25 | D | Asp | 2 | R4-11 | 91.5% | 5-fold |
| 28 | R | Arg | 2 | R4-20 | 99.0% | 6-fold |
| 29 | A | Ala | 2 | R4-5 | 78.0% | 3-fold |
| 31 | S | Ser | 2 | R4-2 | 87.4% | 7-fold |
| 32 | R | Arg | 2 | R4-7 | 89.6% | 9-fold |
| 33 | V | Val | 2 | R4-7 | 91.4% | 3-fold |
| 34 | K | Lys | 2 | R4-7 | 97.3% | 5-fold |
| 35 | T | Thr | 2 | R4-5 | 123.0% | 6-fold |
| 36 | F | Phe | 2 | R4-8 | 79.6% | 3-fold |
| 81 | E | Glu | 2 | R4-12 | 93.6% | 3-fold |
| 86 | D | Asp | 2 | R4-11 | 81.0% | 3-fold |
| 87 | I | Ile | 2 | R4-6 | 95.7% | 4-fold |
| 88 | K | Lys | 2 | R4-7 | 99.0% | 3-fold |
| 89 | A | Ala | 2 | R4-1 | 78.7% | 5-fold |
| 90 | H | His | 2 | R4-19 | 79.0% | 7-fold |
| 92 | N | Asn | 2 | R4-18 | 86.4% | 3-fold |
| 96 | E | Glu | 2 | R4-12 | 98.0% | 3-fold |
| 97 | N | Asn | 2 | R4-18 | 80.7% | 4-fold |
| 99 | K | Lys | 2 | R4-7 | 116.6% | 3-fold |
| 100 | T | Thr | 2 | R4-7 | 90.7% | 8-fold |
| 101 | L | Leu | 2 | R4-6 | 91.0% | 3-fold |
| 102 | R | Arg | 2 | R4-20 | 99.0% | 3-fold |
| 103 | L | Leu | 2 | R4-6 | 89.4% | 3-fold |
| 104 | R | Arg | 2 | R4-20 | 96.6% | 6-fold |
| 106 | R | Arg | 2 | R4-7 | 99.6% | 3-fold |
| 107 | R | Arg | 2 | R4-20 | 84.0% | 5-fold |
| 119 | K | Lys | 2 | R4-7 | 105.9% | 4-fold |
| 122 | E | Glu | 2 | R4-12 | 99.8% | 3-fold |
| 123 | Q | Gln | 2 | R4-19 | 96.5% | 3-fold |
| 126 | N | Asn | 2 | R4-18 | 99.0% | 3-fold |
| 130 | K | Lys | 2 | R4-7 | 99.7% | 3-fold |

### 5. Study on Efficacy of IL10-K34C-S48 on the 4T1 Tumor Model in BALB/C Mice Model.

Test purpose: to investigate the anti-tumor efficacy of IL10-K34C-S48 in BALB/C mice for treatment of the 4T1 tumor model. Test drug: IL10-K34C-S48 and IL-10 injection, diluted to corresponding concentrations by physiological PBS when testing.

### Method and Results

1. Animal: BALB/C mice of 5 weeks old, all female.
2. Production of tumor model
1) 4T1 cells were purchased from American type culture collection (ATCC) and identified according the specification provided by ATCC. Cells were cultivated in RPMI 1640 culture solution containing 10% fetal bovine serum at 37 °C and 5% CO₂. The cells were passaged every three days and cells within the 13th passage were used.
2) Production of tumor model. 4T1 cells were subcutaneously injected to the back of the BALB/C mice. Mice were randomly grouped after the tumor grew to about 100 mm³ and drug treatment began. Mice were killed after anesthesia on day 28.
3) Course of treatment. There were 3 groups with 6 animals in each group. Included were a control group treated daily, and two single agent groups (1 mg/kg IL-10 treated daily or IL-10-K34C-S48daily (1 mg/kg IL-10 equivalents dose).
4) Grouping and test results are shown in Table 74.
5) Results and discussion. As shown in Table 74, comparing with IL-10, the complete regression on the 4T1 tumor of BALB/C mice was greatly improved after injection of IL10-K34C-S48, indicating that IL10-K34C-S48 exhibits a good anti-tumor efficacy on the 4T1 tumor model.

TABLE 74
Effects of IL10-K34C-S48 on the 4T1
tumor model in BALB/C mice model

| Group | Number of animals | Complete Regression |
|---|---|---|
| Control Group | 6 | 0 |
| IL10-K34C-S48 | 6 | 33.33% |
| IL-10 | 6 | 0 |

Tumor volumes were monitored 2-3 times a week and are presented in FIG. 46.

### Example 31: Activation Efficiency of Different R2 for Targeted Activation in Different Tissue

Cleaving effect of conjugates in which R1 was S13, R3 was R3-5, R4 was R4-7 and R2 was each of the groups shown in Table 75 were evaluated in different tissues. The conjugates were each dissolved and diluted ten times to a concentration of 0.1 mM/ml. At 37 °C, conjugates were each added into 100 μg different acidized human tumor tissue homogenates (pH6.0) in a concentration of 0.2 mg/ml. The enzyme in tumor tissue homogenates could release R1. The released R1 was detected by HPLC, thereby comparing the activation efficiency of the linker. Results were shown in Table 75

TABLE 75
Activation ratio (%) of different R2 peptide in homogenates from different tumor tissues

| | Cells | | | | |
|---|---|---|---|---|---|
| Human | producing | | | | |
| tissues | tumor | R2 = LTPRLGPAAN | R2 = GPAAN | R2 = LSGRSDN | R2 = AAN |
| Fibrosarcoma | HT-1080 | 66.4 | 47.8 | 19.8 | 45.9 |
| Breast cancer | MDA-MB231 | 71.8 | 76.7 | 25.4 | 68.4 |
| Liver cancer | Hepg2 | 68.9 | 58.4 | 21.8 | 55.7 |
| Heart | | 1.6 | 1.1 | 4.3 | 0 |
| Lung | | 1.7 | 1.5 | 5.9 | 0 |

According to the results, extended R2, which is activated by multiple enzymes, exhibits a higher activation than AAN. But activation with multiple enzymes may cause stability problems as shown in heart and lung tissue.

### Example 32: Generation and Characterization of Anti-VEGF TMEAbody (Bevacizumab)

The heavy chain and light chain sequences of Bevacizumab were downloaded from Drug Bank (https://www.drugbank.ca/drugs/DB00112) and site screening was performed to identify anti-VEGF TMEAbody with good blocking efficiency. The mutation position in a heavy chain of the anti-VEGF antibody (Bevacizumab) was selected from the group consisting of: Tyr32, Asn35, Tyr54, Tyr60, Lys65, Arg66, Tyr102, Tyr103 and Tyr109; and the mutation position in a light chain was selected from the group consisting of: Ser24, Ser26, Asp28, Tyr32, Tyr49, Thr51, Tyr91, Ser92, and Thr93. His tagged VEGF protein was used for ELISA. The selected sites with good blocking efficiency and good recovery were summarized in Table 76.

TABLE 76
Selected anti-VEGF TMEAbody candidates based
on blocking and recovery efficiency

| | | | | | Blocking | | Restored |
|---|---|---|---|---|---|---|---|
| ID of selected | Heavy or | Mutation | | Conjugation | efficiency | | activity after |
| Mutation | light chain | site | R4 | efficiency | of S47 | | R4 conjugation |
| Beva-se001 | HC | Lys65 | R4-7 | 95% | 9 | fold | Restored |
| Beva-se002 | LC | Ser26 | R4-1 | 91% | 16 | fold | Restored |
| Beva-se003 | LC | Tyr49 | R4-7 | 95% | 26 | fold | Restored |
| Beva -se004 | LC | Thr51 | R4-1 | 87% | 13 | fold | Restored |

### Example 33: Generation and Characterization of Anti-CD20 TMEAbody (Rituximab)

The heavy chain and light chain sequence of Bevacizumab was downloaded from Drug Bank (www.drugbank.ca/drugs/DB00073) and site screening was performed to identify anti-CD20 TMEAbody with good blocking efficiency. The mutation position in a heavy chain of the anti-CD20 antibody (rituximab) was selected from the group consisting of: Tyr32, Asn33, Tyr52, Asn55, Lys63, Lys65, Tyr101, Tyr102 and Tyr107; and the mutation position in a light chain was selected from the group consisting of Ser26, Ser28, Tyr31, Tyr48, Thr50, Asn52, Thr91 and Thr96. His tagged CD20 protein was used for ELISA. The selected sites with good blocking efficiency and good recovery were summarized in Table 77.

TABLE 77
Selected anti-VEGF TMEAbody candidates based
on blocking and recovery efficiency

| | | | | | Blocking | | Restored |
|---|---|---|---|---|---|---|---|
| ID of selected | Heavy or | Mutation | | Conjugation | efficiency | | activity after |
| Mutation | light chain | site | R4 | efficiency | of S47 | | R4 conjugation |
| Ritu-se001 | HC | Tyr32 | R4-7 | 92% | 16 | fold | Restored |
| Ritu-se002 | HC | Lys63 | R4-7 | 98% | 8 | fold | Restored |
| Ritu-se003 | LC | Tyr31 | R4-7 | 91% | 13 | fold | Restored |
| Ritu-se004 | LC | Tyr48 | R4-7 | 92% | 15 | fold | Restored |
| Ritu-se005 | LC | Thr50 | R4-1 | 89% | 21 | fold | Restored |

### Example 34: Blocking and Cleaving Screening of Different R4 and R5 for Drug Candidates

Blocking and cleaving effect of conjugates in indicated conjugation were evaluated. The conjugates were each dissolved and diluted for ten times to a concentration of 0.1 mM/ml. At 37 °C, conjugates were each added into 100 μg MDA-MB231 human tumor tissue homogenates (pH7) in a concentration of 1 mg/ml for 8 hr. The conjugated and released biomolecule was detected binding by ELISA, thereby comparing the activation efficiency of the linker. Results were shown in Table 78.

TABLE 78
Blocking and cleaving effect of S48 to different biomolecules

| | | Binding decrease | Recovery binding |
|---|---|---|---|
| R1-R2-R3-R4 | R5 | by conjugation | after activation (%) |
| S48 | SEQ ID: 13 | 34-fold | 96 |
| S48 | SEQ ID: 14 | 25-fold | 95 |
| S48 | SEQ ID: 15 | 18-fold | 105 |
| S48 | SEQ ID: 16 | 42-fold | 96 |
| S48 | SEQ ID: 17 | 67-fold | 95 |
| S48 | SEQ ID: 18 | 32-fold | 96 |
| S48 | SEQ ID: 19 | 67-fold | 85 |
| S48 | SEQ ID: 26 | 24-fold | 94 |
| S48 | SEQ ID: 27 | 54-fold | 94 |
| S48 | SEQ ID: 28 | 14-fold | 107 |
| S48 | SEQ ID: 29 | 18-fold | 106 |
| S48 | SEQ ID: 30 | 9-fold | 91 |
| S48 | SEQ ID: 31 | 13-fold | 89 |
| S48 | SEQ ID: 32 | 14-fold | 93 |
| S48 | SEQ ID: 44 | 75-fold | 99 |
| S48 | SEQ ID: 54 | 24-fold | 96 |
| S48 | SEQ ID: 60 | 16-fold | 96 |
| S48 | SEQ ID: 61 | 19-fold | 107 |
| S48 | SEQ ID: 62 | 74-fold | 96 |
| S48 | SEQ ID: 63 | 34-fold | 93 |

Blocking and cleaving effect of conjugates in indicated conjugation in Table 79 were evaluated. The conjugates were each dissolved and diluted ten times to a concentration of 0.1 mM/ml. At 37 °C, conjugates were each added into MMP2 (pH6) in a concentration of 1 mg/ml for 16 hr. The conjugated and released biomolecule was detected binding by ELISA, thereby comparing the activation efficiency of the linker. Results were shown in Table 79.

TABLE 79
Blocking and cleaving effect of conjugations
of S65 to different biomolecules

| | | Binding decrease | Recovery binding after |
|---|---|---|---|
| R1-R2-R3-R4 | R5 | by conjugation | activation (%) |
| S65 | SEQ ID: 13 | 19-fold | 96 |
| S65 | SEQ ID: 14 | 21-fold | 99 |
| S65 | SEQ ID: 15 | 58-fold | 95 |
| S65 | SEQ ID: 16 | 124-fold | 97 |
| S65 | SEQ ID: 17 | 164-fold | 99 |
| S65 | SEQ ID: 18 | 26-fold | 95 |
| S65 | SEQ ID: 19 | 35-fold | 89 |
| S65 | SEQ ID: 26 | 43-fold | 94 |
| S65 | SEQ ID: 27 | 17-fold | 95 |
| S65 | SEQ ID: 28 | 12-fold | 99 |
| S65 | SEQ ID: 29 | 25-fold | 105 |
| S65 | SEQ ID: 30 | 48-fold | 101 |
| S65 | SEQ ID: 31 | 75-fold | 97 |
| S65 | SEQ ID: 32 | 34-fold | 93 |
| S65 | SEQ ID: 44 | 19-fold | 96 |
| S65 | SEQ ID: 54 | 25-fold | 94 |
| S65 | SEQ ID: 60 | 75-fold | 97 |
| S65 | SEQ ID: 61 | 34-fold | 93 |
| S65 | SEQ ID: 62 | 19-fold | 96 |
| S65 | SEQ ID: 63 | 25-fold | 94 |

Blocking and Cleaving effect of conjugates in indicated conjugation were evaluated. The conjugates were each dissolved and diluted ten times to a concentration of 0.1 mM/ml. At 37 °C, conjugates were each added into 100 μg MDA-MB231 acidized human tumor tissue homogenates (pH6.5). The conjugated and released biomolecule was detected binding by ELISA, thereby comparing the activation efficiency of the linker. Results were shown in Table 80.

TABLE 80
Blocking and cleaving effect of conjugations
of S27 to different biomolecules

| | | Binding decrease | Recovery binding after |
|---|---|---|---|
| R1-R2-R3-R4 | R5 | by conjugation | activation (%) |
| S27 | SEQ ID: 13 | 43-fold | 91 |
| S27 | SEQ ID: 14 | 11-fold | 95 |
| S27 | SEQ ID: 15 | 8-fold | 98 |
| S27 | SEQ ID: 16 | 14-fold | 95 |
| S27 | SEQ ID: 17 | 19-fold | 98 |
| S27 | SEQ ID: 18 | 33-fold | 95 |
| S27 | SEQ ID: 19 | 45-fold | 96 |
| S27 | SEQ ID: 26 | 28-fold | 99 |
| S27 | SEQ ID: 27 | 37-fold | 95 |
| S27 | SEQ ID: 28 | 36-fold | 97 |
| S27 | SEQ ID: 29 | 106-fold | 106 |
| S27 | SEQ ID: 30 | 135-fold | 102 |
| S27 | SEQ ID: 31 | 75-fold | 107 |
| S27 | SEQ ID: 32 | 58-fold | 97 |
| S27 | SEQ ID: 44 | 74-fold | 99 |
| S27 | SEQ ID: 54 | 35-fold | 97 |

According to the results, these drug candidates exhibited blocking effect and activation effect in the indicated activation conditions.

### Example 35: Study on Efficacy of Indicated S27, S39, S40, S47, S48 and S65 Conjugated with Mouse CTLA-4 Antibody in CT26 Tumor Immune Model

Test purpose: to investigate the anti-tumor efficacy of S27, S39, S40, S47, S48, S65 conjugated with mouse CTLA-4 antibody for immune treatment.

Test drug: S27, S39, S40, S47, S48, S65 conjugated with mouse CTLA-4 antibody (9D9), all used in 20 mg/kg (equimolar of CTLA-4 antibody).

### Production of Tumor Model:

1) CT26 tumor cells were purposed from ATCC. Cells were cultivated in DMEM culture solution containing 10% fetal bovine serum at 37 °C and 5% CO₂. The cells were passaged every three days and cells within the 15th passage were used.
2) Tumor immunization. 5×10⁵ CT26 cancer cells (purchased from ATCC) which were killed by irradiation were intraperitoneally injected to mice. The mice were injected 3 times, once every two weeks. After immunization, mice were injected with tumor cells and the drugs were administered weekly for 4 weeks.
3) Production of tumor. At day 32, 10⁶ live lung tumor cells were subcutaneously injected to the back of the C57 mice immunized by tumor. Treatment began when the tumor grew to 0.3-0.4 cm.
4) Analysis on tumor CD8+ T cells. The tumor tissue was homogenated and individual cells in the tumor were filtered, separated and washed by buffer twice, then cultivated with the leucocyte common antigen CD45-PE and CD8-FITC marked antibodies for 1 hour at ambient temperature. The cells were washed by phosphate buffer containing 1% fetal bovine serum twice and then analyzed for the ratio of the T lymphocyte antigen (CD8) positive cells in the leucocyte common antigen (CD45) positive cells by flow cytometry. Increasement of the ratio indicates increased T lymphocyte cells and thus the animal immunity against the tumor was improved.
5) Grouping and test results are shown in Table 81.

TABLE 81
Effect on inhibition of tumor and immune activation
of S27, S39, S40, S47, S48 and S65 conjugated
with mouse CTLA-4 antibody and control

| | Number | Size of | Cure number | CD8: |
|---|---|---|---|---|
| | of | tumor (mm³) | of mice | CD45 |
| Group | animals | Day 28 | Day 28 | (%) |
| PBS | 8 | 1887.56 ± 323.4 | 0 | 5.2 |
| Mouse CTLA-4 | 8 | 74.46 ± 467.34 | 2 | 13.1 |
| antibody (WT) | | | (Dead 2) | |
| S27-Mouse CTLA-4 | 8 | 37.60 ± 358.57 | 6 | 28.4 |
| Antibody (9D9) | | | | |
| S39-Mouse CTLA-4 | 8 | 31.57 ± 124.45 | 5 | 29.7 |
| Antibody (9D9) | | | | |
| S40-Mouse CTLA-4 | 8 | 57.63 ± 157.32 | 6 | 26.3 |
| Antibody (9D9) | | | | |
| S47-Mouse CTLA-4 | 8 | 53 ± 216.47 | 7 | 38.4 |
| Antibody (9D9) | | | | |
| S48-Mouse CTLA-4 | 8 | 75.78 ± 32.74 | 4 | 23.6 |
| Antibody (9D9) | | | | |
| S65-Mouse CTLA-4 | 8 | 74.28 ± 38.45 | 6 | 26.7 |
| Antibody (9D9) | | | | |

6) Results and discussion. Treatment effects of S27, S39, S40, S47, S48 and S65 conjugated with mouse CTLA-4 antibody were greatly improved as compared to the control group and the WT CTLA-4 antibody treatment groups. WT CTLA-4 antibody Causing 1 death in WT CTLA-4 antibody treatment may be caused by toxicity in high dose treatment. Treatment effects of S27, S39, S40, S47, S48 and S65 conjugated with mouse CTLA-4 antibody show an excellent effect and promote CD8/CD45 T cell ratio in tumor tissue.

### Example 36: Study on Efficacy of Adalimumab (SEQ ID NO:29) Conjugated with S27, S47, S48 or S65 in RA Mouse Model

The TgTC mice were generated using a human TNF/β-globin (TNFglobin) recombinant gene construct, which contained a 2.8 kb fragment with the entire coding region and promoter of the hTNFα gene, fused to a 0.77 kb fragment with the 3′ untranslated region (UTR) and polyadenylation site of human β-globin replacing that of the hTNFα gene. The fragment was then microinjected into pronuclei of FVB/J inbred strain fertilized eggs. Finally, the injected fertilized eggs were implanted into the oviduct of 8-week-old female pseudo-pregnant ICR mice. Transgenic lineages were established by back-crossing the transgenic founder individuals to the FVB/J inbred strain. The genotyping was performed by PCR to screen for transgenic animals as well as routine tail genotyping. The transgene specific PCR primers were:

(forward primer, SEQ ID NO: 87)
5′-GAACTCCCTCGATGTTAACCA-3′;
and
(reverse primer, SEQ ID NO: 88)
5′-TTCAATCCCCAAATCCTAGCC-3′.

The PCR reactions were performed as follows: 94 °C for 4 min; 35 cycles at 95 °C for 30 s, 57 °C for 40 s, and 72 °C for 40 s; 72 °C for 10 min.

Different anti-hTNFα antibody (Adalimumab 2 mg/kg) and conjugated antibody (2 mg/kg equimolar of Adalimumab) dissolved in saline were intraperitoneally administered (2 mg/kg) to TgTC mice weekly from three to ten weeks, with saline-treated TgTC mice serving as control. Clinical assessment Weekly body weight and arthritis scores in all four limbs were recorded after weaning. Clinical severity of arthritis for each paw (fingers, tarsus, and ankle) was quantified by attributing a score ranging from 0 to 3: 0, normal; 1, slight redness and/or swelling; 2, pronounced edematous swelling; 3, joint deformity and rigidity. The arthritis score per mouse was an average of the four limbs. Grouping and test results are shown in Table 82.

TABLE 82
Effect on inhibition of arthritis by S27, S47,
S48 and S65 conjugated with Adalimumab antibody

| | Number of | The arthritis score | The arthritis score |
|---|---|---|---|
| Group | animals | Day 5 | Day 10 |
| PBS | 4 | 3 | 3 |
| Adalimumab | 4 | 3 | 2 |
| S27-Adalimumab | 4 | 1 | 1 |
| S47-Adalimumab | 4 | 2 | 1 |
| S48-Adalimumab | 4 | 1 | 1 |
| S65-Adalimumab | 4 | 1 | 1 |

Results showed that Adalimumab conjugated with S27, S47, S48 and S65 greatly reduced the arthritis scores.

### Example 37: Generation and Characterization of Anti-Her2/Anti-CD3 Bispecific TMEAbody

The heavy chain and light chain sequences of Trastuzumab was downloaded from Drug Bank (www.drugbank.ca/drugs/DB00072) and sites screening was performed to identify scFv form of anti-Her2 with good blocking efficiency. The mutation position in a light chain is selected from the group consisting of: Asp1, Gln3, Gln27, Asp28, Asn30, Tyr49, Tyr55, Arg66, Asp70, and Tyr92. The mutation position in a light chain is selected from the group consisting of Arg19, Lys30, Asp31, Tyr33, Arg50, Tyr62, Asn55, Tyr57, Arg59, Tyr60, Asp62, Lys65, Asp102, and Tyr105. The scFv forms of above selected mutants were expressed in HEK293 with C Terminal 6His tag, purified with Ni-NTA column, and conjugated with corresponding chemical linkers. Binding ELISA was carried out with His-tagged human Her2 protein as antigen and anti-human kappa chain as secondary antibody. The blocking efficiency was summarized in Table 84.

TABLE 83
Selected scFv form of anti-Her2 candidates based on blocking and recovery efficiency

| ID of selected | | | | | Blocking | | Activity |
|---|---|---|---|---|---|---|---|
| Mutation for | Single Heavy/ | Mutation | | Conjugation | efficiency | | after R4 |
| scFv form | Light chain | site | R4 | efficiency | of S47 | | conjugation |
| Trast-se001scFv | Heavy chain | Tyr33 | R4-7 | 95% | 15 | fold | Restored |
| Trast-se002scFv | Heavy chain | Lys65 | R4-7 | 93% | 6 | fold | Restored |
| Trast-se003scFv | Heavy chain | Tyr105 | R4-7 | 92% | 32 | fold | Restored |
| Trast -se004scFv | Light chain | Gln30 | R4-11 | 83% | 15 | fold | Restored |
| Trast -se005scFv | Light chain | Tyr49 | R4-7 | 93% | 43 | fold | Restored |
| Trast -se006scFv | Light chain | Asp70 | R4-11 | 91% | 13 | fold | Restored |

We fused anti-Her2 scFv with selected Cysteine mutation (Tyr49 in light chain) to anti-human CD3 scFv containing C terminal 6His tag to form bispecific antibody targeting to tumor and T cells. These Her2/CD3 bispecific antibodies were produced in HEK293 cells and purified with Ni-NTA column. These Her2/CD3 bispecific antibodies with mutant was further conjugated with S47 and a 38 fold decreased binding activity to human Her2 protein was obtained. After digestion with legumain, both binding activities were restored.

The single chain of Her2/CD3 TMEAbody were produced by conjugating S27, S47 or S48 to a fusion protein anti-Her2 scFV with anti-CD3 or its scFv, as shown in Table 84.

TABLE 84
Selected scFv form of anti-Her2 candidates
based on blocking and recovery efficiency

| Bispecific | | | | |
|---|---|---|---|---|
| TMEAbody | anti-Her2 | anti-CD3 | SEQ ID NO: | R1-R2-R3-R4 |
| Her2/CD3 | anti-Her2 | anti-CD3 | 70 | S27 |
| TMEAbody 1 | scFv | scFv | | |
| Her2/CD3 | anti-Her2 | anti-CD3 | 70 | S47 |
| TMEAbody2 | scFv | scFv | | |
| Her2/CD3 | anti-Her2 | anti-CD3 | 70 | S48 |
| TMEAbody3 | scFv | scFv | | |
| Her2/CD3 | anti-Her2 | anti-CD3 | 82 | S27 |
| TMEAbody4 | scFv | scFv | | |
| Her2/CD3 | anti-Her2 | anti-CD3 | 82 | S47 |
| TMEAbody5 | scFv | scFv | | |
| Her2/CD3 | anti-Her2 | anti-CD3 | 82 | S48 |
| TMEAbody6 | scFv | scFv | | |
| Her2/CD3 | anti-Her2 | anti-CD3 | Anti-Her2: | S27 |
| TMEAbody7 | | | 71 and 72 | |
| | | | Anti-CD3: 75 | |
| Her2/CD3 | anti-Her2 | anti-CD3 | Anti-Her2: | S47 |
| TMEAbody8 | | | 71 and 72 | |
| | | | Anti-CD3:75 | |
| Her2/CD3 | anti-Her2 | anti-CD3 | Anti-Her2: | S48 |
| TMEAbody9 | | | 71 and 72 | |
| | | | Anti-CD3: 75 | |

We fused PD-1 antibody or PD-L1 antibody with mutant IL-2 (IL2-S87C) to form targeting tumor associated antigen PD-L1/IL-2 TMEAkines or PD-1/IL-2 TMEAkines.

These TMEAkines with IL2-T41C mutant was further conjugated with S47. A >435 fold decreased binding activity to human IL-2Rβ was obtained. After digestion with legumain, both binding activity to IL-2Rβ were restored. The fused PD-Li or PD-1 antibody with mutant IL-2 (mutant at binding IL-2Rα S87C) were further conjugated with S47 to get legumain activation fusion TMEAkine as shown in Table 85.

TABLE 85
Fusion protein candidates based on blocking and recovery efficiency

| Fusion | | R1-R2- |
|---|---|---|
| TMEAkine | Fusion protein sequence | R3-R4 |
| 1 | N-teminal-IL2(S87C)-GSGS-PD-1(SEQ ID NO: 15) | S47 |
| 2 | N-teminal-IL2(S87C)-GSGS-PD-1 (SEQ ID NO: 17) | S47 |
| 3 | N-teminal-IL2(S87C)-GSGS-PD-L1(SEQ ID NO: 27) | S47 |
| 4 | N-terminal-PD-1 (SEQ ID NO: 15)-GSGS-IL2(S87C) | S47 |
| 5 | N-terminal-PD-1 (SEQ ID NO: 17)-GSGS-IL2(S87C) | S47 |
| 6 | N-terminal-PD-L1 (SEQ ID NO: 27)-GSGS-IL2(S87C) | S47 |
| 7 | N-terminal-EGFR (SEQ ID NO: 78&79)-GSGS--IL2(S87C) | S47 |
| 8 | N-terminal-VEGFR (SEQ ID NO: 80&81)-GSGS-IL2(S87C) | S47 |
| 9 | N-terminal-Her2 (SEQ ID NO: 82&83)-GSGS-IL2(S87C) | S47 |
| 10 | PD-1/IL-2 fusion protein (SEQ ID NO: 73) | S47 |
| 11 | PD-L1/IL-2 fusion protein (SEQ ID NO: 74) | S47 |

### In Vivo Characterization of Toxicity in Human PBMC-Transferred Mouse Model

Test purpose: to investigate the acute toxicity of the fusion TMEAbody via intravenous injection.

Animal: the first class SCID mouse, weighing 19-21 g and all mice being female.

Method and results: SCID mouse were randomly divided into 21 groups according to their body weights, with 10 mice in each group. As shown in Table 86, the mice were intravenously injected with D1, D7 and D14 for just one time in a dose of 30 mg/kg (equimolar of antibody). Control tests were performed by injecting 30_mg/kg human IgG. Animals were observed for 21 continuous days for presence or absence of the following behaviors on each day: pilo-erection, hair tousle and lackluster, lethargy, stoop and irritable reaction, and body weight and death were recorded as shown in Table 86.

TABLE 86
Comparison of mortality rates of test mice
receiving different fusion TMEAbody

| | | | Number | |
|---|---|---|---|---|
| | | | of dead | |
| | | Number of | animal | Mortality |
| Group | injections | animals | (day 21) | rate (%) |
| 1 | human IgG | 10 | 0 | 0 |
| 2 | Her2/CD3 scFv | 10 | 4 | 40 |
| 3 | Her2/CD3 antibody | 10 | 3 | 30 |
| 4 | Her2/CD3 TMEAbody 1 | 10 | 0 | 0 |
| 5 | Her2/CD3 TMEAbody2 | 10 | 0 | 0 |
| 6 | Her2/CD3 TMEAbody3 | 10 | 0 | 0 |
| 7 | Her2/CD3 TMEAbody4 | 10 | 0 | 0 |
| 8 | Her2/CD3 TMEAbody5 | 10 | 0 | 0 |
| 9 | Her2/CD3 TMEAbody6 | 10 | 0 | 0 |

Results and discussions: no pilo-erection, hair tousle and lackluster, lethargy, stoop, irritable reaction and death were observed in mice receiving 30 mg/kg of group 2, 3, 10, and 14. As shown in Table 86, The MTD of the fusion protein is less than 30 mg/kg, which can be observed toxicity and deaths.

### In Vivo Characterization of Toxicity in Human PBMC-Transferred Mouse Model

Test purpose: to investigate the acute toxicity of the fusion TMEAbody via intravenous injection.

Animal: the first class SCID mouse, weighing 19-21 g and all mice being female.

Method and results: SCID mouse were randomly divided into 21 groups according to their body weights, with 5 mice in each group. As shown in Table 88, the mice were intravenously injected with fusion protein and fusion protein conjugation with S47 at D1, D7 and D14 for just one time in a dose of 30 mg/kg_(equimolar of antibody). Control tests were performed by injecting 30 mg/kg saline. Animals were observed for 21 continuous days for presence or absence of the following behaviors on each day: pilo-erection, hair tousle and lackluster, lethargy, stoop and irritable reaction, and body weight and death were recorded as shown in Table 87.

TABLE 87
Comparison of mortality rates of test mice
receiving different fusion TMEAkine
injections

| | Death | Death number after |
|---|---|---|
| Fusion protein sequence | number | conjugating to S47 |
| saline | 0 | 0 |
| N-teminal-IL2(S87C)-GSGS-PD-1(SEQ ID NO: 15) | 5 | 0 |
| N-teminal-IL2(S87C)-GSGS-PD-1 (SEQ ID NO: 17) | 3 | 0 |
| N-teminal-IL2(S87C)-GSGS-PD-L1(SEQ ID NO: 27) | 5 | 0 |
| N-terminal-PD-1(SEQ ID NO: 15)-GSGS-IL2(S87C) | 2 | 0 |
| N-terminal-PD-1(SEQ ID NO: 17)-GSGS-IL2(S87C) | 1 | 0 |
| N-terminal-PD-L1(SEQ ID NO: 27)-GSGS-IL2(S87C) | 4 | 0 |
| N-terminal-EGFR (SEQ ID NO: 77&79)-GSGS--IL2(S87C) | 3 | 0 |
| N-terminal-VEGFR (SEQ ID NO: 80&81)-GSGS-IL2(S87C) | 4 | 0 |
| N-terminal-Her2 (SEQ ID NO: 82&83)-GSGS-IL2(S87C) | 5 | 1 |
| PD-1/IL-2 fusion protein (SEQ ID NO: 73) | 4 | 0 |
| PD-L1/IL-2 fusion protein(SEQ ID NO: 74) | 5 | 1 |

Results and discussions: no pilo-erection, hair tousle and lackluster, lethargy, stoop, irritable reaction and death were observed in mice receiving 30 mg/kg of all group of fusion TMEAkine. But the toxicity was reduced after fusion with S47.

### In Vivo Characterization of Single Chain of CD3-Her2 TMEAbody in Mouse Tumor Model

To further characterize the in vivo efficacy of single chain of CD3-Her2 TMEAbody in treating tumor in animal model, single chain of CD3-Her2 TMEAbody, as well as single chain CD3-Her2 antibody were administrated into Tumor xenografts. To initiate tumor xenografts, 3×10⁶ KPL-4 cells were implanted orthotopically into the right penultimate inguinal mammary fat pad of female severe combined immunodeficient (SCID) beige mice. Tumors were allowed to growth (20 d for KPL4) after implantation before initiation of treatment. Mice with KPL-4 tumors (100 mm³) were treated with indicated drug (10 mg/kg weekly for 5 weeks) for the duration of the study. Tumor volumes and body weights were measured twice weekly. The tumor volume inhibition rate was summarized in Table 88. Results implied that single chain of CD3-Her2 TMEAbody could be activated in the tumor microenvironment and enhance the efficacy of single chain of CD3-Her2 antibody.

TABLE 88
Tumor growth inhibition rate at day 20 after administration

| Group | Dose | Cure rate (%) | Dead |
|---|---|---|---|
| human IgG | 10 mg/kg | 0 | 0 |
| Her2/CD3 scFv | 10 mg/kg | 16.7 | 0 |
| Her2/CD3 antibody | 10 mg/kg | 33.3 | 2 |
| Her2/CD3 TMEAbody 1 | 10 mg/kg | 66.7% | 0 |
| Her2/CD3 TMEAbody2 | 10 mg/kg | 83.3% | 0 |
| Her2/CD3 TMEAbody3 | 10 mg/kg | 100% | 0 |
| Her2/CD3 TMEAbody4 | 10 mg/kg | 100% | 0 |
| Her2/CD3 TMEAbody5 | 10 mg/kg | 100% | 0 |
| CD3-Her2 TMEAbody6 | 10 mg/kg | 100% | 0 |
| PD-1/IL-2 fusion | 10 mg/kg | 100% | 2 |
| Fusion TMEAkinel | 10 mg/kg | 83.3% | 0 |
| Fusion TMEAkine 2 | 10 mg/kg | 100% | 0 |
| TMEAkine4 | 10 mg/kg | 100% | 0 |
| Fusion TMEAkine5 | 10 mg/kg | 100% | 0 |
| Fusion TMEAkine10 | 10 mg/kg | 100% | 0 |
| PD-L1/IL-2 fusion | 10 mg/kg | 100% | 2 |
| FusionTMEAkine3 | 10 mg/kg | 83.3% | 0 |
| Fusion TMEAkine6 | 10 mg/kg | 100% | 0 |
| Fusion TMEAkine11 | 10 mg/kg | 83.3% | 0 |

As shown in Table 88, inhibition on tumor growth and cure rate by Her2/CD3 TMEAbody were greatly improved as compared with the groups treating by Her2/CD3 scFv or Her2/CD3 antibody by using the same molar concentration. Inhibition on tumor growth and cure rate by PD-L1/IL-2 TMEAkine or antibody show efficacy and cure the mice. But in the PD-L1/IL-2 fusion antibody group, the toxicity caused some mice death.

### Example 38: New IL-2 Variant Expression

The mutated amino acid sequences (SEQ ID No. 106 and SEQ ID No. 44) were optimized for Pichia pastoris, and the target gene sequences for synthesis and production were inserted into the pPICZα-A expression vector. The ligation products were transformed into DH5α competent cells and plated on Low Salt LB plates containing bleomycin to obtain single monoclonal transformant colonies. Eight monoclonal colonies were selected for colony PCR identification. Positive bacterial cultures were amplified, and plasmids were extracted. Recombinant plasmids were introduced into GS115 strain cells via electroporation. Transfected cells were then plated on YPD agar plates containing 1M sorbitol and appropriate concentrations of the antibiotic Zeocin (100 μg/ml, 200 μg/ml, 400 μg/ml, 1000 μg/ml, 2000 μg/ml). The plates were incubated at 30 °C for 72-96 hours. Based on the colony size on YPD agar plates containing 2000 μg/ml Zeocin, eight larger colonies were selected. Each selected colony was further screened on YPD agar plates containing 2000 μg/ml Zeocin to obtain single clone. Single clones were screened based on the presence of the target gene, copy number of the target gene, relative expression level of the AOX1 gene, and other genetic elements highly correlated with target protein expression.

A sterilized 50 ml seed medium was placed in a biosafety cabinet, and the bacterial solution was inoculated into YPD medium at a 0.2% inoculation rate using a pipette. It was then cultured in a constant-temperature shaker at 30 °C and 250 rpm. After 20-24 hours, samples were taken for OD600 measurement, which should be between 2 and 20. In the biosafety cabinet, the primary seed culture was inoculated at a 0.2% rate into 600 ml of YPD seed medium. After sealing and mixing, it was placed in a constant-temperature shaker at 30 °C and 250 rpm for 20-24 hours. Samples were then taken in the biosafety cabinet for OD600 measurement, which should be between 2 and 20.

A prepared 12 L fermentation medium was added to the fermenter. Before sterilization, the pH electrode was calibrated using pH 4.01 and 6.86 standard buffers. The sterilization program was set at 121 °C for 60 minutes. After sterilization, the temperature was set to 30 °C. Filter-sterilized PTM1 solution was added to the fermenter under flame protection. Under flame protection, the seed culture was added to the fermenter at a 5% (600 ml) ratio to initiate fermentation. When the glycerol in the basal medium was depleted, indicated by a rise in dissolved oxygen (DO), a glycerol feed was started at a rate of approximately 18.15 ml/h/L working volume. This phase ended when the wet cell weight reached 330 g/L.

After the glycerol feeding phase, when the DO rose rapidly, a starvation period of about 30 minutes was implemented, followed by the initiation of methanol feeding. For the first 2 hours of induction, the feed rate was approximately 2 ml/h/L. From hours 2-12 of induction, the rate was about 3.6 ml/h/L. From hours 12-16, it was adjusted to 7.3 ml/h/L. After 16 hours of induction, the rate was changed to 8-9 ml/h/L. When the wet weight reached 360-400 g/L, induction was continued for 72 hours before harvesting. The fermentation supernatant was purified via ultrafiltration, cation exchange chromatography, and gel filtration to obtain high-purity protein samples.

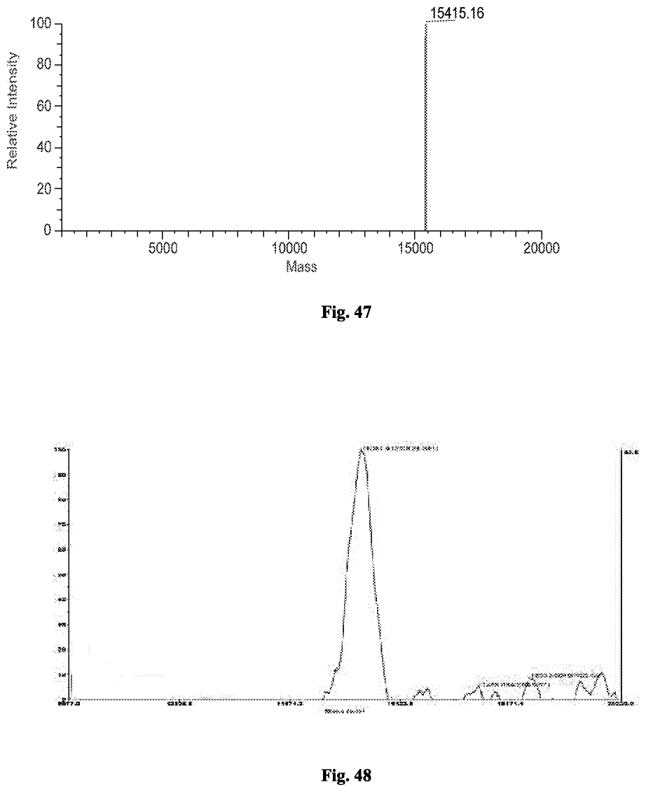

The intact molecular weight of the protein samples was analyzed using a Thermo Fisher Q Exactive LC-MS system. Samples were concentrated with a 3 kDa ultrafiltration tube before injection. LC conditions: Column: C8 2.1×100 mm, 5 μm; Mobile phase A: 0.1% formic acid in water; Mobile phase B: 0.1% formic acid in acetonitrile; Flow rate: 0.4 mL/min; Gradient run: 20 minutes. MS conditions: Positive ion mode, full scan range 500-3000 m/z. Deconvolution was performed using Biopharma Finder 4.1 software to obtain the intact molecular weight of the samples, as shown in FIGS. 47 and 48. The molecular weight detected for SEQ No. 44 was heterogeneous, indicating glycosylation heterogeneity in the protein expressed from SEQ No. 44. In contrast, the molecular weight detected for SEQ No. 106 matched the theoretical molecular weight and was uniform, indicating the absence of glycosylation sites in this protein.

### Example 39: New IL-2 Variant Biological Activity

Biological activity was assessed using an IL-2-dependent cell line (CTLL2) co-cultured with the protein at various concentrations. The number of viable cells post-co-culture was determined using the CellTiter-Glo® Luminescent Cell Viability Assay. This assay measures ATP release upon cell lysis, where ATP quantity is proportional to the number of viable cells. The assay relies on luciferase in the reagent catalyzing the production of light from luciferin in the presence of ATP, Mg²⁺, and oxygen. The resulting luminescence signal was measured using a microplate reader for analysis.

Based on cell counting results, a specific number of cells were first washed once with serum-free IMDM medium and then once with PBS. The cells were resuspended in basal medium, and their density was adjusted to 4×10⁵ cells/ml using the cell count data. The cell suspension was seeded into a 96-well cell culture plate at 100 μl per well. Gradient-diluted drug samples were added at 50 μl per well, followed by incubation in a 5% CO₂, 37 °C cell culture incubator for 20-24 hours.

After incubation, the cell plates were taken out and allowed to equilibrate at room temperature for 15 minutes. Following equilibration, detection reagent was added to each well at 80 μl per well. The plates were then placed in a microplate shaker at 500 rpm for 5 minutes, after which data acquisition was performed using a microplate reader. Data analysis was conducted using SoftMax software, applying a four-parameter logistic fit to generate dose-response curves. The four-parameter fitting equation used was:

Y=(A−D)/[1+(X/C)\hat{ }B]+D

Where: A: Estimated lower asymptote of the curve
B: Slope of the curve
C: EC₅₀ (ng/ml)
D: Estimated upper asymptote of the curve

Utilizing the four-parameter equation, the EC₅₀ values of the samples were calculated and presented below in Table 89.

TABLE 89
Biological Activity Results

| Sample Category | EC₅₀ (ng/mL) |
|---|---|
| SEQ ID No. 106 | 52.32 |
| SEQ ID No. 44 | 51.08 |

Proteins expressed using Pichia pastoris from SEQ No. 106 and SEQ No. 44 sequences exhibited no significant difference in biological activity.

1. A conjugate of a biomolecule having the following structure:
R1-R2-R3-R4-S-cys-R5
wherein,
R5 represents a biomolecule with one or more cysteine residues introduced by mutation;
cys represents the cysteine residue(s) contained in R5;
S represents sulfur atom(s) of the cysteine residue(s);
R1 is a group that prevents R5 from binding to its antigen, ligand or receptor;
R2 is absent, or R2 is a cleavable linker arm capable of being activated by one or more proteolytic enzymes or a chemical bond capable of being acidically activated in a pathologic microenvironment;
R3 is absent, or R3 is a linker arm capable of automatically shedding after R2 is cleaved or a chemical bond capable of being acidically activated in a pathologic microenvironment; with the proviso that when R2 is absent, R3 is the chemical bond capable of being acidically activated in a pathologic microenvironment; and
R4 is a group covalently linked to R5 via the sulfur atom(s) of the cysteine residue(s) contained in R5 that recovers, maintains or promotes the binding capacity of R5 to its antigen, ligand or receptor after the moiety R1-R2-R3 is cleaved.

2. The conjugate of the biomolecule of claim 1, wherein R2 is a peptide capable of being activated or cleaved by one or multiple proteolytic enzyme, protease or peptidase, wherein the protease is selected from the group consisting of cysteine protease, asparagines protease, aspartate protease, glutamic acid protease, threonine protease, gelatinase, metallopro-teinase, or asparagine peptide lyase;
preferably, R2 is a peptide capable of being cleaved by at least one of enzymes selected from the group consisting of kallikrein, hKl, hKlO, hK15, chymotrypsin-like protease, trypsin-like protease, ADAMlO, ADAM12, nenterokinase, dipeptidyl peptidase, and dipeptidyl peptidase IV (DPPIV/CD26).

3. The conjugate of the biomolecule of claim 1, wherein R2 is a bond cleavable in an acidic condition of a pathologic microenvironment and is selected from the group consisting of amide, ester, carbamate, urea and hydrazone bonds;
preferably, wherein the structure of R1-R2-R3-R4 is represented by: 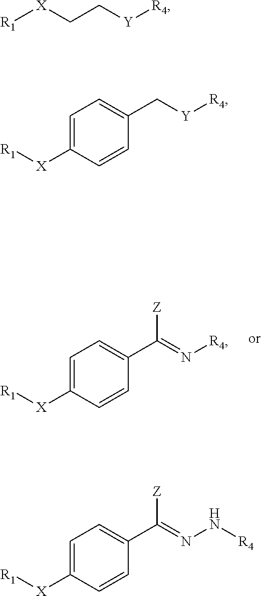
wherein X and Y are each independently NR′ or O, Z is H or C1-10 alkyl, and R′ is H or C₁₋₄alkyl; R3 links to R1 and R4 via X and Y respectively in a linkage manner of amide, ester, carbamate, urea or hydrazone bond.

4. The conjugate of the biomolecule of claim 1, wherein R2 is a bond cleavable in an acidic condition of a pathologic microenvironment and is selected from the group consisting of amide, ester, carbamate, urea and hydrazone bonds, and the 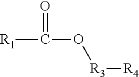
structure of R1-R2-R3-R4 is represented by:

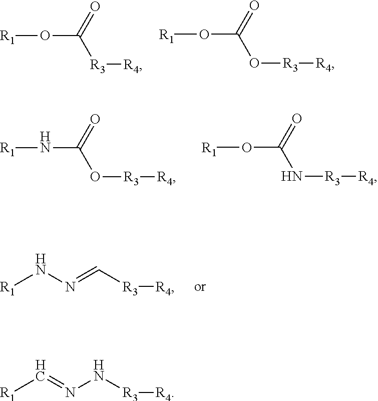

5. The conjugate of the biomolecule of claim 1, wherein R3 is selected from the group consisting of: 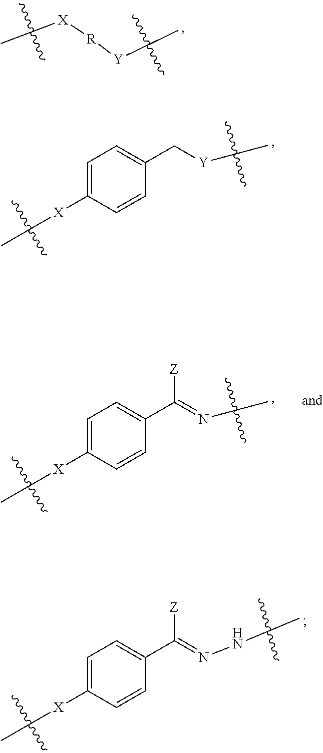
wherein X and Y are each independently NR′ or O, Z is H or C₁₋₁₀ alkyl, R is C₁₋₄ alkyl, and R′ is H or C₁₋₄alkyl; and wherein R4 links to R3 via the Y or N in the above formulae in a linkage manner of amide, ester, carbamate, urea or hydrazone bond.

6. The conjugate of the biomolecule of claim 5, wherein R3 is selected from the group consisting of —NH-phenyl-CH₂O—, —NH-phenyl-CH═N—, —NH-phenyl-C(CH₃)═N—, —O-phenyl-CH═N— and —O-phenyl-C(CH₃)═N—.

7. The conjugate of the biomolecule of claim 1, wherein R4 is represented by —R₄₋ₐ-R_{4-b}-R_{4-c}—, wherein R₄₋ₐ is selected from the group consisting of: 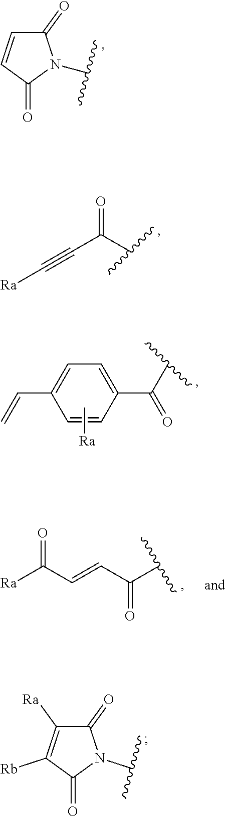
wherein Ra and Rb are each independently selected from the group consisting of H and C₁₋₆ alkyl or C₁₋₆ alkoxyl;
R_{4-b} is selected from the group consisting of: 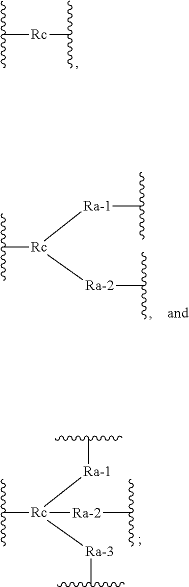
wherein in formula R4-b1, Rc is absent, or is selected from the group consisting of C₁₋₁₂ alkyl, C₁₋₁₂alkoxy-C₁₋₁₂alkyl, C₁₋₁₂alkyl-C₃₋₈cycloalkyl, (C₁₋₄alkyl-O)ₚ—C₁₋₁₂alkyl, C₁₋₁₂alkylcarbonylamino-(C₁₋₄alkyl-O)ₚ—C₁₋₁₂alkyl, phenyl-C₁₋₁₂alkyl, C₃₋₈cycloalkyl, C₁₋₁₂alkyl-C₃₋₈cycloalkyl-C₁₋₁₂alkyl, and C₁₋₁₂alkyl-phenyl-C₁₋₁₂alkyl; in formula R4-b2, Rc is a C₁₋₁₂alkylamino with Ra-1 and Ra-2 substituted at N atom of the amino group, and in formula 4-b3, Rc is a C₁₋₁₂alkyl with the last C atom at the end of the alkyl being substituted by Ra-1, Ra-2 and R2-3, wherein Ra-1, Ra-2 and Ra-3 are each independently selected from the group consisting of C₁₋₁₂alkyl, C₁₋₁₂alkyl-OH, and C₁₋₁₂alkyl-NR″R′″, wherein R″ and R′″ are each independently selected from the group consisting of H and C₁₋₁₂alkyl; wherein in formulae R4-b2 and R4-b3, R4-b links to R4-c via at least one of the Ra-1, Ra-2 and Ra-3;
R_{4-c} is selected from the group consisting of: 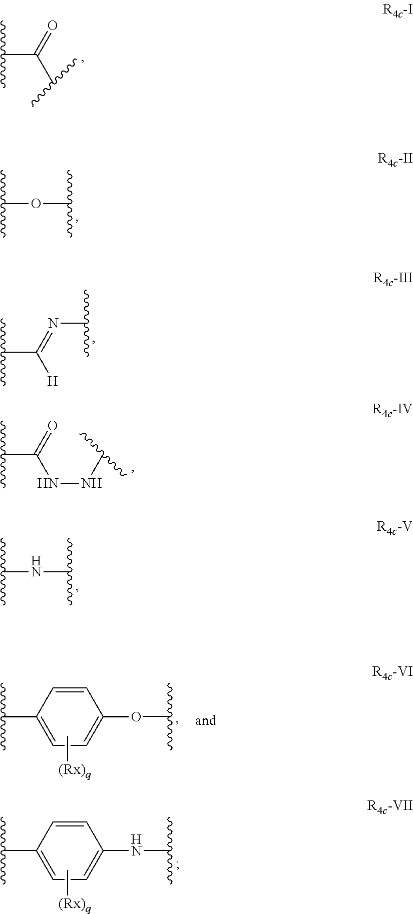
wherein Rx is selected from the group consisting of H, halo and C₁₋₄alkyl; p is an integer in a range of 1 to 10, such as an integer in a range of 1 to 5; and q is an integer in a range of 1 to 4, such as an integer in a range of 1 to 2; with the proviso that R4-c is absent when R4-a is selected from the group consisting of formulae R4-a2, R4-a3 and R4-a4;
wherein R3 links to R4 via the R4-c of R4, and the wave line shown in each formula of R4-a indicates the position at which R4-a links to R4-b.

8. The conjugate of the biomolecule of claim 1, wherein R5 is a protein with one or more of its amino acids being mutated to cysteine, and R5 links to R4 via the thiol group of the cysteine;
R5 is an antibody with one or more of its amino acids being mutated to cysteine;
wherein R5 represents a cytokine with one or more of its amino acids in its protein sequence mutated to cysteine.

9. The conjugate of the biomolecule of claim 1, wherein R5 is an antibody and the mutations in a non-complementarity determining region of a variable region of the antibody molecule; preferably, the conjugate of biomolecule eventually obtained molecular weight increasing via conjugation more than 200.

10. The conjugate of the biomolecule of claim 8, wherein R5 represents an antibody with one or more mutations in a complementarity determining region of a variable region of the antibody molecule, preferably, R5 represents an antibody with one or more of amino acid residues selected from the group consisting of G, A, S, T, L, I, F, E, K, D and Y in the complementarity determining region mutated to cysteine.

11. The conjugate of the biomolecule of claim 8, wherein R5 represents an antibody with one or more mutations in a non-complementarity determining region of a variable region of the antibody, preferably, R5 represents an antibody with one or more of amino acid residues selected from the group consisting of G, A, S, T, L, I, F, E, K, D and Y in the non-complementarity determining region mutated to cysteine.

12. The conjugate of the biomolecule of claim 8, wherein the biomolecule is a cytokine with one or more of amino acid residues selected from the group consisting of G, A, S, T, L, I, F, E, K, D and Y mutated to cysteine.

13. The conjugate of the biomolecule of claim 8, wherein R5 represents an antibody with one or more mutations in a non-complementarity determining region of a variable region of the antibody, preferably, with one or more of Gln3, Ser7, Ser26, Glu46, Thr68, Asp72 in non-complementarity determining region of VH and Thr5, Tyr49, Arg61, Ser63, Ser65, Ser67, Thr72, Thr74, Ser76, Asp82 in non-complementarity determining region of VL mutated to cysteine.

14. The conjugate of the biomolecule of claim 8, wherein before mutation,
R5 corresponds to a biomolecule selected from the group consisting of IL-2, IL-7, IL-10, IL-11, IL-12, IL-15, IL-21, IFN-α, IFN-β, IFN-7, G-CSF, GM-CSF, TNF-α, TRAP and TRAIL;
R5 corresponds to an antibody or functional fragment thereof selected from the group consisting of anti-Her2 antibody, anti-EGFR antibody, anti-VEGFR antibody, anti-CD20 antibody, anti-CD33 antibody, anti-PD-L1 antibody, anti-PD-1 antibody, anti-CTLA-4 antibody, anti-TNFα antibody, anti-CD28 antibody, anti-4-1BB antibody, anti-OX40 antibody, anti-GITR antibody, anti-CD27 antibody, antib-CD40 antibody, or anti-ICOS antibody, anti-CD25 antibody, anti-CD30 antibody, anti-CD3 antibody, anti-CD22 antibody, anti-CCR4 antibody, anti-CD38 antibody, anti-CD52 antibody, anti-Complement C₅ antibody, anti-F protein of RSV, anti-GD2 antibody, anti-GITR antibody, anti-Glycoprotein receptor lib/Illa antibody, anti-ICOS antibody, anti-IL2R antibody, anti-LAG3 antibody, anti-Integrinα4 antibody, anti-lgE antibody, anti-PDGFRα antibody, anti-RANKL antibody, anti-SLAMF7 antibody, anti-LTIGIT antibody, anti-TIM-3 antibody, anti-VEGFR2 antibody, anti-VISTA antibody; or
R5 corresponds to an antibody or functional fragment thereof selected from the group consisting of Utomilumab, Urelumab, ADG106, Poteligeo™ (Mogamulizumab), Poteligeo™ (Mogamulizumab), Bexxar™ (tositumomab), Zevalin™ (ibritumomab tiuxetan), Rituxan™ (rituximab), Arzerra™ (Ofatumumab), Gazyva™ (Obinutuzumab), Besponsa™ (Inotuzumab ozogamicin), Zenapax™ (daclizumab), Varlilumab, Theralizumab, Adcetris™ (Brentuximab vedotin), Myelotarg™ (gemtuzumab), Darzalex™ (Daratumumab), CDX-1140, SEA-CD40, R07009789, JNJ-64457107, APX-005M, Chi Lob 7/4, Campath™ (alemtuzumab), Raptiva™ (efalizumab), Soliris™ (eculizumab), Yervoy™ (ipilimumab), tremelimumab, Erbitux™ (cetuximab), Vectibix™ (panitumumab), Portrazza™ (Necitumumab), TheraCIM™ (Nimotuzumab), Synagis™ (palivizumab), Unituxin™ (Dinutuximab), TRX-518, MK-4166, MK-1248, GWN-323, INCAGN0186, BMS-986156, AMG-228, ReoPro™ (abiciximab), Herceptin™ (trastuzumab), Perjeta™ (Pertuzumab), Kadcyla™ (Ado-trastuzumab emtansine), GSK-3359609, JTX-2011, Simulect™ (basiliximab), Tysabri™ (natalizumab), BMS-986016, REGN3767, LAG525, Xolair™ (omalizumab), Tavolimab, PF-04518600, BMS-986178, MOXR-0916, GSK-3174998, INCAGN01949, IBI-101, Keytruda™ (Pembrolizumab), Opdivo™ (Nivolumab), Lartruvo™ (Olaratumab), Tencentriq™ (Atezolizumab), BMS-936559, Bavencio™ (Avelumab), Imfinzi™ (Duralumab), Prolia™ (Denosumab), Empliciti™ (Elotuzumab), MTIG7192A, TSR-022, MBG-453, Remicade™ (infliximab), Humira™ (adalimumab), Avastin™ (bevacizumab), Lucentis™ (ranibizumab), Cyramza™ (Ramucirumab), and JNJ-61610588.

15. The conjugate of the biomolecule of claim 14, wherein:
the mutation position in IL2 is selected from the group consisting of: Thr3, Lys32, Lys35, Thr37, Met39, Thr41, Lys43, Tyr45, Lys48, Lys49, Lys64, Leu72, Ala73, Ser75, Lys76, Leu94, Thr101, Thr102, Tyr107, Ala108, Thr111, Ala112, Cys125, Leu12, His16, Leu19, Met23, Gly27, Ser75, Arg81, Leu85, Ser87 and Asn88; or
the mutation position in IL10 is selected from the group consisting of: Thr6, Ser8, Ser11. Thr13, Gly17, Arg24, Ser31, Arg32, Lys34, Thr35, Lys40, Leu46, Lys49, Ser51, Lys57, Gly58, Ser66, Tyr72, Lys88, His90, Ser93, Lys99, Thr100, Arg104, Lys117, Ser118, Lys119, Lys125, Lys130, Lys134, Gly135, Tyr137, Tyr149, Thr155, Lys157 and Arg159; or
the mutation position in a heavy chain of the anti-PD-1 antibody Pembrolizumab is selected from the group consisting of: Ser7, Gly8, Gly15, Ala16, Ser17, Ala24, Ser25, Gly26, Tyr27, Thr28, Thr30, Asn31, Tyr32, Tyr33, Tyr35, Ala40, Gly42, Gly44, Leu45, Gly49, Gly50, Ile51, Asn52, Ser54, Asn55, Gly56, Gly57, Thr58, Asn59, Lys63, Lys65, Thr69, Leu70, Thr71, Thr72, Asp73, Ser74, Ser75, Thr76, Thr77, Thr78, Ala79, Leu83, Ser85, Leu86, Thr91, Ala92, Arg99, Asp100, Tyr101, Arg102, Asp104, Gly106, Gly111, Gly113, Thr114, 115Thr, 117Thr, Ser119, Ser120, Ala121, Ser122, Thr123, Lys124, Gly125 and Ser127; the mutation position in a light chain is selected from the group consisting of: Ile2, Thr5, Ser7, Ala9, Thr10, Leu11, Ser12, Leu13, Ser14, Gly16, Ala19, Thr20, Ala25, Ser26, Lys27, Gly28, Ser30, Thr31, Ser32, Gly33, Tyr34, Ser35, Tyr36, Leu37, Gly45, Ala47, Leu50, Leu51, Ile52, Tyr53, Leu54, Ala55, Ser56, Tyr57, Leu58, Ser60, Gly61, Ala64, Ser67, Gly68, Ser69, Gly70, Ser71, Gly72, Thr73, Ala76, Thr78, Ser80, Ser81, Ser95, Arg96, Asp97, Leu98, Leu100, Thr101, Phe102, Gly104, Ile110, Lys111 and K130; or
the mutation position in a heavy chain of the anti-PD-1 antibody Nivolumab is selected from the group consisting of: Gln3, Ser7, Gly8, Gly9, Gly10, Gly15, Ser17, Lys23, Ala24, Ser25, Gly26, Ile27, Asn31, Thr28, Ser30, Ser32, Gly33, Ala40, Gly42, Gly44, Leu45, Ala49, Ile51, Tyr53, Asp54, Gly55, Ser56, Lys57, Tyr59, Tyr60, Ala61, Asp62, Ser63, Lys65, Gly66, Thr69, Ile70, Ser71, Arg72, Asp73, Asn74, Ser75, Lys76, Asn77, Thr78, Leu79, Leu81, Ser85, Leu86, Ala88, Thr91, Ala92, Thr98, Asn99, Asp100, Asp101, Tyr102, Gly104, Gly106, Thr107, Leu108, Thr110, Ser112, Ser113, Ala114, Ser115, Thr116, Lys117, Gly118 and Ser120; the mutation position in a light chain is selected from the group consisting of: Ile2, Leu4, Thr5, Ser7, Ala9, Thr10, Leu11, Ser12, Leu13, Ser14, Gly16, Ala19, Thr20, Leu21, Ala25, Ser26, Ser28, Ser30, Ser31, Tyr32, Leu33, Ala34, Tyr36, Gly41, Ala43, Leu46, Leu47, Ile48, Tyr49, Asp50, Ala51, Ser52, Asn53, Arg54, Ala55, Thr56, Gly57, Ile58, Ala60, Arg61, Ser63, Gly64, Ser65, Gly66, Ser67, Gly68, Thr69, Thr72, Leu73, Thr74, Ile75, Ser76, Ser77, Leu78, Ala84, Ser91, Ser92, Asn93, Arg96, Thr97, Phe98, Gly99, Gly101, Thr102, Ile106, Lys107, Thr109, Ala111, Ala112, Ser114, Ile117 and Ser121;
the mutation position in a heavy chain of the anti-CTLA-4 antibody Ipilimumab is selected from the group consisting of: Gln3, Arg19, Leu20, Ser25, Gly26, Phe27, Thr28, Phe29, Ser30, Ser31, Tyr32, Thr33, Met34, His35, Gly44, Phe50, Ile51, Ser52, Tyr53, Asp54, Gly55, Asn56, Asn57, Lys58, Tyr59, Tyr60, Thr69, Ser71, Arg72, Asp73, Asn74, Ser75, Lys76, Asn77, Thr99, Gly100, Trp101, Leu102, Gly103 and Pro104; the mutation position in a light chain is selected from the group consisting of: Gln6, Arg24, Ala25, Ser26, Gln27, Ser28, Val29, Gly30, Ser31, Ser32, Tyr33, Ile49, Tyr50, Gly51, Ala52, Phe53, Ser54, Arg55, Ala56, Phe53, Ser54, Arg55, Ala56, Thr57, Gly58, Ile59, Pro60, Asp61, Arg62, Ser68, Gly69, Thr70, Gln90, Gln91, Tyr92, Gly93, Ser94, Ser95, Pro96 and Trp 97;
the mutation position in a heavy chain of the anti-TNFα antibody is selected from the group consisting of: Ser7, Gly8, Gly9, Gly10, Leu11, Gly15, Ser17, Leu18, Leu20, Ala24, Ser25, Gly26, Thr28, Asp30, Asp31, Tyr32, Ala33, Ala40, Gly42, Gly44, Leu45, Ser49, Ala50, Ile51, Thr52, Asn54, Ser55, Gly56, Ile58, Asp59, Tyr60, Ala61, Asp62, Ser63, Glu65, Gly66, Phe68, Thr69, Ile70, Ser71, Asp73, Asn74, Ala75, Lys76, Ser78, Leu79, Tyr80, Leu81, Ser85, Leu86, Ala88, Thr91, Ala92, Lys98, Ser100, Tyr101, Leu102, Ser103, Thr104, Ala105, Ser106, Ser107, Leu108, Asp109, Tyr110, Gly112, Gly114, Thr115, Leu116, thr118, Ser120, Ser121, Ala122, Ser123 and Thr124; the mutation position in a light chain is selected from the group consisting of: Asp1, Thr5, Ser7, Ser9, Ser10, Leu11, Ser12, Ala13, Ser14, Gly16, Thr20, Ile21, Ala25, Ser26, Gln27, Gly28, Ile29, Arg30, Asn31, Tyr32, Leu33, Ala34, Tyr36, Lys39, Gly41, Lys42, Ala43, Leu48, Leu47, Ile48, Tyr49, Ala50, Ala51, Ser52, Thr53, Leu54, Gln55, Ser56, Gly57, Ser60, Ser63, Gly64, Ser65, Gly66, Ser67, Gly68, Thr69, Asp70, Thr72, Leu73, Thr74, Ile75, Ser76, Ser77, Leu78, Ala84, Thr85, Tyr91, Asn92, Arg93, Ala94, Tyr96, Thr97, Phe98, Gly99, Gly101, Thr102, Ile106, Lys107, Thr109 and Ala111;
the mutation position in a heavy chain of the anti-CD28 antibody is selected from the group consisting of: Ser7, Gly8, Gly15, Ala16, Ser17, Ser21, Ala24, Ser25, Gly26, Tyr27, Thr28, Thr30, Ser31, Tyr32, Ala40, Gly42, Gly44, Gly49, Tyr52, Gly54, Thr58, Ala68, Thr69, Thr71, Thr74, Ser75, Ser77, Thr78, Ala79, Ser84, Leu86, Ser88, Thr91, Ala92, Thr97, Ser99, Tyr101, Gly102, Leu103, Gly113, Thr114, Thr115, Thr117, Ser119, Ser120, Ala121, Ser122 and Thr123;
the mutation position in a light chain is selected from the group consisting of: Thr5, Ser7, Ser9, Ser10, Ser11, Ser12, Ala13, Ser14, Gly16, Thr20, Thr22, Ala25, Ser26, Ser27, Ile29, Tyr30, Ala43, Leu46, Leu47, Tyr49, Lys50, Ala51, Ser52, Leu54, Thr56, Gly57, Ser60, Ser63, Gly64, Ser65, Gly66, Ser67, Gly68, Thr69, Asp70, Thr72, Thr74, Ser76, Ser77, Ala84, Thr85, Gly91, Thr93, Tyr94, Tyr96, Thr97, Phe98, Gly99, Gly100, Gly101, Thr102, Thr109 and Ala111;
the mutation position in a heavy chain of the anti-4-1BB antibody is selected from the group consisting of: Thr31, Tyr32, Ser35, Lys50, Tyr52, Asp55, Ser56, Tyr57, Thr58, Asn59, Tyr60, Ser61, Gln65, Gly66, Gly99, Tyr100, Gly101, Asp104 and Tyr105; the mutation position in a light chain is selected from the group consisting of: Ser23, Gly24, Asp25, Asn26, Gly28, Asp29, Gln30, Tyr31, Gln49, Asp50, Lys51, Asn52, Arg53, Ser55, Gly56, Thr89, Tyr90, Thr91, Gly92, Gly94 and Ser95; or
the mutation position in a heavy chain of the anti-Her2 antibody is selected from the group consisting of: Arg19, Lys30, Asp31, Tyr33, Arg50, Tyr62, Asn55, Tyr57, Arg59, Tyr60, Asp62, Lys65, Asp102 and Tyr105; the mutation position in a light chain is selected from the group consisting of: Asp1, Gln3, Gln27, Asp28, Asn30, Tyr49, Tyr55, Arg66, Asp70 and Tyr92. the mutation position in a heavy chain of the anti-PD-L1 antibody Atezolizumab is selected from the group consisting of: Gln3, Asp31, Tyr54, Tyr59, Tyr60, Asp62, Lys65, Asp73, Lys76, Asn77 and Arg99; the mutation position in a light chain is selected from the group consisting of: Arg24, Gln27, Asp28, Tyr49, Tyr55, Asp70, Gln89, Gln90, Tyr91 and Tyr93.

16. The conjugate of the biomolecule of claim 1, wherein R5 is a fusion protein containing one or more amino acid residues being mutated to cysteine;
the fusion protein containing an antigen binding domain of an antibody and optionally a cytokine; the fusion protein is a bispecific antibody containing an antigen binding domain to an antigen selected from the group consisting of HER2, CD19, EGFR, CD22, CD3, TROP2, Glycoprotein NMB, Guanylyl cyclase C, CEA, CD79b, PSMA, ENPP3, Mesothelin, CD138, NaPi2b, CD56, CD74, FOLR1, DLL3, CEACAM5, CD142, SLAMF7, CD25, SLTRK6, CD37, CD70, AGS-22, C4.4A, FGFR2, Ly6E, MUC16, BCMA, pCadherin, Ephrin-A, LAMP1, MUC1, CD19, PDL1, HER2, NY-ESO-1, BCMA, WT1, MUC1, CD20, CD23, ROR1, CD123, CD33, CD44v6, CD174, CD30, CD133, cMet, EGFR, FAP, EphA2, GD2, GPC3, IL-13Ra2, LewisY, Mesothelin, SS1, CEA, CD171, EGFR, EGFRvIII, VEGFR2, NY-ESO-1, MUC-1 and MAGE-A3; preferably, the bispecific antibody is a single chain bispecific antibody;
preferably, the fusion protein contains an antigen binding domain of an antibody and a cytokine selected from the group consisting of IL-2, IL-7, IL-10, IL-11, IL-12, IL-15, IL-21, IFN-α, IFN-β, IFN-γ, G-CSF, GM-CSF, TNF-α, TRAP, and TRAIL; preferably, the fusion protein is a fusion protein of anti-PD-1 antibody and IL2.

17. The conjugate of the biomolecule of claim 1, wherein the conjugate of the biomolecule, R1-R2-R3-R4 is

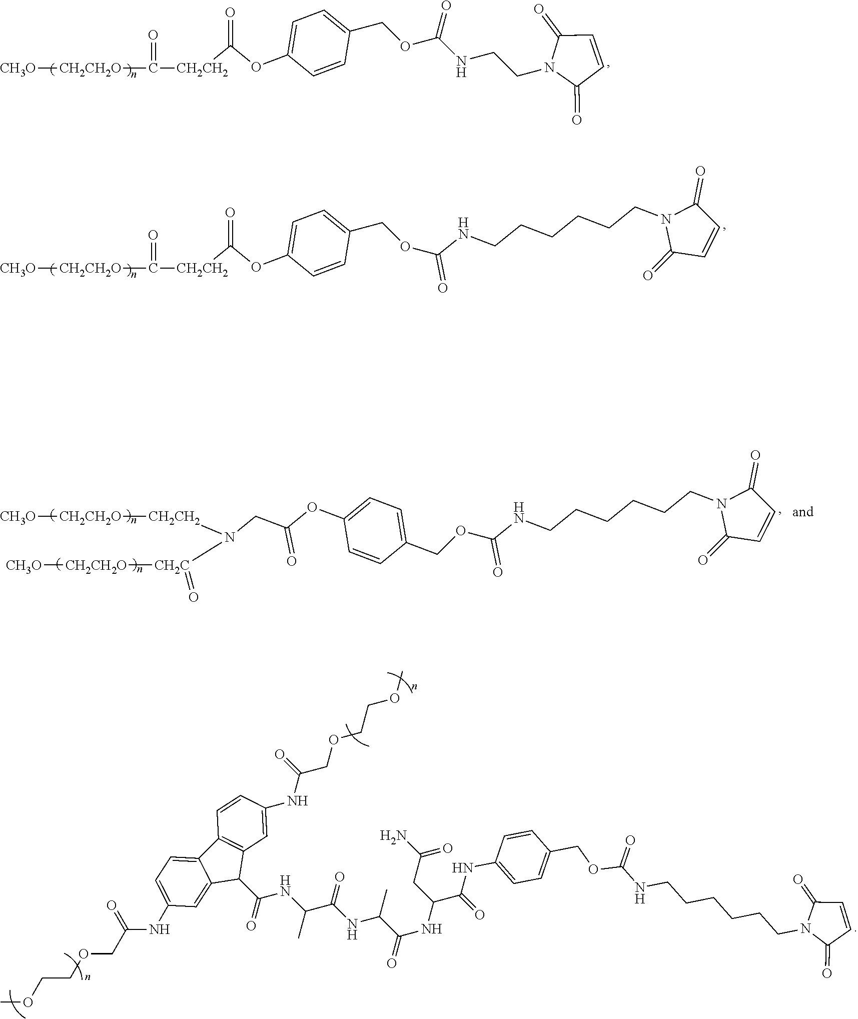

18. The conjugate of the biomolecule of claim 1, wherein in the conjugate of biomolecule, R1-R2-R3-R4 is selected from the group consisting of:

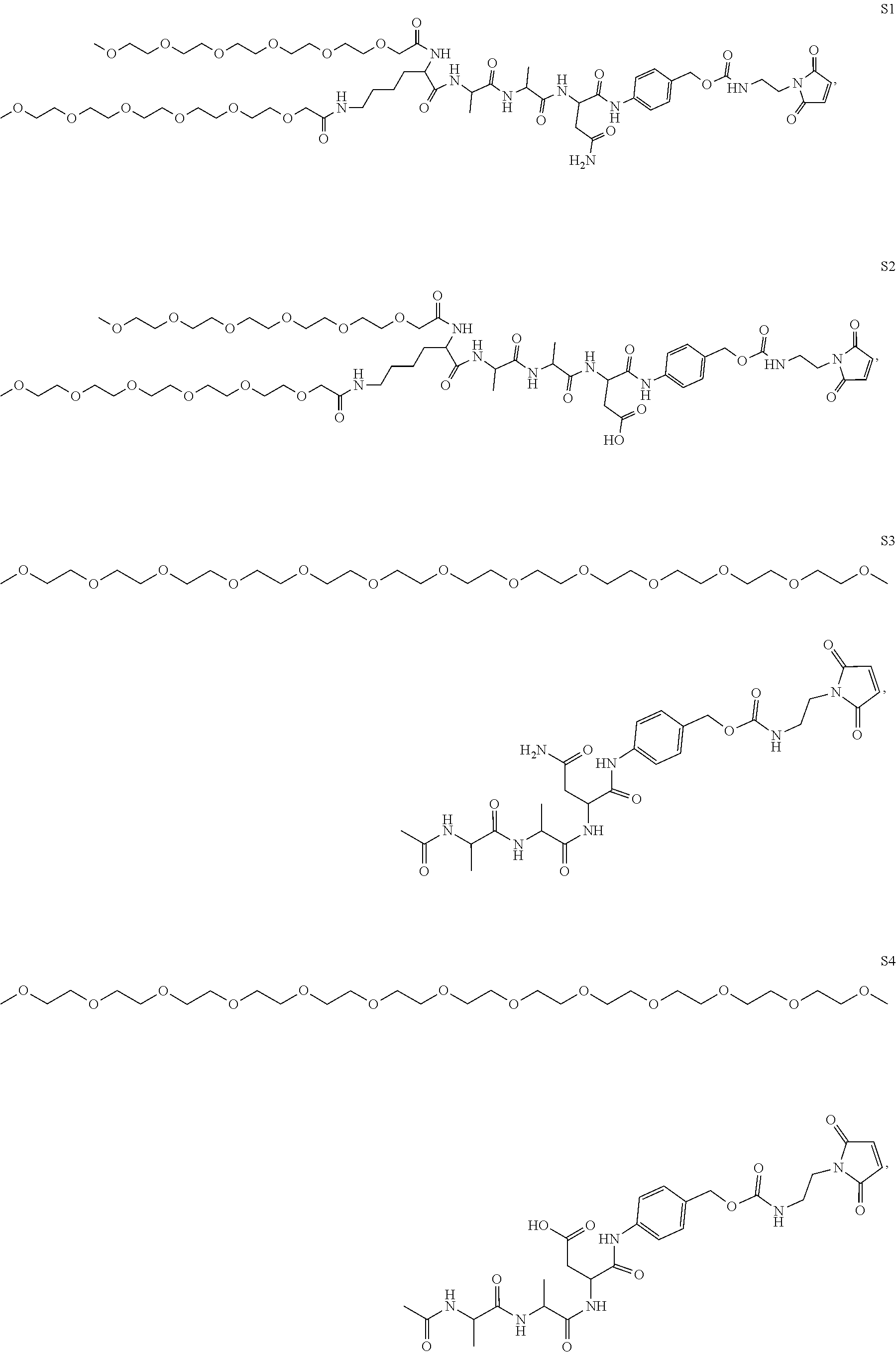

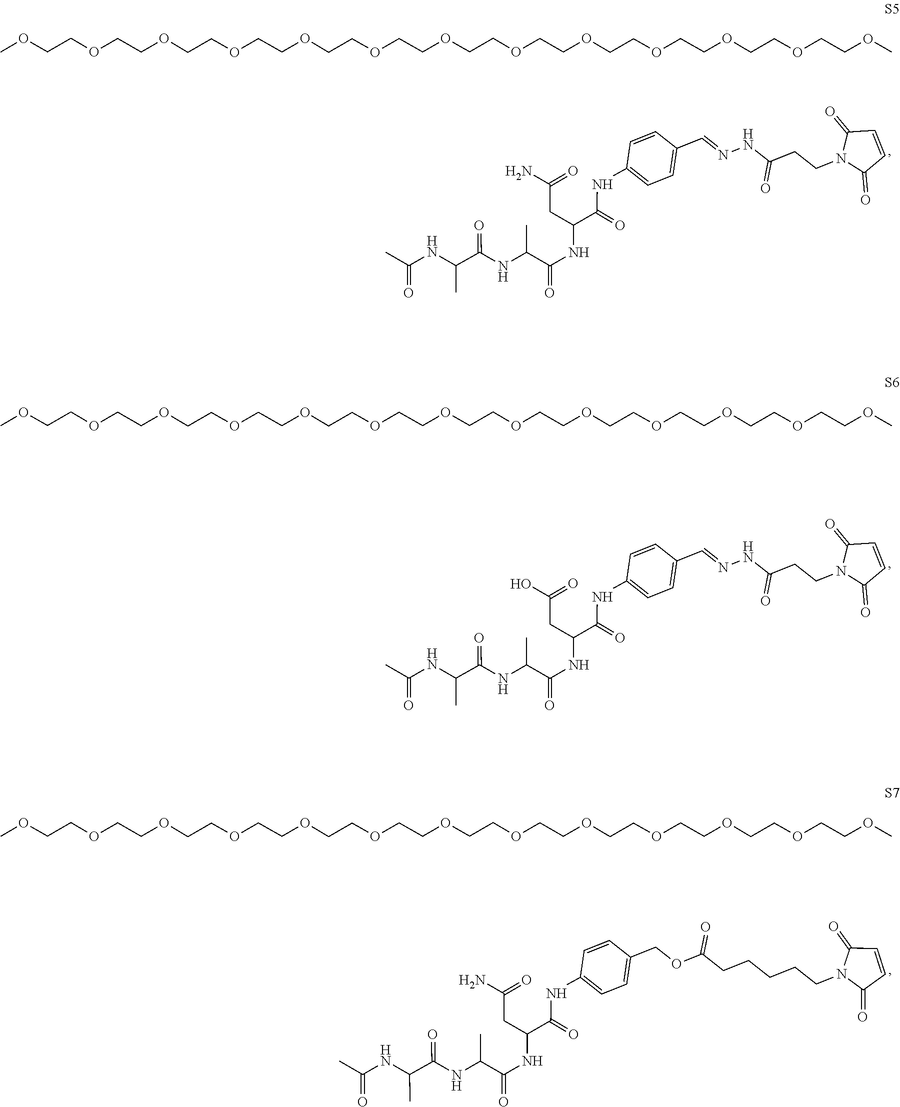

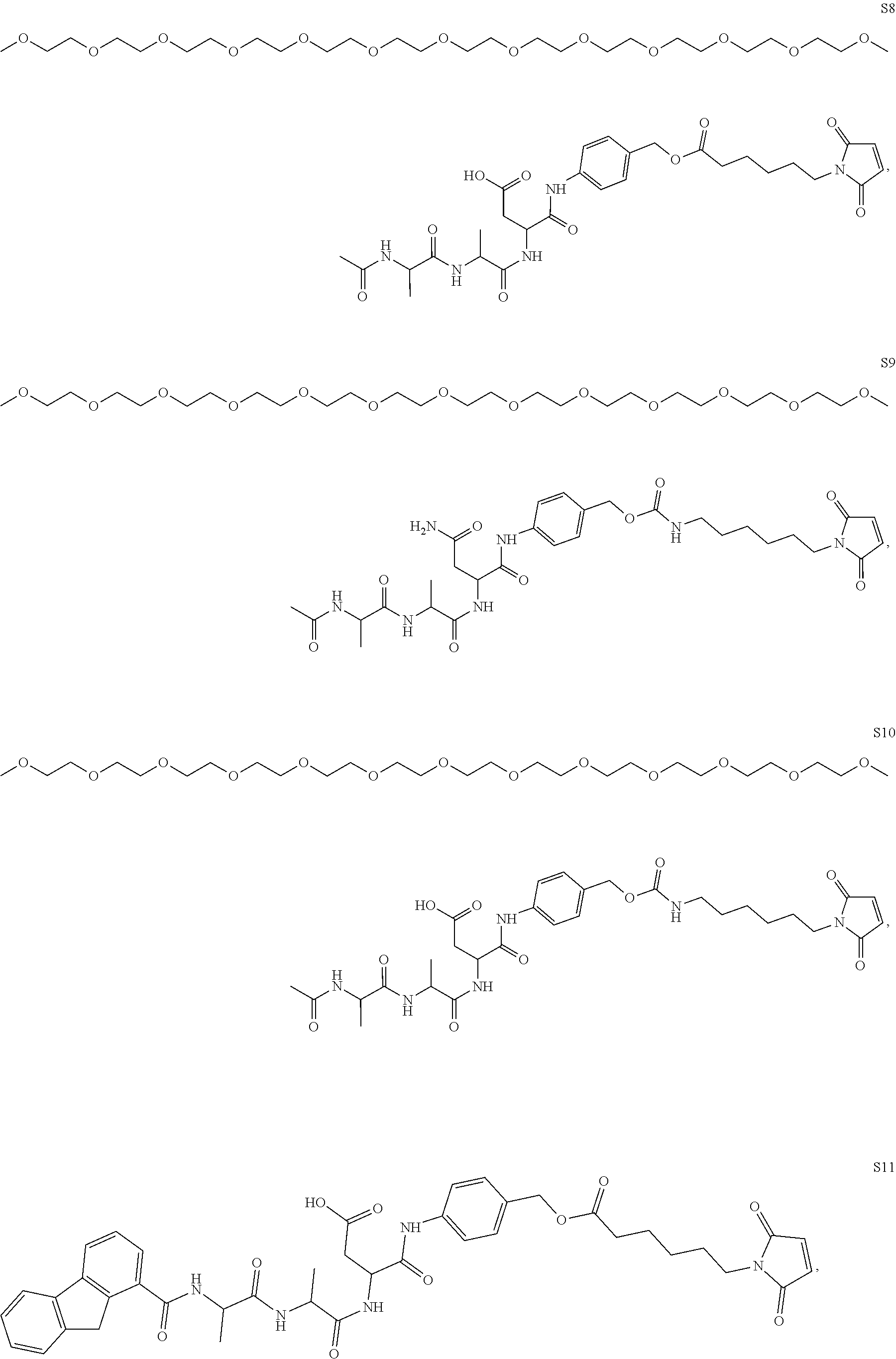

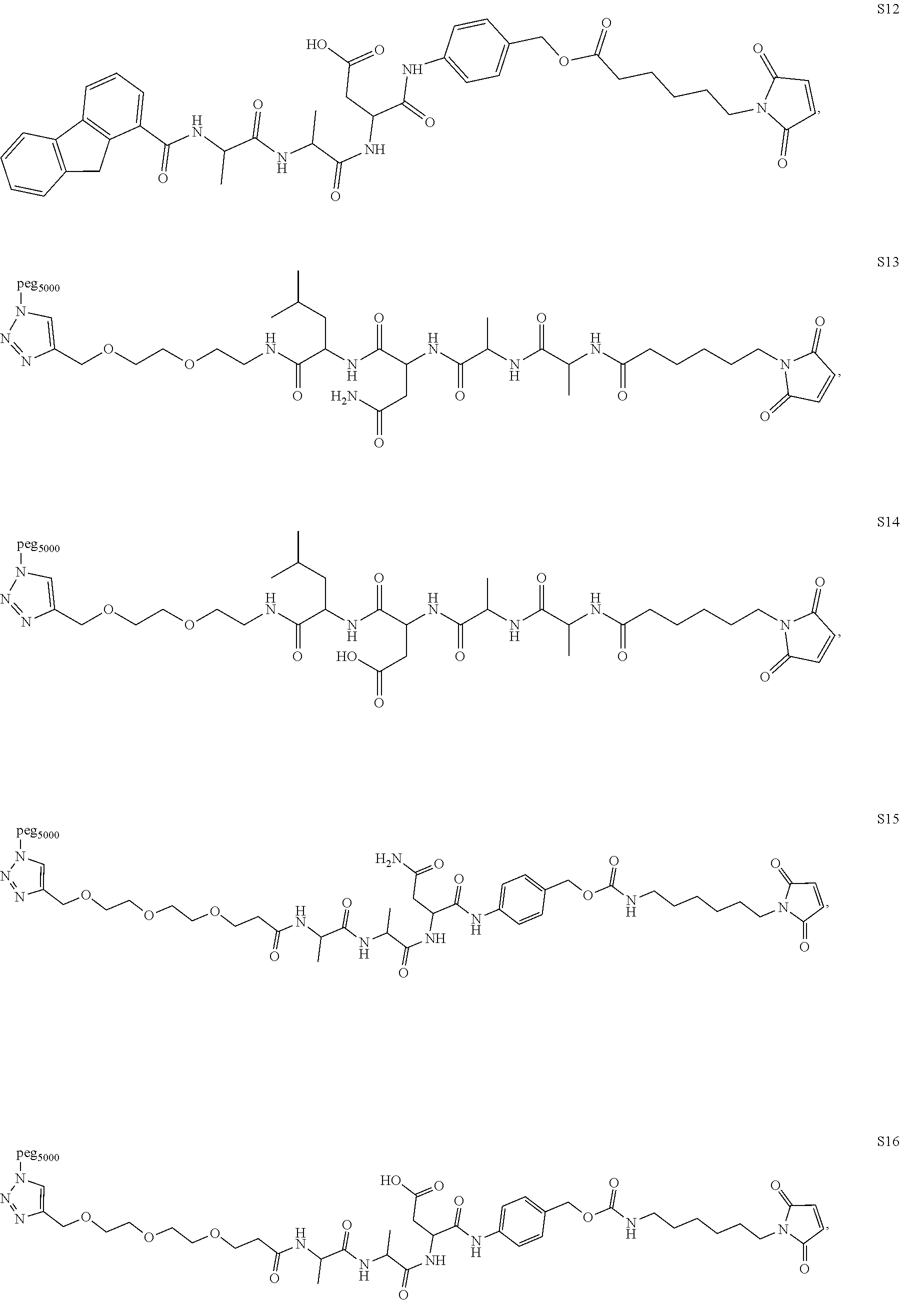

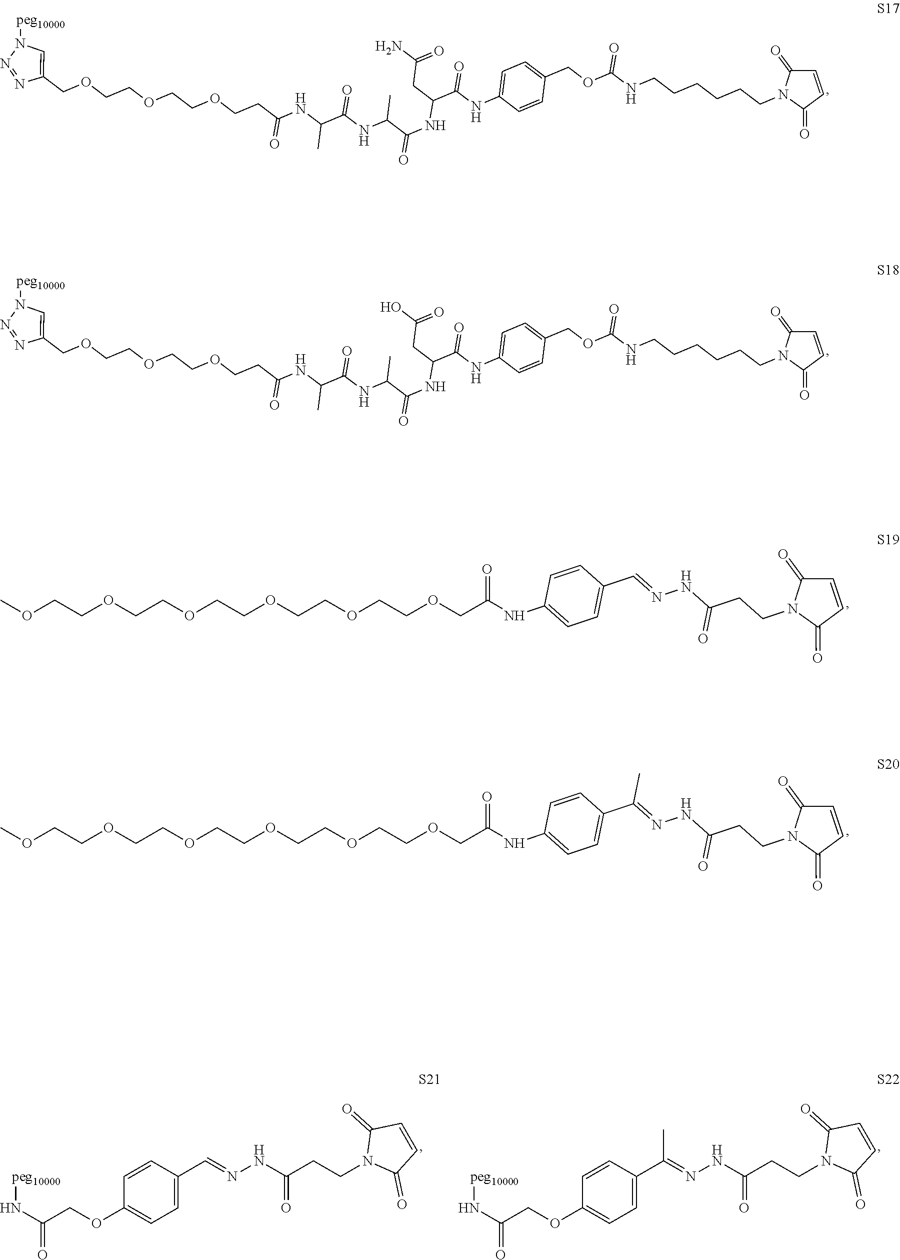

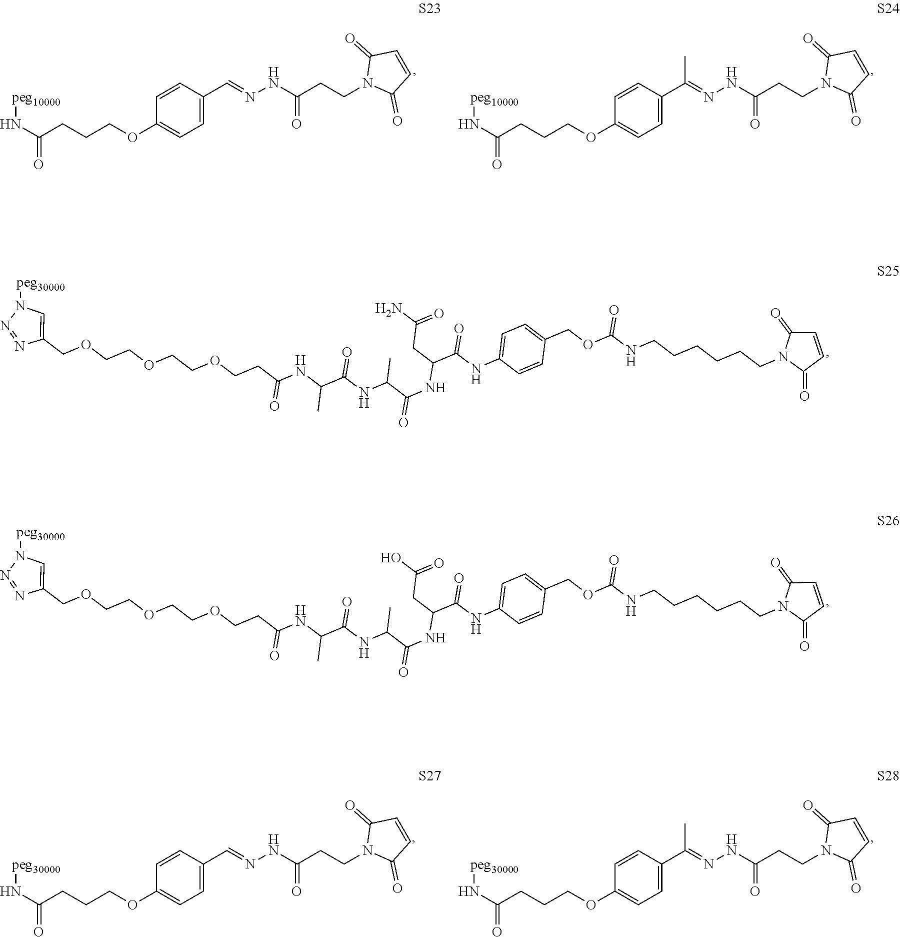

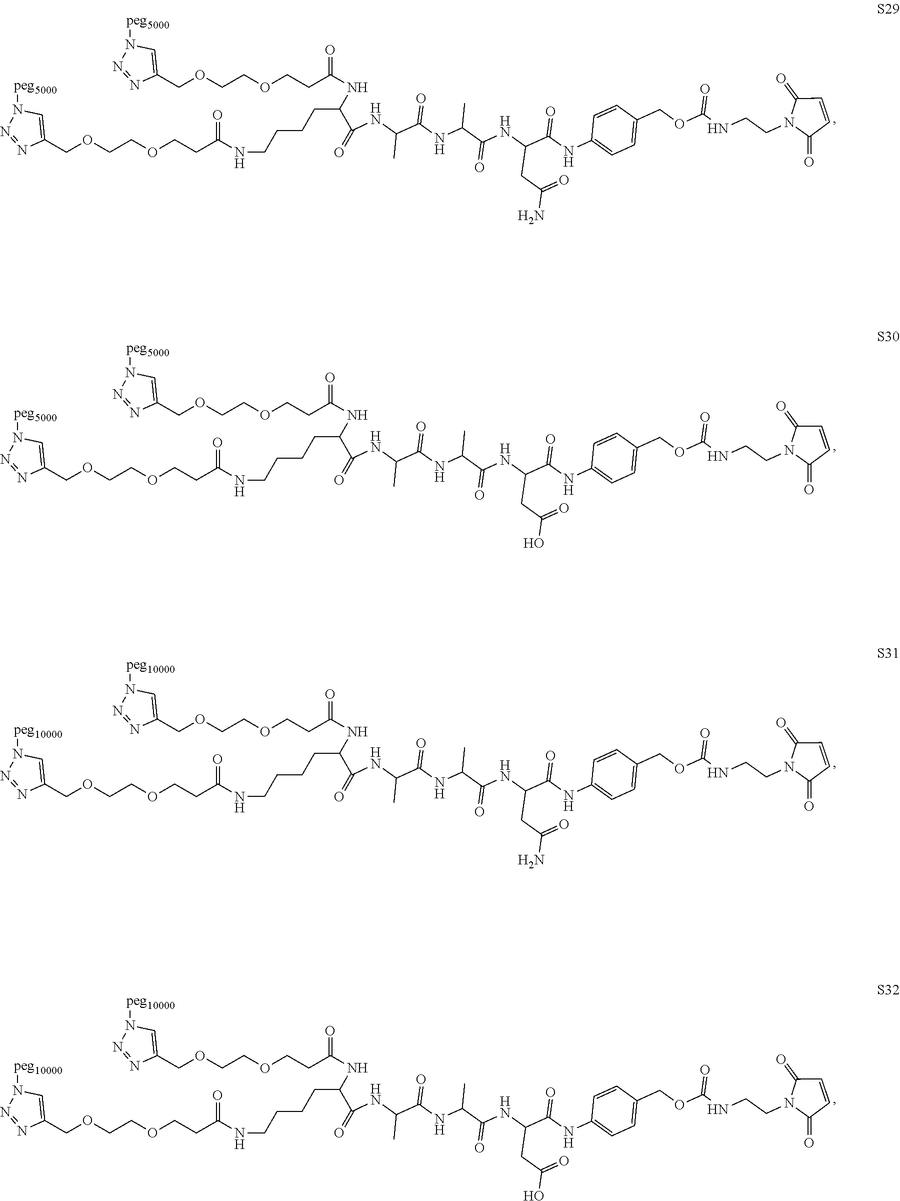

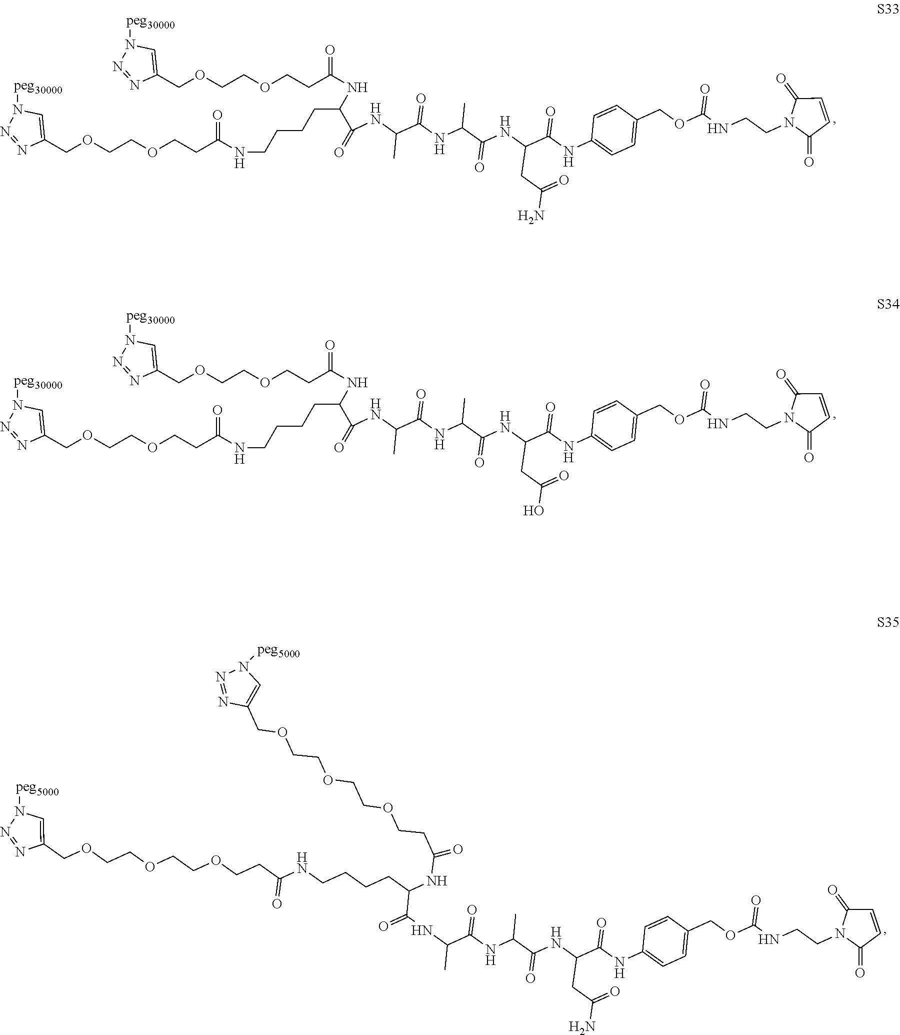

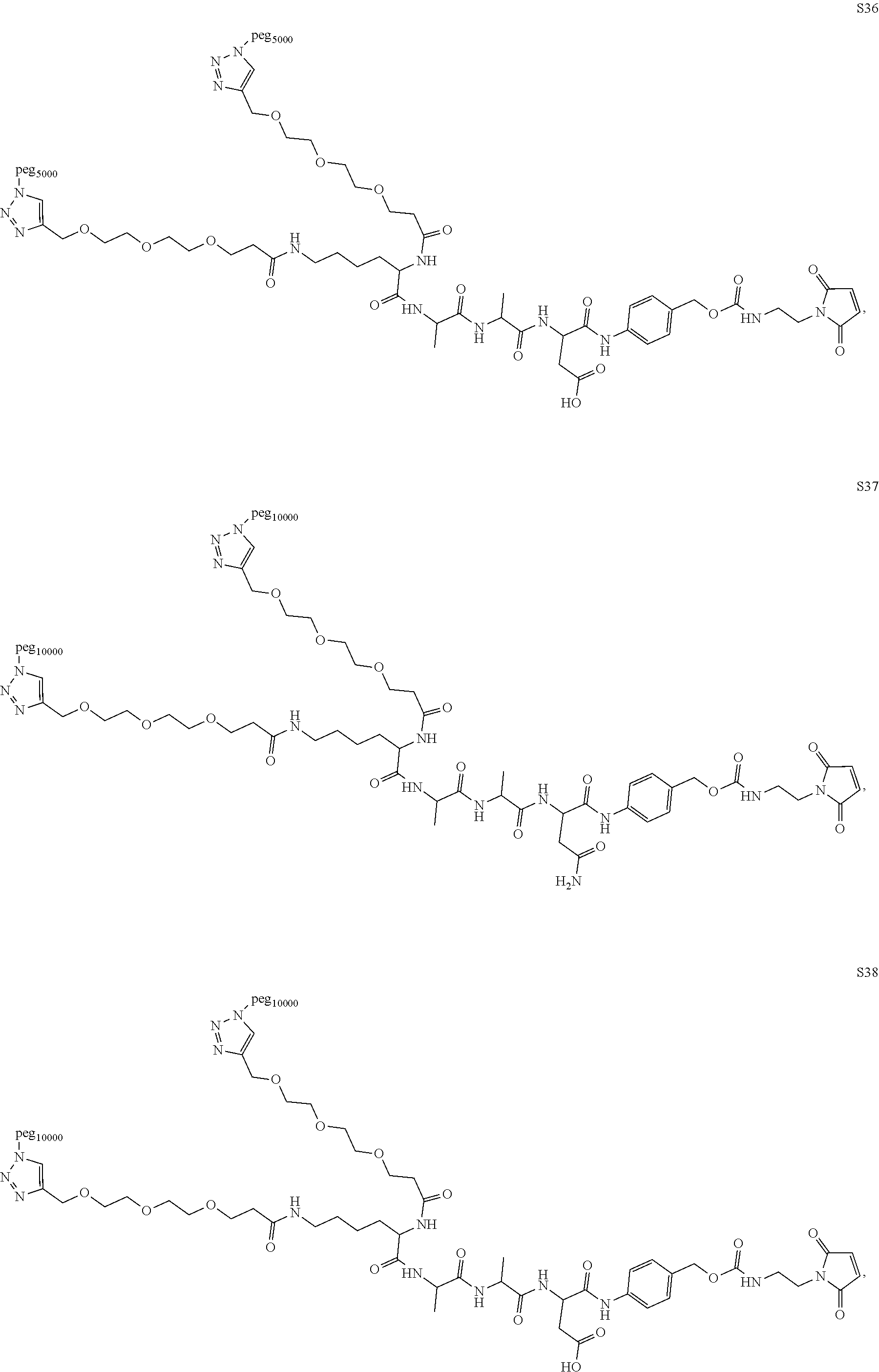

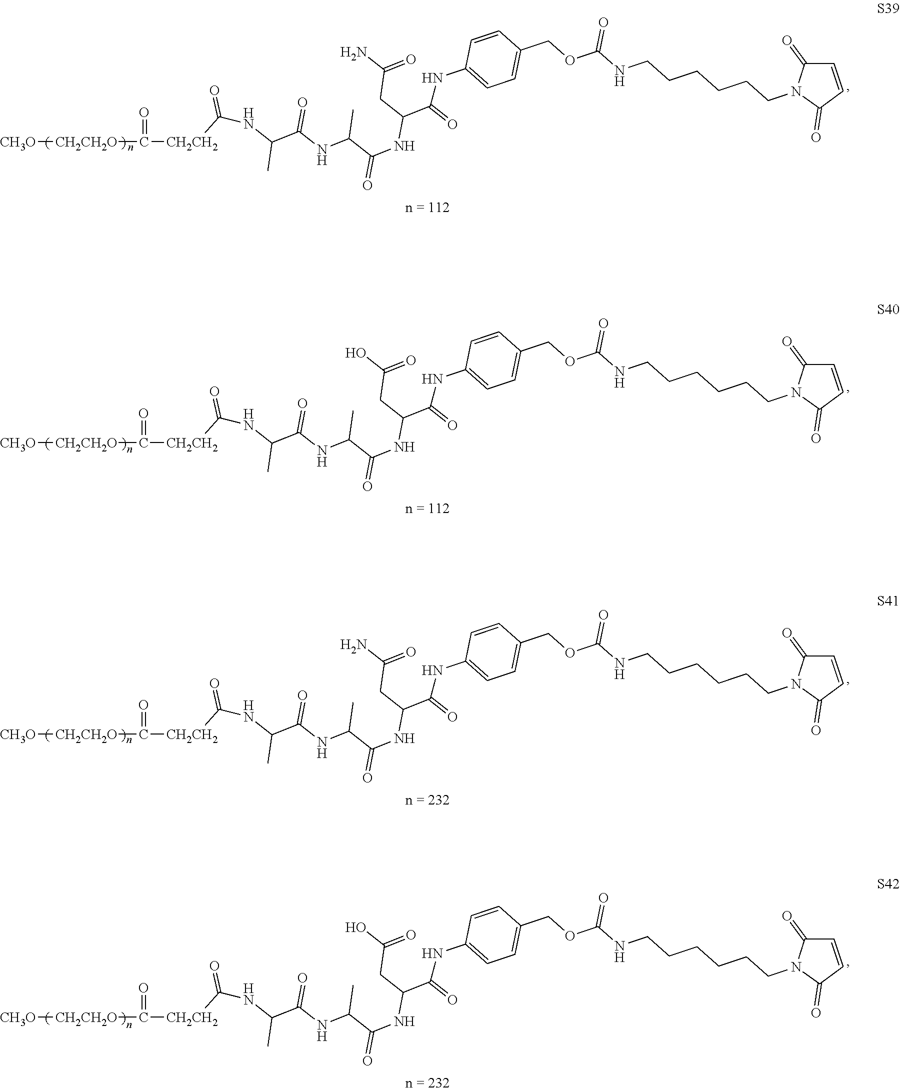

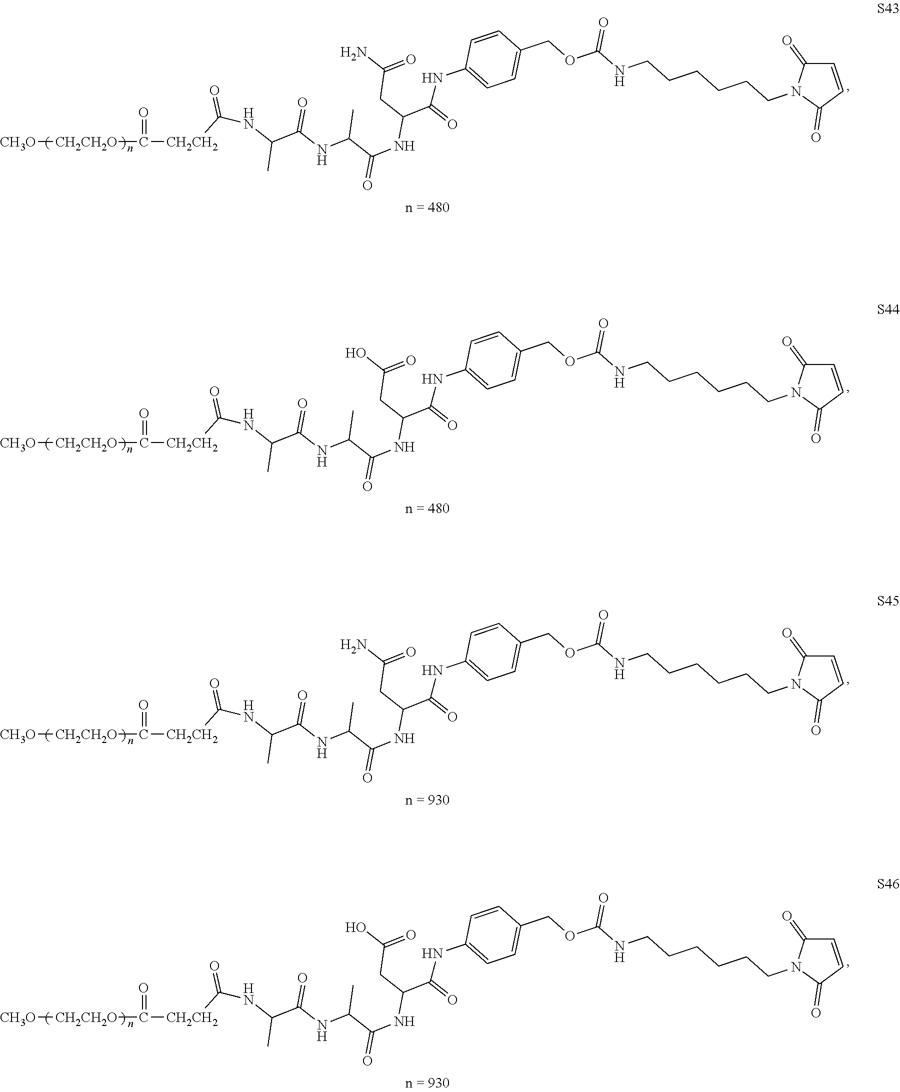

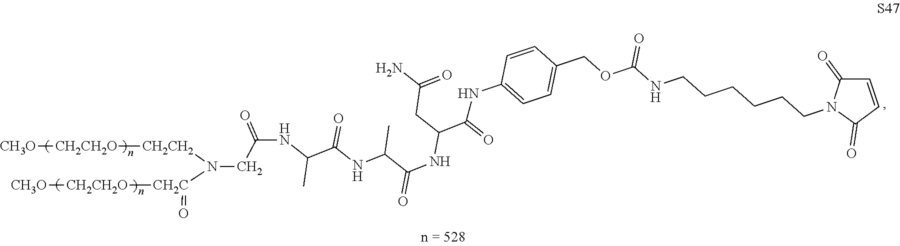

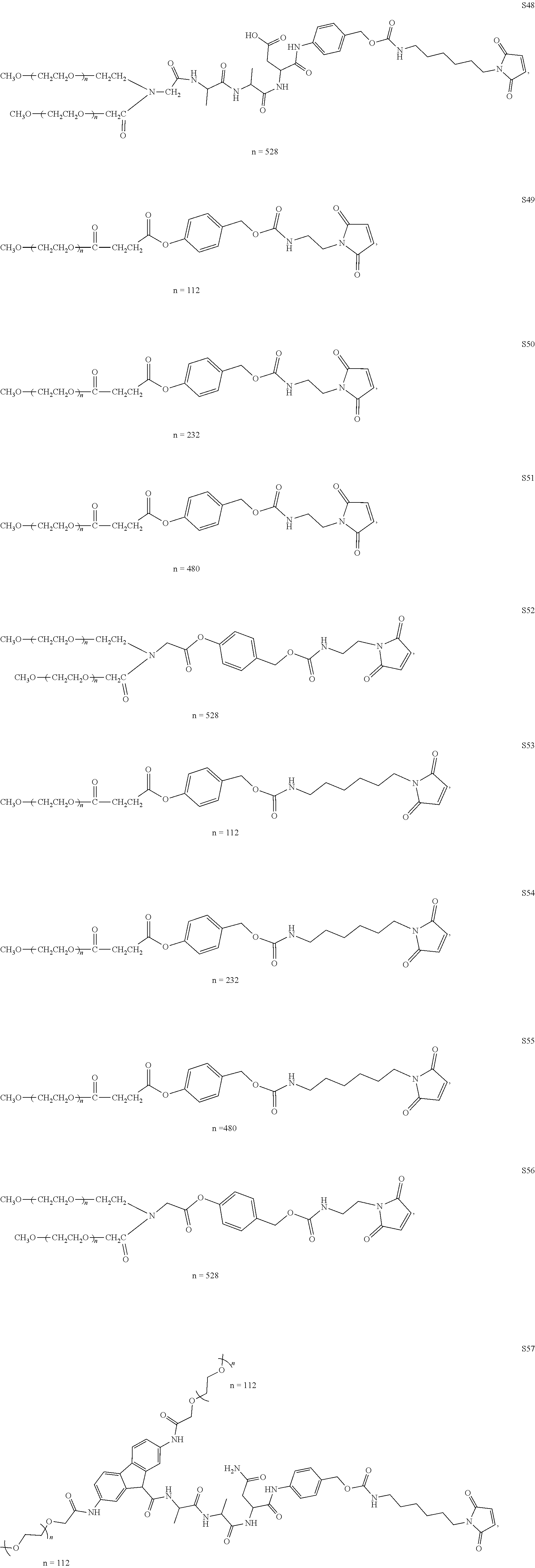

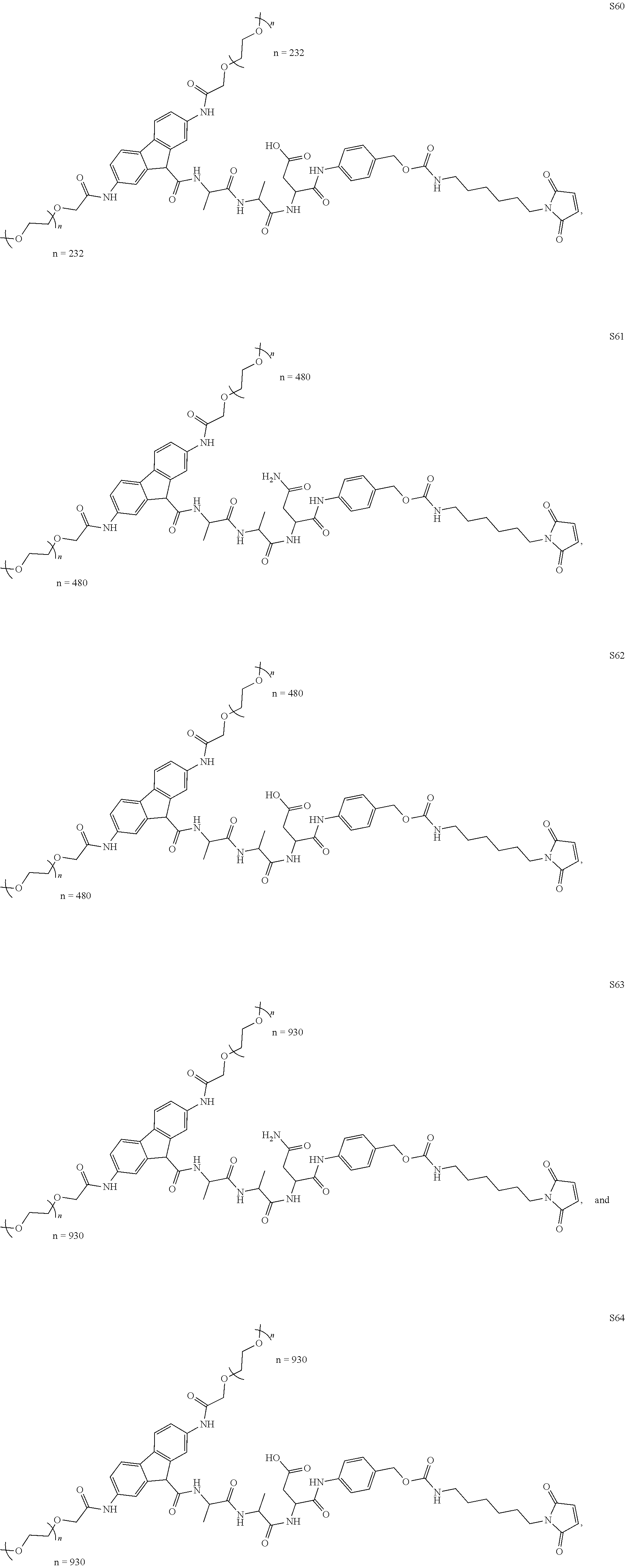

19. A conjugate having the following structure:
R4-S-cys-R5
wherein:
R4 is represented by —R₄₋ₐ-R_{4-b}-R_{4-c}, wherein R₄₋ₐ is selected from the group consisting of: 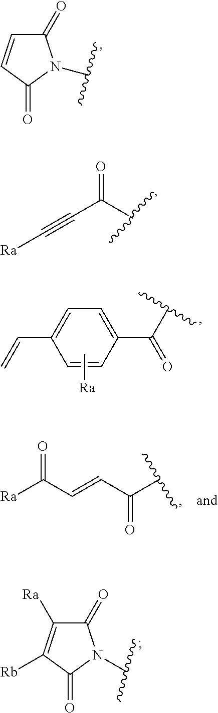
wherein Ra and Rb are each independently selected from the group consisting of H and C₁₋₆ alkyl or C₁₋₆ alkoxyl;
R_{4-b} is selected from the group consisting of: 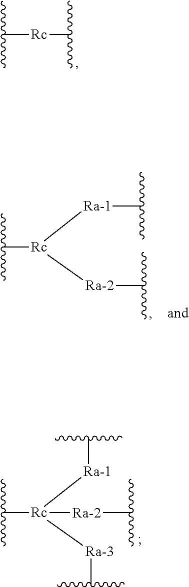
wherein in formula R4-b1, Rc is absent, or is selected from the group consisting of C₁₋₁₂ alkyl, C₁₋₁₂alkoxy-C₁₋₁₂alkyl, C₁₋₁₂alkyl-C₃₋₈cycloalkyl, (C₁₋₄alkyl-O)ₚ—C₁₋₁₂alkyl, C₁₋₁₂alkylcarbonylamino-(C₁₋₄alkyl-O)ₚ—C₁₋₁₂alkyl, phenyl-C₁₋₁₂alkyl, C₃₋₈cycloalkyl, C₁₋₁₂alkyl-C₃₋₈cycloalkyl-C₁₋₁₂alkyl, and C₁₋₁₂alkyl-phenyl-C₁₋₁₂alkyl; in formula R4-b2, Rc is a C₁₋₁₂alkylamino with Ra-1 and Ra-2 substituted at N atom of the amino group, and in formula 4-b3, Rc is a C₁₋₁₂alkyl with the last C atom at the end of the alkyl being substituted by Ra-1, Ra-2 and R2-3, wherein Ra-1, Ra-2 and Ra-3 are each independently selected from the group consisting of C₁₋₁₂alkyl, C₁₋₁₂alkyl-OH, and C₁₋₁₂alkyl-NR″R′″, wherein R″ and R′″ are each independently selected from the group consisting of H and C₁₋₁₂alkyl; wherein in formulae R4-b2 and R4-b3, R4-b links to R4-c via at least one of the Ra-1, Ra-2 and Ra-3;
R_{4-c} is selected from the group consisting of: 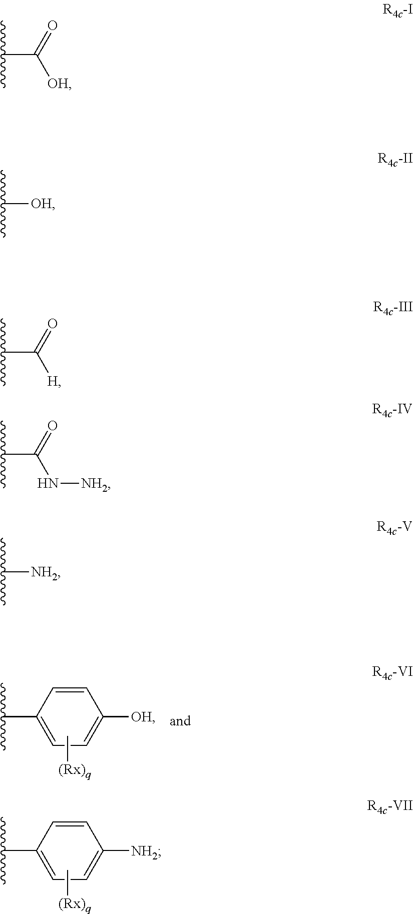
wherein Rx is selected from the group consisting of H, halo and C₁₋₄alkyl; p is an integer in a range of 1 to 10, such as an integer in a range of 1 to 5; and q is an integer in a range of 1 to 4, such as an integer in a range of 1 to 2; with the proviso that R4-c is absent when R4-a is selected from the group consisting of formulae R4-a2, R4-a3 and R4-a4;
wherein the wave line shown in each formula of R4-a indicates the position at which R4-a links to R4-b, and the wave line shown in each formula of R4-c indicates the position at which R4-c links to R4-b.

20. The compound of claim 19, wherein R4 is selected from the group consisting of:

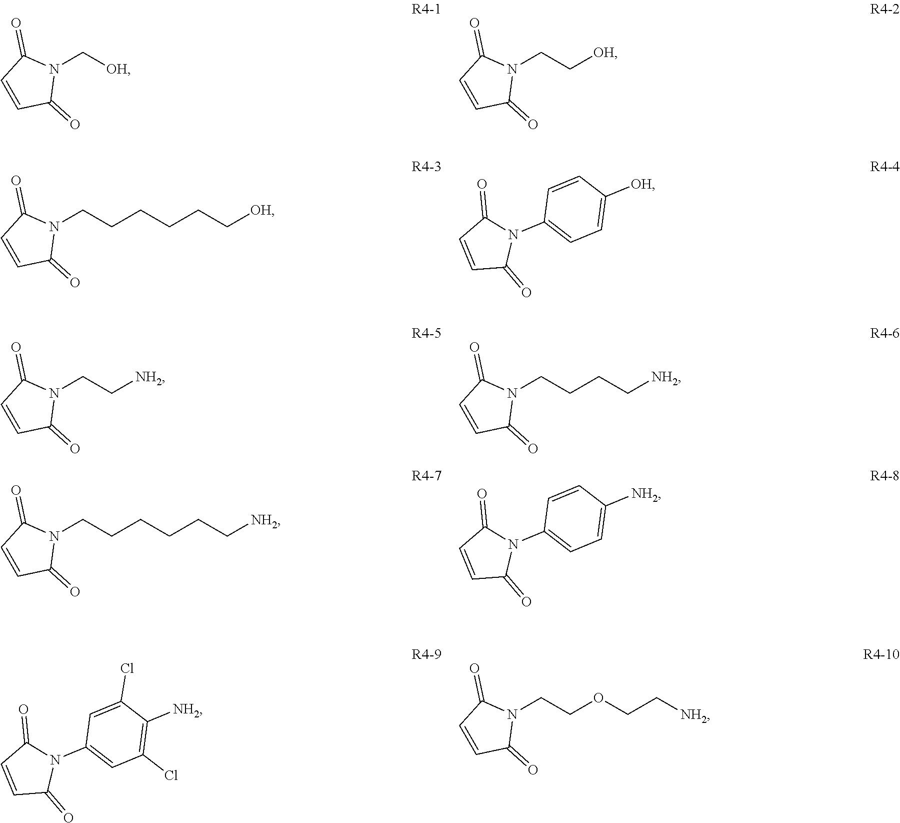

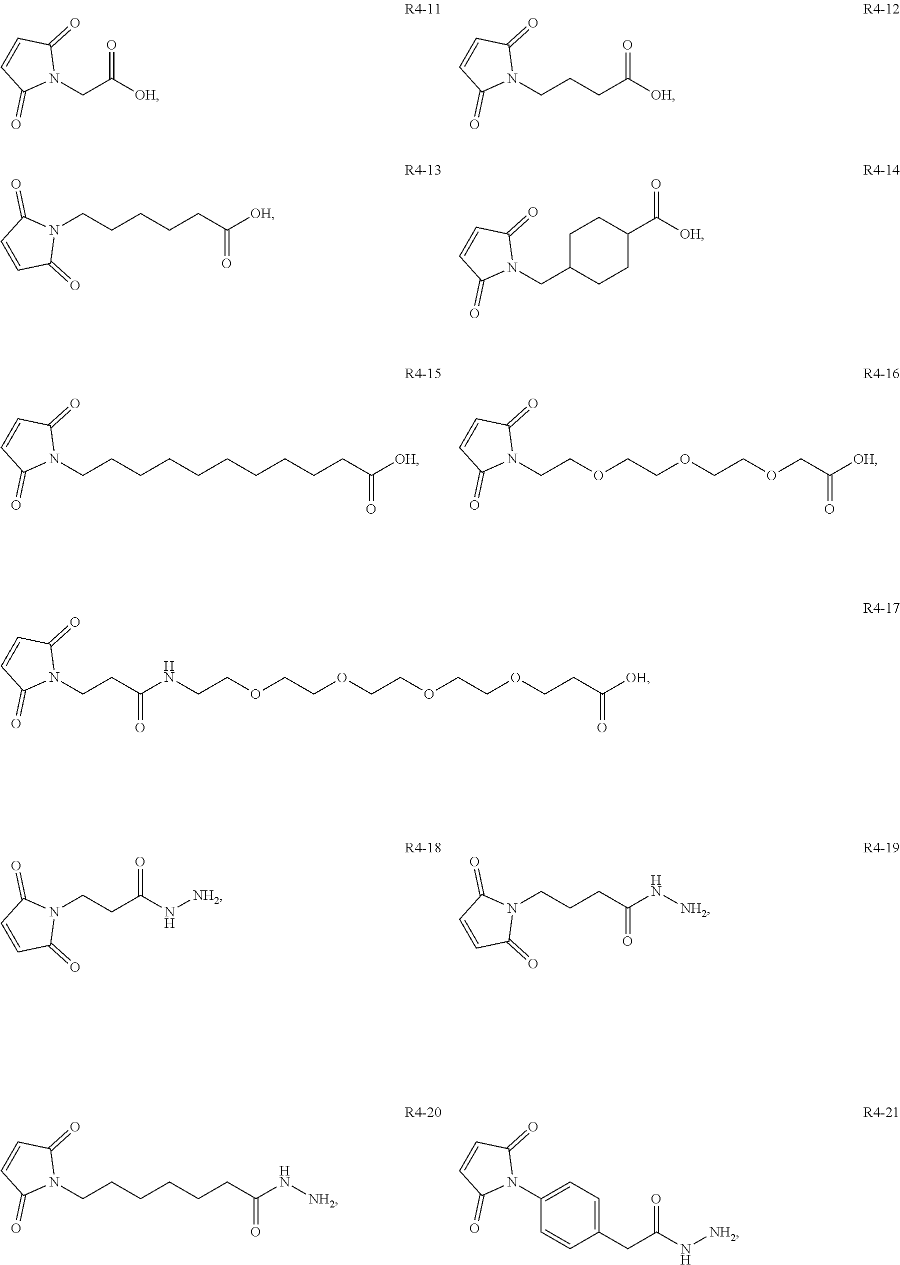

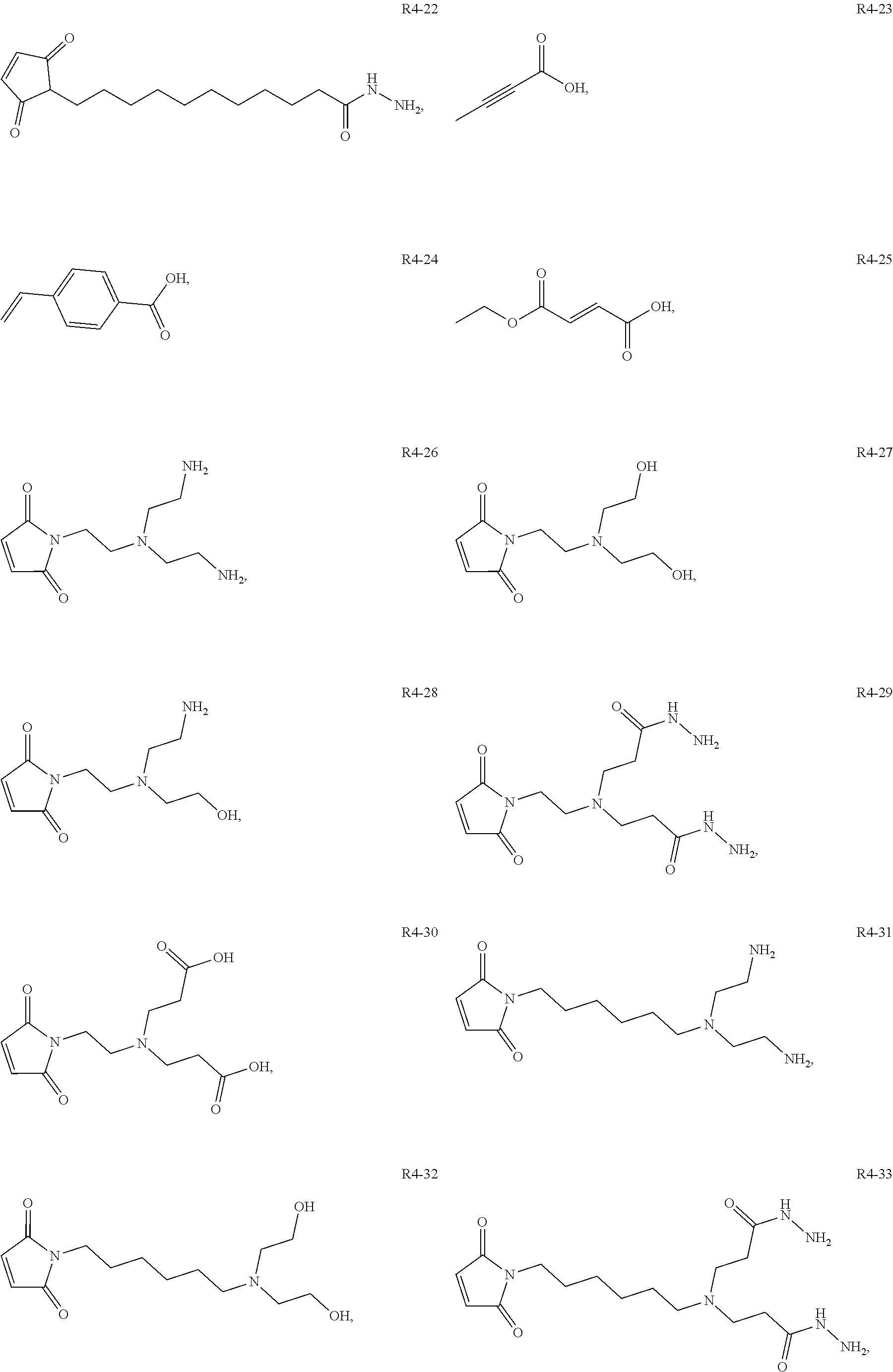

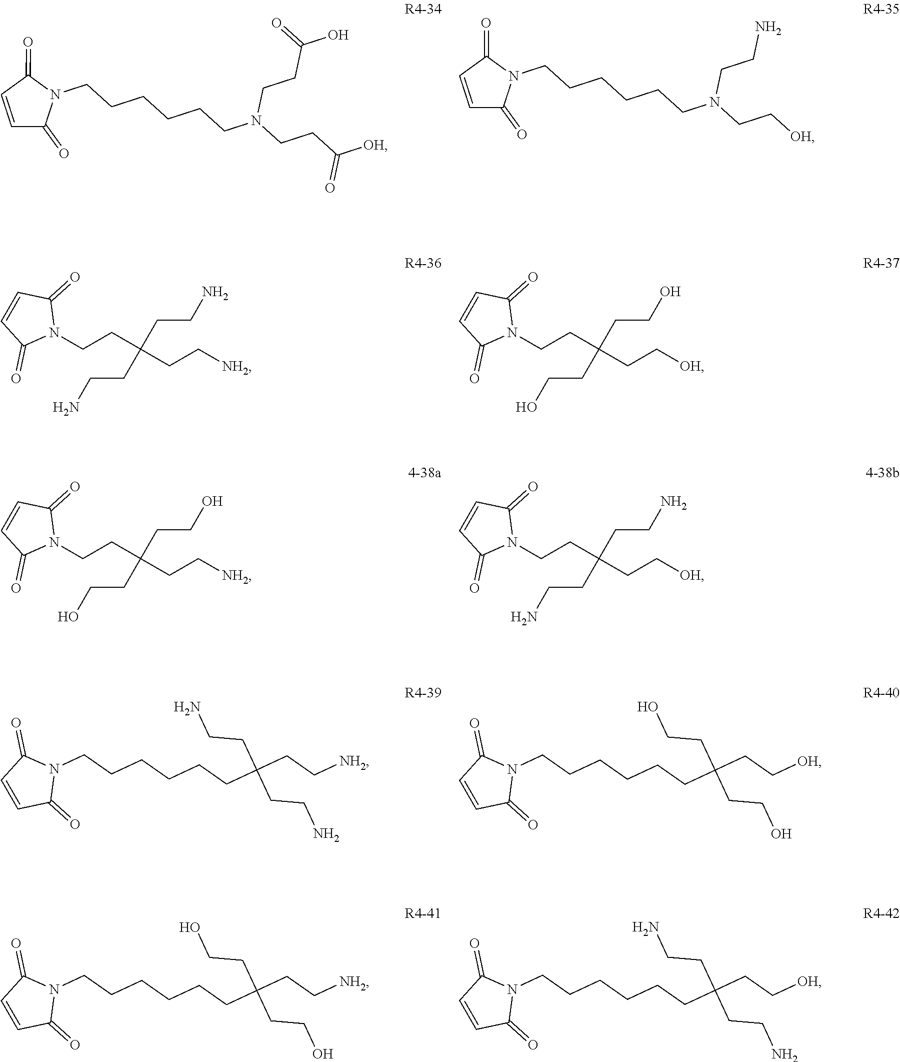

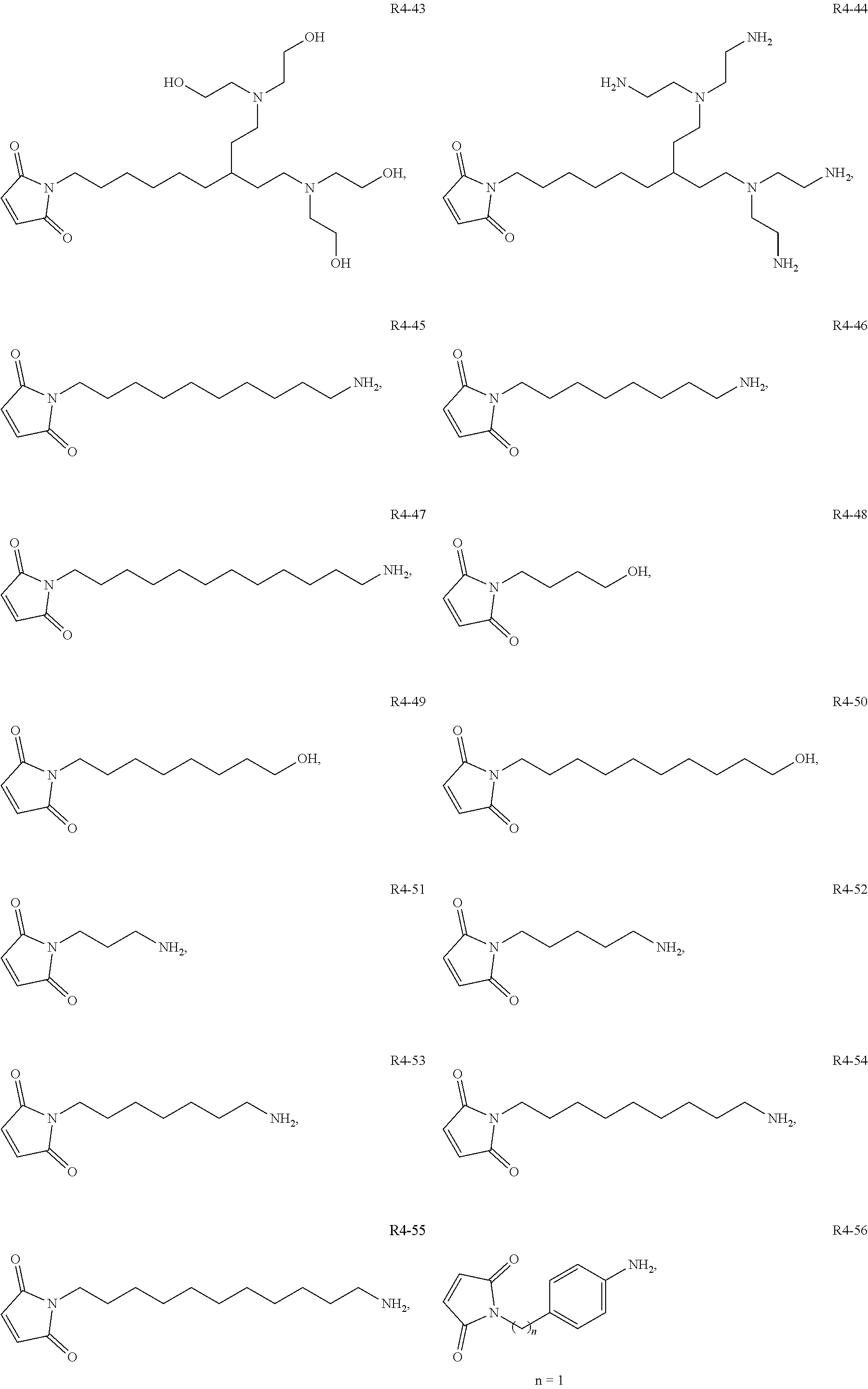

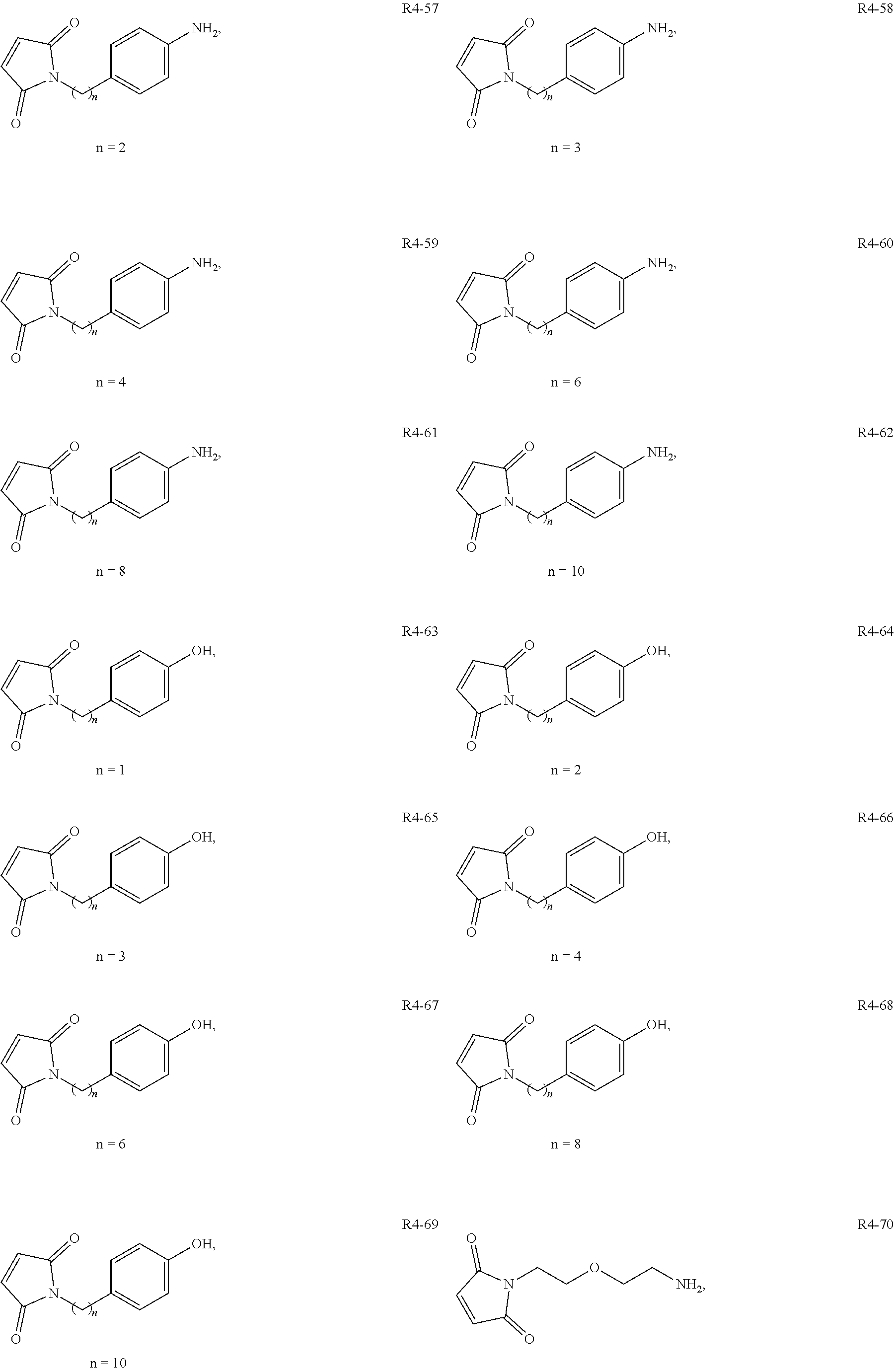

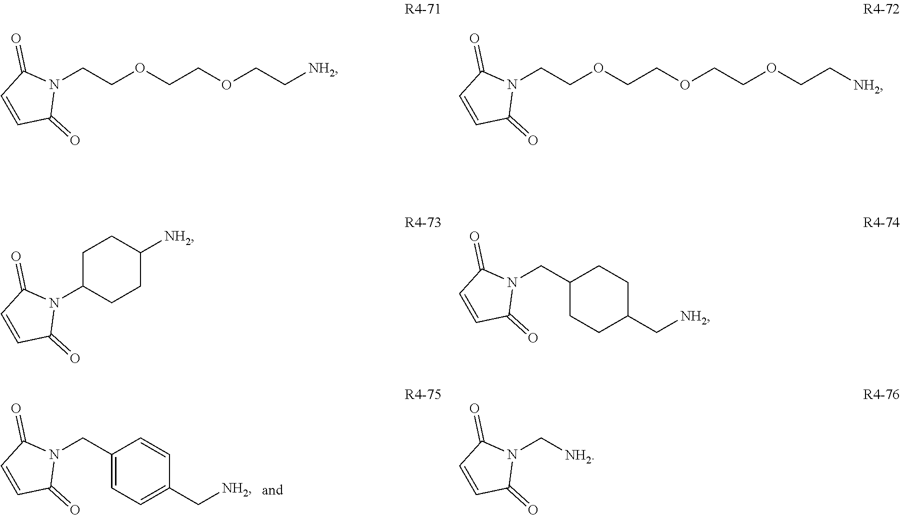 [SMILES: NCCOCCOCCN1C(=O)C=CC1=O ; NCCOCCOCCOCCN1C(=O)C=CC1=O ; NC1CCC(N2C(=O)C=CC2=O)CC1 ; NCC1CCC(CN2C(=O)C=CC2=O)CC1 ; NCc1ccc(CN2C(=O)C=CC2=O)cc1 ; NCN1C(=O)C=CC1=O]

21. A compound represented by R1-R2-R3-R4, wherein the compound is represented by:

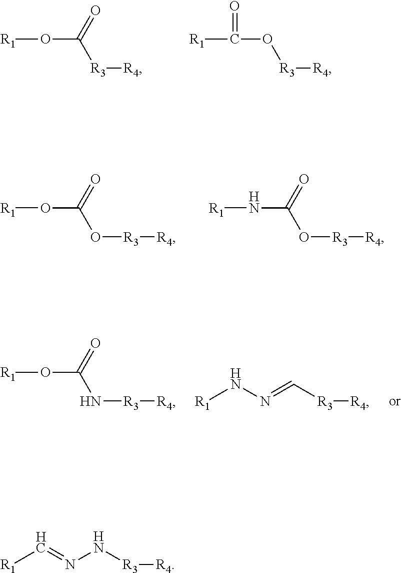

22. A compound selected from the group consisting of: 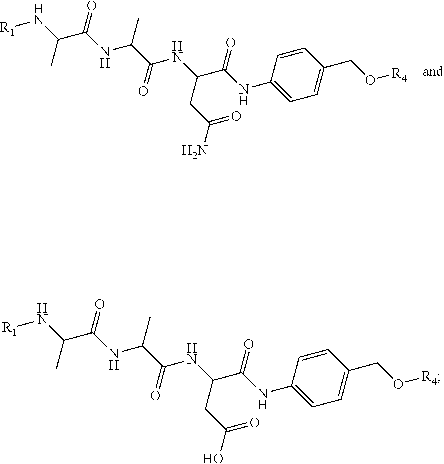
wherein,
R1 is H, or wherein R1 is selected from the group consisting of polyethylene glycol-C₁₋₅ alkylcarbonyl, 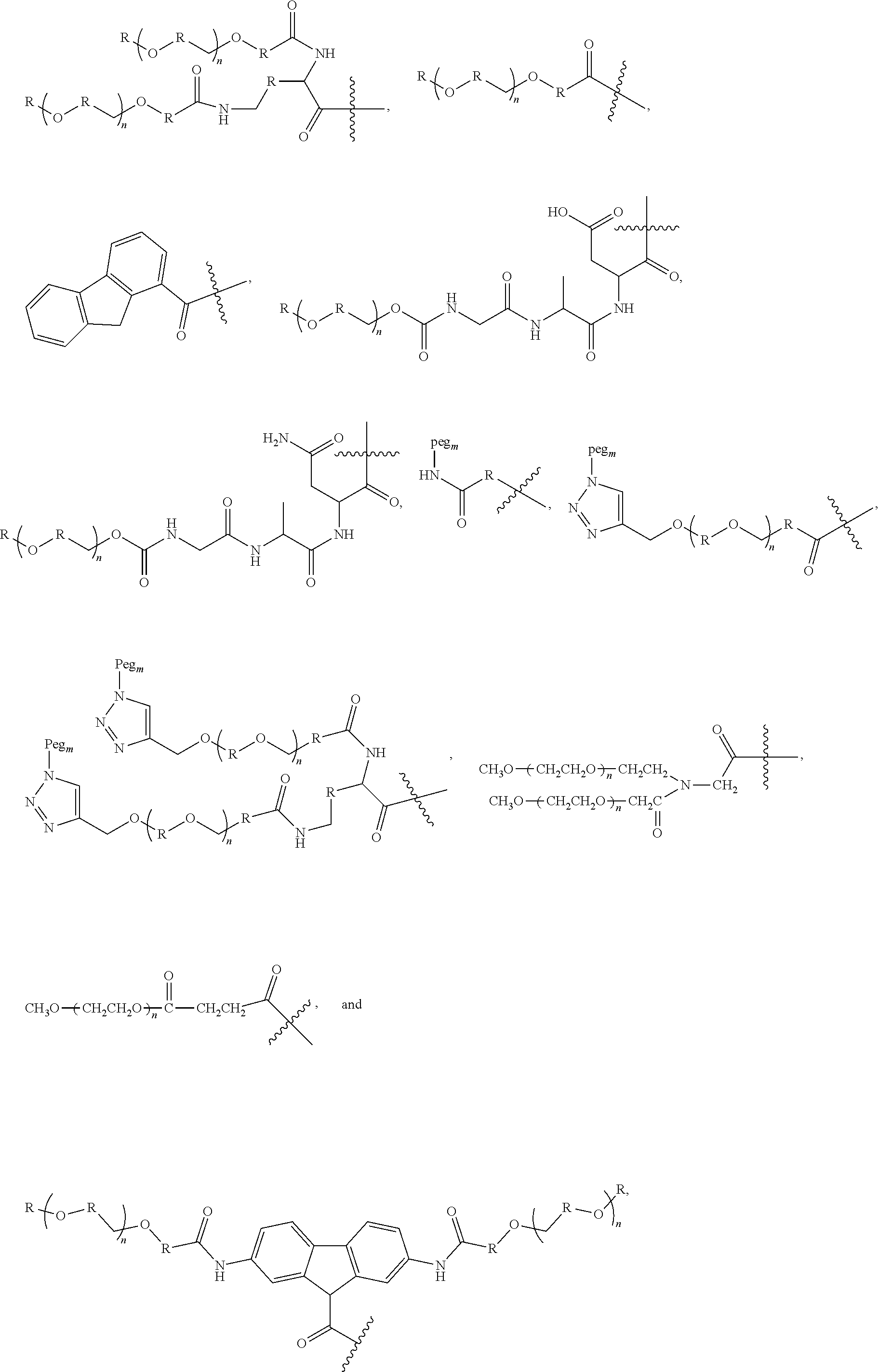
wherein each R is independently C₁₋₄ alkyl; each n is independently an integer in a range of 1 to 30000 kDa; the polyethylene glycol or pegₘ is a polyethylene glycol having a molecular weight in a range of 44 to 132000 kDa; m represents the molecular weight of the polyethylene glycol; and the wave line indicates the position of R1 linking to R2;
wherein R4 is represented by —R₄₋ₐ-R_{4-b}-R_{4-c}—, wherein R4-a is selected from the group consisting of: 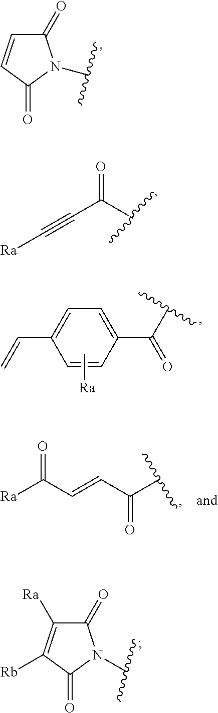
wherein Rα and Rb are each independently selected from the group consisting of H and C₁₋₆ alkyl or C₁₋₆ alkoxyl;
R_{4-b} is selected from the group consisting of: 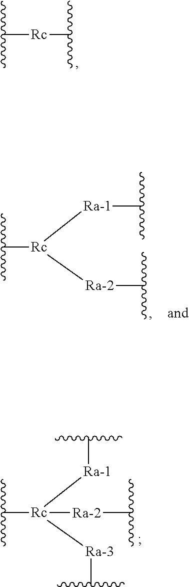
wherein in formula R4-b1, Rc is absent, or is selected from the group consisting of C₁₋₁₂ alkyl, C₁₋₁₂alkoxy-C₁₋₁₂alkyl, C₁₋₁₂alkyl-C₃₋₈cycloalkyl, (C₁₋₄alkyl-O)ₚ—C₁₋₁₂alkyl, C₁₋₁₂alkylcarbonylamino-(C₁₋₄alkyl-O)ₚ—C₁₋₁₂alkyl, phenyl-C₁₋₁₂alkyl, C₃₋₈cycloalkyl, C₁₋₁₂alkyl-C₃₋₈cycloalkyl-C₁₋₁₂alkyl, and C₁₋₁₂alkyl-phenyl-C₁₋₁₂alkyl; in formula R4-b2, Rc is a C₁₋₁₂alkylamino with Ra-1 and Ra-2 substituted at N atom of the amino group, and in formula 4-b3, Rc is a C₁₋₁₂alkyl with the last C atom at the end of the alkyl being substituted by Ra-1, Ra-2 and R2-3, wherein Ra-1, Ra-2 and Ra-3 are each independently selected from the group consisting of C₁₋₁₂alkyl, C₁₋₁₂alkyl-OH, and C₁₋₁₂alkyl-NR″R′″, wherein R″ and R′″ are each independently selected from the group consisting of H and C₁₋₁₂alkyl; wherein in formulae R4-b2 and R4-b3, R4-b links to R4-c via at least one of the Ra-1, Ra-2 and Ra-3;
R_{4-c} is selected from the group consisting of: 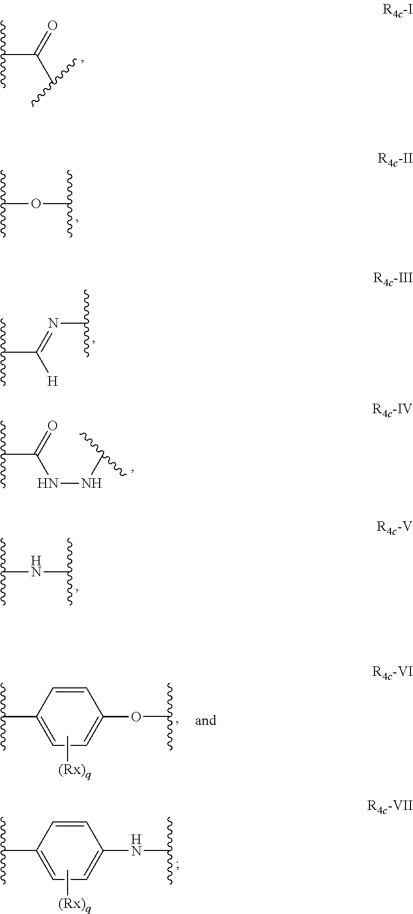
wherein Rx is selected from the group consisting of H, halo and C₁₋₄alkyl; p is an integer in a range of 1 to 10, such as an integer in a range of 1 to 5; and q is an integer in a range of 1 to 4, such as an integer in a range of 1 to 2; with the proviso that R4-c is absent when R4-a is selected from the group consisting of formulae R4-a2, R4-a3 and R4-a4;
wherein R3 links to R4 via the R4-c of R4, and the wave line shown in each formula of R4-a indicates the position at which R4-a links to R4-b.

23. A compound selected from the group consisting of:

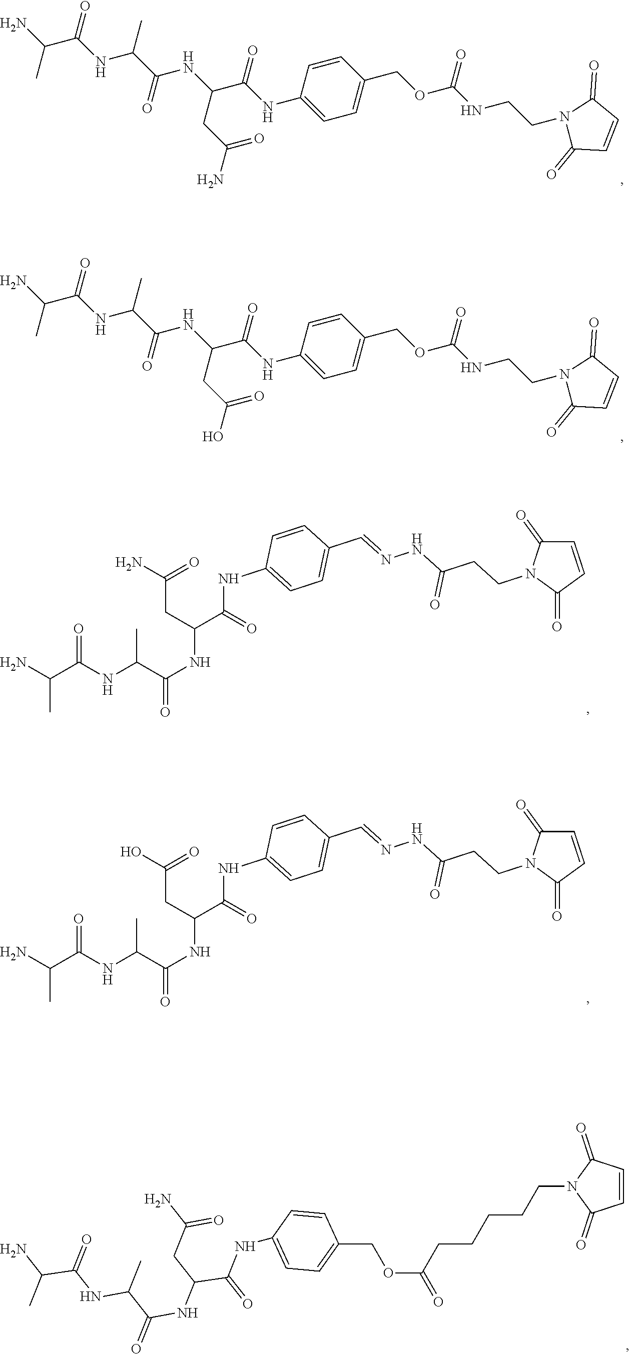 [SMILES: CC(N)C(=O)NC(C)C(=O)NC(CC(N)=O)C(=O)Nc1ccc(COC(=O)NCCN2C(=O)C=CC2=O)cc1 ; CC(N)C(=O)NC(C)C(=O)NC(CC(=O)O)C(=O)Nc1ccc(COC(=O)NCCN2C(=O)C=CC2=O)cc1 ; CC(N)C(=O)NC(C)C(=O)NC(CC(N)=O)C(=O)Nc1ccc(/C=N/NC(=O)CCN2C(=O)C=CC2=O)cc1 ; CC(N)C(=O)NC(C)C(=O)NC(CC(=O)O)C(=O)Nc1ccc(/C=N/NC(=O)CCN2C(=O)C=CC2=O)cc1 ; CC(N)C(=O)NC(C)C(=O)NC(CC(N)=O)C(=O)Nc1ccc(COC(=O)CCCCCN2C(=O)C=CC2=O)cc1]

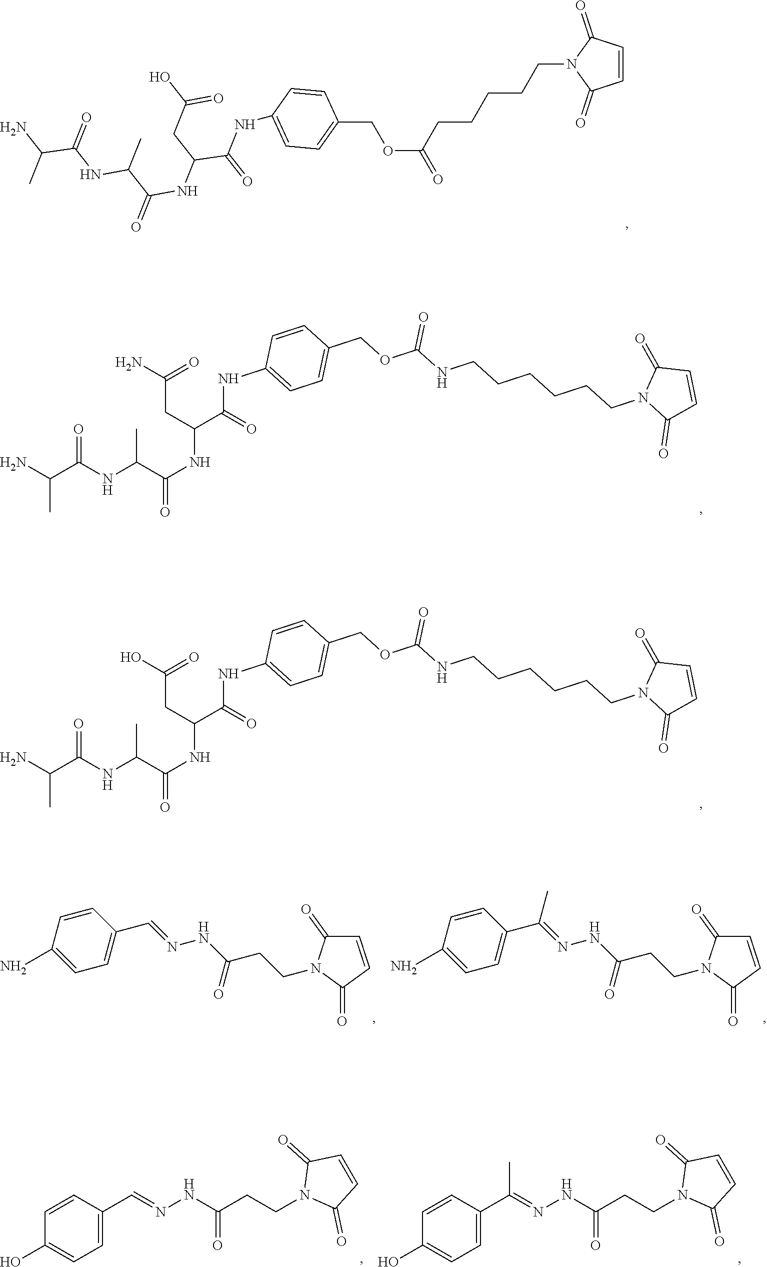

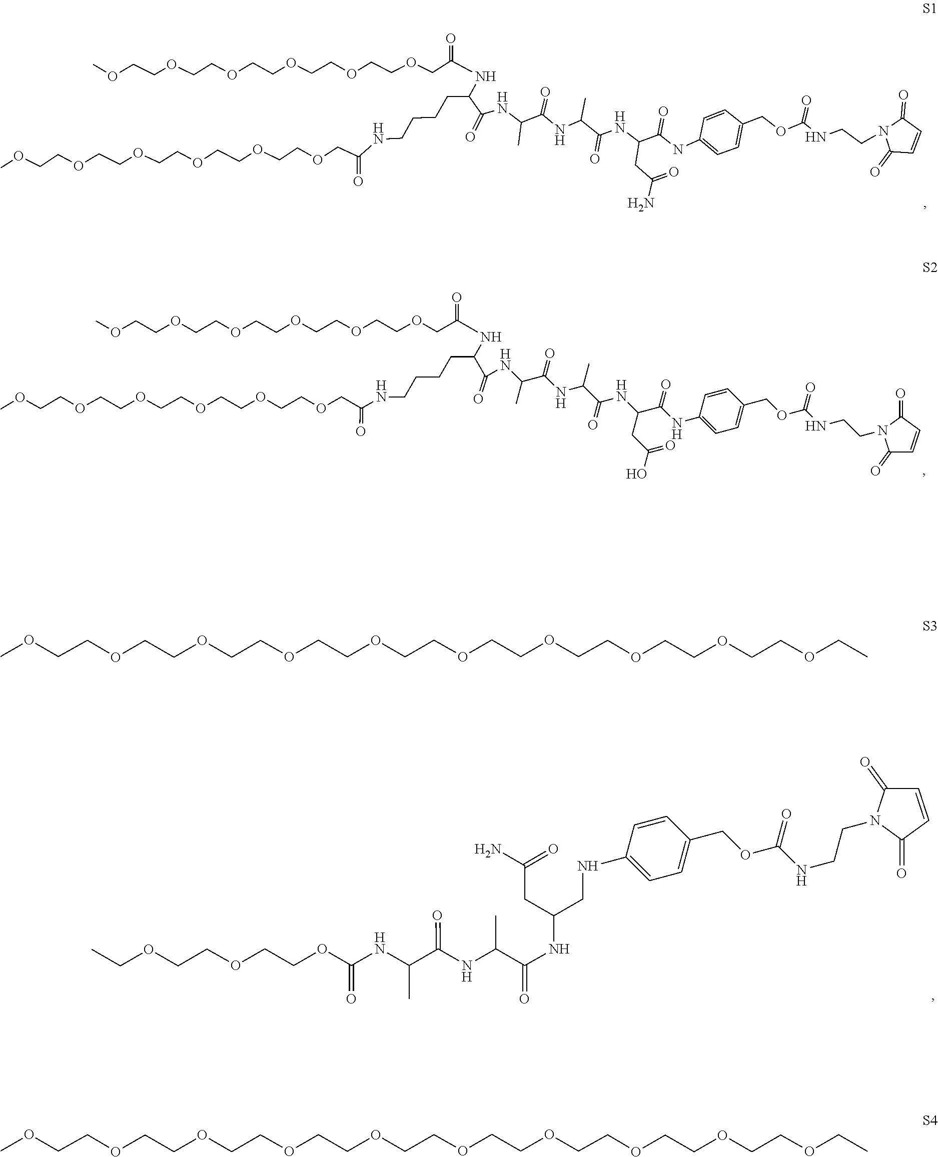

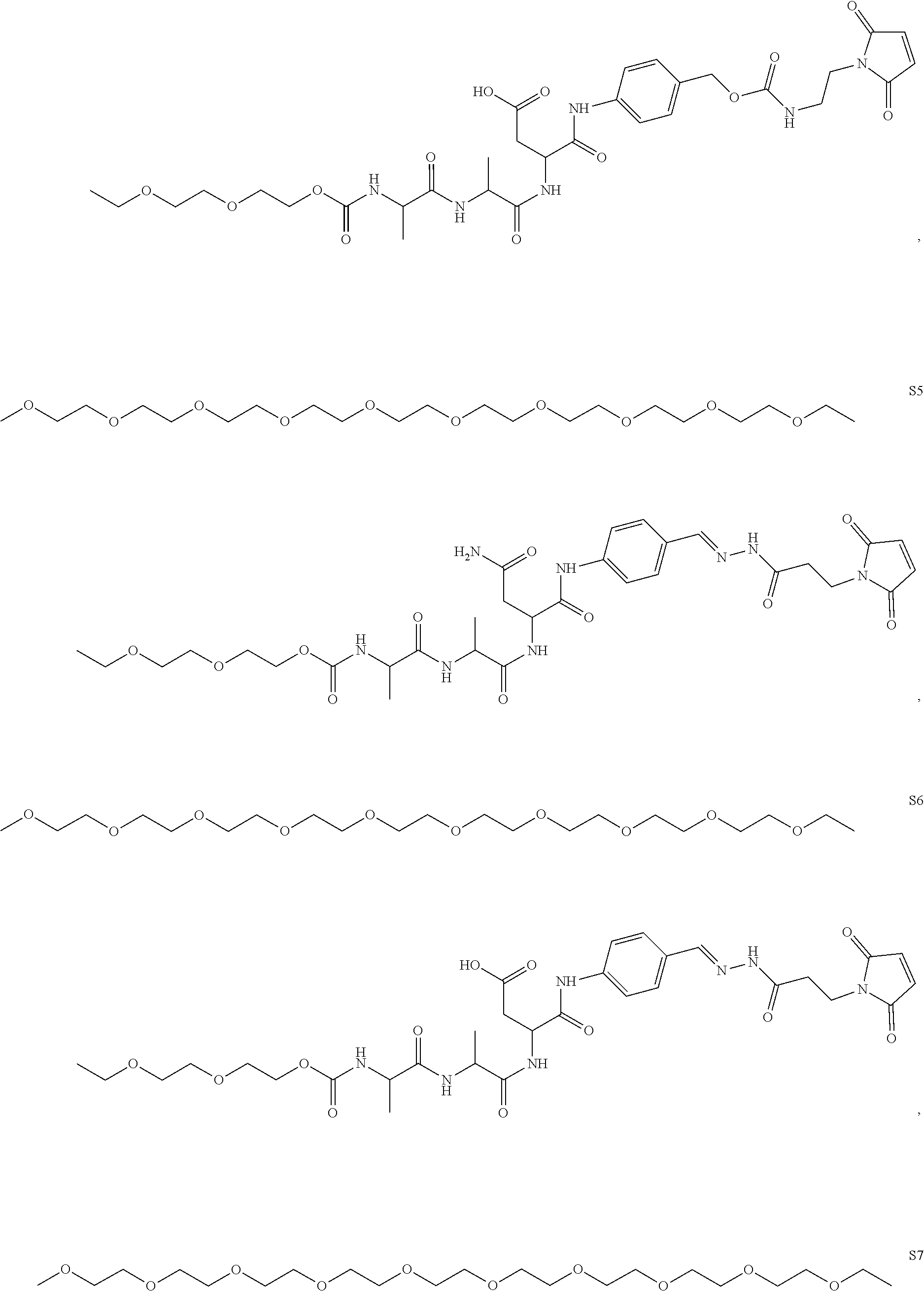

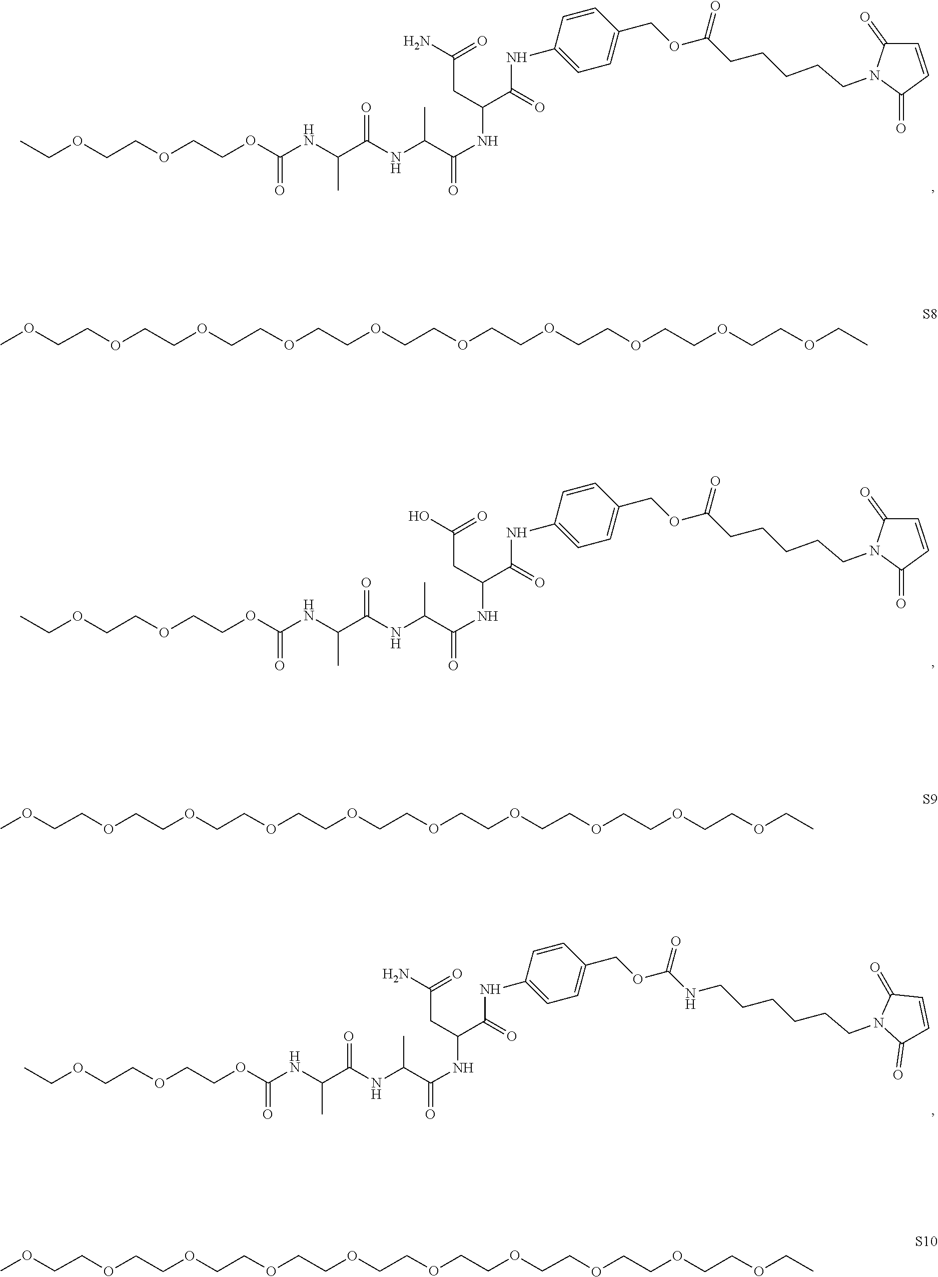

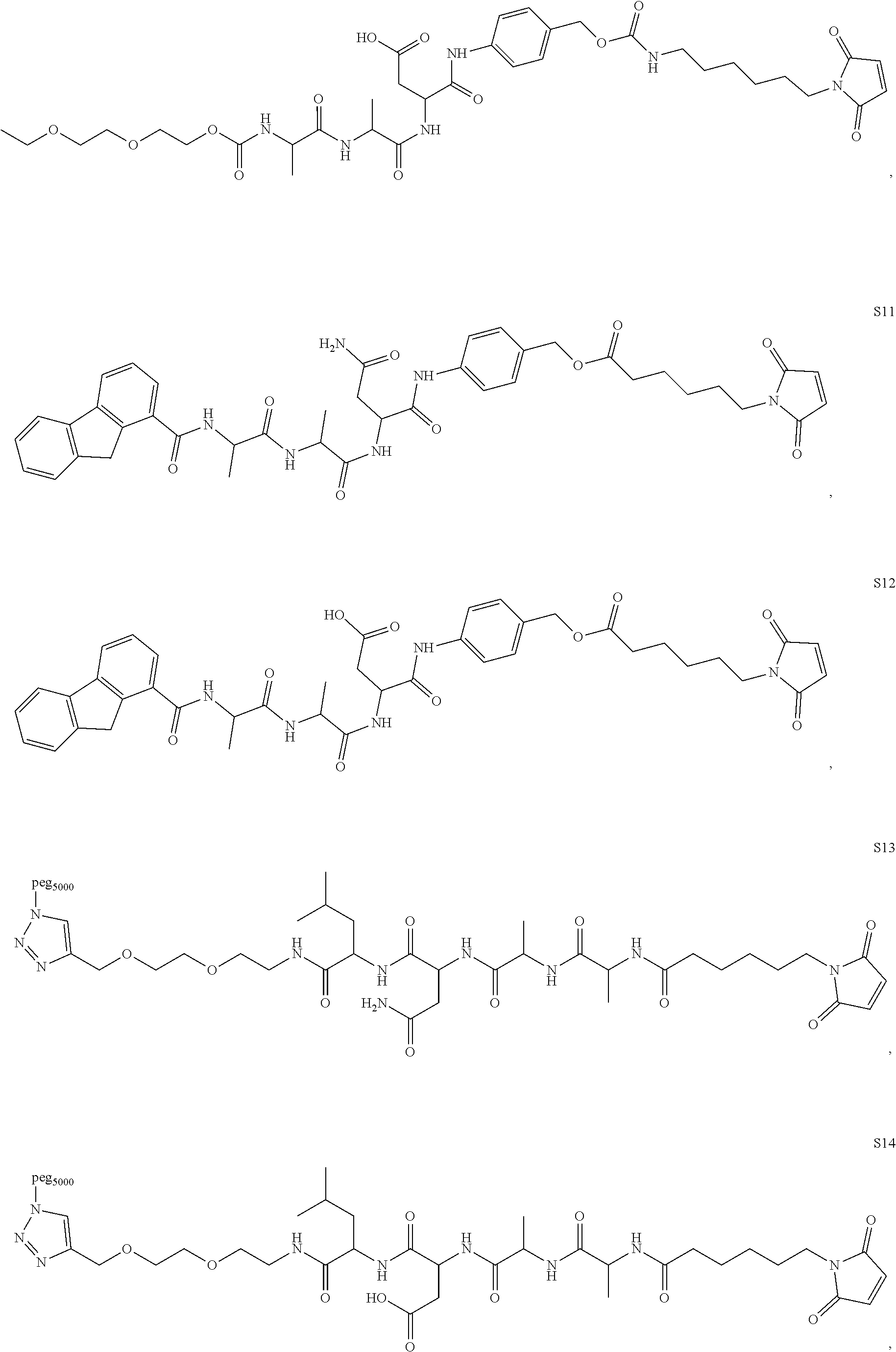

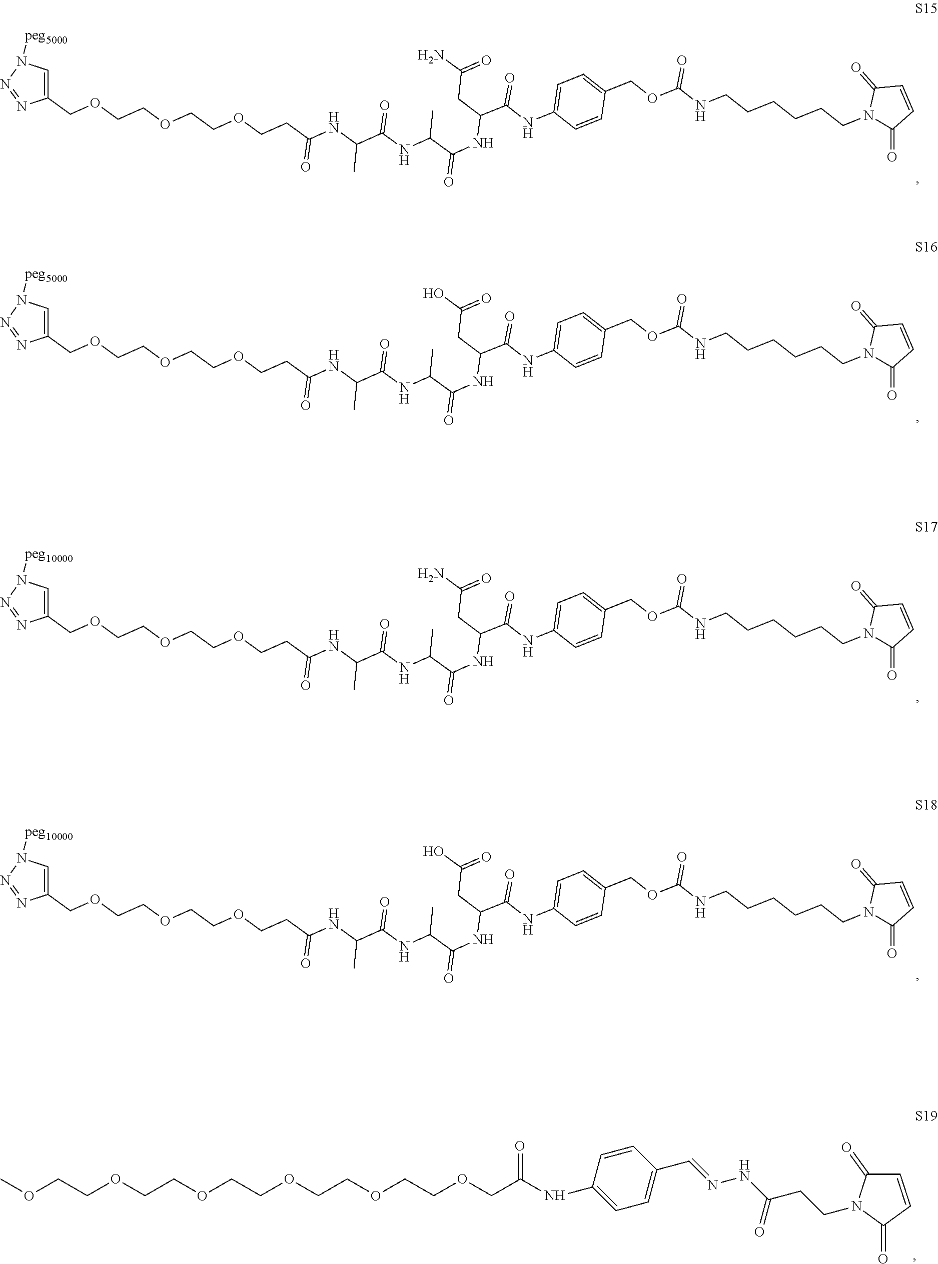

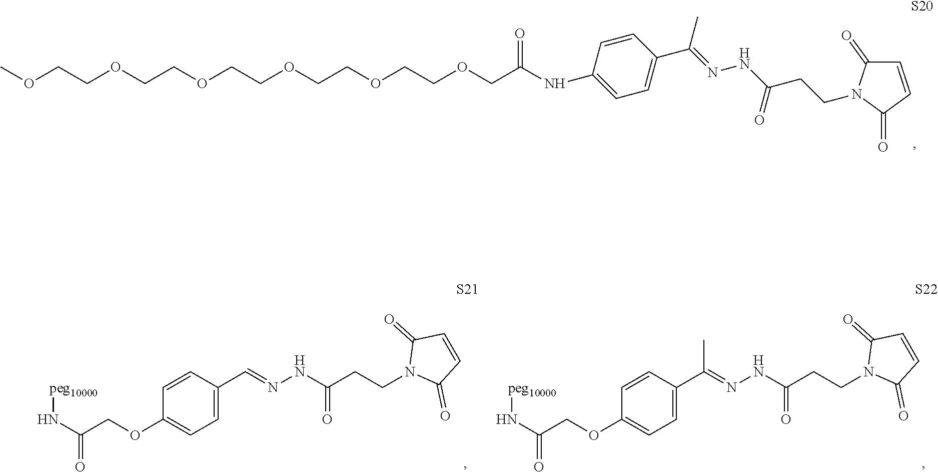

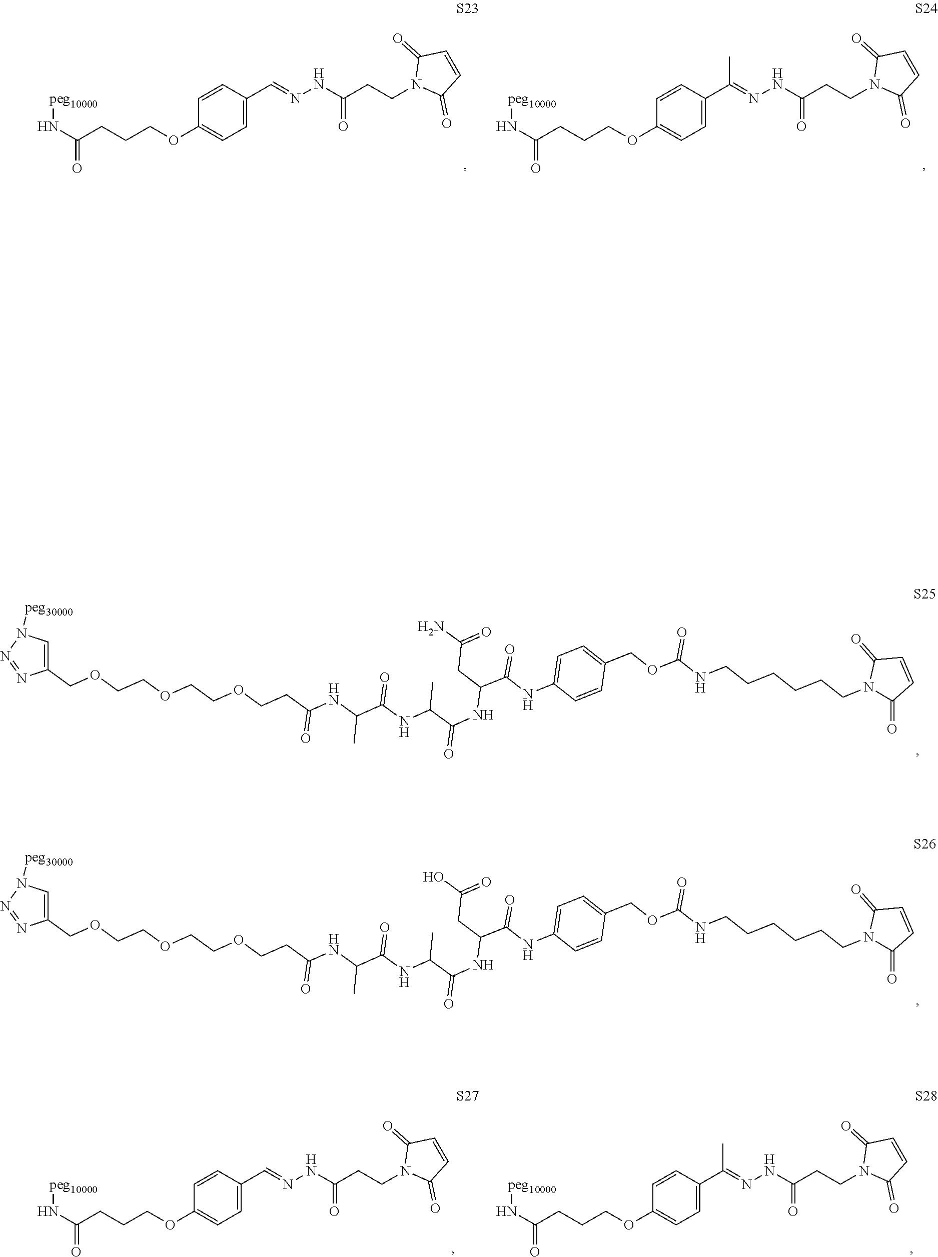

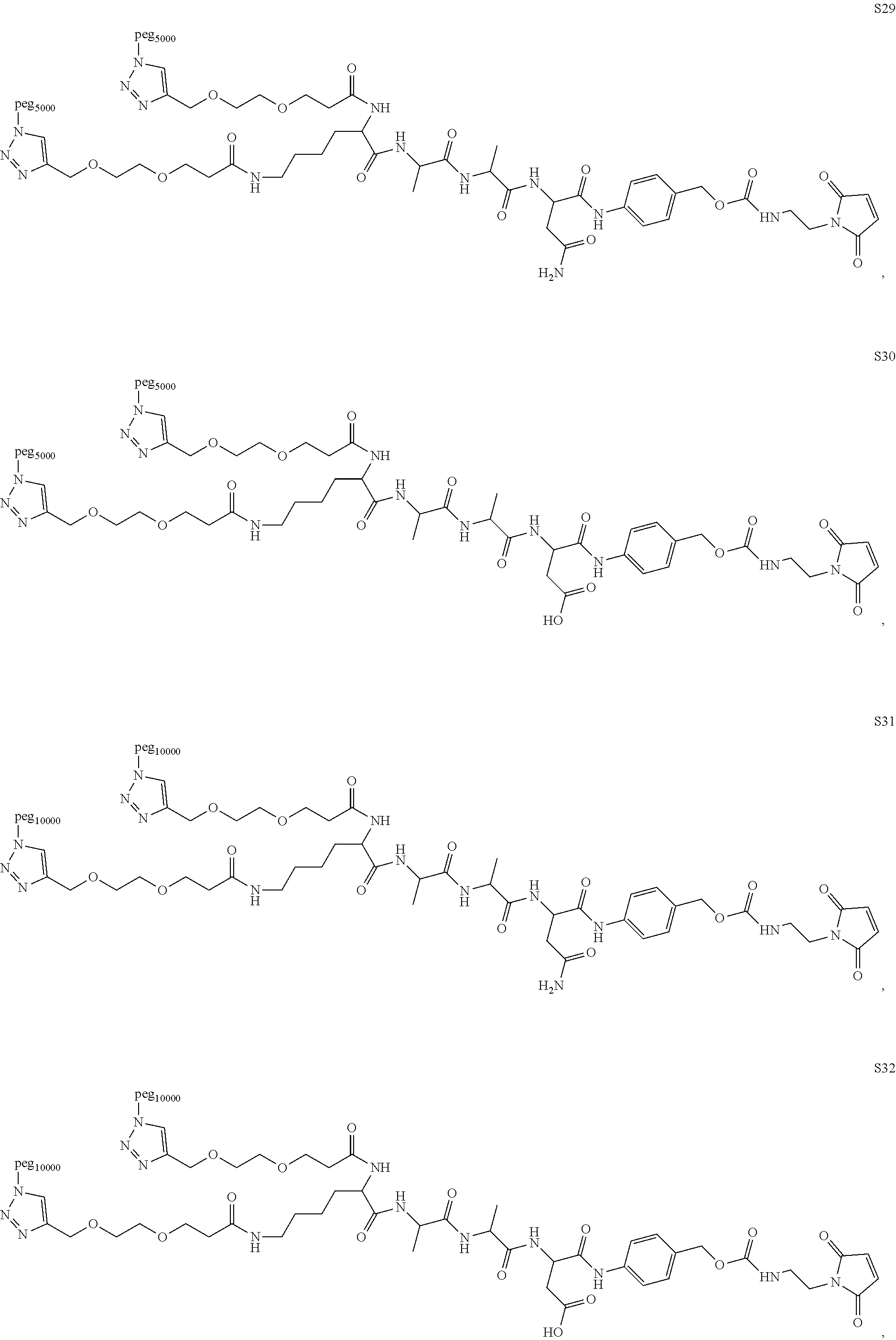

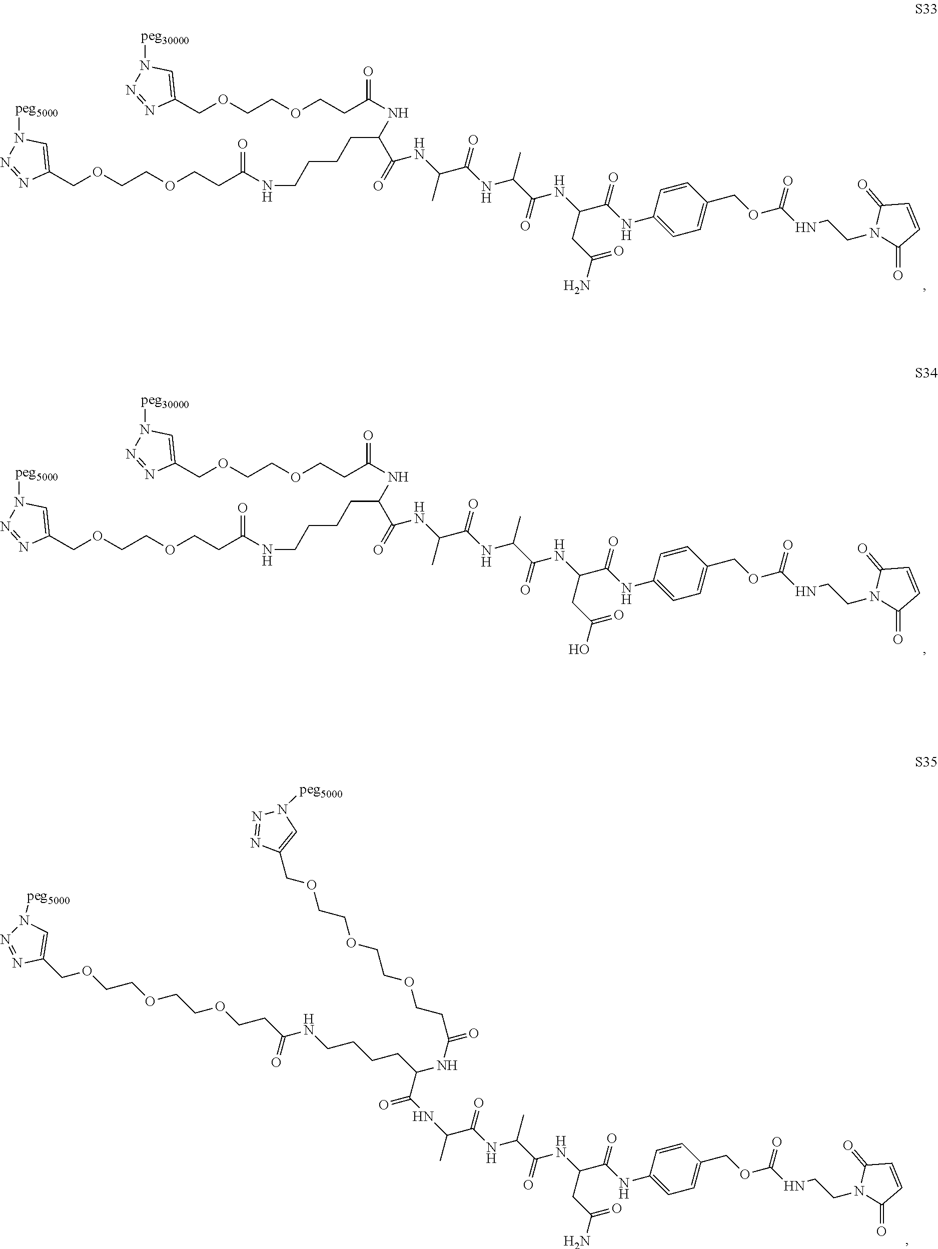

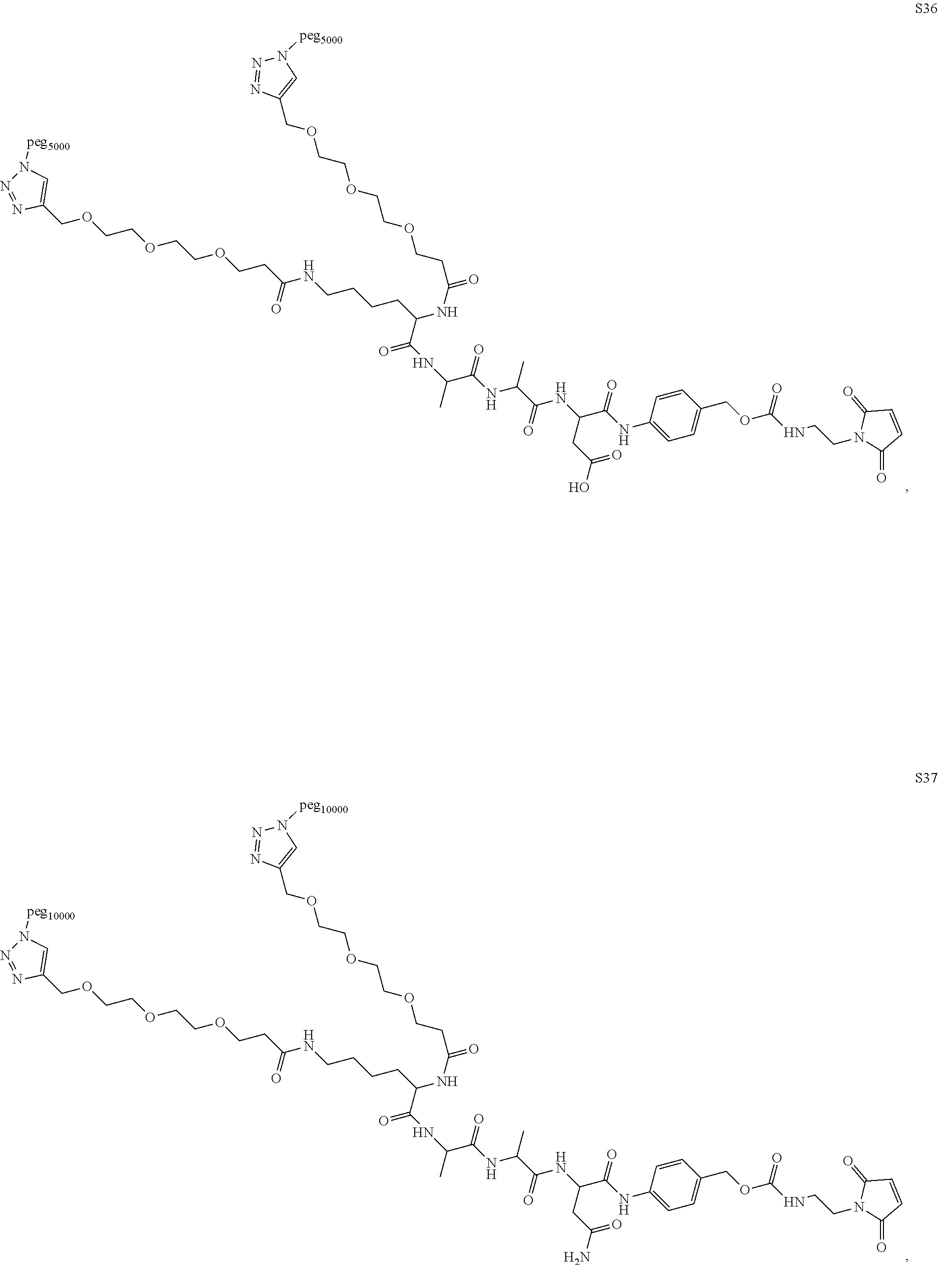

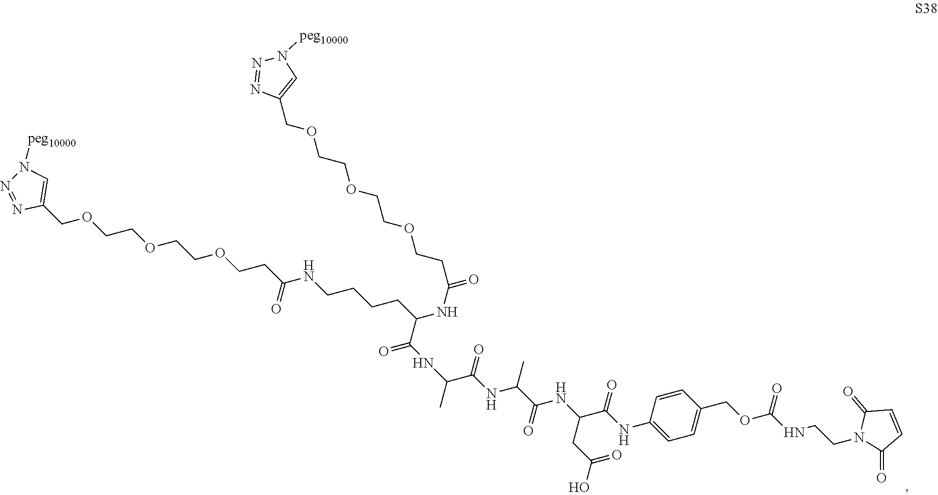

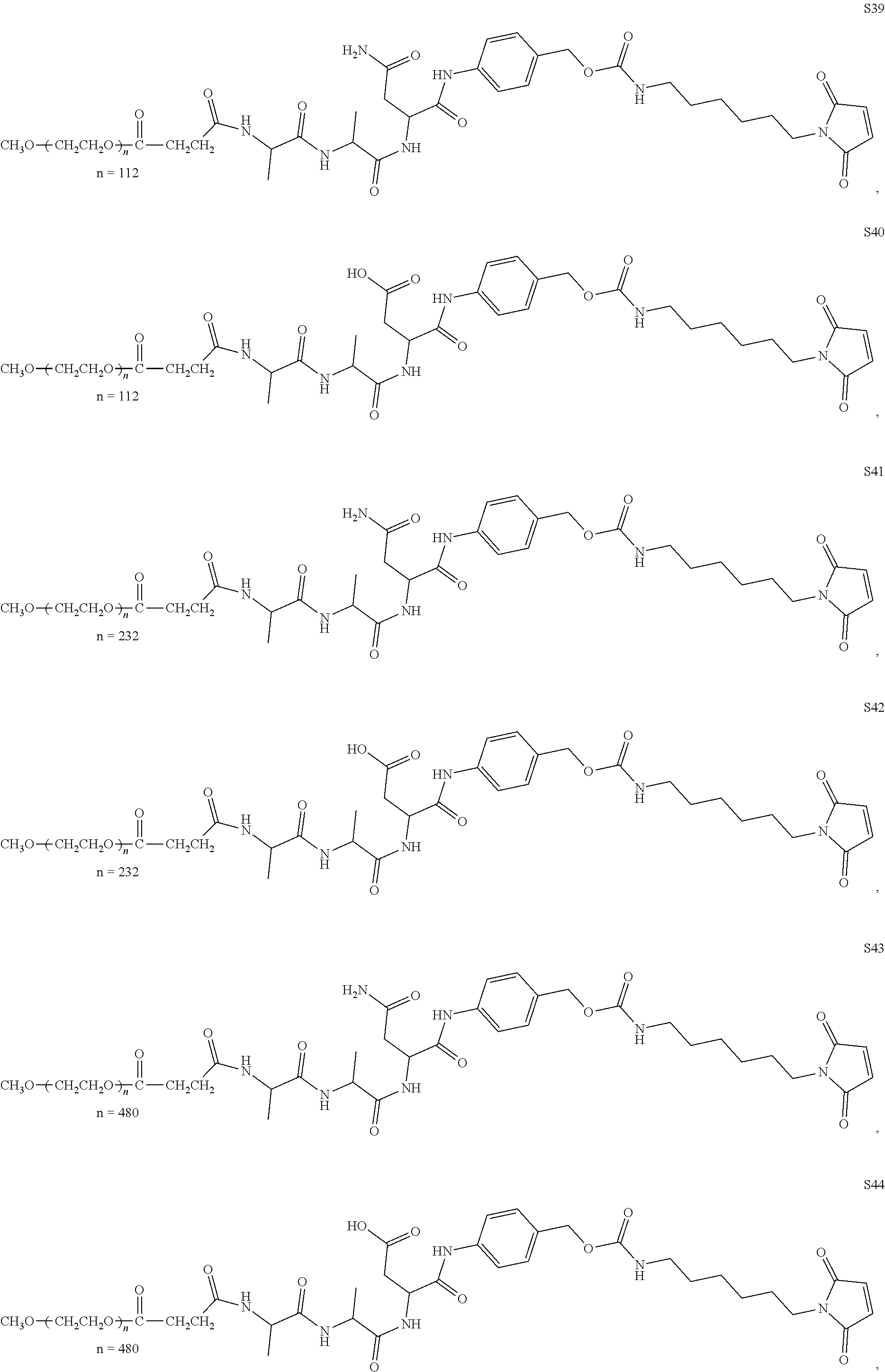

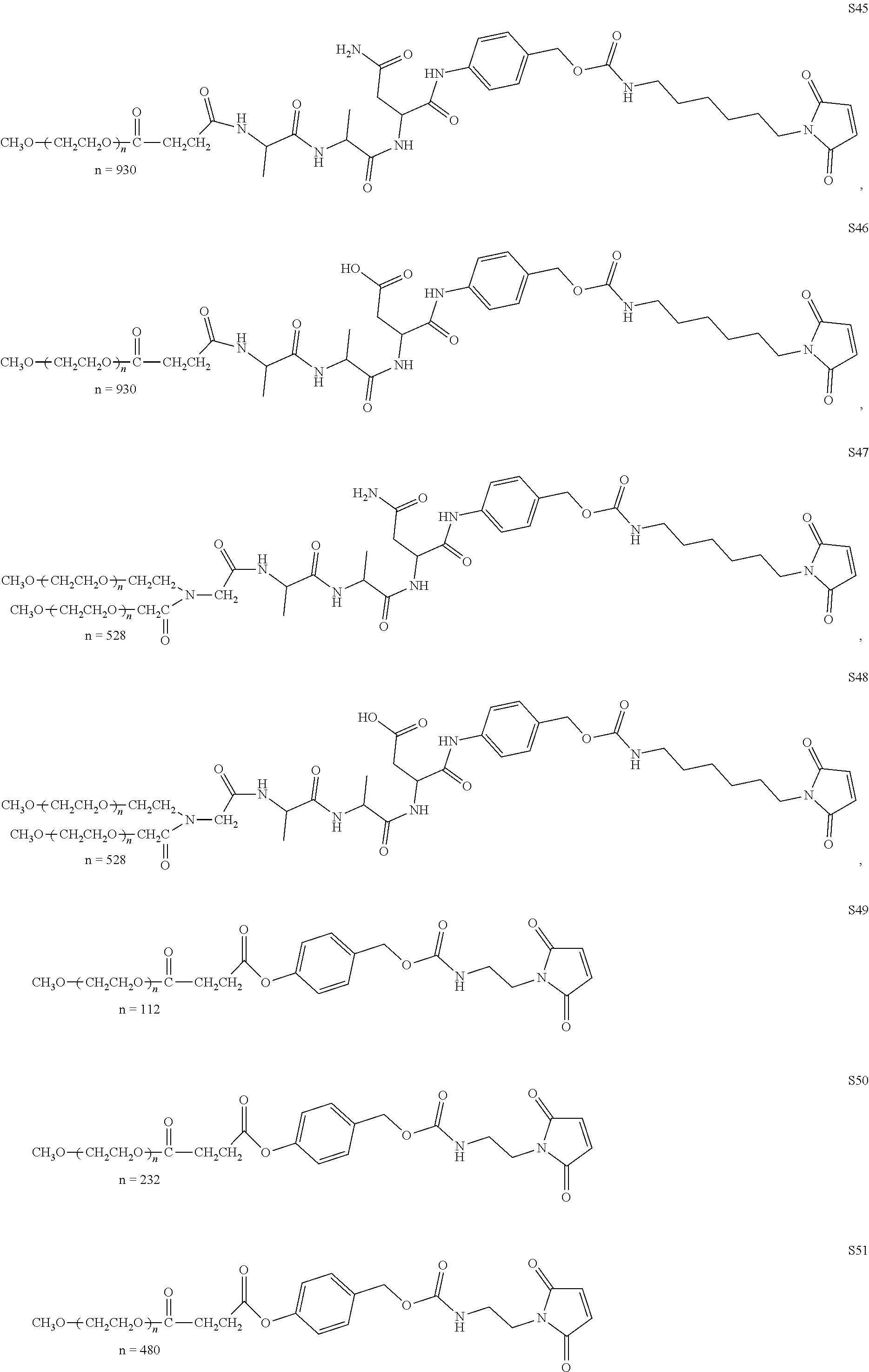

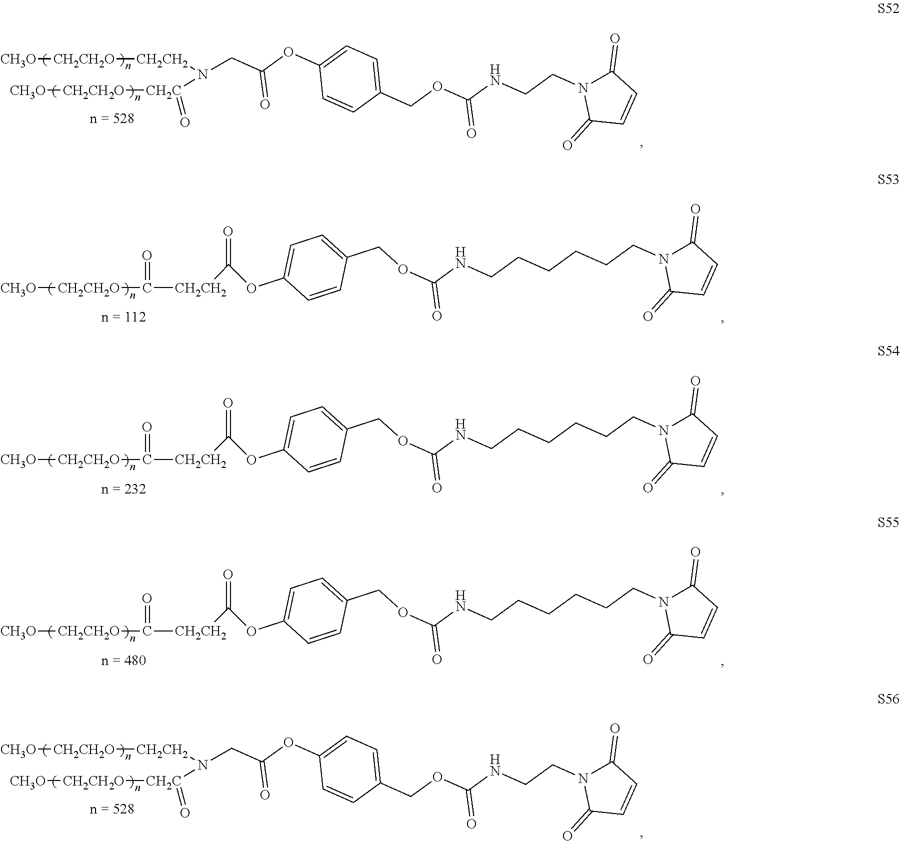

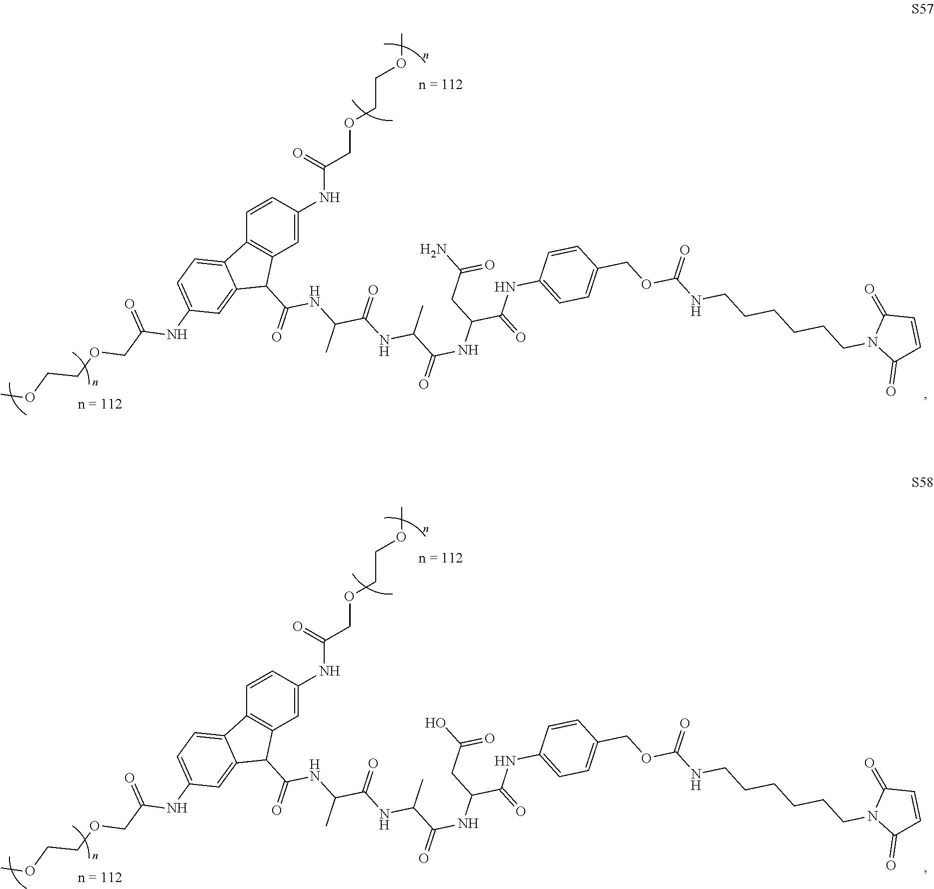

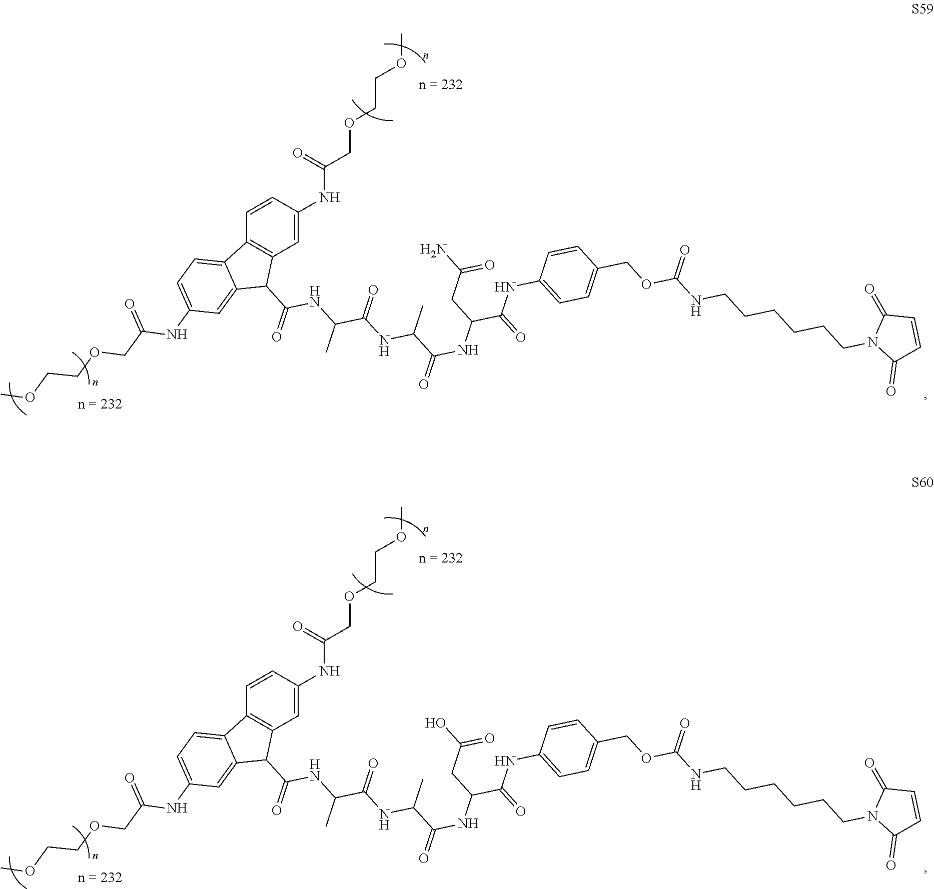

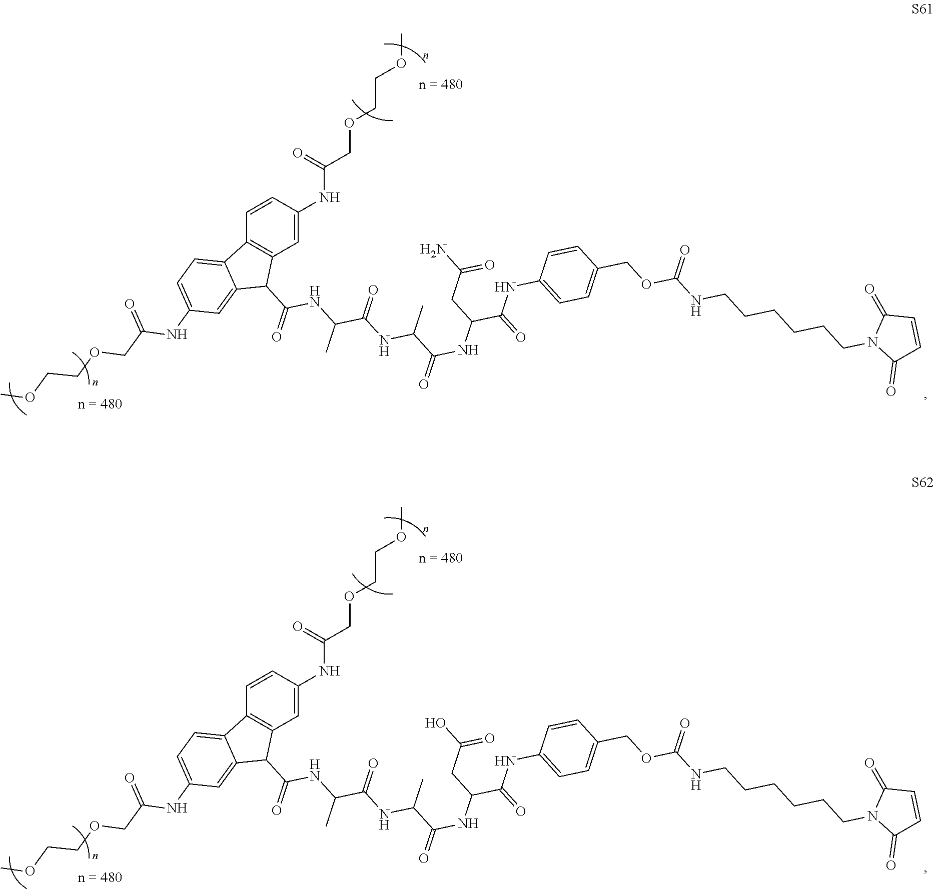

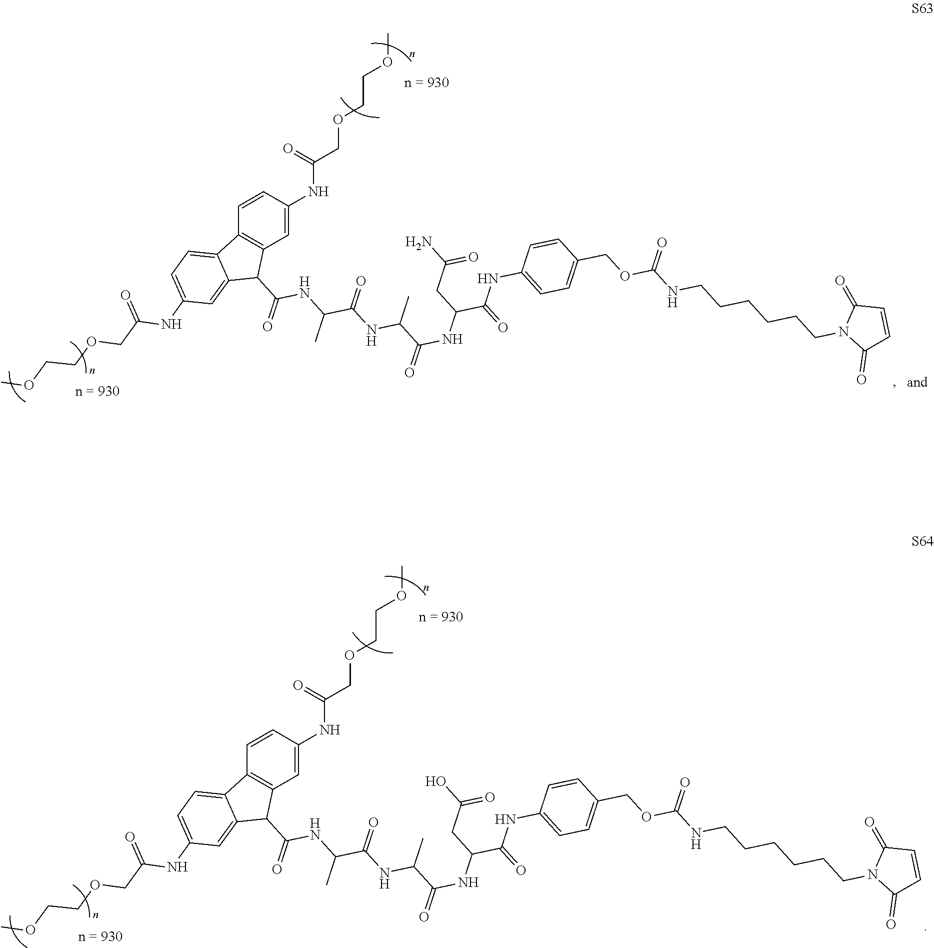

24. An antibody or a functional fragment thereof with one or more of amino acid residues selected from the group consisting of G, A, S, T, L, I, F, E, K, D and Y in the complementarity determining region and/or one or more of amino acid residues selected from the group consisting of G, A, S, T, L, I, F, E, K, D and Y in the non-complementarity determining region mutated to cysteine;
the antibody is selected from the group consisting of anti-Her2 antibody, anti-EGFR antibody, anti-VEGFR antibody, anti-CD20 antibody, anti-CD33 antibody, anti-PD-L1 antibody, anti-PD-1 antibody, anti-CTLA-4 antibody, anti-TNFα antibody, anti-CD28 antibody, anti-4-1BB antibody, anti-OX40 antibody, anti-GITR antibody, anti-CD27 antibody, antib-CD40 antibody, or anti-ICOS antibody, anti-CD25 antibody, anti-CD30 antibody, anti-CD3 antibody, anti-CD22 antibody, anti-CCR4 antibody, anti-CD38 antibody, anti-CD52 antibody, anti-Complement C5 antibody, anti-F protein of RSV, anti-GD2 antibody, anti-GITR antibody, anti-Glycoprotein receptor lib/Illa antibody, anti-ICOS antibody, anti-IL2R antibody, anti-LAG3 antibody, anti-Integrinα4 antibody, anti-lgE antibody, anti-PDGFRα antibody, anti-RANKL antibody, anti-SLAMF7 antibody, anti-LTIGIT antibody, anti-TIM-3 antibody, anti-VEGFR2 antibody, anti-VISTA antibody; or
the antibody is selected from the group consisting of Utomilumab, Urelumab, ADG106, Poteligeo™ (Mogamulizumab), Poteligeo™ (Mogamulizumab), Bexxar™ (tositumomab), Zevalin™ (ibritumomab tiuxetan), Rituxan™ (rituximab), Arzerra™ (Ofatumumab), Gazyva™ (Obinutuzumab), Besponsa™ (Inotuzumab ozogamicin), Zenapax™ (daclizumab), Varlilumab, Theralizumab, Adcetris™ (Brentuximab vedotin), Myelotarg™ (gemtuzumab), Darzalex™ (Daratumumab), CDX-1140, SEA-CD40, R07009789, JNJ-64457107, APX-005M, Chi Lob 7/4, Campath™ (alemtuzumab), Raptiva™ (efalizumab), Soliris™ (eculizumab), Yervoy™ (ipilimumab), tremelimumab, Erbitux™ (cetuximab), Vectibix™ (panitumumab), Portrazza™ (Necitumumab), TheraCIM™ (Nimotuzumab), Synagis™ (palivizumab), Unituxin™ (Dinutuximab), TRX-518, MK-4166, MK-1248, GWN-323, INCAGN0186, BMS-986156, AMG-228, ReoPro™ (abiciximab), Herceptin™ (trastuzumab), Perjeta™ (Pertuzumab), Kadcyla™ (Ado-trastuzumab emtansine), GSK-3359609, JTX-2011, Simulect™ (basiliximab), Tysabri™ (natalizumab), BMS-986016, REGN3767, LAG525, Xolair™ (omalizumab), Tavolimab, PF-04518600, BMS-986178, MOXR-0916, GSK-3174998, INCAGN01949, IBI-101, Keytruda™ (Pembrolizumab), Opdivo™ (Nivolumab), Lartruvo™ (Olaratumab), Tencentriq™ (Atezolizumab), BMS-936559, Bavencio™ (Avelumab), Imfinzi™ (Duralumab), Prolia™ (Denosumab), Empliciti™ (Elotuzumab), MTIG7192A, TSR-022, MBG-453, Remicade™ (infliximab), Humira™ (adalimumab), Avastin™ (bevacizumab), Lucentis™ (ranibizumab), Cyramza™ (Ramucirumab), and JNJ-61610588.

25. A cytokine with one or more amino acid residues selected from the group consisting of G, A, S, T, L, I, F, E, K, D and Y mutated to cysteine;
the cytokine is selected from the group consisting of IL-2, IL-7, IL-10, IL-11, IL-12, IL-15, IL-21, IFN-α, IFN-β, IFN-γ, G-CSF, GM-CSF, TNF-α, TRAP and TRAIL; or
the cytokine contains one or more of the mutations:
the mutation position in IL2 is selected from the group consisting of: Thr3, Lys32, Lys35, Thr37, Met39, Thr41, Lys43, Tyr45, Lys48, Lys49, Lys64, Leu72, Ala73, Ser75, Lys76, Leu94, Thr101, Thr102, Tyr107, Ala108, Thr11, Ala112, Cys125, Leu12, His16, Leu19, Met23, Gly27, Ser75, Arg81, Leu85, Ser87 and Asn88; or
the mutation position in IL10 is selected from the group consisting of: Thr6, Ser8, Ser11. Thr13, Gly17, Arg24, Ser31, Arg32, Lys34, Thr35, Lys40, Leu46, Lys49, Ser51, Lys57, Gly58, Ser66, Tyr72, Lys88, His90, Ser93, Lys99, Thr100, Arg104, Lys117, Ser118, Lys119, Lys125, Lys130, Lys134, Gly135, Tyr137, Tyr149, Thr155, Lys157 and Arg159; or
the mutation position in a heavy chain of the anti-PD-1 antibody Pembrolizumab is selected from the group consisting of: Ser7, Gly8, Gly15, Ala16, Ser17, Ala24, Ser25, Gly26, Tyr27, Thr28, Thr30, Asn31, Tyr32, Tyr33, Tyr35, Ala40, Gly42, Gly44, Leu45, Gly49, Gly50, Ile51, Asn52, Ser54, Asn55, Gly56, Gly57, Thr58, Asn59, Lys63, Lys65, Thr69, Leu70, Thr71, Thr72, Asp73, Ser74, Ser75, Thr76, Thr77, Thr78, Ala79, Leu83, Ser85, Leu86, Thr91, Ala92, Arg99, Asp100, Tyr101, Arg102, Asp104, Gly106, Gly111, Gly113, Thr114, 115Thr, 117Thr, Ser119, Ser120, Ala121, Ser122, Thr123, Lys124, Gly125 and Ser127; the mutation position in a light chain is selected from the group consisting of: Ile2, Thr5, Ser7, Ala9, Thr10, Leu11, Ser12, Leu13, Ser14, Gly16, Ala19, Thr20, Ala25, Ser26, Lys27, Gly28, Ser30, Thr31, Ser32, Gly33, Tyr34, Ser35, Tyr36, Leu37, Gly45, Ala47, Leu50, Leu51, Ile52, Tyr53, Leu54, Ala55, Ser56, Tyr57, Leu58, Ser60, Gly61, Ala64, Ser67, Gly68, Ser69, Gly70, Ser71, Gly72, Thr73, Ala76, Thr78, Ser80, Ser81, Ser95, Arg96, Asp97, Leu98, Leu100, Thr101, Phe102, Gly104, Ile110, Lys111 and K130; or
the mutation position in a heavy chain of the anti-PD-1 antibody Nivolumab is selected from the group consisting of: Gln3, Ser7, Gly8, Gly9, Gly10, Gly15, Ser17, Lys23, Ala24, Ser25, Gly26, Ile27, Asn31, Thr28, Ser30, Ser32, Gly33, Ala40, Gly42, Gly44, Leu45, Ala49, Ile51, Tyr53, Asp54, Gly55, Ser56, Lys57, Tyr59, Tyr60, Ala61, Asp62, Ser63, Lys65, Gly66, Thr69, Ile70, Ser71, Arg72, Asp73, Asn74, Ser75, Lys76, Asn77, Thr78, Leu79, Leu81, Ser85, Leu86, Ala88, Thr91, Ala92, Thr98, Asn99, Asp100, Asp101, Tyr102, Gly104, Gly106, Thr107, Leu108, Thr110, Ser112, Ser113, Ala114, Ser115, Thr116, Lys117, Gly118 and Ser120; the mutation position in a light chain is selected from the group consisting of: Ile2, Leu4, Thr5, Ser7, Ala9, Thr10, Leu11, Ser12, Leu13, Ser14, Gly16, Ala19, Thr20, Leu21, Ala25, Ser26, Ser28, Ser30, Ser31, Tyr32, Leu33, Ala34, Tyr36, Gly41, Ala43, Leu46, Leu47, Ile48, Tyr49, Asp50, Ala51, Ser52, Asn53, Arg54, Ala55, Thr56, Gly57, Ile58, Ala60, Arg61, Ser63, Gly64, Ser65, Gly66, Ser67, Gly68, Thr69, Thr72, Leu73, Thr74, Ile75, Ser76, Ser77, Leu78, Ala84, Ser91, Ser92, Asn93, Arg96, Thr97, Phe98, Gly99, Gly101, Thr102, Ile106, Lys107, Thr109, Ala111, Ala112, Ser114, Ile117 and Ser121;
the mutation position in a heavy chain of the anti-CTLA-4 antibody Ipilimumab is selected from the group consisting of: Gln3, Arg19, Leu20, Ser25, Gly26, Phe27, Thr28, Phe29, Ser30, Ser31, Tyr32, Thr33, Met34, His35, Gly44, Phe50, Ile51, Ser52, Tyr53, Asp54, Gly55, Asn56, Asn57, Lys58, Tyr59, Tyr60, Thr69, Ser71, Arg72, Asp73, Asn74, Ser75, Lys76, Asn77, Thr99, Gly100, Trp101, Leu102, Gly103 and Pro104; the mutation position in a light chain is selected from the group consisting of: Gln6, Arg24, Ala25, Ser26, Gln27, Ser28, Val29, Gly30, Ser31, Ser32, Tyr33, Ile49, Tyr50, Gly51, Ala52, Phe53, Ser54, Arg55, Ala56, Phe53, Ser54, Arg55, Ala56, Thr57, Gly58, Ile59, Pro60, Asp61, Arg62, Ser68, Gly69, Thr70, Gln90, Gln91, Tyr92, Gly93, Ser94, Ser95, Pro96 and Trp 97;
the mutation position in a heavy chain of the anti-TNFα antibody is selected from the group consisting of: Ser7, Gly8, Gly9, Gly10, Leu11, Gly15, Ser17, Leu18, Leu20, Ala24, Ser25, Gly26, Thr28, Asp30, Asp31, Tyr32, Ala33, Ala40, Gly42, Gly44, Leu45, Ser49, Ala50, Ile51, Thr52, Asn54, Ser55, Gly56, Ile58, Asp59, Tyr60, Ala61, Asp62, Ser63, Glu65, Gly66, Phe68, Thr69, Ile70, Ser71, Asp73, Asn74, Ala75, Lys76, Ser78, Leu79, Tyr80, Leu81, Ser85, Leu86, Ala88, Thr91, Ala92, Lys98, Ser100, Tyr101, Leu102, Ser103, Thr104, Ala105, Ser106, Ser107, Leu108, Asp109, Tyr110, Gly112, Gly114, Thr115, Leu116, thr118, Ser120, Ser121, Ala122, Ser123 and Thr124; the mutation position in a light chain is selected from the group consisting of: Asp1, Thr5, Ser7, Ser9, Ser10, Leu11, Ser12, Ala13, Ser14, Gly16, Thr20, Ile21, Ala25, Ser26, Gln27, Gly28, Ile29, Arg30, Asn31, Tyr32, Leu33, Ala34, Tyr36, Lys39, Gly41, Lys42, Ala43, Leu48, Leu47, Ile48, Tyr49, Ala50, Ala51, Ser52, Thr53, Leu54, Gln55, Ser56, Gly57, Ser60, Ser63, Gly64, Ser65, Gly66, Ser67, Gly68, Thr69, Asp70, Thr72, Leu73, Thr74, Ile75, Ser76, Ser77, Leu78, Ala84, Thr85, Tyr91, Asn92, Arg93, Ala94, Tyr96, Thr97, Phe98, Gly99, Gly101, Thr102, Ile106, Lys107, Thr109 and Ala111;
the mutation position in a heavy chain of the anti-CD28 antibody is selected from the group consisting of: Ser7, Gly8, Gly15, Ala16, Ser17, Ser21, Ala24, Ser25, Gly26, Tyr27, Thr28, Thr30, Ser31, Tyr32, Ala40, Gly42, Gly44, Gly49, Tyr52, Gly54, Thr58, Ala68, Thr69, Thr71, Thr74, Ser75, Ser77, Thr78, Ala79, Ser84, Leu86, Ser88, Thr91, Ala92, Thr97, Ser99, Tyr101, Gly102, Leu103, Gly113, Thr114, Thr115, Thr117, Ser119, Ser120, Ala121, Ser122 and Thr123; the mutation position in a light chain is selected from the group consisting of: Thr5, Ser7, Ser9, Ser10, Ser11, Ser12, Ala13, Ser14, Gly16, Thr20, Thr22, Ala25, Ser26, Ser27, Ile29, Tyr30, Ala43, Leu46, Leu47, Tyr49, Lys50, Ala51, Ser52, Leu54, Thr56, Gly57, Ser60, Ser63, Gly64, Ser65, Gly66, Ser67, Gly68, Thr69, Asp70, Thr72, Thr74, Ser76, Ser77, Ala84, Thr85, Gly91, Thr93, Tyr94, Tyr96, Thr97, Phe98, Gly99, Gly100, Gly101, Thr102, Thr109 and Ala111;
the mutation position in a heavy chain of the anti-4-1BB antibody is selected from the group consisting of: Thr31, Tyr32, Ser35, Lys50, Tyr52, Asp55, Ser56, Tyr57, Thr58, Asn59, Tyr60, Ser61, Gln65, Gly66, Gly99, Tyr100, Gly101, Asp104 and Tyr105; the mutation position in a light chain is selected from the group consisting of: Ser23, Gly24, Asp25, Asn26, Gly28, Asp29, Gln30, Tyr31, Gln49, Asp50, Lys51, Asn52, Arg53, Ser55, Gly56, Thr89, Tyr90, Thr91, Gly92, Gly94 and Ser95; or
the mutation position in a heavy chain of the anti-Her2 antibody is selected from the group consisting of: Arg19, Lys30, Asp31, Tyr33, Arg50, Tyr62, Asn55, Tyr57, Arg59, Tyr60, Asp62, Lys65, Asp102 and Tyr105; the mutation position in a light chain is selected from the group consisting of: Asp1, Gln3, Gln27, Asp28, Asn30, Tyr49, Tyr55, Arg66, Asp70 and Tyr92.
the mutation position in a heavy chain of the anti-PD-L1 antibody Atezolizumab is selected from the group consisting of: Gln3, Asp31, Tyr54, Tyr59, Tyr60, Asp62, Lys65, Asp73, Lys76, Asn77 and Arg99; the mutation position in a light chain is selected from the group consisting of: Arg24, Gln27, Asp28, Tyr49, Tyr55, Asp70, Gln89, Gln90, Tyr91 and Tyr93.

26. A conjugate of a biomolecule of the structure: 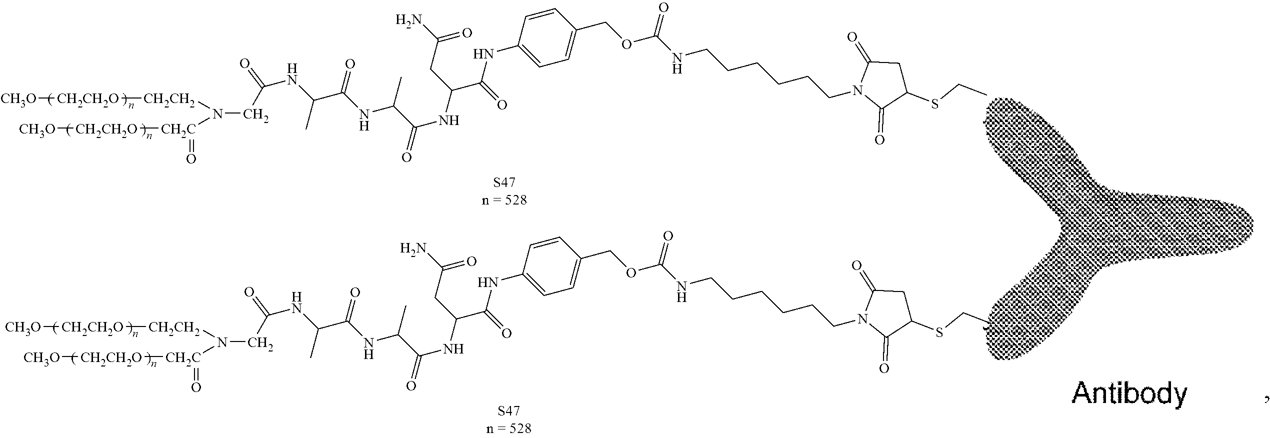 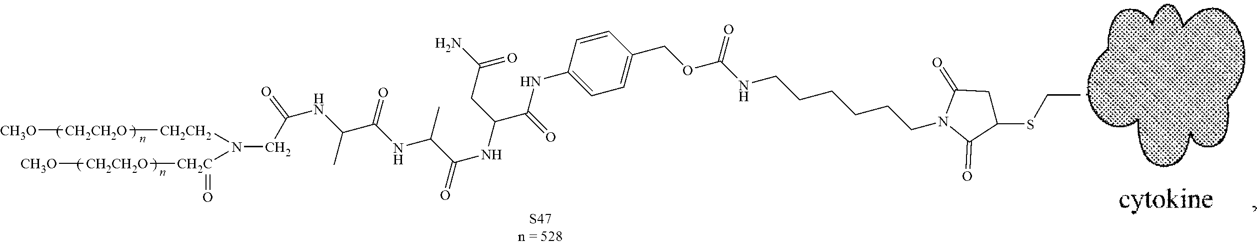 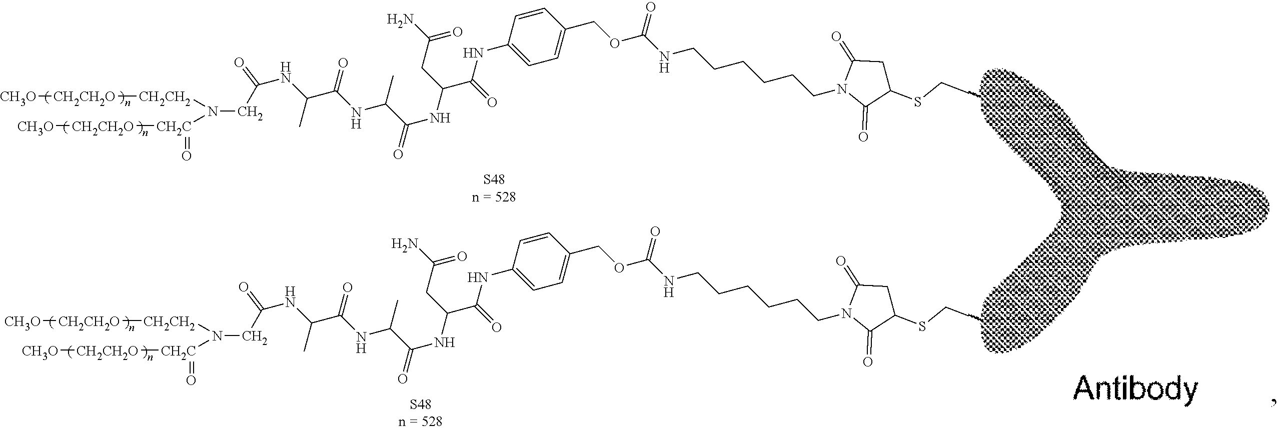 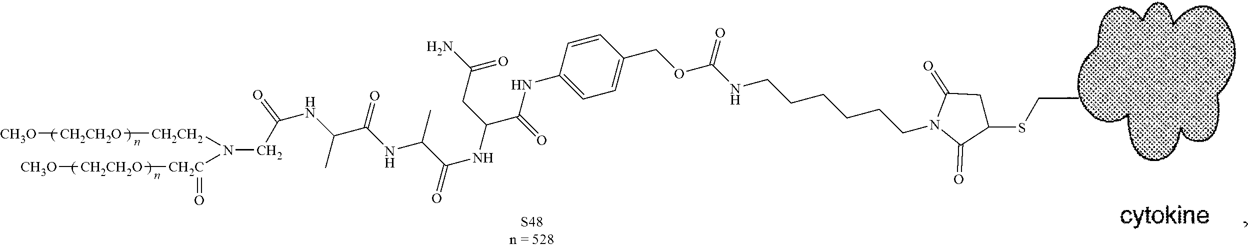 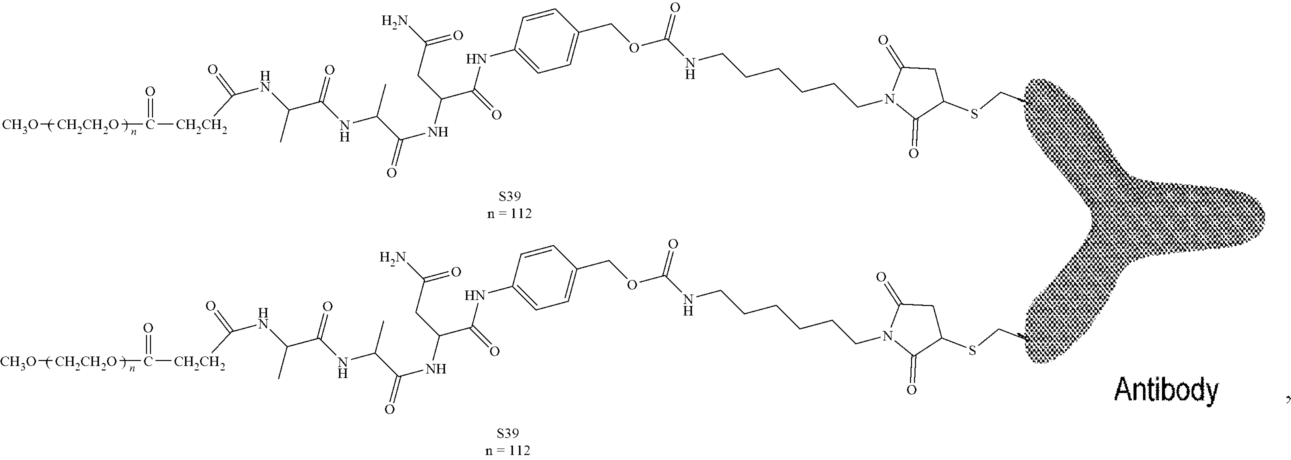 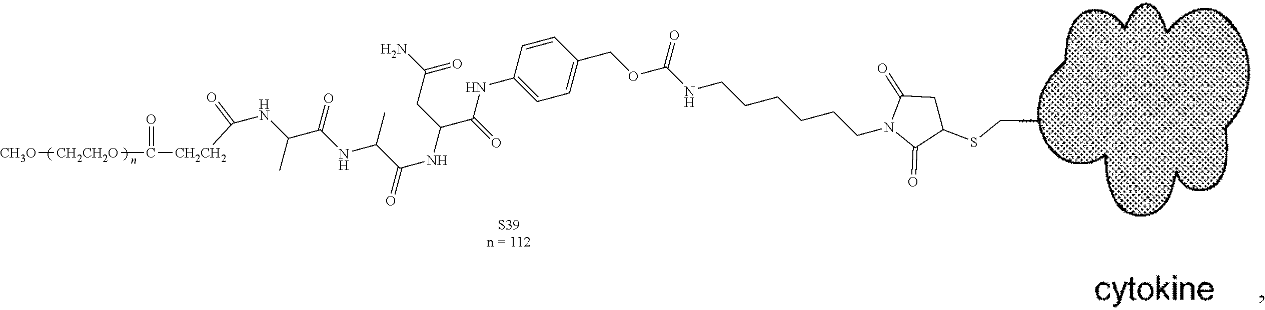 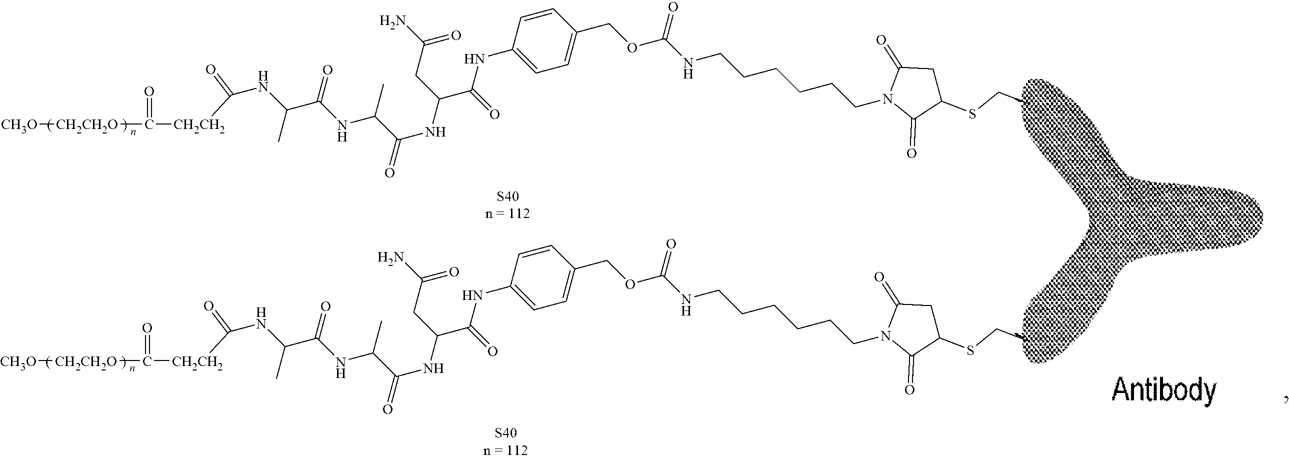 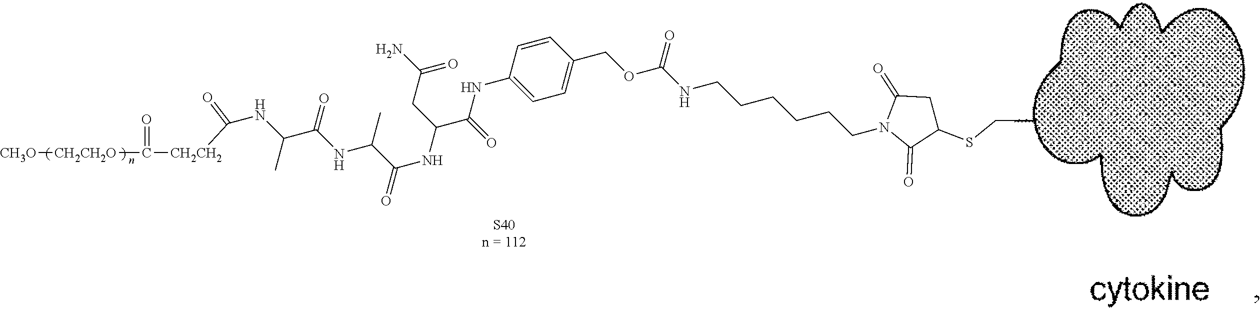 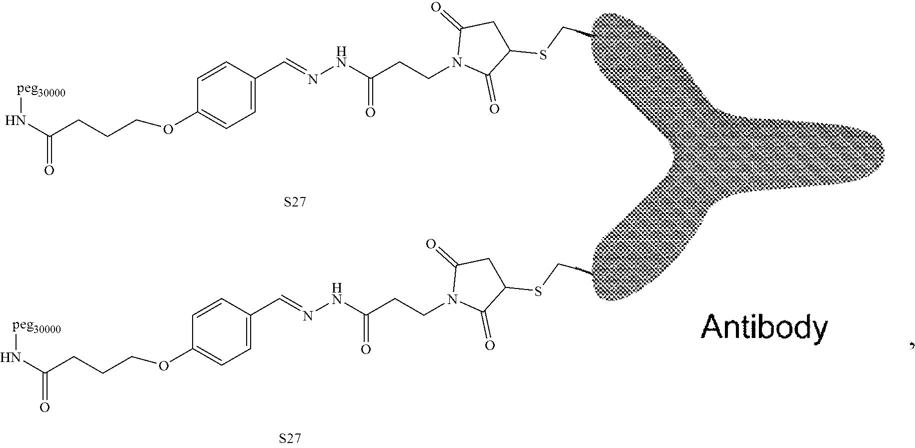 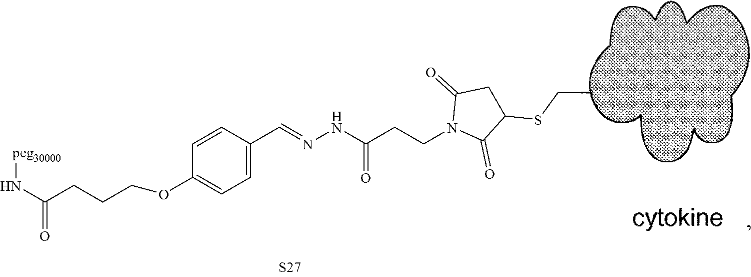 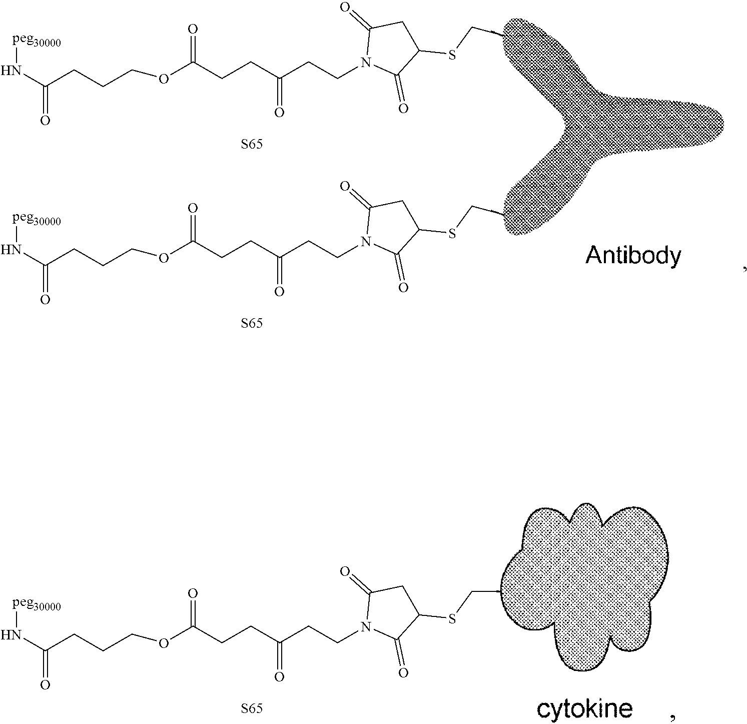 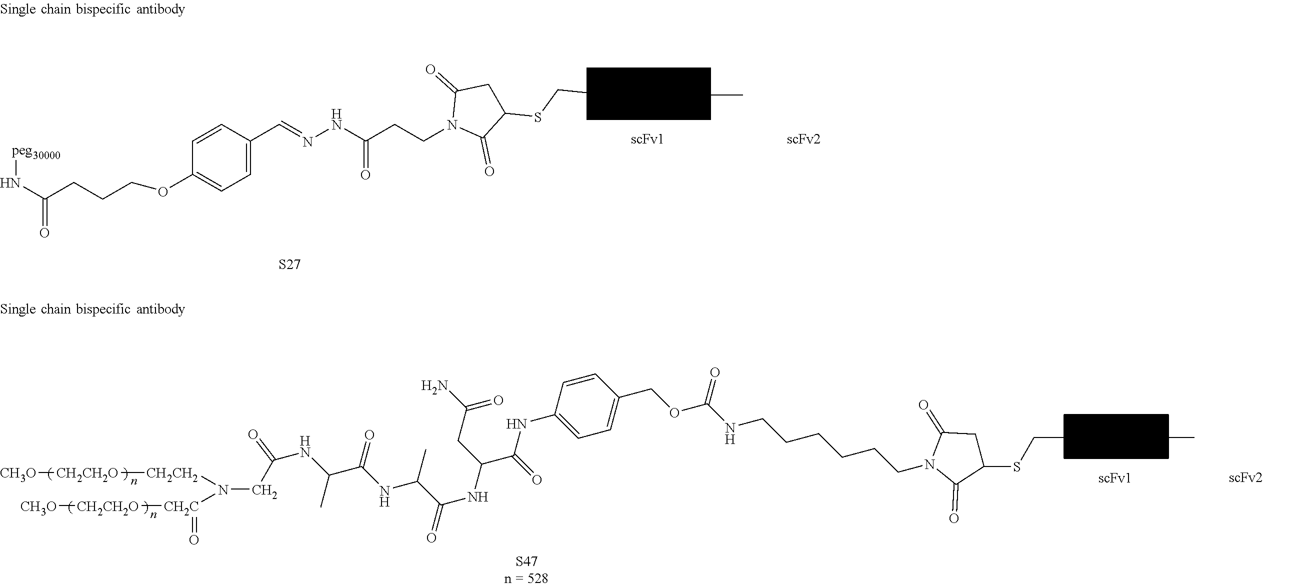 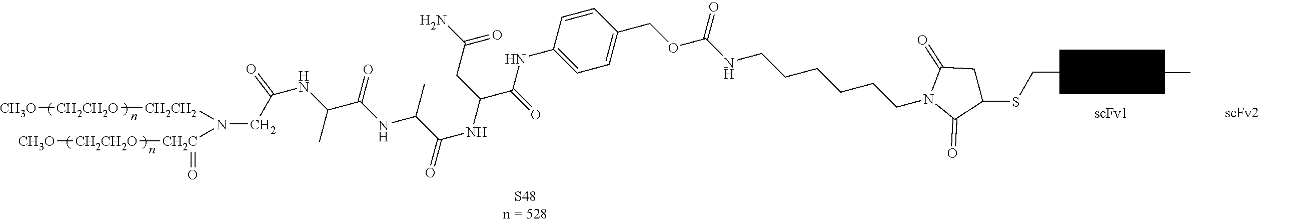 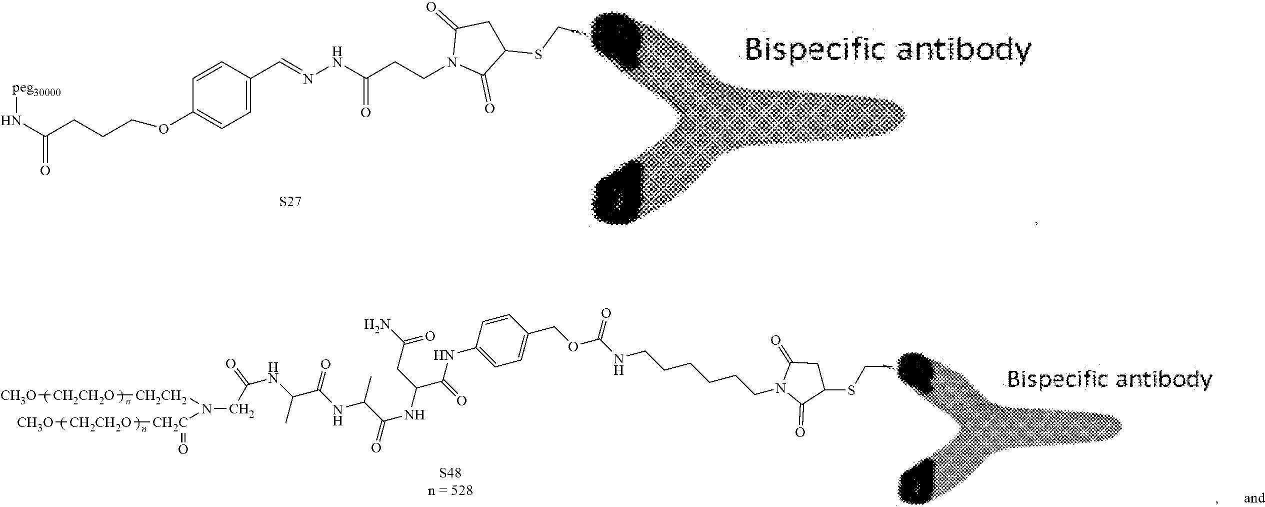 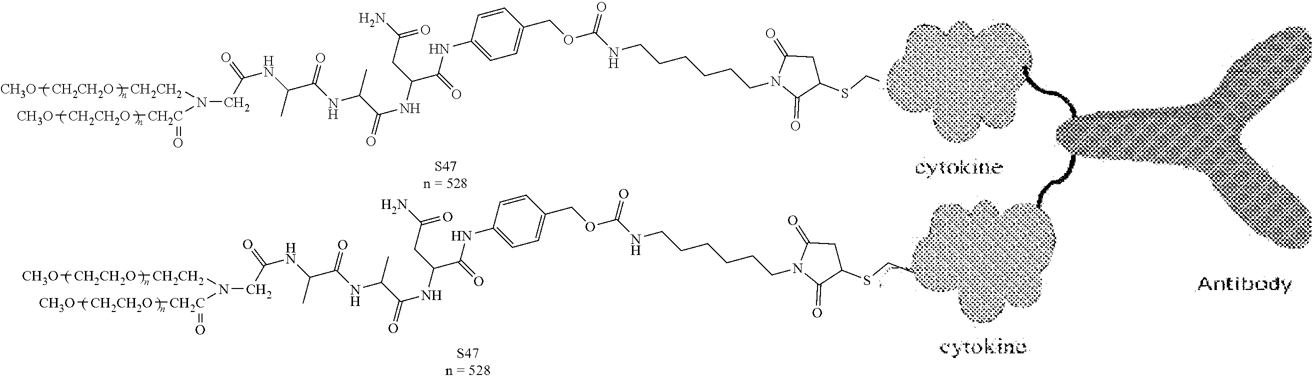
wherein the antibody is selected from the group consisting of any of SEQ ID NO: 1-83 and 106.


SEQUENCE LISTING (106 sequences)

SEQ ID NO: 1 - protein, 447 aa, synthetic construct
  REGION 1..447: note = heavy chain of anti-PD-1 antibody
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 218 aa, synthetic construct
  REGION 1..218: note = light chain of anti-PD-1 antibody
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 440 aa, synthetic construct
  REGION 1..440: note = heavy chain of anti-PD-1 antibody
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 214 aa, synthetic construct
  REGION 1..214: note = light chain anti-PD-1 antibody
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 448 aa, synthetic construct
  REGION 1..448: note = heavy chain of anti-CTLA-4 antibody
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 215 aa, synthetic construct
  REGION 1..215: note = light chain of anti-CTLA-4 antibody
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 224 aa, synthetic construct
  REGION 1..224: note = heavy chain of anti-TNF antibody
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 8 - protein, 214 aa, synthetic construct
  REGION 1..214: note = light chain of anti-TNF antibody
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 446 aa, synthetic construct
  REGION 1..446: note = heavy chain of anti-CD28 antibody
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 214 aa, synthetic construct
  REGION 1..214: note = light chain of anti-CD28 antibody
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 132 aa, synthetic construct
  REGION 1..132: note = amino acid sequence of wild type IL2
APASSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 12 - protein, 131 aa, synthetic construct
  REGION 1..131: note = amino acid sequence of wild type IL2
PTSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 13 - protein, 448 aa, synthetic construct; identical to SEQ ID NO: 5
  REGION 1..448: note = Ipilimumab heavy chain

SEQ ID NO: 14 - protein, 215 aa, synthetic construct; identical to SEQ ID NO: 6
  REGION 1..215: note = Ipilimumab light chain

SEQ ID NO: 15 - protein, 447 aa, synthetic construct; identical to SEQ ID NO: 1
  REGION 1..447: note = Pembrolizumab heavy chain

SEQ ID NO: 16 - protein, 218 aa, synthetic construct; identical to SEQ ID NO: 2
  REGION 1..218: note = Pembrolizumab light chain

SEQ ID NO: 17 - protein, 440 aa, synthetic construct; identical to SEQ ID NO: 3
  REGION 1..440: note = Nivolumab heavy chain

SEQ ID NO: 18 - protein, 214 aa, synthetic construct; identical to SEQ ID NO: 4
  REGION 1..214: note = Nivolumab light chain

SEQ ID NO: 19 - protein, 444 aa, synthetic construct
  REGION 1..444: note = Anti-human PD-1 antibody (WO 2017/124050 A1) heavy chain
QVQLQESGPGLVKPSQTLSLTCSVSGYSITSGYFWNWIRQFPGNKLEWIGYISYDGSKNYNPSLKNRVTIIRDTSKNQFSLKLNSVTAEDTATYYCVRGGLPVMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 220 aa, synthetic construct
  REGION 1..220: note = Anti-human PD-1 antibody (WO 2017/124050 A1) light chain
DIVMTQSPSSLALSVGEKATIQCKSSQSLLDDNNQKNYLAWYQQKPGQPPKLLIFFASTRESGVPDRFIGSGSGTDFTLTISSLQAEDLADYYCQQHYTTPYTFGGGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 451 aa, synthetic construct
  REGION 1..451: note = Anti-mouse PD-1 antibody J43v2 heavy chain
EVRLLESGGGLVKPEGSLKLSCVASGFTFSDYFMSWVRQAPGKGLEWVAHIYTKSYNYATYYSGSVKGRFTISRDDSRSMVYLQMNNLRTEDTATYYCTRDGSGYPSLDFWGQGTQVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 214 aa, synthetic construct
  REGION 1..214: note = Anti-mouse PD-1 antibody J43v2 light chain
YELTQPPSASVNVGETVKITCSGDQLPKYFADWFHQRSDQTILQVIYDDNKRPSGIPERISGSSSGTTATLTIRDVRAEDEGDYYCFSGYVDSDSKLYVFGSGTQLTVLGGPKSSPKVTVFPPSPEELRTNKATLVCLVNDFYPGSATVTWKANGATINDGVKTTKPSKQGQNYMTSSYLSLTADQWKSHNRVSCQVTHEGETVEKSLSPAECL

SEQ ID NO: 23 - protein, 453 aa, synthetic construct
  REGION 1..453: note = Anti-mouse CTLA-4 9D9 antibody heavy chain
EAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 24 - protein, 219 aa, synthetic construct
  REGION 1..219: note = Anti-mouse CTLA-4 9D9 antibody light chain
DIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - protein, 442 aa, synthetic construct
  REGION 1..442: note = Anti-human 4-1BB antibody heavy chain
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 214 aa, synthetic construct
  REGION 1..214: note = Anti-human 4-1BB antibody light chain
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 27 - protein, 450 aa, synthetic construct
  REGION 1..450: note = Trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, synthetic construct
  REGION 1..214: note = Trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 451 aa, synthetic construct
  REGION 1..451: note = Adalimumab heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 214 aa, synthetic construct; identical to SEQ ID NO: 8
  REGION 1..214: note = Adalimumab light chain

SEQ ID NO: 31 - protein, 448 aa, synthetic construct
  REGION 1..448: note = Atezolizumab heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 214 aa, synthetic construct
  REGION 1..214: note = Atezolizumab light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 446 aa, synthetic construct; identical to SEQ ID NO: 9
  REGION 1..446: note = Anti-human CD28 antibody heavy chain

SEQ ID NO: 34 - protein, 214 aa, synthetic construct; identical to SEQ ID NO: 10
  REGION 1..214: note = Anti-human CD28 antibody light chain

SEQ ID NO: 35 - protein, 215 aa, synthetic construct
  REGION 1..215: note = Ipi-se008 light chain
EIVLTQSPGTLSLSPGERATLSCRASQSVGSSYLAWYQQKPGQAPRLLICGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 215 aa, synthetic construct
  REGION 1..215: note = Ipi-se010 light chain
EIVLTQSPGTLSLSPGERATLSCRASQSVGSSYLAWYQQKPGQAPRLLIYGAFSRATGIPDRFSGSGCGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 215 aa, synthetic construct
  REGION 1..215: note = Ipi-se009 light chain
EIVLTQSPGTLSLSPGERATLSCRASQSVGSSYLAWYQQKPGQAPRLLIYGAFCRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 217 aa, synthetic construct
  REGION 1..217: note = Pem-se010 light chain
EIVLTQSPATLSLSPGERATLSCRASKGVSTSGYSYLHWYQQKPGQAPRLLIYLASCESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 218 aa, synthetic construct
  REGION 1..218: note = Pem-se009 light chain
EIVLTQSPATLSLSPGERATLSCRASKGVSTSGYSCLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 218 aa, synthetic construct
  REGION 1..218: note = Pem-se007 light chain
EIVLTQSPATLSLSPGERATLSCRASKCVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 440 aa, synthetic construct
  REGION 1..440: note = Niv-se001 heavy chain variable domain
QVQLVESGGGVVQPGRSLRLDCCASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 214 aa, synthetic construct
  REGION 1..214: note = Niv-se005 light chain
EIVLTQSPATLSLSPGERATLSCRASQSVSSYLAWYQQKPGQAPRLLICDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 214 aa, synthetic construct
  REGION 1..214: note = Niv-se007 light chain
EIVLTQSPATLSLSPGERATLSCRASQSVSSYLAWYQQKPGQAPRLLIYDASNRATGIPARFSGSGSGTDFTLTISSLEPEDFAVYYCQQCSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 133 aa, synthetic construct
  REGION 1..133: note = Interleukin-2 with mutation
APTSSSTKKTQLQLEHLLLDLQMILNGINNYKNPKLTRMLCFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 45 - protein, 133 aa, synthetic construct
  REGION 1..133: note = Interleukin-2 with mutation
APTSSSTKKTQLQLEHLLLDLQMILNGINNYKNPKLCRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 46 - protein, 133 aa, synthetic construct
  REGION 1..133: note = Interleukin-2 with mutation
APTSSSTKKTQLQLEHLLLDLQMILNGINNYKNPKLTRMLTFKFCMPKKATELKHLQCLEEELKPLEEVLNLAQSKNFHLRPRDLISNINVIVLELKGSETTFMCEYADETATIVEFLNRWITFSQSIISTLT

SEQ ID NO: 47 - protein, 133 aa, synthetic construct
  REGION 1..133: note = Interleukin-2 with mutation
APTSSSTKKTQLQLEHLLLDLQMILNGINNYKNPKLTRMLCFKFYMPKKATELKHLQCLEEELKPLEEVLNLAQSKNFHLRPRDLICNINVIVLELKGSETTFMCEYADETATIVEFLNRWITFSQSIISTLT

SEQ ID NO: 48 - protein, 133 aa, synthetic construct
  REGION 1..133: note = Interleukin-2 with mutation
APTSSSTKKTQLQLEHLLCDLQMILNGINNYKNPKLCRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 49 - protein, 133 aa, synthetic construct
  REGION 1..133: note = Interleukin-2 with mutation
APTSSSTKKTQLQLEHLLLDLQMILNGINNYKNPKLTRMLCFKFYMPKKATELKHLQCLEEELKPLEEVLNLAQSKNFHLRPRCLISNINVIVLELKGSETTFMCEYADETATIVEFLNRWITFSQSIISTLT

SEQ ID NO: 50 - protein, 133 aa, synthetic construct
  REGION 1..133: note = Interleukin-2 with mutation
APTSSSTKKTQLQLEHLLLDLQMILNGINNYKNPKLTDMLCFKFYMPKKATELKHLQCLEEELKPLEEVLNLAQSKNFHLRPRDLICNINVIVLELKGSETTFMCEYADETATIVEFLNRWITFSQSIISTLT

SEQ ID NO: 51 - protein, 133 aa, synthetic construct
  REGION 1..133: note = Interleukin-2 with mutation
APTSSSTKKTQLQLEHLLLDLQMILNGINNYKNPKLTDMLCFKFYMPKKATELKHLQCLERELKPLEEVLNLAQSKNFHLRPRDLICNINVIVLELKGSETTFMCEYADETATIVEFLNRWITFSQSIISTLT

SEQ ID NO: 52 - protein, 133 aa, synthetic construct
  REGION 1..133: note = Interleukin-2 with mutation
APTSSSTKKTQLQLEHLLCDLQMILNGINNYKNPKLTDMLCFKFYMPKKATELKHLQCLERELKPLEEVLNLAQSKNFHLRPRDLISNINVIVLELKGSETTFMCEYADETATIVEFLNRWITFSQSIISTLT

SEQ ID NO: 53 - protein, 133 aa, synthetic construct
  REGION 1..133: note = Interleukin-2 with mutation
APTSSSTKKTQLQLEHLLLDLQMILNGINNYKNPKLTDMLCFKFYMPKKATELKHLQCLERELKPLEEVLNLAQSKNFHLRPRCLISNINVIVLELKGSETTFMCEYADETATIVEFLNRWITFSQSIISTLT

SEQ ID NO: 54 - protein, 159 aa, synthetic construct
  REGION 1..159: note = Interleukin-10 with mutation
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEYMTMKIRN

SEQ ID NO: 55 - protein, 159 aa, synthetic construct
  REGION 1..159: note = Interleukin-10 with mutation
SPGQGTQSENSCTHFPGNLPNMLRDLRDAFCRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEYMTMKIRN

SEQ ID NO: 56 - protein, 159 aa, synthetic construct
  REGION 1..159: note = Interleukin-10 with mutation
SPGQGTQSENSCTHFPGNLPNMLRDLRDAFSRVCTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEYMTMKIRN

SEQ ID NO: 57 - protein, 159 aa, synthetic construct
  REGION 1..159: note = Interleukin-10 with mutation
SPGQGTQSENSCTHFPGNLPNMLRDLRDAFSRVKTFFQMKDQLCNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEYMTMKIRN

SEQ ID NO: 58 - protein, 159 aa, synthetic construct
  REGION 1..159: note = Interleukin-10 with mutation
SPGQGTQSENSCTHFPGNLPNMLRDLRDAFSCVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEYMTMKIRN

SEQ ID NO: 59 - protein, 159 aa, synthetic construct
  REGION 1..159: note = Interleukin-10 with mutation
SPGQGTQSENSCTHFPGNLPNMLRDLRDAFSRVKCFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEYMTMKIRN

SEQ ID NO: 60 - protein, 453 aa, synthetic construct
  REGION 1..453: note = Anti-human VEGF antibody heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 214 aa, synthetic construct
  REGION 1..214: note = Anti-human VEGF antibody light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 451 aa, synthetic construct
  REGION 1..451: note = Anti-human CD20 antibody heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 213 aa, synthetic construct
  REGION 1..213: note = Anti-human CD20 antibody light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 118 aa, synthetic construct [sequence omitted]

SEQ ID NO: 65 - protein, 107 aa, synthetic construct
  REGION 1..107: note = VL of Atezolizumab
DIQMTQSPSSLSASVGDRVTITCRASQDVSTAVAWYQQKPGKAPKLLICSASFLYSGVPSRFSGSGSGTDFTLTISSLQPEDFATYYCQQYLYHPATFGQGTKVEIK

SEQ ID NO: 66 - protein, 119 aa, synthetic construct
  REGION 1..119: note = VH of Cetuximab with mutation
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 67 - protein, 107 aa, synthetic construct
  REGION 1..107: note = VL of Cetuximab with mutation
DILLTQSPVILSVSPGERVSFSCRASQSIGTNIHWYQQRTNGSPRLLICYASESISGIPSRFSGSGSGTDFTLSINSVESEDIADYYCQQNNNWPTTFGAGTKLELK

SEQ ID NO: 68 - protein, 116 aa, synthetic construct [sequence omitted]

SEQ ID NO: 69 - protein, 107 aa, synthetic construct
  REGION 1..107: note = VL of Ramucirumab with mutation
DIQMTQSPSSVSASIGDRVTITCRASQGIDNWLGWYQQKPGKAPKLLICDASNLDTGVPSRFSGSGSGTYFTLTISSLQAEDFAVYFCQQAKAFPPTFGGGTKVDIK

SEQ ID NO: 70 - protein, 496 aa, synthetic construct
  REGION 1..496: note = Fusion protein of scFv of anti-Her2 antibody with CD3, with mutation
DIQMTQSPSSLSASVGDRVTITCRASQDVNTAVAWYQQKPGKAPKLLICSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 71 - protein, 120 aa, synthetic construct [sequence omitted]

SEQ ID NO: 72 - protein, 107 aa, synthetic construct
  REGION 1..107: note = VL of anti Her2 antibody with mutation
DIQMTQSPSSLSASVGDRVTITCRASQDVNTAVAWYQQKPGKAPKLLICSASFLYSGVPSRFSGSRSGTDFTLTISSLQPEDFATYYCQQHYTTPPTFGQGTKVEIK

SEQ ID NO: 73 - protein, 590 aa, synthetic construct
  REGION 1..590: note = Fusion protein of scFvs of anti-Her2 antibody with anti-CD3 antibody
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGSGGGSGGGSAPASSSTKKTQLQLEHLLLDLQMILNGINNYKNPKLTRMLCFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 74 - protein, 595 aa, synthetic construct
  REGION 1..595: note = Fusion protein of scFvs of anti-Her2 antibody with anti-CD3 antibody
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGSGGGSGGGSAPASSSTKKTQLQLEHLLLDLQMILNGINNYKNPKLTRMLCFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 75 - protein, 243 aa, synthetic construct
  REGION 1..243: note = anti CD3 antibody
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 76 - protein, 119 aa, synthetic construct; identical to SEQ ID NO: 66
  REGION 1..119: note = VH of Cetuximab

SEQ ID NO: 77 - protein, 107 aa, synthetic construct
  REGION 1..107: note = VL of Cetuximab
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 78 - protein, 116 aa, synthetic construct [sequence omitted]

SEQ ID NO: 79 - protein, 107 aa, synthetic construct [sequence omitted]

SEQ ID NO: 80 - protein, 120 aa, synthetic construct [sequence omitted]

SEQ ID NO: 81 - protein, 107 aa, synthetic construct [sequence omitted]

SEQ ID NO: 82 - protein, 496 aa, synthetic construct
  REGION 1..496: note = Fusion protein of scFvs of anti-Her2 antibody with anti-CD3 antibody, with mutation
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWICDTSKVASGVPYRFSGSGSGTSYSLTISSMEAEDAATYYCQQWSSNPLTFGAGTKLELKHHHHHH

SEQ ID NO: 83 - protein, 496 aa, synthetic construct
  REGION 1..496: note = Fusion protein of scFvs of anti-Her2 antibody with anti-CD3 antibody, with mutation
DIQMTQSPSSLSASVGDRVTITCRASQDVNTAVAWYQQKPGKAPKLLICSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWICDTSKVASGVPYRFSGSGSGTSYSLTISSMEAEDAATYYCQQWSSNPLTFGAGTKLELKHHHHHH

SEQ ID NO: 84 - protein, 10 aa, synthetic construct
  REGION 1..10: note = sequence of R2
LTPRLGPAAN

SEQ ID NO: 85 - protein, 5 aa, synthetic construct
  REGION 1..5: note = sequence of R2
GPAAN

SEQ ID NO: 86 - protein, 7 aa, synthetic construct
  REGION 1..7: note = sequence of R2
LSGRSDN

SEQ ID NO: 87 - DNA, 21 bp, synthetic construct
  misc_feature 1..21: note = forward primer
  source 1..21: mol_type = other DNA
gaactccctcgatgttaacca

SEQ ID NO: 88 - DNA, 21 bp, synthetic construct
  misc_feature 1..21: note = reverse primer
  source 1..21: mol_type = other DNA
ttcaatccccaaatcctagcc

SEQ ID NO: 89 - protein, 214 aa, synthetic construct
  REGION 1..214: note = Light chain of Cetuximab
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 214 aa, synthetic construct
  REGION 1..214: note = Light chain of Pertuzumab
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 448 aa, synthetic construct
  REGION 1..448: note = Heavy chain of Pertuzumab
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 113 aa, synthetic construct [sequence omitted]

SEQ ID NO: 93 - protein, 107 aa, synthetic construct [sequence omitted]

SEQ ID NO: 94 - protein, 120 aa, synthetic construct [sequence omitted]

SEQ ID NO: 95 - protein, 111 aa, synthetic construct [sequence omitted]

SEQ ID NO: 96 - protein, 108 aa, synthetic construct
  REGION 1..108: note = VL of Ipilimumab
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 97 - protein, 118 aa, synthetic construct
  REGION 1..118: note = VH of Ipilimumab
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 98 - protein, 108 aa, synthetic construct
  REGION 1..108: note = VL of Anti-human 4-1BB antibody
STELTQPPSVSVSPGQTASITCSGDNIGDQTAHWTQQLPGQSPVLVITQDLNRPSGIPERFSGSNSGNTATLTISGTQAMDEADTTCATTTGFGSLAVFGGGTLLTVL

SEQ ID NO: 99 - protein, 116 aa, synthetic construct
  REGION 1..116: note = VH of Anti-human 4-1BB antibody
EVQLVQSGAEVLLPGESLRISCLGSGTSFSTTWISWVRQMPGLGLEWMGLITPGDSTTNTSPSFQGQVTISADLSISTATLQWSSLLASDTAMTTCARGTGIFDTWGQGTLVTVSS

SEQ ID NO: 100 - protein, 107 aa, synthetic construct [sequence omitted]

SEQ ID NO: 101 - protein, 121 aa, synthetic construct [sequence omitted]

SEQ ID NO: 102 - protein, 120 aa, synthetic construct [sequence omitted]

SEQ ID NO: 103 - protein, 111 aa, synthetic construct [sequence omitted]

SEQ ID NO: 104 - protein, 105 aa, synthetic construct
  REGION 1..105: note = VL of Trastuzumab
DIQMTQSPSSLSASVGDRVTITCRASQDVNTAVAWYQQKPGKAPKLLIYSASFLGVPSRFSGSRSGTDFTLTISSLQPEDFATYYCQQHYTTPPTFGQGTKVEIK

SEQ ID NO: 105 - protein, 120 aa, synthetic construct [sequence omitted]

SEQ ID NO: 106 - protein, 133 aa, synthetic construct
  REGION 1..133: note = Interleukin-2 with mutation
APVSSSTKKTQLQLEHLLLDLQMILNGINNYKNPKLTRMLCFKFYMPKKATELKHLQCLEEELKPLEEVLNLASQKNFHLRPRDLISNINVIVLELKGSETTFMCEYADETATIVEFLNRWITFSQSIISTLT